(19) United States
(12) Patent Application Publication
(10) Pub. No.: US 2025/0339559 A1
(43) Pub. Date: Nov. 6, 2025

(54) BASE EDITING-MEDIATED READTHROUGH OF PREMATURE TERMINATION CODONS (BERT)

(71) Applicants: The Broad Institute, Inc., Cambridge, MA (US); President and Fellows of Harvard College, Cambridge, MA (US)

(72) Inventors: David R. Liu, Cambridge, MA (US); Steven Erwood, Cambridge, MA (US); Aditya Raguram, Cambridge, MA (US)

(73) Assignees: The Broad Institute, Inc., Cambridge, MA (US); President and Fellows of Harvard College, Cambridge, MA (US)

(21) Appl. No.: 19/271,651

(22) Filed: Jul. 16, 2025

Related U.S. Application Data
(63) Continuation of application No. PCT/US2024/011896, filed on Jan. 17, 2024.
(60) Provisional application No. 63/480,499, filed on Jan. 18, 2023.

(51) Int. Cl. A61K 48/00; C12N 9/02; C12N 9/10; C12N 9/22; C12N 9/78; C12N 15/11

(52) U.S. Cl. CPC A61K 48/005; C12N 9/0036; C12N 9/1007; C12N 9/1048; C12N 9/226; C12N 9/78; C12N 15/111; C07K 2319/80; C12N 2310/20; C12Y 106/03001; C12Y 201/01056; C12Y 201/01063; C12Y 305/04002; C12Y 305/04003; C12Y 305/04005

(57) ABSTRACT

Aspects of the disclosure relate to methods, compositions, and systems for editing a DNA sequence encoding an endogenous tRNA into a suppressor tRNA using base editing (e.g., to treat a disease caused by a premature termination codon or PTC). Additional aspects relate to compositions comprising a gRNA configured to bind to a DNA sequence encoding an endogenous tRNA. Other aspects relate to complexes comprising a base editor and a gRNA that are capable of editing an endogenous tRNA into a suppressor tRNA. In some aspects, the disclosure further relates to polynucleotides encoding one or more nucleic acid sequences encoding the gRNAs, vectors comprising the polynucleotides, and/or cells comprising the polynucleotides, complexes, gRNAs, and/or vectors disclosed herein. Additional aspects further relate to kits comprising any one of the compositions, complexes, gRNAs, polynucleotides, vectors, and/or cells disclosed herein.

### RELATED APPLICATIONS

This application claims priority under 35 U.S.C. § 119(e) to U.S. Provisional Application, U.S. Ser. No. 63/480,499, filed Jan. 18, 2023, which is incorporated herein by reference.

### GOVERNMENT SUPPORT

This invention was made with government support under R35GM118062 awarded by NIH MIRA. The government has certain rights in the invention.

### REFERENCE TO AN ELECTRONIC SEQUENCE LISTING

The contents of the electronic sequence listing (Filename; Size: 2,249,959 bytes; and Date of Creation: Jan. 15, 2024) is herein incorporated by reference in its entirety.

### BACKGROUND OF INVENTION

Nonsense mutations in genomic DNA lead to premature termination codons (PTCs) in mRNAs, which in turn impede translation of full-length proteins. Diminished translation of full-length proteins due to PTCs can induce pathogenic effects in cells and organisms. Indeed, approximately 33% of known human genetic diseases and 11% of known pathogenic gene variants are caused by PTCs (e.g., cystic fibrosis, beta thalassaemia, Hurler syndrome, Dravet syndrome, Duchenne muscular dystrophy, Usher syndrome, and hemophilia). Interestingly, many bacteria and viruses utilize suppressor tRNAs to enable translational stop codon readthrough (e.g., the ribosome goes past the stop codon and continues translating the mRNA into protein). However, suppressor tRNAs do not naturally occur in the human body. Base editing allows for precise editing of the genomic DNA encoding the PTCs and may provide a platform for the treatment of diseases associated with PTCs.

### SUMMARY OF INVENTION

Aspects of the disclosure relate to methods, compositions, and systems for editing a DNA sequence encoding an endogenous tRNA into a suppressor tRNA using base editing (e.g., to treat a disease caused by a premature termination codon or PTC). Additional aspects relate to compositions comprising a gRNA configured to bind to a DNA sequence encoding an endogenous tRNA. Other aspects relate to complexes comprising a base editor and a gRNA that are capable of editing an endogenous tRNA into a suppressor tRNA. In some aspects, the disclosure further relates to polynucleotides encoding one or more nucleic acid sequences encoding the gRNAs, vectors comprising the polynucleotides, and/or cells comprising the polynucleotides, complexes, gRNAs, and/or vectors disclosed herein. Additional aspects further relate to kits comprising any one of the compositions, complexes, gRNAs, polynucleotides, vectors, and/or cells disclosed herein.

As defined elsewhere herein, suppressor tRNAs are tRNAs that are natively charged with their cognate amino acids but possess engineered anticodon loops designed to bind PTCs (e.g., amber, ochre, or opal stop codons). As such, suppressor tRNAs bind to PTCs during the process of translation, leading to incorporation of an amino acid instead of terminating translation. Without wishing to be bound by any particular theory, suppressor tRNAs were recently used to rescue a genetic disease in a mouse model carrying a nonsense mutation^{8,9}, but the suppressor tRNA was delivered via an adeno-associated viral vector (herein “AAV”). Permanent expression of the suppressor tRNA is necessary for continued rescue of the disease, which is challenging to achieve using AAV and requires repeated administration of the suppressor tRNA vector.

Humans possess over 500 interspersed tRNA genes, and many of these genes are redundant and dispensable¹¹. For example, one or both copies of the tRNA^{Lys} CUU gene is deleted in ˜50% of humans¹². Therefore, using base editing to convert the CUU anticodon of the tRNA^{Lys} gene into UUA, UCA, or CUA for ochre, opal, and amber suppression, respectively, would generate an endogenous suppressor tRNA^{Lys}. Thus, in some embodiments, the endogenous tRNA converted into a suppressor tRNA is a tRNA^{Lys} CUU gene. In this particular embodiment, lysine would be installed at the locations of the PTCs. In other embodiments, the tRNA gene is any redundant and dispensable tRNA gene known in the art. In other embodiments, the tRNA gene is any redundant and indispensable gene known in the art. (see Table 1 for a list of all and non human tRNA genes)

In other embodiments, other domains in the tRNA gene may also be edited, either alone or in addition to editing the anticodon. For example, in some embodiments, base editing may be used to alter the (i) the anticodon sequence of a tRNA, (ii) the identity of the amino acid attached to a tRNA, or (iii) both the anticodon sequence of the tRNA and the identity of the amino acid attached to the tRNA. Any known edit in the art may be used to alter the identity of the charged amino acid. For example, in some embodiments, base editing is used to install a C70U mutation in the acceptor stem of tRNA^{Lys}; this mutation is known to change the identity of the charged amino acid to alanine. Other edits within the acceptor stem domain and/or other domains (e.g., D-arm, T-arm, or variable arm) may also be used to alter the identity of the charged amino acid.

In some embodiments, the choice of amino acid inserted at a stop codon is tailored by the choice of tRNA to edit and/or by installing sequences recognized by specific aminoacyl-tRNA synthetases to direct amino acid charging of the newly generated suppressor tRNA. In some embodiments, suppression with widely tolerated amino acids such as glycine, alanine, or serine may be preferable to suppression with more unusual amino acids such as proline or arginine or tryptophan, except when treating diseases caused by premature stop codons that have arisen from mutation of these amino acids. For example, in certain embodiments, arginine to STOP mutations (e.g. 5′-CGA-3′ mutation to 5′-UGA-3′) are a common cause of genetic diseases, and in these cases, base editing to create an arginine-charged suppressor tRNA may be desirable.

As such, some aspects of the present disclosure are related to methods for editing a DNA sequence encoding an endogenous tRNA at a target site. In some embodiments, the target site in the DNA sequence encodes one or more domains of the endogenous tRNA. tRNA domains are known in the art and comprise the D-arm domain, T-arm domain, variable arm domain, acceptor stem domain (e.g., C70U), and an anticodon arm domain comprising an anticodon sequence (FIG. 3).

In some embodiments, the endogenous tRNA anticodon sequence is a single transition mutation away from a nonsense suppressor anticodon. As defined elsewhere herein, a nonsense suppressor anticodon is the complementary sequence to a premature termination codon or PTC. There are currently three known PTCs, each of which, comprises a different sequence. The ochre stop codon has sequence 5′-UAA-3′ and corresponds to nonsense suppressor anticodon with sequence 5′-UUA-3′. The opal stop codon has sequence 5′-UGA-3′ and corresponds to the nonsense suppressor anticodon with sequence 5′-UCA-3′. The amber stop codon has sequence 5′-UAG-3′ and corresponds to nonsense suppressor anticodon with sequence 5′-CUA-3′.

In some embodiments, the endogenous tRNA comprises an anticodon sequence that is a single transversion mutation away from a nonsense suppressor anticodon. The single transversion mutation may be any transversion mutation known in the art.

In some embodiments, the endogenous tRNA comprises an anticodon sequence that is 3′-X1-X2-X3-5′. In some embodiments, the base editor installs the mutation (e.g., transition or transversion) at position XL. In some embodiments, the base editor installs the mutation (e.g., transition or transversion) at position X2. In some embodiments, the base editor installs the mutation (e.g., transition or transversion) at position X3.

Other aspects of the present disclosure relate to edited tRNAs described herein. While it is generally known that translational stop codon readthrough provides a regulatory mechanism of gene expression this extensively utilized by positive-sense ssRNA viruses, no such mechanism has been observed in humans. In other words, suppressor tRNAs are not naturally found and/or naturally occurring in humans. Thus, in some embodiments, the disclosure relates to one or more suppressor tRNAs engineered from endogenous tRNAs. In some embodiments, the suppressor tRNA comprises a nonsense suppressor anticodon sequence selected from the group consisting of 5′-UUA-3′, 5′-UCA-3′ and 5′-CUA-3′. In some embodiments, the suppressor tRNA further comprises an amino acid selected from the group consisting of alanine, arginine, asparagine, aspartic acid, cysteine, glutamine, glutamic acid, glycine, histidine, isoleucine, leucine, methionine, phenylalanine, proline, serine, threonine, tryptophan, tyrosine, valine, pyrrolysine, and selenocysteine.

Additional aspects of the disclosure relate to guide RNAs configured to bind to DNA sequences encoding endogenous tRNA sequences.

Complexes comprising the gRNA and a base editor are also contemplated herein. In some embodiments, the gRNA comprises a spacer sequence configured to bind to a DNA sequence encoding an endogenous tRNA. In some embodiments the spacer sequence is any sequence listed in Table 2.

Other aspects of the disclosure relate to polynucleotides. For example, in some aspects, the disclosure relates to a polynucleotide comprising a first nucleic acid sequence encoding a base editor and a second nucleic acid sequence encoding a guide RNA, wherein the guide RNA comprises a spacer sequence configured to bind to one or more tRNA genes (e.g., see Table 2). In some embodiments, the polynucleotide comprises a first nucleic acid sequence encoding a guide RNA configured to bind to a DNA sequence encoding an endogenous tRNA.

Aspects of the disclosure also relate to vector systems comprising one or more vectors, or vectors as such. Vectors may be designed to clone and/or express the base editors as disclosed herein. Vectors may also be designed to clone and/or express one or more gRNAs having complementarity to the target sequence, as disclosed herein. Vectors may also be designed to transfect the base editors and gRNAs of the disclosure into one or more cells, e.g., a target diseased eukaryotic cell for treatment with the base editor systems and methods disclosed herein.

In some aspects, the disclosure relates to cells comprising any one of the polynucleotides, gRNAs, vectors, edited tRNAs, or complexes disclosed herein. In some embodiments, the cell is an animal cell. In some embodiments, the animal cell is a mammalian cell, a non-human primate cell, or a human cell. In other embodiments, the cell is a plant cell.

In some aspects, the disclosure relates to pharmaceutical compositions comprising any one of pegRNAs, complexes, vectors, edited tRNAs, polynucleotides, and cells disclosed herein, or any combination thereof, and a pharmaceutical excipient.

In some aspects, the disclosure relates to kits comprising any one of the compositions, guide RNAs, complexes, polynucleotides, and cells disclose herein, or any combination thereof, and instructions for editing a one or more DNA sequences encoding one or more domains of a tRNA by base editing, wherein the DNA sequence is any sequence that encodes a tRNA (e.g., see Table 1). In some embodiments, the kit further comprises a pharmaceutical excipient.

Other aspects of the disclosure relate to methods for changing the amino acid that is charged onto an endogenous tRNA using base editing. Without wishing to be bound by any particular theory, it is generally recognized in the art that mutation of select nucleotides within one or more domains of the endogenous tRNA alters the aminoacyl-tRNA synthetase that recognizes the endogenous tRNA, and hence, charges the tRNA with a non-cognate amino acid. See for example, Liu et al., “Engineering a tRNA and aminoacyl-tRNA synthetase for the site specific incorporation of unnatural amino acids into protein in vivo” PNAS, 1997, 94 (19) 10092-10097, which is incorporated herein by reference in its entirety. For example, tRNAs comprising a C70U mutation in the acceptor stem domain are charged alanine, regardless of their anticodon sequence. Thus, in some embodiments, the tRNAs edited with the base editors described herein, comprises an anticodon sequence that encodes for the cognate amino acid but are charged with a non-cognate amino acid.

Additional aspects of the disclosure relate to methods for producing a suppressor tRNA molecules from an endogenous tRNA molecule using base editing in a subject in need thereof, the method comprising administering to the subject: (i) a base editor and (ii) a guide RNA, wherein the base editor and the gRNA install a mutation, as described herein, at a target site in a DNA sequence encoding the tRNA molecule, wherein installation of the mutation converts the endogenous tRNA molecule into the suppressor tRNA molecule.

Other aspects relate to methods of treating a disease caused by premature termination codons in a subject in need thereof, the method comprising administering to the subject (i) a base editor and (ii) a guide RNA, wherein the base editor and guide RNA form a base editor complex, wherein the base editor complex mutates a target DNA sequence encoding one or more domains of a tRNA to produce a suppressor tRNA, wherein the suppressor tRNA comprises an anticodon sequence complementary to an ochre stop codon, an opal stop codon, or an amber stop codon.

### BRIEF DESCRIPTION OF THE DRAWINGS

FIG. 1 illustrates the conversion of Gln-TTG-4-1 and Gln-CTG-6-1 into suppressor tRNAs Gln-TTA-4-1 and Gln-CTA-6-1 using base editors, respectively. Approximately 20% of the sequenced reads had the specified edit.

FIG. 2A illustrates the conversion of GLN-CTG-6-1 into the suppressor tRNA Gln-CTA-6-1. FIG. 2B illustrates the ability of the suppressor tRNA Gln-CTA-6-1 to edit a reported plasmid encoding an eGFP cassette with the corresponding premature termination codon.

FIG. 3 shows a representative schematic of an exemplary endogenous tRNA. Relevant domains include the D-arm domain (e.g., D-loop), acceptor stem domain, T-arm domain (e.g., TΨC loop), variable arm domain (e.g., variable loop), and the anticodon arm domain encoding the anticodon sequence (e.g., anticodon loop) (SEQ ID NO: 2491).

### DEFINITIONS

As used herein and in the claims, the singular forms “a,” “an,” and “the” include the singular and the plural reference unless the context clearly indicates otherwise. Thus, for example, a reference to “an agent” includes a single agent and a plurality of such agents.

The term “base editor (BE)” as used herein, refers to an agent comprising a polypeptide that is capable of making a modification to a base (e.g., A, T, C, G, or U) within a nucleic acid sequence (e.g., DNA or RNA) that converts one base to another (e.g., A to G, A to C, A to T, C to T, C to G, C to A, G to A, G to C, G to T, T to A, T to C, T to G). In some embodiments, the base editor is capable of deaminating a base within a nucleic acid such as a base within a DNA molecule. In the case of an adenine base editor, the base editor is capable of deaminating an adenine (A) in DNA. Such base editors may include a nucleic acid programmable DNA binding protein (napDNAbp) fused to an adenosine deaminase. Some base editors include CRISPR-mediated fusion proteins that are utilized in the base editing methods described herein. In some embodiments, the base editor comprises a nuclease-inactive Cas9 (dCas9) fused to a deaminase which binds a nucleic acid in a guide RNA-programmed manner via the formation of an R-loop, but does not cleave the nucleic acid. For example, the dCas9 domain of the fusion protein may include a D10A and a H840A mutation (which renders Cas9 capable of cleaving only one strand of a nucleic acid duplex), as described in PCT/US2016/058344, which published as WO 2017/070632 on Apr. 27, 2017, and is incorporated herein by reference in its entirety. The DNA cleavage domain of S. pyogenes Cas9 includes two subdomains, the HNH nuclease subdomain and the RuvC1 subdomain. The HNH subdomain cleaves the strand complementary to the gRNA (the “targeted strand”, or the strand in which editing or deamination occurs), whereas the RuvC1 subdomain cleaves the non-complementary strand containing the PAM sequence (the “non-edited strand”). The RuvC1 mutant D10A generates a nick in the targeted strand, while the HNH mutant H840A generates a nick on the non-edited strand (see Jinek et al., Science, 337:816-821(2012); Qi et al., Cell. 28; 152(5):1173-83 (2013)).

In some embodiments, a nucleobase editor is a macromolecule or macromolecular complex that results primarily (e.g., more than 80%, more than 85%, more than 90%, more than 95%, more than 99%, more than 99.9%, or 100%) in the conversion of a nucleobase in a polynucleic acid sequence into another nucleobase (i.e., a transition or transversion) using a combination of 1) a nucleotide-, nucleoside-, or nucleobase-modifying enzyme; and 2) a nucleic acid binding protein that can be programmed to bind to a specific nucleic acid sequence.

In some embodiments, the nucleobase editor comprises a DNA binding domain (e.g., a programmable DNA binding domain such as a dCas9 or nCas9) that directs it to a target sequence. In some embodiments, the nucleobase editor comprises a nucleobase modifying enzyme fused to a programmable DNA binding domain (e.g., a dCas9 or nCas9). A “nucleobase modifying enzyme” is an enzyme that can modify a nucleobase and convert one nucleobase to another (e.g., a deaminase such as a cytidine deaminase or an adenosine deaminase). In some embodiments, the nucleobase editor may target cytosine (C) bases in a nucleic acid sequence and convert the C to thymine (T) base. In some embodiments, the C to T editing is carried out by a deaminase, e.g., a cytidine deaminase. Base editors that can carry out other types of base conversions (e.g., adenosine (A) to guanine (G), C to G) are also contemplated.

Nucleobase editors that convert a C to T, in some embodiments, comprise a cytidine deaminase. A “cytidine deaminase” refers to an enzyme that catalyzes the chemical reaction “cytosine+H₂O→uracil+NH₃” or “5-methyl-cytosine+H2O→thymine+NH₃.” As it may be apparent from the reaction formula, such chemical reactions result in a C to U/T nucleobase change. In the context of a gene, such a nucleotide change, or mutation, may in turn lead to an amino acid change in the protein, which may affect the protein's function, e.g., loss-of-function or gain-of-function. In some embodiments, the C to T nucleobase editor comprises a dCas9 or nCas9 fused to a cytidine deaminase. In some embodiments, the cytidine deaminase domain is fused to the N-terminus of the dCas9 or nCas9. In some embodiments, the nucleobase editor further comprises a domain that inhibits uracil glycosylase, and/or a nuclear localization signal. Such nucleobase editors have been described in the art, e.g., in Rees & Liu, Nat Rev Genet. 2018; 19(12):770-788 and Koblan et al., Nat Biotechnol. 2018; 36(9):843-846; as well as U.S. Patent Publication No. 2018/0073012, published Mar. 15, 2018, which issued as U.S. Pat. No. 10,113,163; on Oct. 30, 2018; U.S. Patent Publication No. 2017/0121693, published May 4, 2017, which issued as U.S. Pat. No. 10,167,457 on Jan. 1, 2019; International Publication No. WO 2017/070633, published Apr. 27, 2017; U.S. Patent Publication No. 2015/0166980, published Jun. 18, 2015; U.S. Pat. No. 9,840,699, issued Dec. 12, 2017; U.S. Pat. No. 10,077,453, issued Sep. 18, 2018; International Publication No. WO 2019/023680, published Jan. 31, 2019; International Publication No. WO 2018/0176009, published Sep. 27, 2018, International Application No PCT/US2019/033848, filed May 23, 2019, International Application No. PCT/US2019/47996, filed Aug. 23, 2019; International Application No. PCT/US2019/049793, filed Sep. 5, 2019; U.S. Provisional Application No. 62/835,490, filed Apr. 17, 2019; International Application No. PCT/US2019/61685, filed Nov. 15, 2019; International Application No. PCT/US2019/57956, filed Oct. 24, 2019; U.S. Provisional Application No. 62/858,958, filed Jun. 7, 2019; International Publication No. PCT/US2019/58678, filed Oct. 29, 2019, the contents of each of which are incorporated herein by reference in their entireties.

In some embodiments, a nucleobase editor converts an A to G. In some embodiments, the nucleobase editor comprises an adenosine deaminase. An “adenosine deaminase” is an enzyme involved in purine metabolism. It is needed for the breakdown of adenosine from food and for the turnover of nucleic acids in tissues. Its primary function in humans is the development and maintenance of the immune system. An adenosine deaminase catalyzes hydrolytic deamination of adenosine (forming inosine, which base pairs as G) in the context of DNA. There are no known adenosine deaminases that act on DNA. Instead, known adenosine deaminase enzymes only act on RNA (tRNA or mRNA). Evolved deoxyadenosine deaminase enzymes that accept DNA substrates and deaminate dA to deoxyinosine have been described, e.g., in PCT Application PCT/US2017/045381, filed Aug. 3, 2017, which published as WO 2018/027078, and PCT Application No. PCT/US2019/033848, which published as WO 2019/226953, each of which is herein incorporated by reference by reference.

Exemplary adenine base editors (ABEs) (or “adenosine base editors”) and cytosine base editors (CBEs) (or “cytosine base editors”) are also described in Rees & Liu, Base editing: precision chemistry on the genome and transcriptome of living cells, Nat. Rev. Genet. 2018; 19(12):770-788; as well as U.S. Patent Publication No. 2018/0073012, published Mar. 15, 2018, which issued as U.S. Pat. No. 10,113,163, on Oct. 30, 2018; U.S. Patent Publication No. 2017/0121693, published May 4, 2017, which issued as U.S. Pat. No. 10,167,457 on Jan. 1, 2019; International Publication No. WO 2017/070633, published Apr. 27, 2017; U.S. Patent Publication No. 2015/0166980, published Jun. 18, 2015; U.S. Pat. No. 9,840,699, issued Dec. 12, 2017; and U.S. Pat. No. 10,077,453, issued Sep. 18, 2018, the contents of each of which are incorporated herein by reference in their entireties.

In principle, there are 12 possible base-to-base changes that may occur via individual or sequential use of transition (i.e., a purine-to-purine change or pyrimidine-to-pyrimidine change) or transversion (i.e., a purine-to-pyrimidine or pyrimidine-to-purine) editors. These include:

### Transition Base Editors:

C-to-T base editor (or “CTBE”). This type of editor converts a C:G Watson-Crick nucleobase pair to a T:A Watson-Crick nucleobase pair. Because the corresponding Watson-Crick paired bases are also interchanged as a result of the conversion, this category of base editor may also be referred to as a G-to-A base editor (or “GABE”).

A-to-G base editor (or “AGBE”). This type of editor converts a A:T Watson-Crick nucleobase pair to a G:C Watson-Crick nucleobase pair. Because the corresponding Watson-Crick paired bases are also interchanged as a result of the conversion, this category of base editor may also be referred to as a T-to-C base editor (or “TCBE”).

### Transversion Base Editors:

C-to-G base editor (or “CGBE”). This type of editor converts a C:G Watson-Crick nucleobase pair to a G:C Watson-Crick nucleobase pair. Because the corresponding Watson-Crick paired bases are also interchanged as a result of the conversion, this category of base editor may also be referred to as a G-to-C base editor (or “GCBE”).

G-to-T base editor (or “ACBE”). This type of editor converts a G:C Watson-Crick nucleobase pair to a T:A Watson-Crick nucleobase pair. Because the corresponding Watson-Crick paired bases are also interchanged as a result of the conversion, this category of base editor may also be referred to as a C-to-A base editor (or “CABE”).

A-to-T base editor (or “TGBE”). This type of editor converts a A:T Watson-Crick nucleobase pair to a T:A Watson-Crick nucleobase pair. Because the corresponding Watson-Crick paired bases are also interchanged as a result of the conversion, this category of base editor may also be referred to as a T-to-A base editor (or “ACBE”).

A-to-C base editor (or “ACBE”). This type of editor converts a A:T Watson-Crick nucleobase pair to a C:G Watson-Crick nucleobase pair. Because the corresponding Watson-Crick paired bases are also interchanged as a result of the conversion, this category of base editor may also be referred to as a T-to-G base editor (or “TGBE”).

The term “base editors (BEs)”, as used herein, refers to the Cas-fusion proteins described herein. In some embodiments, the fusion protein comprises a nuclease-inactive Cas9 (dCas9) fused to an DNA nucleobase modification domain (e.g., adenine deaminase) which binds a nucleic acid in a guide RNA-programmed manner via the formation of an R-loop but does not cleave the nucleic acid. For example, the dCas9 domain of the fusion protein may include a D10A and a H840A mutation (which renders Cas9 capable of cleaving only one strand of a nucleic acid duplex) as described in PCT/US2016/058344 (filed on Oct. 22, 2016 and published as WO 2017/070632 on Apr. 27, 2017), which is incorporated herein by reference in its entirety. The DNA cleavage domain of S. pyogenes Cas9 includes two subdomains, the HNH nuclease subdomain and the RuvC1 subdomain. The HNH subdomain cleaves the strand complementary to the gRNA (the “targeted strand,” or the strand at which editing or oxidation occurs), whereas the RuvC1 subdomain cleaves the non-complementary strand containing the PAM sequence (the “non-targeted strand”, or the strand at which editing or oxidation does not occur). The RuvC1 mutant D10A generates a nick on the targeted strand, while the HNH mutant H840A generates a nick on the non-targeted strand (see Jinek et al., Science. 337:816-821(2012); Qi et al., Cell. 28; 152(5):1173-83 (2013))

In some embodiments, the fusion protein comprises a Cas9 nickase fused to an DNA nucleobase modification domain (e.g., adenine deaminase). The term “base editors” encompasses the base editors described herein as well as any base editor known or described in the art at the time of this filing or developed in the future. Reference is made to Rees & Liu, Base editing: precision chemistry on the genome and transcriptome of living cells, Nat Rev Genet. 2018; 19(12):770-788; as well as U.S. Patent Publication No. 2018/0073012, published Mar. 15, 2018, which issued as U.S. Pat. No. 10,113,163; on Oct. 30, 2018; U.S. Patent Publication No. 2017/0121693, published May 4, 2017, which issued as U.S. Pat. No. 10,167,457 on Jan. 1, 2019, as U.S. Pat. No. 10,167,457; International Publication No. WO 2017/070633, published Apr. 27, 2017; U.S. Patent Publication No. 2015/0166980, published Jun. 18, 2015; U.S. Pat. No. 9,840,699, issued Dec. 12, 2017; and U.S. Pat. No. 10,077,453, issued Sep. 18, 2018, the contents of each of which are incorporated herein by reference in their entireties.

The term “Cas9” or “Cas9 nuclease” or “Cas9 domain” refers to a CRISPR associated protein 9, or variant thereof, and embraces any naturally occurring Cas9 from any organism, any naturally-occurring Cas9, any Cas9 homolog, ortholog, or paralog from any organism, and any variant of a Cas9, naturally-occurring or engineered. More broadly, a Cas9 protein, domain, or domain is a type of “nucleic acid programmable DNA binding protein (napDNAbp)”. The term Cas9 is not meant to be limiting and may be referred to as a “Cas9 or variant thereof.” Exemplary Cas9 proteins are described herein and also described in the art. The present disclosure is unlimited with regard to the particular Cas9 that is employed in the base editors of the invention.

In some embodiments, proteins comprising Cas9 or fragments thereof are referred to as “Cas9 variants.” A Cas9 variant shares homology to Cas9, or a fragment thereof. Cas9 variants include functional fragments of Cas9. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to wild type Cas9. In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a wild type Cas9. In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9.

As used herein, the term “dCas9” refers to a nuclease-inactive Cas9 or nuclease-dead Cas9, or a functional fragment or variant thereof, and embraces any naturally occurring dCas9 from any organism, any naturally-occurring dCas9 equivalent or functional fragment thereof, any dCas9 homolog, ortholog, or paralog from any organism, and any mutant or variant of a dCas9, naturally-occurring or engineered. The term dCas9 is not meant to be particularly limiting and may be referred to as a “dCas9 or equivalent.” Exemplary dCas9 proteins and method for making dCas9 proteins are further described herein and/or are described in the art and are incorporated herein by reference.

As used herein, the term “nCas9” or “Cas9 nickase” refers to a Cas9 or a functional fragment or variant thereof, which cleaves or nicks only one of the strands of a target cut site thereby introducing a nick in a double strand DNA molecule rather than creating a double strand break. This can be achieved by introducing appropriate mutations in a wild-type Cas9 which inactivates one of the two endonuclease activities of the Cas9. Any suitable mutation which inactivates one Cas9 endonuclease activity but leaves the other intact is contemplated, such as one of D10A or H840A mutations in the wild-type Cas9 amino acid sequence (e.g., SEQ ID NO: 1) may be used to form the nCas9.

SpCas9, Streptococcus pyogenes M1, SwissProt Accession
No. Q99ZW2, Wild type
(SEQ ID NO: 1)
MDKKYSIGLDIGTNSVGWAVITDEYKVPSKKFKVLGNTDRHSIKKNLIGALLF
DSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIGDQYADLFL
AAKNLSDAILLSDILRVNTEITKAPLSASMIKRYDEHHQDLTLLKALVRQQLPEKYKEIFF
DQSKNGYAGYIDGGASQEEFYKFIKPILEKMDGTEELLVKLNREDLLRKQRTFDNGSIPH
QIHLGELHAILRRQEDFYPFLKDNREKIEKILTFRIPYYVGPLARGNSRFAWMTRKSEETI
TPWNFEEVVDKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISGVEDRFNASL
GTYHDLLKIIKDKDFLDNEENEDILEDIVLTLTLFEDREMIEERLKTYAHLEDDKVMKQL
KRRRYTGWGRLSRKLINGIRDKQSGKTILDFLKSDGFANRNFMQLIHDDSLTFKEDIQKA
QVSGQGDSLHEHIANLAGSPAIKKGILQTVKVVDELVKVMGRHKPENIVIEMARENQTT
QKGQKNSRERMKRIEEGIKELGSQILKEHPVENTQLQNEKLYLYYLQNGRDMYVDQEL
DINRLSDYDVDHIVPQSFLKDDSIDNKVLTRSDKNRGKSDNVPSEEVVKKMKNYWRQL
LNAKLITQRKFDNLTKAERGGLSELDKAGFIKRQLVETRQITKHVAQILDSRMNTKYDE
NDKLIREVKVITLKSKLVSDFRKDFQFYKVREINNYHHAHDAYLNAVVGTALIKKYPKL
ESEFVYGDYKVYDVRKMIAKSEQEIGKATAKYFFYSNIMNFFKTEITLANGEIRKRPLIET
NGETGEIVWDKGRDFATVRKVLSMPQVNIVKKTEVQTGGFSKESILPKRNSDKLIARKK
DWDPKKYGGFDSPTVAYSVLVVAKVEKGKSKKLKSVKELLGITIMERSSFEKNPIDFLE
AKGYKEVKKDLIIKLPKYSLFELENGRKRMLASAGELQKGNELALPSKYVNFLYLASHY
EKLKGSPEDNEQKQLFVEQHKHYLDEIIEQISEFSKRVILADANLDKVLSAYNKHRDKPI
REQAENIIHLFTLTNLGAPAAFKYFDTTIDRKRYTSTKEVLDATLIHQSITGLYETRIDLSQ
LGGD.

The skilled artisan will understand the above example is for illustration only and is not mean to limit the disclosure in any way. As described above, any Cas9 variant may be inactivated to yield ‘dead’ or ‘nickase’ variants (e.g., dCfp1, nCfp1, etc.).

“CRISPR” is a family of DNA sequences (i.e., CRISPR clusters) in bacteria and archaea that represent snippets of prior infections by a virus that have invaded the prokaryote. The snippets of DNA are used by the prokaryotic cell to detect and destroy DNA from subsequent attacks by similar viruses and effectively constitute, along with an array of CRISPR-associated proteins (including Cas9 and homologs thereof) and CRISPR-associated RNA, a prokaryotic immune defense system. In nature, CRISPR clusters are transcribed and processed into CRISPR RNA (crRNA). In certain types of CRISPR systems (e.g., type II CRISPR systems), correct processing of pre-crRNA requires a trans-encoded small RNA (tracrRNA), endogenous ribonuclease 3 (rnc), and a Cas9 protein. The tracrRNA serves as a guide for ribonuclease 3-aided processing of pre-crRNA. Subsequently, Cas9/crRNA/tracrRNA endonucleolytically cleaves linear or circular nucleic acid target complementary to the RNA. Specifically, the target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate embodiments of both the crRNA and tracrRNA into a single RNA species—the guide RNA. See, e.g., Jinek M., et al., Science 337:816-821(2012), the entire contents of which is herein incorporated by reference. Cas9 recognizes a short motif in the CRISPR repeat sequences (the PAM or protospacer adjacent motif) to help distinguish self versus non-self. CRISPR biology, as well as Cas9 nuclease sequences and structures are well known to those of skill in the art (see, e.g., “Complete genome sequence of an M1 strain of Streptococcus pyogenes.” Ferretti J. J., et al., Proc. Natd. Acad. Sci. U.S.A. 98:4658-4663(2001); “CRISPR RNA maturation by trans-encoded small RNA and host factor RNase III.” Deltcheva E., et al., Nature 471:602-607 (2011); and “A programmable dual-RNA-guided DNA endonuclease in adaptive bacterial immunity.” Jinek M., et al., Science 337:816-821(2012), the entire contents of each of which are incorporated herein by reference). Cas9 orthologs have been described in various species, including, but not limited to, S. pyogenes, S. thermophiles, C. ulcerans, S. diphtheria, S. syrphidicola, P. intermedia, S. taiwanense, S. iniae, B. baltica, P. torquis, S. thermophilus, L. innocua, C. jejuni, and N.. meningitidis. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference.

The term “effective amount,” as used herein, refers to an amount of a biologically active agent that is sufficient to elicit a desired biological response. For example, in some embodiments, an effective amount of a base editor may refer to the amount of the base editor that is sufficient to edit a target site nucleotide sequence, e.g., a genome. In some embodiments, an effective amount of a base editor provided herein, e.g., of a fusion protein comprising a nuclease-inactive Cas9 domain and a nucleobase modification domain (e.g., an cytidine and/or adenosine deaminases) may refer to the amount of the fusion protein that is sufficient to induce editing of a target site specifically bound and edited by the fusion protein. In some embodiments, an effective amount of a base editor provided herein may refer to the amount of the fusion protein sufficient to induce editing having the following characteristics: >50% product purity, <5% indels, and an editing window of 2-8 nucleotides. As will be appreciated by the skilled artisan, the effective amount of an agent, e.g., a fusion protein, a nuclease, a deaminase, a hybrid protein, a protein dimer, a complex of a protein (or protein dimer) and a polynucleotide, or a polynucleotide, may vary depending on various factors as, for example, on the desired biological response, e.g., on the specific allele, genome, or target site to be edited, on the target cell or tissue (i.e., the cell or tissue to be edited), and on the agent being used.

The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a nucleic-acid editing protein. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the entire contents of which are incorporated herein by reference.

The term “linker,” as used herein, refers to a chemical group or a molecule linking two molecules or domains, e.g., nCas9 and an cytidine and/or adenosine deaminase. In some embodiments, a linker joins a dCas9 and modification domain (e.g., an cytidine and/or adenosine deaminase). Typically, the linker is positioned between, or flanked by, two groups, molecules, or other domains and connected to each one via a covalent bond, thus connecting the two. In some embodiments, the linker is an amino acid or a plurality of amino acids (e.g., a peptide or protein). In some embodiments, the linker is an organic molecule, group, polymer, or chemical domain. Chemical domains include, but are not limited to, disulfide, hydrazone, thiol and azo domains. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length.

Longer or shorter linkers are also contemplated.

The term “mutation,” as used herein, refers to a substitution of a residue within a sequence, e.g., a nucleic acid or amino acid sequence, with another residue; a deletion or insertion of one or more residues within a sequence; or a substitution of a residue within a sequence of a genome in a subject to be corrected. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)). Mutations can include a variety of categories, such as single base polymorphisms, microduplication regions, indel, and inversions, and is not meant to be limiting in any way. Mutations can include “loss-of-function” mutations which is the normal result of a mutation that reduces or abolishes a protein activity. Most loss-of-function mutations are recessive, because in a heterozygote the second chromosome copy carries an unmutated version of the gene coding for a fully functional protein whose presence compensates for the effect of the mutation. There are some exceptions where a loss-of-function mutation is dominant, one example being haploinsufficiency, where the organism is unable to tolerate the approximately 50% reduction in protein activity suffered by the heterozygote. This is the explanation for a few genetic diseases in humans, including Marfan syndrome which results from a mutation in the gene for the connective tissue protein called fibrillin. Mutations also embrace “gain-of-function” mutations, which is one which confers an abnormal activity on a protein or cell that is otherwise not present in a normal condition. Many gain-of-function mutations are in regulatory sequences rather than in coding regions, and can therefore have a number of consequences. For example, a mutation might lead to one or more genes being expressed in the wrong tissues, these tissues gaining functions that they normally lack. Alternatively the mutation could lead to overexpression of one or more genes involved in control of the cell cycle, thus leading to uncontrolled cell division and hence to cancer. Because of their nature, gain-of-function mutations are usually dominant.

The terms “non-naturally occurring” or “engineered” are used interchangeably and indicate the involvement of the hand of man. The terms, when referring to nucleic acid molecules or polypeptides (e.g., Cas9 or cytidine and/or adenosine deaminases) mean that the nucleic acid molecule or the polypeptide is at least substantially free from at least one other component with which they are naturally associated in nature and/or as found in nature (e.g., an amino acid sequence not found in nature). The terms, when referring to edited endogenous tRNA molecules refer to endogenous tRNAs comprising a nonsense suppressor anticodon.

The term “nucleic acid,” as used herein, refers to RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The term “nucleic acid programmable DNA binding protein (napDNAbp)” refers to any protein that may associate (e.g., form a complex) with one or more nucleic acid molecules (i.e., which may broadly be referred to as a “napDNAbp-programming nucleic acid molecule” and includes, for example, guide RNA in the case of Cas systems) which direct or otherwise program the protein to localize to a specific target nucleotide sequence (e.g., a gene locus of a genome) that is complementary to the one or more nucleic acid molecules (or a portion or region thereof) associated with the protein, thereby causing the protein to bind to the nucleotide sequence at the specific target site. This term napDNAbp embraces CRISPR Cas9 proteins, as well as Cas9 equivalents, homologs, orthologs, or paralogs, whether naturally occurring or non-naturally occurring (e.g., engineered or modified), and may include a Cas9 equivalent from any type of CRISPR system (e.g., type II, V, VI), including Cpf1 (a type-V CRISPR-Cas systems), C2c1 (a type V CRISPR-Cas system), C2c2 (a type VI CRISPR-Cas system), C2c3 (a type V CRISPR-Cas system), dCas9, GeoCas9, CjCas9, Cas12a, Cas12b, Cas12c, Cas12d, Cas12g, Cas12h, Cas12i, Cas13d, Cas14, Argonaute, and nCas9. Further Cas-equivalents are described in Makarova et al., “C2c2 is a single-component programmable RNA-guided RNA-targeting CRISPR effector,” Science 2016; 353 (6299), the contents of which are incorporated herein by reference. However, the nucleic acid programmable DNA binding protein (napDNAbp) that may be used in connection with this invention are not limited to CRISPR-Cas systems. The invention embraces any such programmable protein, such as the Argonaute protein from Natronobacterium gregoryi (NgAgo) which may also be used for DNA-guided genome editing. NgAgo-guide DNA system does not require a PAM sequence or guide RNA molecules, which means genome editing can be performed simply by the expression of generic NgAgo protein and introduction of synthetic oligonucleotides on any genomic sequence. See Gao et al., DNA-guided genome editing using the Natronobacterium gregoryi Argonaute. Nature Biotechnology 2016; 34(7):768-73, which is incorporated herein by reference.

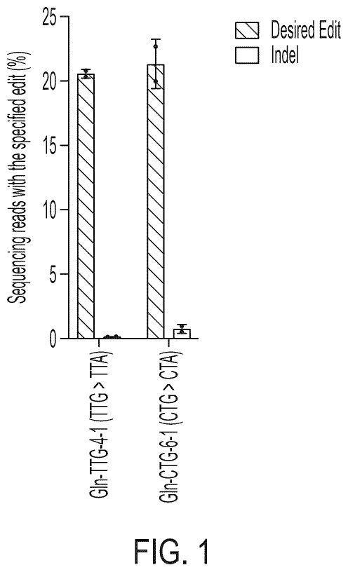

In some embodiments, the napDNAbp is a RNA-programmable nuclease, when in a complex with an RNA, may be referred to as a nuclease:RNA complex. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeably to refer to guide RNAs that exist as either single molecules or as a complex of two or more molecules. Typically, gRNAs that exist as single RNA species comprise two domains: (1) a domain that shares homology to a target nucleic acid (e.g., and directs binding of a Cas9 (or equivalent) complex to the target); and (2) a domain that binds a Cas9 protein. In some embodiments, domain (2) corresponds to a sequence known as a tracrRNA, and comprises a stem-loop structure. For example, in some embodiments, domain (2) is homologous to a tracrRNA as depicted in FIG. 1E of Jinek et al., Science 337:816-821(2012), the entire contents of which is incorporated herein by reference. Other examples of gRNAs (e.g., those including domain 2) can be found in U.S. Pat. No. 9,340,799, entitled “mRNA-Sensing Switchable gRNAs,” and International Patent Application No. PCT/US2014/054247, filed Sep. 6, 2013, published as WO 2015/035136 and entitled “Delivery System For Functional Nucleases,” the entire contents of each are herein incorporated by reference. In some embodiments, a gRNA comprises two or more of domains (1) and (2), and may be referred to as an “extended gRNA.” For example, an extended gRNA will, e.g., bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease:RNA complex. In some embodiments, the RNA-programmable nuclease is the (CRISPR-associated system) Cas9 endonuclease, for example Cas9 (Csnl) from Streptococcus pyogenes (see, e.g., “Complete genome sequence of an M1 strain of Streptococcus pyogenes.” Ferretti J. J. et al., Proc. Natl. Acad. Sci. U.S.A. 98:4658-4663(2001); “CRISPR RNA maturation by trans-encoded small RNA and host factor RNase III.” Deltcheva E. et al., Nature 471:602-607(2011); and “A programmable dual-RNA-guided DNA endonuclease in adaptive bacterial immunity.” Jinek M. et al., Science 337:816-821(2012), the entire contents of each of which are incorporated herein by reference.

The napDNAbp nucleases (e.g., Cas9) use RNA:DNA hybridization to target DNA cleavage sites, these proteins are able to be targeted, in principle, to any sequence specified by the guide RNA. Methods of using napDNAbp nucleases, such as Cas9, for site-specific cleavage (e.g., to modify a genome) are known in the art (see e.g., Cong, L. et al. Multiplex genome engineering using CRISPR/Cas systems. Science 339, 819-823 (2013); Mali, P. et al. RNA-guided human genome engineering via Cas9. Science 339, 823-826 (2013); Hwang, W. Y. et al. Efficient genome editing in zebrafish using a CRISPR-Cas system. Nature Biotechnology 31, 227-229 (2013); Jinek, M. et al. RNA-programmed genome editing in human cells. eLife 2, e00471 (2013); Dicarlo, J. E. et al., Genome engineering in Saccharomyces cerevisiae using CRISPR-Cas systems. Nucleic Acid Res. (2013); Jiang, W. et al. RNA-guided editing of bacterial genomes using CRISPR-Cas systems. Nature Biotechnology 31, 233-239 (2013); the entire contents of each of which are incorporated herein by reference).

The term “napDNAbp-programming nucleic acid molecule” or equivalently “guide sequence” refers the one or more nucleic acid molecules which associate with and direct or otherwise program a napDNAbp protein to localize to a specific target nucleotide sequence (e.g., a gene locus of a genome) that is complementary to the one or more nucleic acid molecules (or a portion or region thereof) associated with the protein, thereby causing the napDNAbp protein to bind to the nucleotide sequence at the specific target site. A non-limiting example is a guide RNA of a Cas protein of a CRISPR-Cas genome editing system.

A nuclear localization signal or sequence (NLS) is an amino acid sequence that tags, designates, or otherwise marks a protein for import into the cell nucleus by nuclear transport. Typically, this signal consists of one or more short sequences of positively charged lysines or arginines exposed on the protein surface. Different nuclear localized proteins may share the same NLS. An NLS has the opposite function of a nuclear export signal (NES), which targets proteins out of the nucleus. Thus, a single nuclear localization signal can direct the entity with which it is associated to the nucleus of a cell. Such sequences can be of any size and composition, for example more than 25, 25, 15, 12, 10, 8, 7, 6, 5 or 4 amino acids, but will preferably comprise at least a four to eight amino acid sequence known to function as a nuclear localization signal (NLS).

The term, as used herein, “nucleobase modification domain” or “modification domain” embraces any protein, enzyme, or polypeptide (or functional fragment thereof) which is capable of modifying a DNA or RNA molecule. Nucleobase modification domains may be naturally occurring, or may be engineered. For example, a nucleobase modification domain can include one or more DNA repair enzymes, for example, and an enzyme or protein involved in base excision repair (BER), nucleotide excision repair (NER), homology-dependent recombinational repair (HR), non-homologous end-joining repair (NHEJ), microhomology end-joining repair (MMEJ), mismatch repair (MMR), direct reversal repair, or other known DNA repair pathway. A nucleobase modification domain can have one or more types of enzymatic activities, including, but not limited to, endonuclease activity, polymerase activity, ligase activity, replication activity, and proofreading activity. Nucleobase modification domains can also include DNA or RNA-modifying enzymes and/or mutagenic enzymes, such as DNA oxidizing enzymes (i.e., cytidine and/or adenosine deaminases), which covalently modify nucleobases leading in some cases to mutagenic corrections by way of normal cellular DNA repair and replication processes. Exemplary nucleobase modification domains include, but are not limited to, an cytidine and/or adenosine deaminase, a nuclease, a nickase, a recombinase, a methyltransferase, a methylase, an acetylase, an acetyltransferase, a transcriptional activator, or a transcriptional repressor domain. In some embodiments the nucleobase modification domain is an cytidine and/or adenosine deaminase (e.g., AlkBH1).

As used herein, the terms “oligonucleotide” and “polynucleotide” can be used interchangeably to refer to a polymer of nucleotides (e.g., a string of at least three nucleotides).

The term “promoter” is art-recognized and refers to a nucleic acid molecule with a sequence recognized by the cellular transcription machinery and able to initiate transcription of a downstream gene. A promoter can be constitutively active, meaning that the promoter is always active in a given cellular context, or conditionally active, meaning that the promoter is only active in the presence of a specific condition. For example, a conditional promoter may only be active in the presence of a specific protein that connects a protein associated with a regulatory element in the promoter to the basic transcriptional machinery, or only in the absence of an inhibitory molecule. A subclass of conditionally active promoters are inducible promoters that require the presence of a small molecule “inducer” for activity. Examples of inducible promoters include, but are not limited to, arabinose-inducible promoters, Tet-on promoters, and tamoxifen-inducible promoters. A variety of constitutive, conditional, and inducible promoters are well known to the skilled artisan, and the skilled artisan will be able to ascertain a variety of such promoters useful in carrying out the instant invention, which is not limited in this respect. In various embodiments, the specification provides vectors with appropriate promoters for driving expression of the nucleic acid sequences encoding the base editor fusion proteins (or one more individual components thereof).

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, engineered, or synthetic, or any combination thereof. The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a recombinase. In some embodiments, a protein comprises a proteinaceous part, e.g., an amino acid sequence constituting a nucleic acid binding domain, and an organic compound, e.g., a compound that can act as a nucleic acid cleavage agent. In some embodiments, a protein is in a complex with, or is in association with, a nucleic acid, e.g., RNA. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

The term “recombinant” as used herein in the context of proteins or nucleic acids refers to proteins or nucleic acids that do not occur in nature, but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is an experimental organism. In some embodiments, the subject is a plant. In some embodiments, the subject is genetically engineered, e.g., a genetically engineered non-human subject. The subject may be of either sex and at any stage of development.

The term “target site” refers to a sequence within a nucleic acid molecule that is edited by a base editor (e.g., a dCas9-cytidine and/or adenosine deaminase fusion protein provided herein). The target site further refers to the sequence within a nucleic acid molecule to which a complex of the base editor and gRNA binds.

The term “vector,” as used herein, may refer to a nucleic acid that has been modified to encode the base editor and/or gRNA. Exemplary suitable vectors include viral vectors, such as retroviral vectors or bacteriophages and filamentous phage, and conjugative plasmids.

The term “viral particle,” as used herein, refers to a viral genome, for example, a DNA or RNA genome, that is associated with a coat of a viral protein or proteins, and, in some cases, with an envelope of lipids. For example, a phage particle comprises a phage genome packaged into a protein encoded by the wild type phage genome.

The term “viral vector,” as used herein, refers to a nucleic acid comprising a viral genome that, when introduced into a suitable host cell, can be replicated and packaged into viral particles able to transfer the viral genome into another host cell. The term “viral vector” extends to vectors comprising truncated or partial viral genomes. For example, in some embodiments, a viral vector is provided that lacks a gene encoding a protein essential for the generation of infectious viral particles. In suitable host cells, for example, host cells comprising the lacking gene under the control of a conditional promoter, however, such truncated viral vectors can replicate and generate viral particles able to transfer the truncated viral genome into another host cell. In some embodiments, the viral vector is an adeno-associated virus (AAV) vector.

The terms “treatment,” “treat,” and “treating,” refer to a clinical intervention aimed to reverse, alleviate, delay the onset of, or inhibit the progress of a disease, disorder, or condition, or one or more symptoms thereof, as described herein. As used herein, the terms “treatment,” “treat,” and “treating” refer to a clinical intervention aimed to reverse, alleviate, delay the onset of, or inhibit the progress of a disease, disorder, or condition, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their prevention or recurrence.

As used herein, the term “variant” refers to a protein having characteristics that deviate from what occurs in nature, e.g., a “variant” is at least about 70% identical, at least about 80% identical, at least about 85% identical, at least about 90% identical, at least about 95% identical, at least about 96% identical, at least about 97% identical, at least about 98% identical, at least about 99% identical, at least about 99.5% identical, or at least about 99.9% identical to the wild type protein. For instance, a variant nucleobase modification domain is a nucleobase modification domain comprising one or more changes in amino acid residues of an cytidine and/or adenosine deaminase, as compared to the wild type amino acid sequences thereof. These changes include chemical modifications, including substitutions of different amino acid residues, as well as truncations. This term embraces functional fragments of the wild type amino acid sequence.

As used herein, the term “wild type” is a term of the art understood by skilled persons and means the typical form of an organism, strain, gene or characteristic as it occurs in nature as distinguished from mutant or variant forms.

As used herein, the term “non-cognate amino acid” refers to an amino acid that pairs with a tRNA molecule that does not comprise an anticodon sequence encoding said amino acid.

As used herein, the term “nonsense mutation” refers to a mutation in which a sense codon that corresponds to one of the twenty amino acids specified by the genetic code is changed to a chain-terminating codon (e.g., an opal stop codon, an amber stop codon, or a ochre stop codon).

As used herein the term “nonsense suppressor anticodon sequence” refers to an anticodon sequence that is complementary to an opal stop codon (e.g., 5′-UCA-3′), an amber codon (e.g., 5′-CUA-3′), or an ochre stop codon (e.g., 5′-UUA-3′).

As used herein, the term “premature termination stop codon” or “PTC” refers to a nonsense mutation in a mRNA sequence, wherein the stop codon occurs earlier in the sequence, relative to the non-mutated mRNA sequence, and thus impedes translation of the full-length protein encoded by the mRNA sequence. Premature termination codon may be an ochre stop codon comprising a 5′-UAA-3′ codon sequence, an opal stop codon comprising a 5′-UGA-3′ codon sequence, or an amber stop codon comprising a 5′-UAG-3′ codon sequence.

As used herein, the term “redundant and DNA sequence” refers to a DNA sequence encoding a tRNA gene that has codon degeneracy. Codon degeneracy means that there is more than one codon, and hence anticodon, that specifies a single amino acid (see Table 1)

As used herein, the term “suppressor tRNA” refers to a tRNA (defined elsewhere herein) charged with an amino acid comprising a mutation in the anticodon that allows it to recognize a premature stop codon (defined elsewhere herein as either an amber, ochre, or opal stop codon) on an mRNA and to and insert an amino acid into the amino acid sequence encoded by the mRNA, thus preventing truncation of the amino acid sequence.

As used herein the terms “tRNA” or “endogenous tRNA” or “unedited tRNA” collectively refer to a transfer RNA as found in nature. tRNA is an art recognized term that refers to a molecule composed of RNA that serves as the physical link between mRNA and the amino acid sequence of proteins. The tRNA structure consists of the following: (i) a 5′-terminal phosphate group, (ii) an acceptor stem made by the base pairing of the 5′-terminal new nucleotide with the 3′-terminal nucleotide (which contains the CCA 3′-terminal group used to attach the amino acid), (iii) a CCA tail at the 3′-end of the tRNA molecule that is covalently bound to an amino acid (herein “aminoacyl-tRNA), (iv) a D arm domain, (v) an anticodon arm comprising an anticodon sequence. The tRNA 5′-to-3′ primary structure contains the anticodon but in reverse order, since 3′-to-5′ directionality is required to read the mRNA from 5′-to-3′, (vi) a T-arm domain, and (vii) a variable arm domain

The term “deaminase” or “deaminase domain” refers to a protein or enzyme that catalyzes a deamination reaction. In some embodiments, the deaminase is an adenosine (or adenine) deaminase, which catalyzes the hydrolytic deamination of adenine or adenosine. In some embodiments, the adenosine deaminase catalyzes the hydrolytic deamination of adenine or adenosine in deoxyribonucleic acid (DNA) to inosine. In other embodiments, the deaminase is a cytidine (or cytosine) deaminase, which catalyzes the hydrolytic deamination of cytidine or cytosine.

The deaminases provided herein may be from any organism, such as a bacterium. In some embodiments, the deaminase or deaminase domain is a variant of a naturally-occurring deaminase from an organism. In some embodiments, the deaminase or deaminase domain does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase.

As used herein, the term “adenosine deaminase” or “adenosine deaminase domain” refers to a protein or enzyme that catalyzes a deamination reaction of an adenosine (or adenine). The terms “adenosine” and “adenine” are used interchangeably for purposes of the present disclosure. For example, for purposes of the disclosure, reference to an “adenine base editor” (ABE) refers to the same entity as an “adenosine base editor” (ABE). Similarly, for purposes of the disclosure, reference to an “adenine deaminase” refers to the same entity as an “adenosine deaminase.” However, the person having ordinary skill in the art will appreciate that “adenine” refers to the purine base whereas “adenosine” refers to the larger nucleoside molecule that includes the purine base (adenine) and sugar moiety (e.g., either ribose or deoxyribose). In certain embodiments, the disclosure provides base editor fusion proteins comprising one or more adenosine deaminase domains. For instance, an adenosine deaminase domain may comprise a heterodimer of a first adenosine deaminase and a second deaminase domain, connected by a linker. Adenosine deaminases (e.g., engineered adenosine deaminases or evolved adenosine deaminases) provided herein may be enzymes that convert adenine (A) to inosine (I) in DNA or RNA. Such adenosine deaminase can lead to an A:T to G:C base pair conversion. In some embodiments, the deaminase is a variant of a naturally-occurring deaminase from an organism. In some embodiments, the deaminase does not occur in nature. For example, in some embodiments, the deaminase is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase.

In some embodiments, the adenosine deaminase is derived from a bacterium, such as, E. coli, S. aureus, S. typhi, S. putrefaciens, H. influenzae, or C. crescentus. In some embodiments, the adenosine deaminase is a TadA deaminase. In some embodiments, the TadA deaminase is an E. coli TadA deaminase (ecTadA). In some embodiments, the TadA deaminase is a truncated E. coli TadA deaminase. For example, the truncated ecTadA may be missing one or more N-terminal amino acids relative to a full-length ecTadA. In some embodiments, the truncated ecTadA may be missing 1, 2, 3, 4, 5, 6, 7, 8, 9, 10, 11, 12, 13, 14, 15, 6, 17, 18, 19, or 20 N-terminal amino acid residues relative to the full length ecTadA. In some embodiments, the truncated ecTadA may be missing 1, 2, 3, 4, 5, 6, 7, 8, 9, 10, 11, 12, 13, 14, 15, 6, 17, 18, 19, or 20 C-terminal amino acid residues relative to the full length ecTadA. In some embodiments, the ecTadA deaminase does not comprise an N-terminal methionine. Reference is made to U.S. Patent Publication No. 2018/0073012, published Mar. 15, 2018, which is incorporated herein by reference.

As used herein, the term “cytidine deaminase” or “cytidine deaminase domain” refers to a protein or enzyme that catalyzes a deamination reaction of a cytidine or cytosine. The terms “cytidine” and “cytosine” are used interchangeably for purposes of the present disclosure. For example, for purposes of the disclosure, reference to an “cytosine base editor” (CBE) refers to the same entity as an “cytosine base editor” (CBE). Similarly, for purposes of the disclosure, reference to an “cytidine deaminase” refers to the same entity as an “cytosine deaminase.” However, the person having ordinary skill in the art will appreciate that “cytosine” refers to the pyrimidine base whereas “cytidine” refers to the larger nucleoside molecule that includes the pyrimidine base (cytosine) and sugar moiety (e.g., either ribose or deoxyribose). A cytidine deaminase is encoded by the CDA gene and is an enzyme that catalyzes the removal of an amine group from cytidine (i.e., the base cytosine when attached to a ribose ring, i.e., the nucleoside referred to as cytidine) to uridine (C to U) and deoxycytidine to deoxyuridine (C to U). A non-limiting example of a cytidine deaminase is APOBEC1 (“apolipoprotein B mRNA editing enzyme, catalytic polypeptide 1”). Another example is AID (“activation-induced cytidine deaminase”). Under standard Watson-Crick hydrogen bond pairing, a cytosine base hydrogen bonds to a guanine base. When cytidine is converted to uridine (or deoxycytidine is converted to deoxyuridine), the uridine (or the uracil base of uridine) undergoes hydrogen bond pairing with the base adenine. Thus, a conversion of “C” to uridine (“U”) by cytidine deaminase will cause the insertion of “A” instead of a “G” during cellular repair and/or replication processes. Since the adenine “A” pairs with thymine “T”, the cytidine deaminase in coordination with DNA replication causes the conversion of an C-G pairing to a T-A pairing in the double-stranded DNA molecule.

The term “guide RNA” is a particular type of guide nucleic acid which is mostly commonly associated with a Cas protein of a CRISPR-Cas9 and which associates with Cas9, directing the Cas9 protein to a specific sequence in a DNA molecule that includes complementarity to protospacer sequence of the guide RNA. However, this term also embraces the equivalent guide nucleic acid molecules that associate with Cas9 equivalents, homologs, orthologs, or paralogs, whether naturally-occurring or non-naturally-occurring (e.g., engineered or recombinant), and which otherwise program the Cas9 equivalent to localize to a specific target nucleotide sequence. The Cas9 equivalents may include other napDNAbp from any type of CRISPR system (e.g., type II, V, VI), including Cpf1 (a type-V CRISPR-Cas systems), C2c1 (a type V CRISPR-Cas system), C2c2 (a type VI CRISPR-Cas system) and C2c3 (a type V CRISPR-Cas system). Further Cas-equivalents are described in Makarova et al., “C2c2 is a single-component programmable RNA-guided RNA-targeting CRISPR effector,” Science 2016; 353(6299), the contents of which are incorporated herein by reference. Exemplary sequences are and structures of guide RNAs are provided herein.

Guide RNAs may comprise various structural elements that include, but are not limited to (a) a spacer sequence—the sequence in the guide RNA (having ˜20 nts in length) which binds to a complementary strand of the target DNA (and has the same sequence as the protospacer of the DNA) and (b) a gRNA core (or gRNA scaffold or backbone sequence)—refers to the sequence within the gRNA that is responsible for Cas9 binding, it does not include the ˜20 bp spacer sequence that is used to guide Cas9 to target DNA.

As used herein, the “guide RNA target sequence” refers to the ˜20 nucleotides that are complementary to the protospacer sequence in the PAM strand. The target sequence is the sequence that anneals to or is targeted by the spacer sequence of the guide RNA. The spacer sequence of the guide RNA and the protospacer have the same sequence (except the spacer sequence is RNA and the protospacer is DNA).

As used herein, the “guide RNA scaffold sequence” refers to the sequence within the gRNA that is responsible for Cas9 binding, it does not include the 20 bp spacer/targeting sequence that is used to guide Cas9 to target DNA.

The term “uracil glycosylase inhibitor” or “UGI,” as used herein, refers to a protein that is capable of inhibiting a uracil-DNA glycosylase base-excision repair enzyme. In some embodiments, a UGI domain comprises a wild-type UGI or a UGI as set forth in SEQ ID NO: 2. In some embodiments, the UGI proteins provided herein include fragments of UGI and proteins homologous to a UGI or a UGI fragment. For example, in some embodiments, a UGI domain comprises a fragment of the amino acid sequence set forth in SEQ ID NO: 2. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence as set forth in SEQ ID NO: 2. In some embodiments, a UGI comprises an amino acid sequence homologous to the amino acid sequence set forth in SEQ ID NO: 2, or an amino acid sequence homologous to a fragment of the amino acid sequence set forth in SEQ ID NO: 2. In some embodiments, proteins comprising UGI or fragments of UGI or homologs of UGI or UGI fragments are referred to as “UGI variants.” A UGI variant shares homology to UGI, or a fragment thereof. For example, a UGI variant is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a wild type UGI or a UGI as set forth in SEQ ID NO: 2. In some embodiments, the UGI variant comprises a fragment of UGI, such that the fragment is at least 70% identical, at least 80% identical, at least 90% identical, at least 95% identical, at least 96% identical, at least 97% identical, at least 98% identical, at least 99% identical, at least 99.5% identical, or at least 99.9% to the corresponding fragment of wild-type UGI or a UGI as set forth in SEQ ID NO: 2. In some embodiments, the UGI comprises the following amino acid sequence:

(SEQ ID NO: 2)
MTNLSDIIEKETGKQLVIQESILMLPEEVEEVIGNKPESDILVHTAYDES
TDENVMLLTSDAPEYKPWALVIQDSNGENKIKML
(P14739|UNGI_BPPB2 Uracil-DNA glycosylase
inhibitor).

### DETAILED DESCRIPTION

Aspects of the disclosure relate to methods, compositions, and systems for editing a DNA sequence encoding an endogenous tRNA into a suppressor tRNA using base editing (e.g., to treat a disease caused by a premature termination codon or PTC). Additional aspects relate to compositions comprising a gRNA configured to bind to a DNA sequence encoding an endogenous tRNA. Other aspects relate to complexes comprising a base editor and a gRNA that are capable of editing an endogenous tRNA into a suppressor tRNA. In some aspects, the disclosure further relates to polynucleotides encoding one or more nucleic acid sequences encoding the gRNAs, vectors comprising the polynucleotides, and/or cells comprising the polynucleotides, complexes, gRNAs, and/or vectors disclosed herein. Additional aspects further relate to kits comprising any one of the compositions, complexes, gRNAs, polynucleotides, vectors, and/or cells disclosed herein.

As defined elsewhere herein, suppressor tRNAs are tRNAs that are natively charged with their cognate amino acids but possess engineered anticodon loops designed to bind PTCs (e.g., amber, ochre, or opal stop codons). As such, suppressor tRNAs bind to PTCs during the process of translation, leading to incorporation of an amino acid instead of terminating translation. Without wishing to be bound by theory, suppressor tRNAs were recently used to rescue a genetic disease in a mouse model carrying a nonsense mutation, but the suppressor tRNA was delivered via an adeno-associated viral vector (herein “AAV”). It is generally known in the art that permanent expression of the suppressor tRNA is necessary for continued rescue of the disease, which is challenging to achieve using AAV and requires repeated administration of the suppressor tRNA vector.

It is generally recognized in the art that humans possess over 500 interspersed tRNA genes, and many of these genes are redundant and dispensable. For example, one or both copies of the tRNA^{Lys} CUU gene is deleted in ˜50% of humans¹². Therefore, using base editing to convert the CUU anticodon of this tRNA^{Lys} gene into UUA, UCA, or CUA for ochre, opal, and amber suppression, respectively, would generate an endogenous suppressor tRNA^{Lys}. Thus, in some embodiments, the endogenous, tRNA is a tRNA^{Lys} CUU gene. In this particular embodiment, lysine would be installed at the locations of the PTCs. In other embodiments, the tRNA gene is any gene sequence known in the art (e.g., human tRNA genes are listed in Table 1).

In other embodiments, other domains in the tRNA gene may be edited to modify the identity of the amino acid that is charged onto the suppressor tRNA. For example, base editing may be used to install a C70U mutation in the acceptor stem of tRNA^{Lys}; this mutation is known to change the identity of the charged amino acid to alanine¹³. Other edits within the acceptor stem domain and/or other domains (e.g., D-arm, T-arm, anticodon arm, or variable arm) may also be used to alter the identity of the charged amino acid.

In some embodiments, the choice of amino acid inserted in response to a stop codon is tailored by the choice of tRNA to edit and/or by installing sequences recognized by specific aminoacyl-tRNA synthetase enzymes to direct amino acid charging of the newly generated suppressor tRNA. In some embodiments, suppression with widely tolerated amino acids such as glycine, alanine, or serine may be preferable to suppression with more unusual amino acids such as proline or arginine or tryptophan, except when treating diseases caused by premature stop codons that have arisen from mutation of these amino acids. For example, Arg to STOP mutations are a common cause of genetic diseases, and in these cases, base editing to create an arginine-charged suppressor tRNA may be especially desirable.

As such, some aspects of the present disclosure are related to methods for editing a DNA sequence encoding an endogenous tRNA at a target site. In some embodiments, the target site in the DNA sequence encodes one or more domains of the endogenous tRNA. tRNA domains are known in the art and comprise the D-arm domain, T-arm domain, variable arm domain, acceptor stem domain and a anticodon arm domain comprising an anticodon sequence.

As used herein, the term “D arm domain” refers to a feature in the tertiary structure of tRNA. Without wishing to be bound by theory, it comprises two D stems and the D loop. The D loop further comprises the base dihydrouridine, for which the arm is named. The D-loops main function is recognition. It is widely believed that it acts as a recognition site for aminoacyl-tRNA synthetase, an enzyme involved in the aminoacylation of the tRNA molecule.

As used herein, the term “T-arm domain” refers to a specialized region of the tRNA which acts as a special recognition site for the ribosome to form a tRNA-ribosome complex during protein biosynthesis (e.g., translation). The T-arm domain is generally believed to have two components: a T-stem and T-loop. There are two T-stems of five base pairs each. The T-loop is often referred to as the TTC arm due to the presence of thymidine, pseudouridine and cytidine.

As used herein, the term “anticodon arm domain” refers to a 5-bp stem whose loop contains the anticodon. The anticodon portion of the tRNA binds to the codon sequence in mRNA during translation.

As used herein, the term “variable arm domain” refers to a loop that present between the anticodon arm and the TTC arm. The length of the variable arm domain is important in the recognition of the aminoacyl-tRNA synthetase for the tRNA. In some embodiments, the tRNA lacks the variable arm domain.

In some embodiments, the endogenous tRNA anticodon sequence is a single transition mutation away from a nonsense suppressor anticodon. As defined elsewhere herein, a nonsense suppressor anticodon is the complementary sequence to a premature termination codon or PTC. There are currently 3 known PTCs, each of which, comprises a different sequence. The ochre stop codon has sequence 5′ UAA 3′ and corresponds to nonsense suppressor anticodon with sequence 5′-UUA-3′. The opal stop codon has sequence 5′ UGA 3′ and corresponds to the nonsense suppressor anticodon with sequence 5′-UCA-3′. The amber stop codon has sequence 5′ UAG 3 and corresponds to nonsense suppressor anticodon with sequence 5′-CUA-3′.

The single transition mutation may be any transition mutation known in the art. For example, in some embodiments, the single transition mutation consists of a C>T (e.g., C-to-T) mutation, a T>C mutation (e.g., T-to-C) mutation, an A>G (e.g., A-to-G) mutation, and a G>A (G-to-A) mutation.

In some embodiments, the endogenous tRNA comprises an anticodon sequence that is a single transversion mutation away from a nonsense suppressor anticodon. The single transversion mutation may be any transversion mutation known in the art. For example, in some embodiments, the single transversion mutation is selected from the group consisting of an A>C (e.g., A-to-C) mutation, T>G (T-to-G) mutation, G>T (G-to-T) mutation, C>A (C-to-A) mutation, C>G (C-to-G) mutation, G>C (G-to-C) mutation, A>T (A-to-T) mutation, and T>A (T-to-A) mutation.

In some embodiments, the endogenous tRNA comprises an anticodon sequence that is 3′-X1-X2-X3-5′. In some embodiments, the base editor installs the mutation (e.g., transition or transversion) at position XL. In some embodiments, the mutation is selected from the group consisting of G>A, C>A, and U>A, relative to the endogenous tRNA. In some embodiments, the anticodon sequence comprises a N>A mutation at X1, C at X2, and U at X3, wherein N is G, C, or U (e.g., which is configured to bind to the PTC 5′-UGA-3′). In some embodiments, the anticodon sequence comprises a N>A mutation at X1, U at X2, and C at X3, wherein N is G, C, or U (e.g., which is configured to bind to the PTC 5′-UAG-3′). In some embodiments, the anticodon sequence comprises a N>A mutation at X1, U at X2, and U at X3, wherein N is G, C, or U (e.g., which is configured to bind to the PTC 5′-UAA-3′).

In some embodiments, the base editor installs the mutation (e.g., transition or transversion) at position X2. In some embodiments, the mutation is selected from the group consisting of A>C, G>C, and U>C, relative to the endogenous tRNA. In some embodiments, the anticodon sequence comprises an A at X1, an N>C mutation at X2, and a U at X3, wherein N is A, G, U (e.g., which is configured to bind to PTC 5′-UGA-3′).

In some embodiments, the mutation is selected from the group consisting of A>U, G>U, or C>U at position X2, relative to the endogenous tRNA. In some embodiments, the anticodon sequence comprises an A at X1, an N>U mutation at X2, and a C at X3, wherein N is A, G, or C (e.g., which is configured to bind to PTC 5′-UAG-3′). In some embodiments, the anticodon sequence comprises an A at X1, a N>U mutation at X2, and C at X3, wherein N is A, G, or C (e.g., which is configured to bind to PTC 5′-UAG-3′). In some embodiments, the anticodon sequence comprises an A at X1, a N>U mutation at X2, and a U at X3, wherein N is A, G, or C (e.g., which is configured to bind to PTC 5′-UAA-3′).

In some embodiments, the base editor installs the mutation (e.g., transition or transversion) at position X3. In some embodiments, the mutation is selected from the group consisting of A>U, G>U, and C>U, relative to the endogenous tRNA. In some embodiments, the anticodon sequence comprises an A at X1, a C at X2, and a N>U at X3, wherein N is an A, G, or C (e.g., which is configured to bind to PTC 5′-UGA-3′). In some embodiments, the anticodon sequence comprises an A at X1, a U at X2 and a N>U at X3, wherein N is an A, G, or C (e.g., which is configured to bind to PTC 5′-UAA-3′).

In some embodiments, the mutation is selected from the group consisting of U>C, A>C, and G>C at position X3, relative to the endogenous tRNA. In some embodiments, the anticodon sequence comprises an A at X1, a U at X2 and a N>C at X3, wherein N is U, A, or G (e.g., which is configured to bind to PTC 5′-UAG-3′)

Other aspects of the present disclosure relate to compositions comprising the edited tRNAs described herein. While it is generally known that translational stop codon readthrough provides a regulatory mechanism of gene expression this extensively utilized by positive-sense ssRNA viruses, no such mechanism has been observed in humans. In other words, suppressor tRNAs are not naturally found and/or naturally occurring in humans. Thus, in some embodiments, the compositions comprise one or more suppressor tRNA engineered from endogenous tRNAs. In some embodiments, the suppressor tRNA comprise a nonsense suppressor anticodon sequence selected from the group consisting of 5′-UUA-3′, 5′-UCA-3′ and 5′-CUA-3′. In some embodiments, the suppressor tRNA further comprises an amino acid selected from the group consisting of alanine, arginine, asparagine, aspartic acid, cysteine, glutamine, glutamic acid, glycine, histidine, isoleucine, leucine, methionine, phenylalanine, proline, serine, threonine, tryptophan, tyrosine, valine, pyrrolysine, and selenocysteine.

Some aspects of the disclosure further relate to guide RNA comprising a spacer sequence that binds to a complementary strand of a target DNA and a gRNA core that mediates binding of a base editor to the DNA, wherein the spacer sequence is any sequence listed in Table 2.

In some embodiments, the gRNA comprises a spacer sequence with at least 90%, at least 95%, at least 98%, at least 99%, at least 99.5%, or at least 99.8% sequence identity to any sequence listed in Table 2.

In some embodiments, the spacer sequence comprises least 90%, at least 95%, at least 98%, at least 99%, at least 99.5%, or at least 99.8% sequence identity to CTGATCCGAAGTCAGACGCC (SEQ ID NO: 3).

In some embodiments, the spacer sequence comprises least 90%, at least 95%, at least 98%, at least 99%, at least 99.5%, or at least 99.8% sequence identity to TCTGCAGTCAAATGCTCTAC (SEQ ID NO. 4).

In some embodiments, the spacer sequence comprises least 90%, at least 95%, at least 98%, at least 99%, at least 99.5%, or at least 99.8% sequence identity to TTGATTTGCAGTCAAATGCTC (SEQ ID NO: 5).

In some embodiments, the spacer sequence comprises least 90%, at least 95%, at least 98%, at least 99%, at least 99.5%, or at least 99.8% sequence identity to GGATTCAGAGTCCAGAGTGC (SEQ ID NO: 6).

In some embodiments, the spacer sequence comprises least 90%, at least 95%, at least 98%, at least 99%, at least 99.5%, or at least 99.8% sequence identity to TGGATTCAAAGCCCAGAGTG (SEQ ID NO: 7).In some embodiments, the spacer sequence comprises least 90%, at least 95%, at least 98%, at least 99%, at least 99.5%, or at least 99.8% sequence identity to CGCTCTCACCGCCGCGGCCC (SEQ ID NO: 8).

In some embodiments, the spacer sequence comprises least 90%, at least 95%, at least 98%, at least 99%, at least 99.5%, or at least 99.8% sequence identity to GGTTTTCACCCAGGTGGCCC (SEQ ID NO: 9).

In some embodiments, the spacer sequence comprises least 90%, at least 95%, at least 98%, at least 99%, at least 99.5%, or at least 99.8% sequence identity to TTGCCTTCCAAGCAGTTGAC (SEQ ID NO: 10).

In some embodiments, the spacer sequence comprises least 90%, at least 95%, at least 98%, at least 99%, at least 99.5%, or at least 99.8% sequence identity to GACTCCAGATCAGAAGGCTG (SEQ ID NO. 11).

In some embodiments, the spacer sequence comprises least 90%, at least 95%, at least 98%, at least 99%, at least 99.5%, or at least 99.8% sequence identity to CTACAGTCCTCCGCTCTACC (SEQ ID NO: 12).

In some embodiments, the spacer sequence comprises least 90%, at least 95%, at least 98%, at least 99%, at least 99.5%, or at least 99.8% sequence identity to GATTTCAAGTCCAACGCCTT (SEQ ID NO: 13).

In some embodiments, the spacer sequence comprises least 90%, at least 95%, at least 98%, at least 99%, at least 99.5%, or at least 99.8% sequence identity to GATTTCGAGTCCAACACCTT (SEQ ID NO: 14).

In some embodiments, the spacer sequence comprises least 90%, at least 95%, at least 98%, at least 99%, at least 99.5%, or at least 99.8% sequence identity to ACTATAGCTACTTCCTCAGT (SEQ ID NO: 15).

In some embodiments, the spacer sequence comprises least 90%, at least 95%, at least 98%, at least 99%, at least 99.5%, or at least 99.8% sequence identity to GGACTTAAGATCCAATGGGC (SEQ ID NO: 16).

Other spacer sequences are also possible in other embodiments.

Additional aspects of the disclosure relate to compositions comprising a base editor and a guide RNA and any complexes formed thereof. In some embodiments, the guide RNA comprises a spacer sequence configured to bind to one or more tRNA genes.

Other aspects of the disclosure relate to polynucleotides, cells, pharmaceutical compositions and kits. For example, in some aspects, the disclosure relates to a polynucleotide comprising a first nucleic acid sequence encoding a base editor and a second nucleic acid sequence encoding a guide RNA, wherein the guide RNA comprises a spacer sequence configured to bind to one or more tRNA genes (e.g., see Table 2).

In some aspects, the disclosure relates to cells comprising any one of the polynucleotides disclosed herein. In some embodiments, the cell is an animal cell. In some embodiments, the animal cell is a mammalian cell, a non-human primate cell, or a human cell. In other embodiments, the cell is a plant cell.

In some aspects, the disclosure relates to pharmaceutical compositions comprising any one of the compositions, pegRNAs, complexes, polynucleotides, and cells disclose herein, or any combination thereof, and a pharmaceutical excipient.

In some aspects, the disclosure relates to kits comprising any one of the compositions, guide RNAs, complexes, polynucleotides, and cells disclose herein, or any combination thereof, and a pharmaceutical excipient, and instructions for editing a one or more DNA sequences encoding one or more domains of a tRNA by base editing, wherein the DNA sequence is any sequence that encodes a tRNA (e.g., see Table 1).

Other aspects of the disclosure relate to methods for changing the amino acid that is charged onto an endogenous tRNA. Without wishing to be bound by theory, it is generally recognized in the art that mutation of select nucleotides within one or more domains of the endogenous tRNA alters the aminoacyl-tRNA synthetase that recognizes the endogenous tRNA, and hence, charges the tRNA with a non-cognate amino acid. For example, tRNAs comprising a C70U mutation in the acceptor stem domain are charged alanine, regardless of their anticodon sequence. Thus, in some embodiments, the tRNAs edited with the base editors described herein, comprises an anticodon sequence that encodes for the cognate amino acid but are charged with a non-cognate amino acid.

In some embodiments, the methods comprise installing one or more edits in one or more domains, wherein the one or more edits changes the identity of the charged amino acid on the tRNA. Any tRNA domain known in the art may be edited, including, for example, the D-arm domain, T-arm domain, variable arm domain, acceptor stem domain, and the anticodon arm domain. In some embodiments, the base editor installs a transition mutation in the one or more domains. In other embodiments, the base editor installs a transversion mutation in the one or more domains.

In some embodiments, the cognate amino acid of the endogenous tRNA is selected from the group consisting of alanine, arginine, asparagine, aspartic acid, cysteine, glutamine, glutamic acid, glycine, histidine, isoleucine, leucine, methionine, phenylalanine, proline, serine, threonine, tryptophan, tyrosine, valine, pyrrolysine, selenocysteine.

In some embodiments, the non-cognate amino acid of the endogenous tRNA is selected from the group consisting of alanine, arginine, asparagine, aspartic acid, cysteine, glutamine, glutamic acid, glycine, histidine, isoleucine, leucine, methionine, phenylalanine, proline, serine, threonine, tryptophan, tyrosine, valine, pyrrolysine, and selenocysteine.

Additional aspects of the disclosure relate to methods for producing a suppressor tRNA molecules from an endogenous tRNA molecule using base editing in a subject in need thereof, the method comprising administering to the subject: (i) a base editor and (ii) a guide RNA, wherein the base editor and the gRNA install a mutation at a target site in a DNA sequence encoding the tRNA molecule, wherein installation of the mutation converts the endogenous tRNA molecule into the suppressor tRNA molecule.

Other aspects relate to methods of treating a disease caused by premature termination codons in a subject in need thereof, the method comprising administering to the subject (i) a base editor and (ii) a guide RNA, wherein the base editor and guide RNA form a base editor complex, wherein the base editor complex mutates a target DNA sequence encoding one or more domains of a tRNA to produce a suppressor tRNA, wherein the suppressor tRNA comprises an anticodon sequence complementary to an ochre stop codon, an opal stop codon, or an amber stop codon.

In some embodiments, the endogenous tRNA comprises an anticodon sequence that is 3′-X1-X2-X3-5′. In some embodiments, the base editor installs the mutation (e.g., transition or transversion) at position XL. In some embodiments, the mutation is selected from the group consisting of G>A, C>A, and U>A, relative to the endogenous tRNA. In some embodiments, the anticodon sequence comprises a N>A mutation at X1, C at X2, and U at X3, wherein N is G, C, or U (e.g., which is configured to bind to the PTC 5′-UGA-3′). In some embodiments, the anticodon sequence comprises a N>A mutation at X1, U at X2, and C at X3, wherein N is G, C, or U (e.g., which is configured to bind to the PTC 5′-UAG-3′). In some embodiments, the anticodon sequence comprises a N>A mutation at X1, U at X2, and U at X3, wherein N is G, C, or U (e.g., which is configured to bind to the PTC 5′-UAA-3′).

In some embodiments, the base editor installs the mutation (e.g., transition or transversion) at position X2. In some embodiments, the mutation is selected from the group consisting of A>C, G>C, and U>C, relative to the endogenous tRNA. In some embodiments, the anticodon sequence comprises an A at X1, an N>C mutation at X2, and a U at X3, wherein N is A, G, U (e.g., which is configured to bind to PTC 5′-UGA-3′).

In some embodiments, the mutation is selected from the group consisting of A>U, G>U, or C>U at position X2, relative to the endogenous tRNA. In some embodiments, the anticodon sequence comprises an A at X1, an N>U mutation at X2, and a C at X3, wherein N is A, G, or C (e.g., which is configured to bind to PTC 5′-UAG-3′). In some embodiments, the anticodon sequence comprises an A at X1, a N>U mutation at X2, and C at X3, wherein N is A, G, or C (e.g., which is configured to bind to PTC 5′-UAG-3′). In some embodiments, the anticodon sequence comprises an A at X1, a N>U mutation at X2, and a U at X3, wherein N is A, G, or C (e.g., which is configured to bind to PTC 5′-UAA-3′).

In some embodiments, the base editor installs the mutation (e.g., transition or transversion) at position X3. In some embodiments, the mutation is selected from the group consisting of A>U, G>U, and C>U, relative to the endogenous tRNA. In some embodiments, the anticodon sequence comprises an A at X1, a C at X2, and a N>U at X3, wherein N is an A, G, or C (e.g., which is configured to bind to PTC 5′-UGA-3′). In some embodiments, the anticodon sequence comprises an A at X1, a U at X2 and a N>U at X3, wherein N is an A, G, or C (e.g., which is configured to bind to PTC 5′-UAA-3′).

In some embodiments, the mutation is selected from the group consisting of U>C, A>C, and G>C at position X3, relative to the endogenous tRNA. In some embodiments, the anticodon sequence comprises an A at X1, a U at X2 and a N>C at X3, wherein N is U, A, or G (e.g., which is configured to bind to PTC 5′-UAG-3′).

In some embodiments, the anticodon sequence complementary to the ochre stop codon is 5′-UUA-3′. In some embodiments, the anticodon sequence complementary to the opal stop codon is 5′-UCA-3′. In some embodiments, the anticodon sequence complementary to the amber stop codon is 5′-CUA-3′.

Other aspects relate to methods for treating a disease caused by premature termination codons, the method comprising mutating an endogenous tRNA gene into a suppressor tRNA gene using base editing, the method comprising administering to a subject (i) a base editor and (ii) a guide RNA, wherein the suppressor tRNA gene encodes a suppressor tRNA molecule comprising an anticodon sequence configured to bind to an ochre stop codon, an opal stop codon, or an amber stop codon.

Non-limiting examples of diseases caused by premature termination codons (e.g., nonsense mutations) include cystic fibrosis, beta thalassemia, Hurler syndrome, Dravet syndrome, Duchenne muscular dystrophy, Usher syndrome, and hemophilia. These examples are meant to be nonlimiting and the skilled artisan will understand that the methods disclosed herein may be used to treat any disease (e.g., known or yet to be determined) caused by premature termination codons (e.g., nonsense mutations).

TABLE 1
Exemplary embodiments of human tRNA gene sequences
(hg38 genome assembly) that may be edited using any
of the base editors/gRNAs disclosed herein.

| tRNA | | | SEQ |
|---|---|---|---|
| gene | Genomic | | ID |
| name | coordinates | Sequence | NO: |
| Homo_ | chr6: | GGGGGTATAGCTCAGTGGTAGAGCGCGTGC | 167 |
| sapiens_ | 28795964- | TTAGCATGCACGAGGTCCTGGGTTCGATCC | |
| tRNA- | 28796035 | CCAGTACCTCCA | |
| Ala- | (−) | | |
| AGC- | | | |
| 1- | | | |
| 1 | | | |
| Homo_ | chr6: | GGGGAATTAGCTCAAGTGGTAGAGCGCTTG | 168 |
| sapiens_ | 26687257- | CTTAGCACGCAAGAGGTAGTGGGATCGATG | |
| tRNA- | 26687329 | CCCACATTCTCCA | |
| Ala- | (+) | | |
| AGC- | | | |
| 10- | | | |
| 1 | | | |
| Homo_ | chr6: | GGGGAATTAGCTCAAGTGGTAGAGCGCTTG | 169 |
| sapiens_ | 26814339- | CTTAGCACGCAAGAGGTAGTGGGATCGATG | |
| tRNA- | 26814411 | CCCACATTCTCCA | |
| Ala- | (−) | | |
| AGC- | | | |
| 10- | | | |
| 2 | | | |
| Homo_ | chr6: | GGGGAATTAGCTCAAATGGTAGAGCGCTCG | 170 |
| sapiens_ | 26571864- | CTTAGCATGCGAGAGGTAGCGGGATCGATG | |
| tRNA- | 26571936 | CCCGCATTCTCCA | |
| Ala- | (−) | | |
| AGC- | | | |
| 11- | | | |
| 1 | | | |
| Homo_ | chr6: | GGGGAATTAGCTCAAGTGGTAGAGCGCTTG | 171 |
| sapiens_ | 26682487- | CTTAGCATGCAAGAGGTAGTGGGATCGATG | |
| tRNA- | 26682559 | CCCACATTCTCCA | |
| Ala- | (+) | | |
| AGC- | | | |
| 12- | | | |
| 1 | | | |
| Homo_ | chr6: | GGGGAATTAGCTCAAGTGGTAGAGCGCTTG | 172 |
| sapiens_ | 26819109- | CTTAGCATGCAAGAGGTAGTGGGATCGATG | |
| tRNA- | 26819181 | CCCACATTCTCCA | |
| Ala- | (−) | | |
| AGC- | | | |
| 12- | | | |
| 2 | | | |
| Homo_ | chr6: | GGGGAATTAGCTCAAGTGGTAGAGCGCTTG | 173 |
| sapiens_ | 57856401- | CTTAGCATGCAAGAGGTAGTGGGATCGATG | |
| tRNA- | 57856473 | CCCACATTCTCCA | |
| Ala- | (−) | | |
| AGC- | | | |
| 12- | | | |
| 3 | | | |
| Homo_ | chr6: | GGGGAATTAGCTCAAGCGGTAGAGCGCTTG | 174 |
| sapiens_ | 26705377- | CTTAGCATGCAAGAGGTAGTGGGATCGATG | |
| tRNA- | 26705449 | CCCACATTCTCCA | |
| Ala- | (+) | | |
| AGC- | | | |
| 13- | | | |
| 1 | | | |
| Homo_ | chr6: | GGGGAATTAGCTCAAGCGGTAGAGCGCTTG | 175 |
| sapiens_ | 57838350- | CTTAGCATGCAAGAGGTAGTGGGATCGATG | |
| tRNA- | 57838422 | CCCACATTCTCCA | |
| Ala- | (−) | | |
| AGC- | | | |
| 13- | | | |
| 2 | | | |
| Homo_ | chr6: | GGGGAATTAGCTCAAGCGGTAGAGCGCTTG | 176 |
| sapiens_ | 26796209- | CTTAGCATGCAAGAGGTAGTGGGATCGATG | |
| tRNA- | 26796281 | CCCACATTCTCCA | |
| Ala- | (−) | | |
| AGC- | | | |
| 13- | | | |
| 3 | | | |
| Homo_ | chr6: | GGGGAATTAGCTCAAGTGGTAGAGCGCTTG | 177 |
| sapiens_ | 26673362- | CTTAGCATGCAAGAGGTAGTGGGATCAATG | |
| tRNA- | 26673434 | CCCACATTCTCCA | |
| Ala- | (+) | | |
| AGC- | | | |
| 14- | | | |
| 1 | | | |
| Homo_ | chr6: | GGGGAATTAGCTCAAGTGGTAGAGCGCTTG | 178 |
| sapiens_ | 26828227- | CTTAGCATGCAAGAGGTAGTGGGATCAATG | |
| tRNA- | 26828299 | CCCACATTCTCCA | |
| Ala- | (−) | | |
| AGC- | | | |
| 14- | | | |
| 2 | | | |
| Homo_ | chr14: | GGGGAATTAGCTCAAGTGGTAGAGCGCTCG | 179 |
| sapiens_ | 88979098- | CTTAGCATGCGAGAGGTAGTGGGATCGATG | |
| tRNA- | 88979170 | CCCGCATTCTCCA | |
| Ala- | (+) | | |
| AGC- | | | |
| 15- | | | |
| 1 | | | |
| Homo_ | chr6: | GGGGAATTAGCCCAAGTGGTAGAGCGCTTG | 180 |
| sapiens_ | 57870345- | CTTAGCATGCAAGAGGTAGTGGGATCGATG | |
| tRNA- | 57870417 | CCCACATTCTCCA | |
| Ala- | (−) | | |
| AGC- | | | |
| 16- | | | |
| 1 | | | |
| Homo_ | chr6: | GGGGGTGTAGCTCAGTGGTAGAGCGCGTGC | 181 |
| sapiens_ | 28838444- | TTAGCATGCACGAGGCCCCGGGTTCAATCC | |
| tRNA- | 28838515 | CCGGCACCTCCA | |
| Ala- | (−) | | |
| AGC- | | | |
| 2- | | | |
| 1 | | | |
| Homo_ | chr6: | GGGGGTGTAGCTCAGTGGTAGAGCGCGTGC | 182 |
| sapiens_ | 28863685- | TTAGCATGCACGAGGCCCCGGGTTCAATCC | |
| tRNA- | 28863756 | CCGGCACCTCCA | |
| Ala- | (−) | | |
| AGC- | | | |
| 2- | | | |
| 2 | | | |
| Homo_ | chr6: | GGGGAATTAGCTCAAGCGGTAGAGCGCTTG | 183 |
| sapiens_ | 57815974- | CTTAGCATGCAAGAGGTAGCAGGATCGATG | |
| tRNA- | 57816046 | CCTGCATTCTCCA | |
| Ala- | (−) | | |
| AGC- | | | |
| 24- | | | |
| 1 | | | |
| Homo_ | chr6: | GGGGGTGTAGCTCAGTGGTAGAGCGCGTGC | 184 |
| sapie | 2860 | TTAGCATGTACGAGGTCCCGGGTTCAATCC | |
| ns_ | 7156- | CCGGCACCTCCA | |
| tRNA- | 28607227 | | |
| Ala- | (+) | | |
| AGC- | | | |
| 3- | | | |
| 1 | | | |
| Homo_ | chr6: | GGGGATGTAGCTCAGTGGTAGAGCGCATGC | 185 |
| sapiens_ | 28658237- | TTAGCATGCATGAGGTCCCGGGTTCGATCC | |
| tRNA- | 28658308 | CCAGCATCTCCA | |
| Ala- | (−) | | |
| AGC- | | | |
| 4- | | | |
| 1 | | | |
| Homo_ | chr6: | GGGGGTGTAGCTCAGTGGTAGAGCGCGTGC | 186 |
| sapiens_ | 28710589- | TTAGCATGCACGAGGCCCTGGGTTCAATCC | |
| tRNA- | 28710660 | CCAGCACCTCCA | |
| Ala- | (+) | | |
| AGC- | | | |
| 5- | | | |
| 1 | | | |
| Homo_ | chr6: | GGGGGTATAGCTCAGCGGTAGAGCGCGTGC | 187 |
| sapiens_ | 28812072- | TTAGCATGCACGAGGTCCTGGGTTCAATCC | |
| tRNA- | 28812143 | CCAATACCTCCA | |
| Ala- | (−) | | |
| AGC- | | | |
| 6- | | | |
| 1 | | | |
| Homo_ | chr6: | GGGGGTGTAGCTCAGTGGTAGAGCGCGTGC | 188 |
| sapiens_ | 28719704- | TTAGCATGCACGAGGCCCCGGGTTCAATCC | |
| tRNA- | 28719775+) | CCGGCACCTCCA | |
| Ala- | | | |
| AGC- | | | |
| 7- | | | |
| 1 | | | |
| Homo_ | chr2: | GGGGGATTAGCTCAAATGGTAGAGCGCTCG | 189 |
| sapiens_ | 27051214- | CTTAGCATGCGAGAGGTAGCGGGATCGATG | |
| tRNA- | 27051286 | CCCGCATCCTCCA | |
| Ala- | (+) | | |
| AGC- | | | |
| 8- | | | |
| 1 | | | |
| Homo_ | chr8: | GGGGGATTAGCTCAAATGGTAGAGCGCTCG | 190 |
| sapiens_ | 66114189- | CTTAGCATGCGAGAGGTAGCGGGATCGATG | |
| tRNA- | 66114261 | CCCGCATCCTCCA | |
| Ala- | | | |
| AGC- | | | |
| 8- | | | |
| 2 | | | |
| Homo_ | chr6: | GGGGAATTAGCTCAGGCGGTAGAGCGCTCG | 191 |
| sapiens_ | 26730534- | CTTAGCATGCGAGAGGTAGCGGGATCGACG | |
| tRNA- | 26730606 | CCCGCATTCTCCA | |
| Ala- | (+) | | |
| AGC- | | | |
| 9- | | | |
| 1 | | | |
| Homo_ | chr6: | GGGGAATTAGCTCAGGCGGTAGAGCGCTCG | 192 |
| sapiens_ | 26771080- | CTTAGCATGCGAGAGGTAGCGGGATCGACG | |
| tRNA- | 26771152 | CCCGCATTCTCCA | |
| Ala- | (−) | | |
| AGC- | | | |
| 9- | | | |
| 2 | | | |
| Homo_ | chr6: | GGGGATGTAGCTCAGTGGTAGAGCGCATGC | 193 |
| sapiens_ | 26553503- | TTCGCATGTATGAGGTCCCGGGTTCGATCC | |
| tRNA- | 26553574 | CCGGCATCTCCA | |
| Ala- | (+) | | |
| CGC- | | | |
| 1- | | | |
| 1 | | | |
| Homo_ | chr6: | GGGGATGTAGCTCAGTGGTAGAGCGCATGC | 194 |
| sapiens_ | 28673836- | TTCGCATGTATGAGGCCCCGGGTTCGATCC | |
| tRNA- | 28673907 | CCGGCATCTCCA | |
| Ala- | (−) | | |
| CGC- | | | |
| 2- | | | |
| 1 | | | |
| Homo_ | chr2: | GGGGATGTAGCTCAGTGGTAGAGCGCGCGC | 195 |
| sapiens_ | 156400769- | TTCGCATGTGTGAGGTCCCGGGTTCAATCC | |
| tRNA- | 156400840 | CCGGCATCTCCA | |
| Ala- | (+) | | |
| CGC- | | | |
| 3- | | | |
| 1 | | | |
| Homo_ | chr6: | GGGGGTGTAGCTCAGTGGTAGAGCGCGTGC | 196 |
| sapiens_ | 28729315- | TTCGCATGTACGAGGCCCCGGGTTCGACCC | |
| tRNA- | 28729386 | CCGGCTCCTCCA | |
| Ala- | (+) | | |
| CGC- | | | |
| 4- | | | |
| 1 | | | |
| Homo_ | chr6: | GGGGGTGTAGCTCAGTGGTAGAGCGCATGC | 197 |
| sapiens_ | 28789770- | TTTGCATGTATGAGGTCCCGGGTTCGATCC | |
| tRNA- | 28789841 | CCGGCACCTCCA | |
| Ala- | (−) | | |
| TGC- | | | |
| 1- | | | |
| 1 | | | |
| Homo_ | chr6: | GGGGATGTAGCTCAGTGGTAGAGCGCATGC | 198 |
| sapiens_ | 28643445- | TTTGCATGTATGAGGTCCCGGGTTCGATCC | |
| tRNA- | 28643516 | CCGGCATCTCCA | |
| Ala- | (+) | | |
| TGC- | | | |
| 2- | | | |
| 1 | | | |
| Homo_ | chr5: | GGGGATGTAGCTCAGTGGTAGAGCGCATGC | 199 |
| sapiens_ | 181206868- | TTTGCATGTATGAGGCCCCGGGTTCGATCC | |
| tRNA- | 181206939 | CCGGCATCTCCA | |
| Ala- | (+) | | |
| TGC- | | | |
| 3- | | | |
| 1 | | | |
| Homo_ | chr12: | GGGGATGTAGCTCAGTGGTAGAGCGCATGC | 200 |
| sapiens_ | 124921755- | TTTGCATGTATGAGGCCCCGGGTTCGATCC | |
| tRNA- | 124921826 | CCGGCATCTCCA | |
| Ala- | (−) | | |
| TGC- | | | |
| 3- | | | |
| 2 | | | |
| Homo_ | chr12: | GGGGATGTAGCTCAGTGGTAGAGCGCATGC | 201 |
| sapiens_ | 124939966- | TTTGCACGTATGAGGCCCCGGGTTCAATCC | |
| tRNA- | 124940037 | CCGGCATCTCCA | |
| Ala- | (+) | | |
| TGC- | | | |
| 4- | | | |
| 1 | | | |
| Homo_ | chr6: | GGGGGTGTAGCTCAGTGGTAGAGCGCATGC | 202 |
| sapiens_ | 28817235- | TTTGCATGTATGAGGCCTCGGGTTCGATCC | |
| tRNA- | 28817306 | CCGACACCTCCA | |
| Ala- | (−) | | |
| TGC- | | | |
| 5- | | | |
| 1 | | | |
| Homo_ | chr6: | GGGGGTGTAGCTCAGTGGTAGAGCACATGC | 203 |
| sapiens_ | 28758364- | TTTGCATGTGTGAGGCCCCGGGTTCGATCC | |
| tRNA- | 28758435 | CCGGCACCTCCA | |
| Ala- | (−) | | |
| TGC- | | | |
| 6- | | | |
| 1 | | | |
| Homo_ | chr6: | GGGGGTGTAGCTCAGTGGTAGAGCGCATGC | 204 |
| sapiens_ | 28802800- | TTTGCATGTATGAGGCCTCGGTTCGATCCC | |
| tRNA- | 28802870 | CGACACCTCCA | |
| Ala- | (−) | | |
| TGC- | | | |
| 7- | | | |
| 1 | | | |
| Homo_ | chr6: | GGGCCAGTGGCGCAATGGATAACGCGTCTG | 205 |
| sapiens_ | 26328140- | ACTACGGATCAGAAGATTCCAGGTTCGACT | |
| tRNA- | 26328212 | CCTGGCTGGCTCG | |
| Arg- | (+) | | |
| ACG- | | | |
| 1- | | | |
| 1 | | | |
| Homo_ | chr6: | GGGCCAGTGGCGCAATGGATAACGCGTCTG | 206 |
| sapiens_ | 26537498- | ACTACGGATCAGAAGATTCCAGGTTCGACT | |
| tRNA- | 26537570 | CCTGGCTGGCTCG | |
| Arg- | (+) | | |
| ACG- | | | |
| 1- | | | |
| 2 | | | |
| Homo_ | chr14: | GGGCCAGTGGCGCAATGGATAACGCGTCTG | 207 |
| sapiens_ | 22929701- | ACTACGGATCAGAAGATTCCAGGTTCGACT | |
| tRNA- | 22929773 | CCTGGCTGGCTCG | |
| Arg- | (+) | | |
| ACG- | | | |
| 1- | | | |
| 3 | | | |
| Homo_ | chr3: | GGGCCAGTGGCGCAATGGATAACGCGTCTG | 208 |
| sapiens_ | 45688999- | ACTACGGATCAGAAGATTCTAGGTTCGACT | |
| tRNA- | 45689071 | CCTGGCTGGCTCG | |
| Arg- | (−) | | |
| ACG- | | | |
| 2- | | | |
| 1 | | | |
| Homo_ | chr6: | GGGCCAGTGGCGCAATGGATAACGCGTCTG | 209 |
| sapiens_ | 27213844- | ACTACGGATCAGAAGATTCTAGGTTCGACT | |
| tRNA- | 27213916 | CCTGGCTGGCTCG | |
| Arg- | (−) | | |
| ACG- | | | |
| 2- | | | |
| 2 | | | |
| Homo_ | chr6: | GGGCCAGTGGCGCAATGGATAACGCGTCTG | 210 |
| sapiens_ | 27215173- | ACTACGGATCAGAAGATTCTAGGTTCGACT | |
| tRNA- | 27215245 | CCTGGCTGGCTCG | |
| Arg- | (+) | | |
| ACG- | | | |
| 2- | | | |
| 3 | | | |
| Homo_ | chr6: | GGGCCAGTGGCGCAATGGATAACGCGTCTG | 211 |
| sapiens_ | 27670565- | ACTACGGATCAGAAGATTCTAGGTTCGACT | |
| tRNA- | 27670637 | CCTGGCTGGCTCG | |
| Arg- | (−) | | |
| ACG- | | | |
| 2- | | | |
| 4 | | | |
| Homo_ | chr6: | GGCCGCGTGGCCTAATGGATAAGGCGTCTG | 212 |
| sapiens_ | 28742952- | ATTCCGGATCAGAAGATTGAGGGTTCGAGT | |
| tRNA- | 28743024 | CCCTTCGTGGTCG | |
| Arg- | (−) | | |
| CCG- | | | |
| 1- | | | |
| 1 | | | |
| Homo_ | chr6: | GGCCGCGTGGCCTAATGGATAAGGCGTCTG | 213 |
| sapiens_ | 28881388- | ATTCCGGATCAGAAGATTGAGGGTTCGAGT | |
| tRNA- | 28881460 | CCCTTCGTGGTCG | |
| Arg- | (+) | | |
| CCG- | | | |
| 1- | | | |
| 2 | | | |
| Homo_ | chr16: | GGCCGCGTGGCCTAATGGATAAGGCGTCTG | 214 |
| sapiens_ | 3150674- | ATTCCGGATCAGAAGATTGAGGGTTCGAGT | |
| tRNA- | 3150746 | CCCTTCGTGGTCG | |
| Arg- | (+) | | |
| CCG- | | | |
| 1- | | | |
| 3 | | | |
| Homo_ | chr17: | GACCCAGTGGCCTAATGGATAAGGCATCAG | 215 |
| sapiens_ | 68019897- | CCTCCGGAGCTGGGGATTGTGGGTTCGAGT | |
| tRNA- | 68019969 | CCCATCTGGGTCG | |
| Arg- | (−) | | |
| CCG- | | | |
| 2- | | | |
| 1 | | | |
| Homo_ | chr17: | GCCCCAGTGGCCTAATGGATAAGGCACTGG | 216 |
| sapiens_ | 75033906- | CCTCCTAAGCCAGGGATTGTGGGTTCGAGT | |
| tRNA- | 75033978 | CCCACCTGGGGTA | |
| Arg- | (+) | | |
| CCT- | | | |
| 1- | | | |
| 1 | | | |
| Homo_ | chr17: | GCCCCAGTGGCCTAATGGATAAGGCACTGG | 217 |
| sapiens_ | 75034431- | CCTCCTAAGCCAGGGATTGTGGGTTCGAGT | |
| tRNA- | 75034503 | CCCACCTGGGGTG | |
| Arg- | (−) | | |
| CCT- | | | |
| 2- | | | |
| 1 | | | |
| Homo_ | chr16: | GCCCCGGTGGCCTAATGGATAAGGCATTGG | 218 |
| sapiens_ | 3152900- | CCTCCTAAGCCAGGGATTGTGGGTTCGAGT | |
| tRNA- | 3152972 | CCCACCCGGGGTA | |
| Arg- | (+) | | |
| CCT- | | | |
| 3- | | | |
| 1 | | | |
| Homo_ | chr7: | GCCCCAGTGGCCTAATGGATAAGGCATTGG | 219 |
| sapiens_ | 139340700- | CCTCCTAAGCCAGGGATTGTGGGTTCGAGT | |
| tRNA- | 139340772 | CCCATCTGGGGTG | |
| Arg- | (+) | | |
| CCT- | | | |
| 4- | | | |
| 1 | | | |
| Homo_ | chr16: | GCCCCAGTGGCCTGATGGATAAGGTACTGG | 220 |
| sapiens_ | 3193918- | CCTCCTAAGCCAGGGATTGTGGGTTCGAGT | |
| tRNA- | 3193990 | TCCACCTGGGGTA | |
| Arg- | (+) | | |
| CCT- | | | |
| 5- | | | |
| 1 | | | |
| Homo_ | chr15: | GGCCGCGTGGCCTAATGGATAAGGCGTCTG | 221 |
| sapiens_ | 89335073- | ACTTCGGATCAGAAGATTGCAGGTTCGAGT | |
| tRNA- | 89335145 | CCTGCCGCGGTCG | |
| Arg- | (+) | | |
| TCG- | | | |
| 1- | | | |
| 1 | | | |
| Homo_ | chr6: | GACCACGTGGCCTAATGGATAAGGCGTCTG | 222 |
| sapiens_ | 26322818- | ACTTCGGATCAGAAGATTGAGGGTTCGAAT | |
| tRNA- | 26322890 | CCCTCCGTGGTTA | |
| Arg- | (+) | | |
| TCG- | | | |
| 2- | | | |
| 1 | | | |
| Homo_ | chr17: | GACCGCGTGGCCTAATGGATAAGGCGTCTG | 223 |
| sapiens_ | 75035113- | ACTTCGGATCAGAAGATTGAGGGTTCGAGT | |
| tRNA- | 75035185 | CCCTTCGTGGTCG | |
| Arg- | (+) | | |
| TCG- | | | |
| 3- | | | |
| 1 | | | |
| Homo_ | chr6: | GACCACGTGGCCTAATGGATAAGGCGTCTG | 224 |
| sapiens_ | 26299677- | ACTTCGGATCAGAAGATTGAGGGTTCGAAT | |
| tRNA- | 26299749 | CCCTTCGTGGTTA | |
| Arg- | (+) | | |
| TCG- | | | |
| 4- | | | |
| 1 | | | |
| Homo_ | chr6: | GACCACGTGGCCTAATGGATAAGGCGTCTG | 225 |
| sapiens_ | 28543114- | ACTTCGGATCAGAAGATTGAGGGTTCGAAT | |
| tRNA- | 28543186 | CCCTTCGTGGTTG | |
| Arg- | (−) | | |
| TCG- | | | |
| 5- | | | |
| 1 | | | |
| Homo_ | chr9: | GGCCGTGTGGCCTAATGGATAAGGCGTCTG | 226 |
| sapiens_ | 110198523- | ACTTCGGATCAAAAGATTGCAGGTTTGAGT | |
| tRNA- | 110198595 | TCTGCCACGGTCG | |
| Arg- | (+) | | |
| TCG- | | | |
| 6- | | | |
| 1 | | | |
| Homo_ | chr1: | GGCTCCGTGGCGCAATGGATAGCGCATTGG | 227 |
| sapiens_ | 93847573- | ACTTCTAGAGGCTGAAGGCATTCAAAGGTT | |
| tRNA- | 93847657 | CCGGGTTCGAGTCCCGGCGGAGTCG | |
| Arg- | (+) | | |
| TCT- | | | |
| 1- | | | |
| 1 | | | |
| Homo_ | chr17: | GGCTCTGTGGCGCAATGGATAGCGCATTGG | 228 |
| sapiens_ | 8120925- | ACTTCTAGTGACGAATAGAGCAATTCAAAG | |
| tRNA- | 8121012 | GTTGTGGGTTCGAATCCCACCAGAGTCG | |
| Arg- | (+) | | |
| TCT- | | | |
| 2- | | | |
| 1 | | | |
| Homo_ | chr9: | GGCTCTGTGGCGCAATGGATAGCGCATTGG | 229 |
| sapiens_ | 128340076- | ACTTCTAGCTGAGCCTAGTGTGGTCATTCA | |
| tRNA- | 128340166 | AAGGTTGTGGGTTCGAGTCCCACCAGAGTC | |
| Arg- | (−) | G | |
| TCT- | | | |
| 3- | | | |
| 1 | | | |
| Homo_ | chr11: | GGCTCTGTGGCGCAATGGATAGCGCATTGG | 230 |
| sapiens_ | 59551294- | ACTTCTAGATAGTTAGAGAAATTCAAAGGT | |
| tRNA- | 59551379 | TGTGGGTTCGAGTCCCACCAGAGTCG | |
| Arg- | (+) | | |
| TCT- | | | |
| 3- | | | |
| 2 | | | |
| Homo_ | chr1: | GTCTCTGTGGCGCAATGGACGAGCGCGCTG | 231 |
| sapiens_ | 159141611- | GACTTCTAATCCAGAGGTTCCGGGTTCGAG | |
| tRNA- | 159141684 | TCCCGGCAGAGATG | |
| Arg- | (−) | | |
| TCT- | | | |
| 4- | | | |
| 1 | | | |
| Homo_ | chr6: | GGCTCTGTGGCGCAATGGATAGCGCATTGG | 232 |
| sapiens_ | 27562184- | ACTTCTAGCCTAAATCAAGAGATTCAAAGG | |
| tRNA- | 27562270 | TTGCGGGTTCGAGTCCCTCCAGAGTCG | |
| Arg- | (+) | | |
| TCT- | | | |
| 5- | | | |
| 1 | | | |
| Homo_ | chr1: | GTCTCTGTGGCGCAATCGGTTAGCGCGTTC | 233 |
| sapiens_ | 161540241- | GGCTGTTAACCGAAAGGTTGGTGGTTCGAT | |
| tRNA- | 161540314 | CCCACCCAGGGACG | |
| Asn- | (+) | | |
| GTT- | | | |
| 1- | | | |
| 1 | | | |
| Homo_ | chr1: | GTCTCTGTGGCGCAATCGGCTAGCGCGTTT | 234 |
| sapiens_ | 145129239- | GGCTGTTAACTAAAAGGTTGGCGGTTCGAA | |
| tRNA- | 145129312 | CCCACCCAGAGGCG | |
| Asn- | (+) | | |
| GTT- | | | |
| 10- | | | |
| 1 | | | |
| Homo_ | chr1: | GTCTCTGTGGTGCAATCGGTTAGCGCGTTC | 235 |
| sapiens_ | 120952291- | CGCTGTTAACCGAAAGCTTGGTGGTTCGAG | |
| tRNA- | 120952364 | CCCACCCAGGGATG | |
| Asn- | (−) | | |
| GTT- | | | |
| 11- | | | |
| 1 | | | |
| Homo_ | chr1: | GTCTCTGTGGTGCAATCGGTTAGCGCGTTC | 236 |
| sapiens_ | 149646451- | CGCTGTTAACCGAAAGCTTGGTGGTTCGAG | |
| tRNA- | 149646524 | CCCACCCAGGGATG | |
| Asn- | (−) | | |
| GTT- | | | |
| 11- | | | |
| 2 | | | |
| Homo_ | chr1: | GTCTCTGTGGCGCAATCGGCTAGCGCGTTT | 237 |
| sapiens_ | 143831708- | GGCTGTTAACTAAAAAGTTGGTGGTTCGAA | |
| tRNA- | 143831781 | CACACCCAGAGGCG | |
| Asn- | (−) | | |
| GTT- | | | |
| 12- | | | |
| 1 | | | |
| Homo_ | chr1: | GTCTCTGTGGCGCAATCGGTTAGCGCGTTC | 238 |
| sapiens_ | 148529257- | GGCTGTTAACCGAAAGGTTGGTGGTTCGAG | |
| tRNA- | 148529330 | CCCACCCAGGGACG | |
| Asn- | (+) | | |
| GTT- | | | |
| 2- | | | |
| 1 | | | |
| Homo_ | chr1: | GTCTCTGTGGCGCAATCGGTTAGCGCGTTC | 239 |
| sapiens_ | 161428077- | GGCTGTTAACCGAAAGGTTGGTGGTTCGAG | |
| tRNA- | 161428150 | CCCACCCAGGGACG | |
| Asn- | (−) | | |
| GTT- | | | |
| 2- | | | |
| 2 | | | |
| Homo_ | chr10: | GTCTCTGTGGCGCAATCGGTTAGCGCGTTC | 240 |
| sapiens_ | 22229509- | GGCTGTTAACCGAAAGGTTGGTGGTTCGAG | |
| tRNA- | 22229582 | CCCACCCAGGGACG | |
| Asn- | (−) | | |
| GTT- | | | |
| 2- | | | |
| 3 | | | |
| Homo_ | chr13: | GTCTCTGTGGCGCAATCGGTTAGCGCGTTC | 241 |
| sapiens_ | 30673964- | GGCTGTTAACCGAAAGGTTGGTGGTTCGAG | |
| tRNA- | 30674037 | CCCACCCAGGGACG | |
| Asn- | (−) | | |
| GTT- | | | |
| 2- | | | |
| 4 | | | |
| Homo_ | chr17: | GTCTCTGTGGCGCAATCGGTTAGCGCGTTC | 242 |
| sapiens_ | 38751781- | GGCTGTTAACCGAAAGGTTGGTGGTTCGAG | |
| tRNA- | 38751854 | CCCACCCAGGGACG | |
| Asn- | (−) | | |
| GTT- | | | |
| 2- | | | |
| 5 | | | |
| Homo_ | chr19: | GTCTCTGTGGCGCAATCGGTTAGCGCGTTC | 243 |
| sapiens_ | 1383563- | GGCTGTTAACCGAAAGGTTGGTGGTTCGAG | |
| tRNA- | 1383636 | CCCACCCAGGGACG | |
| Asn- | (+) | | |
| GTT- | | | |
| 2- | | | |
| 6 | | | |
| Homo_ | chr1: | GTCTCTGTGGCGCAATCGGTTAGCGCGTTC | 244 |
| sapiens_ | 145287766- | GGCTGTTAACCGAAAGGTTGGTGGTTCGAG | |
| tRNA- | 145287839 | CCCACCCAGGGACG | |
| Asn- | (+) | | |
| GTT- | | | |
| 2- | | | |
| 7 | | | |
| Homo_ | chr1: | GTCTCTGTGGCGCAATCGGTTAGCGCGTTC | 245 |
| sapiens_ | 144567515- | GGCTGTTAACCGAAAGGTTGGTGGTTCGAG | |
| tRNA- | 144567588 | CCCACCCAGGGACG | |
| Asn- | (−) | | |
| GTT- | | | |
| 2- | | | |
| 8 | | | |
| Homo_ | chr1: | GTCTCTGTGGCGCAATCGGTTAGCGCGTTC | 246 |
| sapiens_ | 146370101- | GGCTGTTAACCGCAAGGTTGGTGGTTCCAG | |
| tRNA- | 146370174 | CCCACCCAGGGACG | |
| Asn- | (+) | | |
| GTT- | | | |
| 24- | | | |
| 1 | | | |
| Homo_ | chr1: | GTCTCTGTGGCGCAATTGGTTAGCGCGTTC | 247 |
| sapiens_ | 149558419- | GGTTGTTAACCGTAAAGGTTGGTGGTTCGA | |
| tRNA- | 149558493 | GCCCACCCAGGAACG | |
| Asn- | (−) | | |
| GTT- | | | |
| 25- | | | |
| 1 | | | |
| Homo_ | chr1: | GTCTCTGTGGCGCAATCGGCTAGCGCTTTT | 248 |
| sapiens_ | 121048432- | GGCTGTTAACTAAAAGGTTGGTGGTTTGAA | |
| tRNA- | 121048505 | CCCACCCAGAGGCG | |
| Asn- | (−) | | |
| GTT- | | | |
| 27- | | | |
| 1 | | | |
| Homo_ | chr1: | GTCTCTGTGGCGCAATCGGTTAGCGCATTC | 249 |
| sapiens_ | 144419267- | GGCTGTTAACCGAAAGGTTGGTGGTTCGAG | |
| tRNA- | 144419340 | CCCACCCAGGGACG | |
| Asn- | (+) | | |
| GTT- | | | |
| 3- | | | |
| 1 | | | |
| Homo_ | chr1: | GTCTCTGTGGCGCAATCGGTTAGCGCGTTC | 250 |
| sapiens_ | 16889677- | GGCTGTTAACCGAAAGATTGGTGGTTCGAG | |
| tRNA- | 16889750 | CCCACCCAGGGACG | |
| Asn- | (+) | | |
| GTT- | | | |
| 4- | | | |
| 1 | | | |
| Homo_ | chr1: | GTCTCTGTGGCGCAATCGGTTAGCGCGTTC | 251 |
| sapiens_ | 16520585- | GGCTGTTAACTGAAAGGTTGGTGGTTCGAG | |
| tRNA- | 16520658 | CCCACCCAGGGACG | |
| Asn- | (−) | | |
| GTT- | | | |
| 5- | | | |
| 1 | | | |
| Homo_ | chr1: | GTCTCTGTGGCGCAATGGGTTAGCGCGTTC | 252 |
| sapiens_ | 143735920- | GGCTGTTAACCGAAAGGTTGGTGGTTCGAG | |
| tRNA- | 143735993 | CCCATCCAGGGACG | |
| Asn- | (−) | | |
| GTT- | | | |
| 6- | | | |
| 1 | | | |
| Homo_ | chr1: | GTCTCTGTGGCGTAGTCGGTTAGCGCGTTC | 253 |
| sapiens_ | 120844262- | GGCTGTTAACCGAAAGGTTGGTGGTTCGAG | |
| tRNA- | 120844335 | CCCACCCAGGAACG | |
| Asn- | (−) | | |
| GTT- | | | |
| 7- | | | |
| 1 | | | |
| Homo_ | chr1: | GTCTCTGTGGCGCAATCGGCTAGCGCGTTT | 254 |
| sapiens_ | 149740248- | GGCTGTTAACTAAAAGGTTGGTGGTTCGAA | |
| tRNA- | 149740321 | CCCACCCAGAGGCG | |
| Asn- | (−) | | |
| GTT- | | | |
| 8- | | | |
| 1 | | | |
| Homo_ | chr1: | GTCTCTGTGGCGCAATCGGTTAGCGCGTTC | 255 |
| sapiens_ | 145475381- | GGCTGTTAACTGAAAGGTTGGTGGTTCGAG | |
| tRNA- | 145475454 | CCCACCCGGGGACG | |
| Asn- | (−) | | |
| GTT- | | | |
| 9- | | | |
| 1 | | | |
| Homo_ | chr1: | GTCTCTGTGGCGCAATCGGTTAGCGCGTTC | 256 |
| sapiens_ | 148048516- | GGCTGTTAACTGAAAGGTTAGTGGTTCGAG | |
| tRNA- | 148048589 | CCCACCCGGGGACG | |
| Asn- | (−) | | |
| GTT- | | | |
| 9- | | | |
| 2 | | | |
| Homo_ | chr12: | TCCTCGTTAGTATAGTGGTTAGTATCCCCG | 257 |
| sapiens_ | 98503503- | CCTGTCACGCGGGAGACCGGGGTTCAATTC | |
| tRNA- | 98503574 | CCCGACGGGGAG | |
| Asp- | (+) | | |
| GTC- | | | |
| 1- | | | |
| 1 | | | |
| Homo_ | chr1: | TCCTCGTTAGTATAGTGGTGAGTATCCCCG | 258 |
| sapiens_ | 161440825- | CCTGTCACGCGGGAGACCGGGGTTCGATTC | |
| tRNA- | 161440896 | CCCGACGGGGAG | |
| Asp- | (−) | | |
| GTC- | | | |
| 2- | | | |
| 1 | | | |
| Homo_ | chr12: | TCCTCGTTAGTATAGTGGTGAGTATCCCCG | 259 |
| sapiens_ | 124939647- | CCTGTCACGCGGGAGACCGGGGTTCGATTC | |
| tRNA- | 124939718 | CCCGACGGGGAG | |
| Asp- | (−) | | |
| GTC- | | | |
| 2- | | | |
| 10 | | | |
| Homo_ | chr17: | TCCTCGTTAGTATAGTGGTGAGTATCCCCG | 260 |
| sapiens_ | 8222238- | CCTGTCACGCGGGAGACCGGGGTTCGATTC | |
| tRNA- | 8222309 | CCCGACGGGGAG | |
| Asp- | (−) | | |
| GTC- | | | |
| 2- | | | |
| 11 | | | |
| Homo_ | chr1: | TCCTCGTTAGTATAGTGGTGAGTATCCCCG | 261 |
| sapiens_ | 161448243- | CCTGTCACGCGGGAGACCGGGGTTCGATTC | |
| tRNA- | 161448314 | CCCGACGGGGAG | |
| Asp- | (−) | | |
| GTC- | | | |
| 2- | | | |
| 2 | | | |
| Homo_ | chr1: | TCCTCGTTAGTATAGTGGTGAGTATCCCCG | 262 |
| sapiens_ | 161455624- | CCTGTCACGCGGGAGACCGGGGTTCGATTC | |
| tRNA- | 161455695 | CCCGACGGGGAG | |
| Asp- | (−) | | |
| GTC- | | | |
| 2- | | | |
| 3 | | | |
| Homo_ | chr1: | TCCTCGTTAGTATAGTGGTGAGTATCCCCG | 263 |
| sapiens_ | 161463034- | CCTGTCACGCGGGAGACCGGGGTTCGATTC | |
| tRNA- | 161463105 | CCCGACGGGGAG | |
| Asp- | (−) | | |
| GTC- | | | |
| 2- | | | |
| 4 | | | |
| Homo_ | chr1: | TCCTCGTTAGTATAGTGGTGAGTATCCCCG | 264 |
| sapiens_ | 161470415- | CCTGTCACGCGGGAGACCGGGGTTCGATTC | |
| tRNA- | 161470486 | CCCGACGGGGAG | |
| Asp- | (−) | | |
| GTC- | | | |
| 2- | | | |
| 5 | | | |
| Homo_ | chr6: | TCCTCGTTAGTATAGTGGTGAGTATCCCCG | 265 |
| sapiens_ | 27479674- | CCTGTCACGCGGGAGACCGGGGTTCGATTC | |
| tRNA- | 27479745 | CCCGACGGGGAG | |
| Asp- | (+) | | |
| GTC- | | | |
| 2- | | | |
| 6 | | | |
| Homo_ | chr6: | TCCTCGTTAGTATAGTGGTGAGTATCCCCG | 266 |
| sapiens_ | 27503744- | CCTGTCACGCGGGAGACCGGGGTTCGATTC | |
| tRNA- | 27503815 | CCCGACGGGGAG | |
| Asp- | (+) | | |
| GTC- | | | |
| 2- | | | |
| 7 | | | |
| Homo_ | chr12: | TCCTCGTTAGTATAGTGGTGAGTATCCCCG | 267 |
| sapiens_ | 96036021- | CCTGTCACGCGGGAGACCGGGGTTCGATTC | |
| tRNA- | 96036092 | CCCGACGGGGAG | |
| Asp- | (+) | | |
| GTC- | | | |
| 2- | | | |
| 8 | | | |
| Homo_ | chr12: | TCCTCGTTAGTATAGTGGTGAGTATCCCCG | 268 |
| sapiens_ | 124927345- | CCTGTCACGCGGGAGACCGGGGTTCGATTC | |
| tRNA- | 124927416 | CCCGACGGGGAG | |
| Asp- | (−) | | |
| GTC- | | | |
| 2- | | | |
| 9 | | | |
| Homo_ | chr6: | TCCTCGTTAGTATAGTGGTGAGTGTCCCCG | 269 |
| sapiens_ | 27583457- | TCTGTCACGCGGGAGACCGGGGTTCGATTC | |
| tRNA- | 27583528 | CCCGACGGGGAG | |
| Asp- | (−) | | |
| GTC- | | | |
| 3- | | | |
| 1 | | | |
| Homo_ | chr7: | GGGGGCATAGCTCAGTGGTAGAGCATTTGA | 270 |
| sapiens_ | 149310190- | CTGCAGATCAAGAGGTCCCTGGTTCAAATC | |
| tRNA- | 149310261 | CAGGTGCCCCCT | |
| Cys- | (+) | | |
| GCA- | | | |
| 1- | | | |
| 1 | | | |
| Homo_ | chr7: | GGGGGTATAGCTCAGGGGTAGAGCATTTGA | 271 |
| sapiens_ | 149377510- | CTGCAGATCAAGAGGTCCCTGGTTCAAATC | |
| tRNA- | 149377581 | CAGGTGCCCCCC | |
| Cys- | (−) | | |
| GCA- | | | |
| 10- | | | |
| 1 | | | |
| Homo_ | chr7: | GGGGGTATAGCTTAGCGGTAGAGCATTTGA | 272 |
| sapiens_ | 149415138- | CTGCAGATCAAGAGGTCCCCGGTTCAAATC | |
| tRNA- | 149415209 | CGGGTGCCCCCT | |
| Cys- | (−) | | |
| GCA- | | | |
| 11- | | | |
| 1 | | | |
| Homo_ | chr7: | GGGGGTATAGCTTAGGGGTAGAGCATTTGA | 273 |
| sapiens_ | 149646955- | CTGCAGATCAAAAGGTCCCTGGTTCAAATC | |
| tRNA- | 149647026 | CAGGTGCCCCTT | |
| Cys- | (−) | | |
| GCA- | | | |
| 12- | | | |
| 1 | | | |
| Homo_ | chr7: | GGGGGTATAGCTCAGGGGTAGAGCATTTGA | 274 |
| sapiens_ | 149355675- | CTGCAGATCAAGAGGTCCCCAGTTCAAATC | |
| tRNA- | 149355746 | TGGGTGCCCCCT | |
| Cys- | (−) | | |
| GCA- | | | |
| 13- | | | |
| 1 | | | |
| Homo_ | chr17: | GGGGGTATAGCTCAGGGGTAGAGCATTTGA | 275 |
| sapiens_ | 38861684- | CTGCAGATCAAGAAGTCCCCGGTTCAAATC | |
| tRNA- | 38861755 | CGGGTGCCCCCT | |
| Cys- | (−) | | |
| GCA- | | | |
| 14- | | | |
| 1 | | | |
| Homo_ | chr7: | GGGGGTATAGCTCAGGGGTAGAGCATTTGA | 276 |
| sapiens_ | 149584725- | CTGCAGATCAAGAGGTCTCTGGTTCAAATC | |
| tRNA- | 149584796 | CAGGTGCCCCCT | |
| Cys- | (+) | | |
| GCA- | | | |
| 15- | | | |
| 1 | | | |
| Homo_ | chr7: | GGGGGTATAGCTCAGGGGTAGAGCACTTGA | 277 |
| sapiens_ | 149546540- | CTGCAGATCAAGAAGTCCTTGGTTCAAATC | |
| tRNA- | 149546611 | CAGGTGCCCCCT | |
| Cys- | (+) | | |
| GCA- | | | |
| 16- | | | |
| 1 | | | |
| Homo_ | chr7: | GGGGATATAGCTCAGGGGTAGAGCATTTGA | 278 |
| sapiens_ | 149691181- | CTGCAGATCAAGAGGTCCCCGGTTCAAATC | |
| tRNA- | 149691252 | CGGGTGCCCCCC | |
| Cys- | (−) | | |
| GCA- | | | |
| 17- | | | |
| 1 | | | |
| Homo_ | chr7: | GGGGGTATAGTTCAGGGGTAGAGCATTTGA | 279 |
| sapiens_ | 149375759- | CTGCAGATCAAGAGGTCCCTGGTTCAAATC | |
| tRNA- | 149375830 | CAGGTGCCCCCT | |
| Cys- | (−) | | |
| GCA- | | | |
| 18- | | | |
| 1 | | | |
| Homo_ | chr7: | GGGGGTATAGCTCAGGGGTAGAGCATTTGA | 280 |
| sapiens_ | 149613065- | CTGCAAATCAAGAGGTCCCTGATTCAAATC | |
| tRNA- | 149613136 | CAGGTGCCCCCT | |
| Cys- | (−) | | |
| GCA- | | | |
| 19- | | | |
| 1 | | | |
| Homo_ | chr4: | GGGGGTATAGCTCAGTGGTAGAGCATTTGA | 281 |
| sapiens_ | 123508850- | CTGCAGATCAAGAGGTCCCCGGTTCAAATC | |
| tRNA- | 123508921 | CGGGTGCCCCCT | |
| Cys- | (−) | | |
| GCA- | | | |
| 2- | | | |
| 1 | | | |
| Homo_ | chr17: | GGGGGTATAGCTCAGTGGTAGAGCATTTGA | 282 |
| sapiens_ | 38867645- | CTGCAGATCAAGAGGTCCCCGGTTCAAATC | |
| tRNA- | 38867716 | CGGGTGCCCCCT | |
| Cys- | (+) | | |
| GCA- | | | |
| 2- | | | |
| 2 | | | |
| Homo_ | chr17: | GGGGGTATAGCTCAGTGGTAGAGCATTTGA | 283 |
| sapiens_ | 39153734- | CTGCAGATCAAGAGGTCCCCGGTTCAAATC | |
| tRNA- | 39153805 | CGGGTGCCCCCT | |
| Cys- | (−) | | |
| GCA- | | | |
| 2- | | | |
| 3 | | | |
| Homo_ | chr17: | GGGGGTATAGCTCAGTGGTAGAGCATTTGA | 284 |
| sapiens_ | 39154491- | CTGCAGATCAAGAGGTCCCCGGTTCAAATC | |
| tRNA- | 39154562 | CGGGTGCCCCCT | |
| Cys- | (−) | | |
| GCA- | | | |
| 2- | | | |
| 4 | | | |
| Homo_ | chr7: | GGGCGTATAGCTCAGGGGTAGAGCATTTGA | 285 |
| sapiens_ | 149597955- | CTGCAGATCAAGAGGTCCCCAGTTCAAATC | |
| tRNA- | 149598026 | TGGGTGCCCCCT | |
| Cys- | (+) | | |
| GCA- | | | |
| 20- | | | |
| 1 | | | |
| Homo_ | chr7: | GGGGGTATAGCTCACAGGTAGAGCATTTGA | 286 |
| sapiens_ | 149664824- | CTGCAGATCAAGAGGTCCCCGGTTCAAATC | |
| tRNA- | 149664895 | TGGGTGCCCCCT | |
| Cys- | (+) | | |
| GCA- | | | |
| 21- | | | |
| 1 | | | |
| Homo_ | chr7: | GGGCGTATAGCTCAGGGGTAGAGCATTTGA | 287 |
| sapiens_ | 149556711- | CTGCAGATCAAGAGGTCCCCAGTTCAAATC | |
| tRNA- | 149556780 | TGGGTGCCCA | |
| Cys- | (+) | | |
| GCA- | | | |
| 22- | | | |
| 1 | | | |
| Homo_ | chr7: | GGGGGTATAGCTCACAGGTAGAGCATTTGA | 288 |
| sapiens_ | 149595214- | CTGCAGATCAAGAGGTCCCCGGTTCAAATC | |
| tRNA- | 149595285 | CGGTTACTCCCT | |
| Cys- | (−) | | |
| GCA- | | | |
| 23- | | | |
| 1 | | | |
| Homo_ | chr7: | GGGGGTATAGCTCAGGGGTAGAGCACTTGA | 289 |
| sapiens_ | 149589073- | CTGCAGATCAAGAGGTCCCTGGTTCAAATC | |
| tRNA- | 149589144 | CAGGTGCCCCCT | |
| Cys- | (−) | | |
| GCA- | | | |
| 3- | | | |
| 1 | | | |
| Homo_ | chr17: | GGGGGTATAGCTCAGTGGTAGAGCATTTGA | 290 |
| sapiens_ | 38869292- | CTGCAGATCAAGAGGTCCCTGGTTCAAATC | |
| tRNA- | 38869363 | CGGGTGCCCCCT | |
| Cys- | (−) | | |
| GCA- | | | |
| 4- | | | |
| 1 | | | |
| Homo_ | chr15: | GGGGGTATAGCTCAGTGGGTAGAGCATTTG | 291 |
| sapiens_ | 79744655- | ACTGCAGATCAAGAGGTCCCCGGTTCAAAT | |
| tRNA- | 79744727 | CCGGGTGCCCCCT | |
| Cys- | (+) | | |
| GCA- | | | |
| 5- | | | |
| 1 | | | |
| Homo_ | chr3: | GGGGGTGTAGCTCAGTGGTAGAGCATTTGA | 292 |
| sapiens_ | 132229100- | CTGCAGATCAAGAGGTCCCTGGTTCAAATC | |
| tRNA- | 132229171 | CAGGTGCCCCCT | |
| Cys- | (−) | | |
| GCA- | | | |
| 6- | | | |
| 1 | | | |
| Homo_ | chr1: | GGGGGTATAGCTCAGGTGGTAGAGCATTTG | 293 |
| sapiens_ | 93516277- | ACTGCAGATCAAGAGGTCCCCGGTTCAAAT | |
| tRNA- | 93516349 | CCGGGTGCCCCCT | |
| Cys- | (−) | | |
| GCA- | | | |
| 7- | | | |
| 1 | | | |
| Homo_ | chr14: | GGGGGTATAGCTCAGGGGTAGAGCATTTGA | 294 |
| sapiens_ | 72962971- | CTGCAGATCAAGAGGTCCCCGGTTCAAATC | |
| tRNA- | 72963042 | CGGGTGCCCCCT | |
| Cys- | (+) | | |
| GCA- | | | |
| 8- | | | |
| 1 | | | |
| Homo_ | chr3: | GGGGGTATAGCTCAGGGGTAGAGCATTTGA | 295 |
| sapiens_ | 132231798- | CTGCAGATCAAGAGGTCCCTGGTTCAAATC | |
| tRNA- | 132231869 | CAGGTGCCCCCT | |
| Cys- | (−) | | |
| GCA- | | | |
| 9- | | | |
| 1 | | | |
| Homo_ | chr7: | GGGGGTATAGCTCAGGGGTAGAGCATTTGA | 296 |
| sapiens_ | 149331129- | CTGCAGATCAAGAGGTCCCTGGTTCAAATC | |
| tRNA- | 149331200 | CAGGTGCCCCCT | |
| Cys- | (+) | | |
| GCA- | | | |
| 9- | | | |
| 2 | | | |
| Homo_ | chr7: | GGGGGTATAGCTCAGGGGTAGAGCATTTGA | 297 |
| sapiens_ | 149635687- | CTGCAGATCAAGAGGTCCCTGGTTCAAATC | |
| tRNA- | 149635758 | CAGGTGCCCCCT | |
| Cys- | (+) | | |
| GCA- | | | |
| 9- | | | |
| 3 | | | |
| Homo_ | chr7: | GGGGGTATAGCTCAGGGGTAGAGCATTTGA | 298 |
| sapiens_ | 149707669- | CTGCAGATCAAGAGGTCCCTGGTTCAAATC | |
| tRNA- | 149707740 | CAGGTGCCCCCT | |
| Cys- | (+) | | |
| GCA- | | | |
| 9- | | | |
| 4 | | | |
| Homo_ | chr6: | GGTTCCATGGTGTAATGGTTAGCACTCTGG | 299 |
| sapiens_ | 18836171- | ACTCTGAATCCAGCGATCCGAGTTCAAATC | |
| tRNA- | 18836242 | TCGGTGGAACCT | |
| Gln- | (+) | | |
| CTG- | | | |
| 1- | | | |
| 1 | | | |
| Homo_ | chr6: | GGTTCCATGGTGTAATGGTTAGCACTCTGG | 300 |
| sapiens_ | 27519529- | ACTCTGAATCCAGCGATCCGAGTTCAAATC | |
| tRNA- | 27519600 | TCGGTGGAACCT | |
| Gln- | (+) | | |
| CTG- | | | |
| 1- | | | |
| 2 | | | |
| Homo_ | chr6: | GGTTCCATGGTGTAATGGTTAGCACTCTGG | 301 |
| sapiens_ | 28941601- | ACTCTGAATCCAGCGATCCGAGTTCAAATC | |
| tRNA- | 28941672 | TCGGTGGAACCT | |
| Gln- | (−) | | |
| CTG- | | | |
| 1- | | | |
| 3 | | | |
| Homo_ | chr15: | GGTTCCATGGTGTAATGGTTAGCACTCTGG | 302 |
| sapiens_ | 65869062- | ACTCTGAATCCAGCGATCCGAGTTCAAATC | |
| tRNA- | 65869133 | TCGGTGGAACCT | |
| Gln- | (−) | | |
| CTG- | | | |
| 1- | | | |
| 4 | | | |
| Homo_ | chr17: | GGTTCCATGGTGTAATGGTTAGCACTCTGG | 303 |
| sapiens_ | 8119752- | ACTCTGAATCCAGCGATCCGAGTTCAAATC | |
| tRNA- | 8119823 | TCGGTGGAACCT | |
| Gln- | (+) | | |
| CTG- | | | |
| 1- | | | |
| 5 | | | |
| Homo_ | chr6: | GGTTCCATGGTGTAATGGTTAGCACTCTGG | 304 |
| sapiens_ | 27547752- | ACTCTGAATCCAGCGATCCGAGTTCAAGTC | |
| tRNA- | 27547823 | TCGGTGGAACCT | |
| Gln- | (−) | | |
| CTG- | | | |
| 2- | | | |
| 1 | | | |
| Homo_ | chr1: | GGTTCCATGGTGTAATGGTGAGCACTCTGG | 305 |
| sapiens_ | 145459658- | ACTCTGAATCCAGCGATCCGAGTTCGAGTC | |
| tRNA- | 145459729 | TCGGTGGAACCT | |
| Gln- | (+) | | |
| CTG- | | | |
| 3- | | | |
| 1 | | | |
| Homo_ | chr1: | GGTTCCATGGTGTAATGGTGAGCACTCTGG | 306 |
| sapiens_ | 148032790- | ACTCTGAATCCAGCGATCCGAGTTCGAGTC | |
| tRNA- | 148032861 | TCGGTGGAACCT | |
| Gln- | (+) | | |
| CTG- | | | |
| 3- | | | |
| 2 | | | |
| Homo_ | chr1: | GGTTCCATGGTGTAATGGTAAGCACTCTGG | 307 |
| sapiens_ | 148265108- | ACTCTGAATCCAGCGATCCGAGTTCGAGTC | |
| tRNA- | 148265179 | TCGGTGGAACCT | |
| Gln- | (−) | | |
| CTG- | | | |
| 4- | | | |
| 1 | | | |
| Homo_ | chr1: | GGTTCCATGGTGTAATGGTAAGCACTCTGG | 308 |
| sapiens_ | 143691474- | ACTCTGAATCCAGCGATCCGAGTTCGAGTC | |
| tRNA- | 143691545 | TCGGTGGAACCT | |
| Gln- | (+) | | |
| CTG- | | | |
| 4- | | | |
| 2 | | | |
| Homo_ | chr6: | GGTTCCATGGTGTAATGGTTAGCACTCTGG | 309 |
| sapiens_ | 27295433- | ACTCTGAATCCGGTAATCCGAGTTCAAATC | |
| tRNA- | 27295504 | TCGGTGGAACCT | |
| Gln- | (+) | | |
| CTG- | | | |
| 5- | | | |
| 1 | | | |
| Homo_ | chr6: | GGCCCCATGGTGTAATGGTCAGCACTCTGG | 310 |
| sapiens_ | 27791356- | ACTCTGAATCCAGCGATCCGAGTTCAAATC | |
| tRNA- | 27791427 | TCGGTGGGACCC | |
| Gln- | (−) | | |
| CTG- | | | |
| 6- | | | |
| 1 | | | |
| Homo_ | chr1: | GGTTCCATGGTGTAATGGTAAGCACTCTGG | 311 |
| sapiens_ | 148328812- | ACTCTGAATCCAGCCATCTGAGTTCGAGTC | |
| tRNA- | 148328883 | TCTGTGGAACCT | |
| Gln- | (+) | | |
| CTG- | | | |
| 7- | | | |
| 1 | | | |
| Homo_ | chr17: | GGTCCCATGGTGTAATGGTTAGCACTCTGG | 312 |
| sapiens_ | 49192528- | ACTTTGAATCCAGCGATCCGAGTTCAAATC | |
| tRNA- | 49192599 | TCGGTGGGACCT | |
| Gln- | (+) | | |
| TTG- | | | |
| 1- | | | |
| 1 | | | |
| Homo_ | chr6: | GGTCCCATGGTGTAATGGTTAGCACTCTGG | 313 |
| sapiens_ | 28589379- | ACTTTGAATCCAGCAATCCGAGTTCGAATC | |
| tRNA- | 28589450 | TCGGTGGGACCT | |
| Gln- | (+) | | |
| TTG- | | | |
| 2- | | | |
| 1 | | | |
| Homo_ | chr6: | GGCCCCATGGTGTAATGGTTAGCACTCTGG | 314 |
| sapiens_ | 26311196- | ACTTTGAATCCAGCGATCCGAGTTCAAATC | |
| tRNA- | 26311267 | TCGGTGGGACCT | |
| Gln- | (−) | | |
| TTG- | | | |
| 3- | | | |
| 1 | | | |
| Homo_ | chr6: | GGCCCCATGGTGTAATGGTTAGCACTCTGG | 315 |
| sapiens_ | 26311747- | ACTTTGAATCCAGCGATCCGAGTTCAAATC | |
| tRNA- | 26311818 | TCGGTGGGACCT | |
| Gln- | (−) | | |
| TTG- | | | |
| 3- | | | |
| 2 | | | |
| Homo_ | chr6: | GGCCCCATGGTGTAATGGTTAGCACTCTGG | 316 |
| sapiens_ | 27795861- | ACTTTGAATCCAGCGATCCGAGTTCAAATC | |
| tRNA- | 27795932 | TCGGTGGGACCT | |
| Gln- | (−) | | |
| TTG- | | | |
| 3- | | | |
| 3 | | | |
| Homo_ | chr6: | GGTCCCATGGTGTAATGGTTAGCACTCTGG | 317 |
| sapiens_ | 145182723- | GCTTTGAATCCAGCAATCCGAGTTCGAATC | |
| tRNA- | 145182794 | TTGGTGGGACCT | |
| Gln- | (+) | | |
| TTG- | | | |
| 4- | | | |
| 1 | | | |
| Homo_ | chr1: | TCCCTGGTGGTCTAGTGGTTAGGATTCGGC | 318 |
| sapiens_ | 146035692- | GCTCTCACCGCCGCGGCCCGGGTTCGATTC | |
| tRNA- | 146035763 | CCGGTCAGGGAA | |
| Glu- | (+) | | |
| CTC- | | | |
| 1- | | | |
| 1 | | | |
| Homo_ | chr1: | TCCCTGGTGGTCTAGTGGTTAGGATTCGGC | 319 |
| sapiens_ | 161447228- | GCTCTCACCGCCGCGGCCCGGGTTCGATTC | |
| tRNA- | 161447299 | CCGGTCAGGGAA | |
| Glu- | (−) | | |
| CTC- | | | |
| 1- | | | |
| 2 | | | |
| Homo_ | chr1: | TCCCTGGTGGTCTAGTGGTTAGGATTCGGC | 320 |
| sapiens_ | 161454608- | GCTCTCACCGCCGCGGCCCGGGTTCGATTC | |
| tRNA- | 161454679 | CCGGTCAGGGAA | |
| Glu- | (−) | | |
| CTC- | | | |
| 1- | | | |
| 3 | | | |
| Homo_ | chr1: | TCCCTGGTGGTCTAGTGGTTAGGATTCGGC | 321 |
| sapiens_ | 161462019- | GCTCTCACCGCCGCGGCCCGGGTTCGATTC | |
| tRNA- | 161462090 | CCGGTCAGGGAA | |
| Glu- | (−) | | |
| CTC- | | | |
| 1- | | | |
| 4 | | | |
| Homo_ | chr1: | TCCCTGGTGGTCTAGTGGTTAGGATTCGGC | 322 |
| sapiens_ | 161469399- | GCTCTCACCGCCGCGGCCCGGGTTCGATTC | |
| tRNA- | 161469470 | CCGGTCAGGGAA | |
| Glu- | (−) | | |
| CTC- | | | |
| 1- | | | |
| 5 | | | |
| Homo_ | chr6: | TCCCTGGTGGTCTAGTGGTTAGGATTCGGC | 323 |
| sapiens_ | 28982199- | GCTCTCACCGCCGCGGCCCGGGTTCGATTC | |
| tRNA- | 28982270 | CCGGTCAGGGAA | |
| Glu- | (+) | | |
| CTC- | | | |
| 1- | | | |
| 6 | | | |
| Homo_ | chr6: | TCCCTGGTGGTCTAGTGGTTAGGATTCGGC | 324 |
| sapiens_ | 125780247- | GCTCTCACCGCCGCGGCCCGGGTTCGATTC | |
| tRNA- | 125780318 | CCGGTCAGGGAA | |
| Glu- | (−) | | |
| CTC- | | | |
| 1- | | | |
| 7 | | | |
| Homo_ | chr1: | TCCCTGGTGGTCTAGTGGTTAGGATTCGGC | 325 |
| sapiens_ | 248874248- | GCTCTCACCGCCGCGGCCCGGGTTCGATTC | |
| tRNA- | 248874319 | CCGGTCAGGAAA | |
| Glu- | (+) | | |
| CTC- | | | |
| 2- | | | |
| 1 | | | |
| Homo_ | chr2: | TCCCATATGGTCTAGCGGTTAGGATTCCTG | 326 |
| sapiens_ | 130337128- | GTTTTCACCCAGGTGGCCCGGGTTCGACTC | |
| tRNA- | 130337199 | CCGGTATGGGAA | |
| Glu- | (−) | | |
| TTC- | | | |
| 1- | | | |
| 1 | | | |
| Homo_ | chr13: | TCCCATATGGTCTAGCGGTTAGGATTCCTG | 327 |
| sapiens_ | 41060738- | GTTTTCACCCAGGTGGCCCGGGTTCGACTC | |
| tRNA- | 41060809 | CCGGTATGGGAA | |
| Glu- | (−) | | |
| TTC- | | | |
| 1- | | | |
| 2 | | | |
| Homo_ | chr13: | TCCCACATGGTCTAGCGGTTAGGATTCCTG | 328 |
| sapiens_ | 44917927- | GTTTTCACCCAGGCGGCCCGGGTTCGACTC | |
| tRNA- | 44917998 | CCGGTGTGGGAA | |
| Glu- | (−) | | |
| TTC- | | | |
| 2- | | | |
| 1 | | | |
| Homo_ | chr15: | TCCCACATGGTCTAGCGGTTAGGATTCCTG | 329 |
| sapiens_ | 26082234- | GTTTTCACCCAGGCGGCCCGGGTTCGACTC | |
| tRNA- | 26082305 | CCGGTGTGGGAA | |
| Glu- | (−) | | |
| TTC- | | | |
| 2- | | | |
| 2 | | | |
| Homo_ | chr1: | TCCCTGGTGGTCTAGTGGCTAGGATTCGGC | 330 |
| sapiens_ | 16872583- | GCTTTCACCGCCGCGGCCCGGGTTCGATTC | |
| tRNA- | 16872654 | CCGGCCAGGGAA | |
| Glu- | (+) | | |
| TTC- | | | |
| 3- | | | |
| 1 | | | |
| Homo_ | chr1: | TCCCTGGTGGTCTAGTGGCTAGGATTCGGC | 331 |
| sapiens_ | 16535279- | GCTTTCACCGCCGCGGCCCGGGTTCGATTC | |
| tRNA- | 16535350 | CCGGTCAGGGAA | |
| Glu- | (−) | | |
| TTC- | | | |
| 4- | | | |
| 1 | | | |
| Homo_ | chr1: | TCCCTGGTGGTCTAGTGGCTAGGATTCGGC | 332 |
| sapiens_ | 161422093- | GCTTTCACCGCCGCGGCCCGGGTTCGATTC | |
| tRNA- | 161422164 | CCGGTCAGGGAA | |
| Glu- | (−) | | |
| TTC- | | | |
| 4- | | | |
| 2 | | | |
| Homo_ | chr1: | GCATTGGTGGTTCAGTGGTAGAATTCTCGC | 333 |
| sapiens_ | 16545939- | CTCCCACGCGGGAGACCCGGGTTCAATTCC | |
| tRNA- | 16546009 | CGGCCAATGCA | |
| Gly- | (−) | | |
| CCC- | | | |
| 1- | | | |
| 1 | | | |
| Homo_ | chr1: | GCATTGGTGGTTCAGTGGTAGAATTCTCGC | 334 |
| sapiens_ | 16861921- | CTCCCACGCGGGAGACCCGGGTTCAATTCC | |
| tRNA- | 16861991 | CGGCCAATGCA | |
| Gly- | (+) | | |
| CCC- | | | |
| 1- | | | |
| 2 | | | |
| Homo_ | chr2: | GCGCCGCTGGTGTAGTGGTATCATGCAAGA | 335 |
| sapiens_ | 70248991- | TTCCCATTCTTGCGACCCGGGTTCGATTCC | |
| tRNA- | 70249061 | CGGGCGGCGCA | |
| Gly- | (−) | | |
| CCC- | | | |
| 2- | | | |
| 1 | | | |
| Homo_ | chr16: | GCGCCGCTGGTGTAGTGGTATCATGCAAGA | 336 |
| sapiens_ | 636736- | TTCCCATTCTTGCGACCCGGGTTCGATTCC | |
| tRNA- | 636806 | CGGGCGGCGCA | |
| Gly- | (−) | | |
| CCC- | | | |
| 2- | | | |
| 2 | | | |
| Homo_ | chr17: | GCATTGGTGGTTCAATGGTAGAATTCTCGC | 337 |
| sapiens_ | 19860862- | CTCCCACGCAGGAGACCCAGGTTCGATTCC | |
| tRNA- | 19860932+) | TGGCCAATGCA | |
| Gly- | | | |
| CCC- | | | |
| 3- | | | |
| 1 | | | |
| Homo_ | chr1: | GCATGGGTGGTTCAGTGGTAGAATTCTCGC | 338 |
| sapiens_ | 161443304- | CTGCCACGCGGGAGGCCCGGGTTCGATTCC | |
| tRNA- | 161443374 | CGGCCCATGCA | |
| Gly- | (+) | | |
| GCC- | | | |
| 1- | | | |
| 1 | | | |
| Homo_ | chr1: | GCATGGGTGGTTCAGTGGTAGAATTCTCGC | 339 |
| sapiens_ | 161450677- | CTGCCACGCGGGAGGCCCGGGTTCGATTCC | |
| tRNA- | 161450747 | CGGCCCATGCA | |
| Gly- | (+) | | |
| GCC- | | | |
| 1- | | | |
| 2 | | | |
| Homo_ | chr1: | GCATGGGTGGTTCAGTGGTAGAATTCTCGC | 340 |
| sapiens_ | 161458108- | CTGCCACGCGGGAGGCCCGGGTTCGATTCC | |
| tRNA- | 161458178 | CGGCCCATGCA | |
| Gly- | (+) | | |
| GCC- | | | |
| 1- | | | |
| 3 | | | |
| Homo_ | chr1: | GCATGGGTGGTTCAGTGGTAGAATTCTCGC | 341 |
| sapiens_ | 161465468- | CTGCCACGCGGGAGGCCCGGGTTCGATTCC | |
| tRNA- | 161465538 | CGGCCCATGCA | |
| Gly- | (+) | | |
| GCC- | | | |
| 1- | | | |
| 4 | | | |
| Homo_ | chr21: | GCATGGGTGGTTCAGTGGTAGAATTCTCGC | 342 |
| sapiens_ | 17454789- | CTGCCACGCGGGAGGCCCGGGTTCGATTCC | |
| tRNA- | 17454859 | CGGCCCATGCA | |
| Gly- | (−) | | |
| GCC- | | | |
| 1- | | | |
| 5 | | | |
| Homo_ | chr1: | GCATTGGTGGTTCAGTGGTAGAATTCTCGC | 343 |
| sapiens_ | 161523847- | CTGCCACGCGGGAGGCCCGGGTTCGATTCC | |
| tRNA- | 161523917 | CGGCCAATGCA | |
| Gly- | (−) | | |
| GCC- | | | |
| 2- | | | |
| 1 | | | |
| Homo_ | chr2: | GCATTGGTGGTTCAGTGGTAGAATTCTCGC | 344 |
| sapiens_ | 156401147- | CTGCCACGCGGGAGGCCCGGGTTCGATTCC | |
| tRNA- | 156401217 | CGGCCAATGCA | |
| Gly- | (−) | | |
| GCC- | | | |
| 2- | | | |
| 2 | | | |
| Homo_ | chr6: | GCATTGGTGGTTCAGTGGTAGAATTCTCGC | 345 |
| sapiens_ | 27902908- | CTGCCACGCGGGAGGCCCGGGTTCGATTCC | |
| tRNA- | 27902978 | CGGCCAATGCA | |
| Gly- | (−) | | |
| GCC- | | | |
| 2- | | | |
| 3 | | | |
| Homo_ | chr16: | GCATTGGTGGTTCAGTGGTAGAATTCTCGC | 346 |
| sapiens_ | 70779039- | CTGCCACGCGGGAGGCCCGGGTTCGATTCC | |
| tRNA- | 70779109 | CGGCCAATGCA | |
| Gly- | (−) | | |
| GCC- | | | |
| 2- | | | |
| 4 | | | |
| Homo_ | chr16: | GCATTGGTGGTTCAGTGGTAGAATTCTCGC | 347 |
| sapiens_ | 70789507- | CTGCCACGCGGGAGGCCCGGGTTCGATTCC | |
| tRNA- | 70789577 | CGGCCAATGCA | |
| Gly- | (+) | | |
| GCC- | | | |
| 2- | | | |
| 5 | | | |
| Homo_ | chr17: | GCATTGGTGGTTCAGTGGTAGAATTCTCGC | 348 |
| sapiens_ | 8125746- | CTGCCACGCGGGAGGCCCGGGTTCGATTCC | |
| tRNA- | 8125816 | CGGCCAATGCA | |
| Gly- | (+) | | |
| GCC- | | | |
| 2- | | | |
| 6 | | | |
| Homo_ | chr16: | GCATTGGTGGTTCAGTGGTAGAATTCTCGC | 349 |
| sapiens_ | 70778211- | CTGCCACGCGGGAGGCCCGGGTTTGATTCC | |
| tRNA- | 70778281 | CGGCCAGTGCA | |
| Gly- | (−) | | |
| GCC- | | | |
| 3- | | | |
| 1 | | | |
| Homo_ | chr1: | GCATAGGTGGTTCAGTGGTAGAATTCTTGC | 350 |
| sapiens_ | 161480566- | CTGCCACGCAGGAGGCCCAGGTTTGATTCC | |
| tRNA- | 161480636 | TGGCCCATGCA | |
| Gly- | (+) | | |
| GCC- | | | |
| 4- | | | |
| 1 | | | |
| Homo_ | chr16: | GCATTGGTGGTTCAGTGGTAGAATTCTCGC | 351 |
| sapiens_ | 70788694- | CTGCCATGCGGGCGGCCGGGCTTCGATTCC | |
| tRNA- | 70788764 | TGGCCAATGCA | |
| Gly- | (+) | | |
| GCC- | | | |
| 5- | | | |
| 1 | | | |
| Homo_ | chr19: | GCGTTGGTGGTATAGTGGTTAGCATAGCTG | 352 |
| sapiens_ | 4724070- | CCTTCCAAGCAGTTGACCCGGGTTCGATTC | |
| tRNA- | 4724141 | CCGGCCAACGCA | |
| Gly- | (+) | | |
| TCC- | | | |
| 1- | | | |
| 1 | | | |
| Homo_ | chr1: | GCGTTGGTGGTATAGTGGTGAGCATAGCTG | 353 |
| sapiens_ | 146037061- | CCTTCCAAGCAGTTGACCCGGGTTCGATTC | |
| tRNA- | 146037132 | CCGGCCAACGCA | |
| Gly- | (+) | | |
| TCC- | | | |
| 2- | | | |
| 1 | | | |
| Homo_ | chr1: | GCGTTGGTGGTATAGTGGTGAGCATAGCTG | 354 |
| sapiens_ | 161447585- | CCTTCCAAGCAGTTGACCCGGGTTCGATTC | |
| tRNA- | 161447656 | CCGGCCAACGCA | |
| Gly- | (−) | | |
| TCC- | | | |
| 2- | | | |
| 2 | | | |
| Homo_ | chr1: | GCGTTGGTGGTATAGTGGTGAGCATAGCTG | 355 |
| sapiens_ | 161454966- | CCTTCCAAGCAGTTGACCCGGGTTCGATTC | |
| tRNA- | 161455037 | CCGGCCAACGCA | |
| Gly- | (−) | | |
| TCC- | | | |
| 2- | | | |
| 3 | | | |
| Homo_ | chr1: | GCGTTGGTGGTATAGTGGTGAGCATAGCTG | 356 |
| sapiens_ | 161462376- | CCTTCCAAGCAGTTGACCCGGGTTCGATTC | |
| tRNA- | 161462447 | CCGGCCAACGCA | |
| Gly- | (−) | | |
| TCC- | | | |
| 2- | | | |
| 4 | | | |
| Homo_ | chr1: | GCGTTGGTGGTATAGTGGTGAGCATAGCTG | 357 |
| sapiens_ | 161469757- | CCTTCCAAGCAGTTGACCCGGGTTCGATTC | |
| tRNA- | 161469828 | CCGGCCAACGCA | |
| Gly- | (−) | | |
| TCC- | | | |
| 2- | | | |
| 5 | | | |
| Homo_ | chr1: | GCGTTGGTGGTATAGTGGTGAGCATAGCTG | 358 |
| sapiens_ | 161531113- | CCTTCCAAGCAGTTGACCCGGGTTCGATTC | |
| tRNA- | 161531184 | CCGGCCAACGCA | |
| Gly- | (+) | | |
| TCC- | | | |
| 2- | | | |
| 6 | | | |
| Homo_ | chr17: | GCGTTGGTGGTATAGTGGTAAGCATAGCTG | 359 |
| sapiens_ | 8221548- | CCTTCCAAGCAGTTGACCCGGGTTCGATTC | |
| tRNA- | 8221619 | CCGGCCAACGCA | |
| Gly- | (+) | | |
| TCC- | | | |
| 3- | | | |
| 1 | | | |
| Homo_ | chr1: | GCGTTGGTGGTATAGTGGTGAGCATAGTTG | 360 |
| sapiens_ | 161440171- | CCTTCCAAGCAGTTGACCCGGGCTCGATTC | |
| tRNA- | 161440242 | CCGCCCAACGCA | |
| Gly- | (−) | | |
| TCC- | | | |
| 4- | | | |
| 1 | | | |
| Homo_ | chr1: | GCCGTGATCGTATAGTGGTTAGTACTCTGC | 361 |
| sapiens_ | 146038044- | GTTGTGGCCGCAGCAACCTCGGTTCGAATC | |
| tRNA- | 146038115 | CGAGTCACGGCA | |
| His- | (+) | | |
| GTG- | | | |
| 1- | | | |
| 1 | | | |
| Homo_ | chr1: | GCCGTGATCGTATAGTGGTTAGTACTCTGC | 362 |
| sapiens_ | 147073225- | GTTGTGGCCGCAGCAACCTCGGTTCGAATC | |
| tRNA- | 147073296 | CGAGTCACGGCA | |
| His- | (+) | | |
| GTG- | | | |
| 1- | | | |
| 2 | | | |
| Homo_ | chr1: | GCCGTGATCGTATAGTGGTTAGTACTCTGC | 363 |
| sapiens_ | 148281365- | GTTGTGGCCGCAGCAACCTCGGTTCGAATC | |
| tRNA- | 148281436 | CGAGTCACGGCA | |
| His- | (+) | | |
| GTG- | | | |
| 1- | | | |
| 3 | | | |
| Homo_ | chr1: | GCCGTGATCGTATAGTGGTTAGTACTCTGC | 364 |
| sapiens_ | 148302734- | GTTGTGGCCGCAGCAACCTCGGTTCGAATC | |
| tRNA- | 148302805 | CGAGTCACGGCA | |
| His- | (−) | | |
| GTG- | | | |
| 1- | | | |
| 4 | | | |
| Homo_ | chr6: | GCCGTGATCGTATAGTGGTTAGTACTCTGC | 365 |
| sapiens_ | 27158127- | GTTGTGGCCGCAGCAACCTCGGTTCGAATC | |
| tRNA- | 27158198 | CGAGTCACGGCA | |
| His- | (+) | | |
| GTG- | | | |
| 1- | | | |
| 5 | | | |
| Homo_ | chr9: | GCCGTGATCGTATAGTGGTTAGTACTCTGC | 366 |
| sapiens_ | 14433940- | GTTGTGGCCGCAGCAACCTCGGTTCGAATC | |
| tRNA- | 14434011 | CGAGTCACGGCA | |
| His- | (−) | | |
| GTG- | | | |
| 1- | | | |
| 6 | | | |
| Homo_ | chr15: | GCCGTGATCGTATAGTGGTTAGTACTCTGC | 367 |
| sapiens_ | 45198606- | GTTGTGGCCGCAGCAACCTCGGTTCGAATC | |
| tRNA- | 45198677 | CGAGTCACGGCA | |
| His- | (−) | | |
| GTG- | | | |
| 1- | | | |
| 7 | | | |
| Homo_ | chr15: | GCCGTGATCGTATAGTGGTTAGTACTCTGC | 368 |
| sapiens_ | 45200413- | GTTGTGGCCGCAGCAACCTCGGTTCGAATC | |
| tRNA- | 45200484 | CGAGTCACGGCA | |
| His- | (−) | | |
| GTG- | | | |
| 1- | | | |
| 8 | | | |
| Homo_ | chr15: | GCCGTGATCGTATAGTGGTTAGTACTCTGC | 369 |
| sapiens_ | 45201151- | GTTGTGGCCGCAGCAACCTCGGTTCGAATC | |
| tRNA- | 45201222 | CGAGTCACGGCA | |
| His- | (+) | | |
| GTG- | | | |
| 1- | | | |
| 9 | | | |
| Homo_ | chr6: | GGCCGGTTAGCTCAGTTGGTTAGAGCGTGG | 370 |
| sapiens_ | 57822973- | CGCTAATAACGCCAAGGTCGCGGGTTCGAT | |
| tRNA- | 57823046 | CCCCGTACGGGCCA | |
| Ile- | (+) | | |
| AAT- | | | |
| 1- | | | |
| 1 | | | |
| Homo_ | chr6: | GGCCGGTTAGCTCAGTCGGTTAGAGCGTGG | 371 |
| sapiens_ | 57800211- | TGCTAATAACGCCAAGGTCGCGGGTTCGAT | |
| tRNA- | 57800284 | CCCCGTGCCGGTCA | |
| Ile- | (+) | | |
| AAT- | | | |
| 12- | | | |
| 1 | | | |
| Homo_ | chr6: | GGCCGGTTAGCTCAGTTGGTTAGAGCGTGG | 372 |
| sapiens_ | 27688188- | TGCTAATAACGCCAAGGTCGCGGGTTCGAT | |
| tRNA- | 27688261 | CCCCGTACTGGCCA | |
| Ile- | (+) | | |
| AAT- | | | |
| 2- | | | |
| 1 | | | |
| Homo_ | chr6: | GGCTGGTTAGCTCAGTTGGTTAGAGCGTGG | 373 |
| sapiens_ | 27275211- | TGCTAATAACGCCAAGGTCGCGGGTTCGAT | |
| tRNA- | 27275284 | CCCCGTACTGGCCA | |
| Ile- | (−) | | |
| AAT- | | | |
| 3- | | | |
| 1 | | | |
| Homo_ | chr17: | GGCCGGTTAGCTCAGTTGGTTAGAGCGTGG | 374 |
| sapiens_ | 8226991- | TGCTAATAACGCCAAGGTCGCGGGTTCGAA | |
| tRNA- | 8227064 | CCCCGTACGGGCCA | |
| Ile- | (−) | | |
| AAT- | | | |
| 4- | | | |
| 1 | | | |
| Homo_ | chr6: | GGCCGGTTAGCTCAGTTGGTTAGAGCGTGG | 375 |
| sapiens_ | 26554122- | TGCTAATAACGCCAAGGTCGCGGGTTCGAT | |
| tRNA- | 26554195 | CCCCGTACGGGCCA | |
| Ile- | (+) | | |
| AAT- | | | |
| 5- | | | |
| 1 | | | |
| Homo_ | chr6: | GGCCGGTTAGCTCAGTTGGTTAGAGCGTGG | 376 |
| sapiens_ | 27177215- | TGCTAATAACGCCAAGGTCGCGGGTTCGAT | |
| tRNA- | 27177288 | CCCCGTACGGGCCA | |
| Ile- | (−) | | |
| AAT- | | | |
| 5- | | | |
| 2 | | | |
| Homo_ | chr6: | GGCCGGTTAGCTCAGTTGGTTAGAGCGTGG | 377 |
| sapiens_ | 27237571- | TGCTAATAACGCCAAGGTCGCGGGTTCGAT | |
| tRNA- | 27237644 | CCCCGTACGGGCCA | |
| Ile- | (−) | | |
| AAT- | | | |
| 5- | | | |
| 3 | | | |
| Homo_ | chr14: | GGCCGGTTAGCTCAGTTGGTTAGAGCGTGG | 378 |
| sapiens_ | 102317092- | TGCTAATAACGCCAAGGTCGCGGGTTCGAT | |
| tRNA- | 102317165 | CCCCGTACGGGCCA | |
| Ile- | (+) | | |
| AAT- | | | |
| 5- | | | |
| 4 | | | |
| Homo_ | chr17: | GGCCGGTTAGCTCAGTTGGTTAGAGCGTGG | 379 |
| sapiens_ | 8187593- | TGCTAATAACGCCAAGGTCGCGGGTTCGAT | |
| tRNA- | 8187666 | CCCCGTACGGGCCA | |
| Ile- | (+) | | |
| AAT- | | | |
| 5- | | | |
| 5 | | | |
| Homo_ | chr6: | GGCCGGTTAGCTCAGTTGGTTAGAGCGTGG | 380 |
| sapiens_ | 26756552- | TGCTAATAACGCTAAGGTCGCGGGTTCGAT | |
| tRNA- | 26756625 | CCCCGTACTGGCCA | |
| Ile- | (+) | | |
| AAT- | | | |
| 6- | | | |
| 1 | | | |
| Homo_ | chr6: | GGCCGGTTAGCTCAGTTGGTCAGAGCGTGG | 381 |
| sapiens_ | 26720992- | TGCTAATAACGCCAAGGTCGCGGGTTCGAT | |
| tRNA- | 26721065 | CCCCGTACGGGCCA | |
| Ile- | (−) | | |
| AAT- | | | |
| 7- | | | |
| 1 | | | |
| Homo_ | chr6: | GGCCGGTTAGCTCAGTTGGTCAGAGCGTGG | 382 |
| sapiens_ | 26780622- | TGCTAATAACGCCAAGGTCGCGGGTTCGAT | |
| tRNA- | 26780695 | CCCCGTACGGGCCA | |
| Ile- | (+) | | |
| AAT- | | | |
| 7- | | | |
| 2 | | | |
| Homo_ | chr6: | GGCCGGTTAGCTCAGTCGGCTAGAGCGTGG | 383 |
| sapiens_ | 27668583- | TGCTAATAACGCCAAGGTCGCGGGTTCGAT | |
| tRNA- | 27668656 | CCCCGTACGGGCCA | |
| Ile- | (+) | | |
| AAT- | | | |
| 8- | | | |
| 1 | | | |
| Homo_ | chr6: | GGCTGGTTAGTTCAGTTGGTTAGAGCGTGG | 384 |
| sapiens_ | 27273960- | TGCTAATAACGCCAAGGTCGTGGGTTCGAT | |
| tRNA- | 27274033 | CCCCATATCGGCCA | |
| Ile- | (+) | | |
| AAT- | | | |
| 9- | | | |
| 1 | | | |
| Homo_ | chrX: | GGCCGGTTAGCTCAGTTGGTAAGAGCGTGG | 385 |
| sapiens_ | 3838377- | TGCTGATAACACCAAGGTCGCGGGCTCGAC | |
| tRNA- | 3838450 | TCCCGCACCGGCCA | |
| Ile- | (−) | | |
| GAT- | | | |
| 1- | | | |
| 1 | | | |
| Homo_ | chrX: | GGCCGGTTAGCTCAGTTGGTAAGAGCGTGG | 386 |
| sapiens_ | 3876801- | TGCTGATAACACCAAGGTCGCGGGCTCGAC | |
| tRNA- | 3876874 | TCCCGCACCGGCCA | |
| Ile- | (−) | | |
| GAT- | | | |
| 1- | | | |
| 2 | | | |
| Homo_ | chrX: | GGCCGGTTAGCTCAGTTGGTAAGAGCGTGG | 387 |
| sapiens_ | 3915230- | TGCTGATAACACCAAGGTCGCGGGCTCGAC | |
| tRNA- | 3915303 | TCCCGCACCGGCCA | |
| Ile- | (−) | | |
| GAT- | | | |
| 1- | | | |
| 3 | | | |
| Homo_ | chr19: | GCTCCAGTGGCGCAATCGGTTAGCGCGCGG | 388 |
| sapiens_ | 39412168- | TACTTATATGACAGTGCGAGCGGAGCAATG | |
| tRNA- | 39412260 | CCGAGGTTGTGAGTTCGATCCTCACCTGGA | |
| Ile- | (−) | GCA | |
| TAT- | | | |
| 1- | | | |
| 1 | | | |
| Homo_ | chr2: | GCTCCAGTGGCGCAATCGGTTAGCGCGCGG | 389 |
| sapiens_ | 42810536- | TACTTATACAGCAGTACATGCAGAGCAATG | |
| tRNA- | 42810628 | CCGAGGTTGTGAGTTCGAGCCTCACCTGGA | |
| Ile- | (+) | GCA | |
| TAT- | | | |
| 2- | | | |
| 1 | | | |
| Homo_ | chr6: | GCTCCAGTGGCGCAATCGGTTAGCGCGCGG | 390 |
| sapiens_ | 27020346- | TACTTATATGGCAGTATGTGTGCGAGTGAT | |
| tRNA- | 27020439 | GCCGAGGTTGTGAGTTCGAGCCTCACCTGG | |
| Ile- | (+) | AGCA | |
| TAT- | | | |
| 2- | | | |
| 2 | | | |
| Homo_ | chr6: | GCTCCAGTGGCGCAATCGGTTAGCGCGCGG | 391 |
| sapiens_ | 27631421- | TACTTATACAACAGTATATGTGCGGGTGAT | |
| tRNA- | 27631514 | GCCGAGGTTGTGAGTTCGAGCCTCACCTGG | |
| Ile- | (+) | AGCA | |
| TAT- | | | |
| 2- | | | |
| 3 | | | |
| Homo_ | chr6: | GCTCCAGTGGCGCAATCGGTTAGCGCGCGG | 392 |
| sapiens_ | 28537590- | TACTTATAAGACAGTGCACCTGTGAGCAAT | |
| tRNA- | 28537683 | GCCGAGGTTGTGAGTTCAAGCCTCACCTGG | |
| Ile- | (+) | AGCA | |
| TAT- | | | |
| 3- | | | |
| 1 | | | |
| Homo_ | chr5: | GGTAGCGTGGCCGAGCGGTCTAAGGCGCTG | 393 |
| sapiens_ | 181097474- | GATTAAGGCTCCAGTCTCTTCGGAGGCGTG | |
| tRNA- | 181097555 | GGTTCGAATCCCACCGCTGCCA | |
| Leu- | (−) | | |
| AAG- | | | |
| 1- | | | |
| 1 | | | |
| Homo_ | chr5: | GGTAGCGTGGCCGAGCGGTCTAAGGCGCTG | 394 |
| sapiens_ | 181101840- | GATTAAGGCTCCAGTCTCTTCGGAGGCGTG | |
| tRNA- | 181101921 | GGTTCGAATCCCACCGCTGCCA | |
| Leu- | (+) | | |
| AAG- | | | |
| 1- | | | |
| 2 | | | |
| Homo_ | chr5: | GGTAGCGTGGCCGAGCGGTCTAAGGCGCTG | 395 |
| sapiens_ | 181174044- | GATTAAGGCTCCAGTCTCTTCGGAGGCGTG | |
| tRNA- | 181174125 | GGTTCGAATCCCACCGCTGCCA | |
| Leu- | (−) | | |
| AAG- | | | |
| 1- | | | |
| 3 | | | |
| Homo_ | chr5: | GGTAGCGTGGCCGAGCGGTCTAAGGCGCTG | 396 |
| sapiens_ | 181187701- | GATTAAGGCTCCAGTCTCTTCGGGGGCGTG | |
| tRNA- | 181187782 | GGTTCGAATCCCACCGCTGCCA | |
| Leu- | (+) | | |
| AAG- | | | |
| 2- | | | |
| 1 | | | |
| Homo_ | chr6: | GGTAGCGTGGCCGAGCGGTCTAAGGCGCTG | 397 |
| sapiens_ | 28943622- | GATTAAGGCTCCAGTCTCTTCGGGGGCGTG | |
| tRNA- | 28943703 | GGTTCGAATCCCACCGCTGCCA | |
| Leu- | (−) | | |
| AAG- | | | |
| 2- | | | |
| 2 | | | |
| Homo_ | chr14: | GGTAGCGTGGCCGAGCGGTCTAAGGCGCTG | 398 |
| sapiens_ | 20610132- | GATTAAGGCTCCAGTCTCTTCGGGGGCGTG | |
| tRNA- | 20610213 | GGTTCGAATCCCACCGCTGCCA | |
| Leu- | (+) | | |
| AAG- | | | |
| 2- | | | |
| 3 | | | |
| Homo_ | chr16: | GGTAGCGTGGCCGAGCGGTCTAAGGCGCTG | 399 |
| sapiens_ | 22297140- | GATTAAGGCTCCAGTCTCTTCGGGGGCGTG | |
| tRNA- | 22297221 | GGTTCGAATCCCACCGCTGCCA | |
| Leu- | (+) | | |
| AAG- | | | |
| 2- | | | |
| 4 | | | |
| Homo_ | chr6: | GGTAGCGTGGCCGAGCGGTCTAAGGCGCTG | 400 |
| sapiens_ | 28989002- | GATTAAGGCTCCAGTCTCTTCGGGGGCGTG | |
| tRNA- | 28989083 | GGTTCAAATCCCACCGCTGCCA | |
| Leu- | (+) | | |
| AAG- | | | |
| 3- | | | |
| 1 | | | |
| Homo_ | chr6: | GGTAGCGTGGCCGAGTGGTCTAAGACGCTG | 401 |
| sapiens_ | 28478623- | GATTAAGGCTCCAGTCTCTTCGGGGGCGTG | |
| tRNA- | 28478704 | GGTTTGAATCCCACCGCTGCCA | |
| Leu- | (−) | | |
| AAG- | | | |
| 4- | | | |
| 1 | | | |
| Homo_ | chr6: | GTCAGGATGGCCGAGTGGTCTAAGGCGCCA | 402 |
| sapiens_ | 28896223- | GACTCAAGCTAAGCTTCCTCCGCGGTGGGG | |
| tRNA- | 28896328 | ATTCTGGTCTCCAATGGAGGCGTGGGTTCG | |
| Leu- | (−) | AATCCCACTTCTGACA | |
| CAA- | | | |
| 1- | | | |
| 1 | | | |
| Homo_ | chr6: | GTCAGGATGGCCGAGTGGTCTAAGGCGCCA | 403 |
| sapiens_ | 28941053- | GACTCAAGCTTGGCTTCCTCGTGTTGAGGA | |
| tRNA- | 28941157 | TTCTGGTCTCCAATGGAGGCGTGGGTTCGA | |
| Leu- | (+) | ATCCCACTTCTGACA | |
| CAA- | | | |
| 1- | | | |
| 2 | | | |
| Homo_ | chr6: | GTCAGGATGGCCGAGTGGTCTAAGGCGCCA | 404 |
| sapiens_ | 27605638- | GACTCAAGCTTACTGCTTCCTGTGTTCGGG | |
| tRNA- | 27605745 | TCTTCTGGTCTCCGTATGGAGGCGTGGGTT | |
| Leu- | (−) | CGAATCCCACTTCTGACA | |
| CAA- | | | |
| 2- | | | |
| 1 | | | |
| Homo_ | chr6: | GTCAGGATGGCCGAGTGGTCTAAGGCGCCA | 405 |
| sapiens_ | 27602569- | GACTCAAGTTGCTACTTCCCAGGTTTGGGG | |
| tRNA- | 27602675 | CTTCTGGTCTCCGCATGGAGGCGTGGGTTC | |
| Leu- | (−) | GAATCCCACTTCTGACA | |
| CAA- | | | |
| 3- | | | |
| 1 | | | |
| Homo_ | chr1: | GTCAGGATGGCCGAGTGGTCTAAGGCGCCA | 406 |
| sapiens_ | 248873855- | GACTCAAGGTAAGCACCTTGCCTGCGGGCT | |
| tRNA- | 248873960 | TTCTGGTCTCCGGATGGAGGCGTGGGTTCG | |
| Leu- | (+) | AATCCCACTTCTGACA | |
| CAA- | | | |
| 4- | | | |
| 1 | | | |
| Homo_ | chr11: | GCCTCCTTAGTGCAGTAGGTAGCGCATCAG | 407 |
| sapiens_ | 9275243- | TCTCAAAATCTGAATGGTCCTGAGTTCAAG | |
| tRNA- | 9275316 | CCTCAGAGGGGGCA | |
| Leu- | (+) | | |
| CAA- | | | |
| 5- | | | |
| 1 | | | |
| Homo_ | chr1: | GTCAGGATGGCCGAGCAGTCTTAAGGCGCT | 408 |
| sapiens_ | 161611946- | GCGTTCAAATCGCACCCTCCGCTGGAGGCG | |
| tRNA- | 161612029 | TGGGTTCGAATCCCACTTTTGACA | |
| Leu- | (−) | | |
| CAA- | | | |
| 6- | | | |
| 1 | | | |
| Homo_ | chr1: | GTCAGGATGGCCGAGCGGTCTAAGGCGCTG | 409 |
| sapiens_ | 161441533- | CGTTCAGGTCGCAGTCTCCCCTGGAGGCGT | |
| tRNA- | 161441615 | GGGTTCGAATCCCACTCCTGACA | |
| Leu- | (+) | | |
| CAG- | | | |
| 1- | | | |
| 1 | | | |
| Homo_ | chr1: | GTCAGGATGGCCGAGCGGTCTAAGGCGCTG | 410 |
| sapiens_ | 161448951- | CGTTCAGGTCGCAGTCTCCCCTGGAGGCGT | |
| tRNA- | 161449033 | GGGTTCGAATCCCACTCCTGACA | |
| Leu- | (+) | | |
| CAG- | | | |
| 1- | | | |
| 2 | | | |
| Homo_ | chr1: | GTCAGGATGGCCGAGCGGTCTAAGGCGCTG | 411 |
| sapiens_ | 161456332- | CGTTCAGGTCGCAGTCTCCCCTGGAGGCGT | |
| tRNA- | 161456414 | GGGTTCGAATCCCACTCCTGACA | |
| Leu- | (+) | | |
| CAG- | | | |
| 1- | | | |
| 3 | | | |
| Homo_ | chr1: | GTCAGGATGGCCGAGCGGTCTAAGGCGCTG | 412 |
| sapiens_ | 161463742- | CGTTCAGGTCGCAGTCTCCCCTGGAGGCGT | |
| tRNA- | 161463824 | GGGTTCGAATCCCACTCCTGACA | |
| Leu- | (+) | | |
| CAG- | | | |
| 1- | | | |
| 4 | | | |
| Homo_ | chr1: | GTCAGGATGGCCGAGCGGTCTAAGGCGCTG | 413 |
| sapiens_ | 161471123- | CGTTCAGGTCGCAGTCTCCCCTGGAGGCGT | |
| tRNA- | 161471205 | GGGTTCGAATCCCACTCCTGACA | |
| Leu- | (+) | | |
| CAG- | | | |
| 1- | | | |
| 5 | | | |
| Homo_ | chr1: | GTCAGGATGGCCGAGCGGTCTAAGGCGCTG | 414 |
| sapiens_ | 161530342- | CGTTCAGGTCGCAGTCTCCCCTGGAGGCGT | |
| tRNA- | 161530424 | GGGTTCGAATCCCACTCCTGACA | |
| Leu- | (−) | | |
| CAG- | | | |
| 1- | | | |
| 6 | | | |
| Homo_ | chr6: | GTCAGGATGGCCGAGCGGTCTAAGGCGCTG | 415 |
| sapiens_ | 26521208- | CGTTCAGGTCGCAGTCTCCCCTGGAGGCGT | |
| tRNA- | 26521290 | GGGTTCGAATCCCACTCCTGACA | |
| Leu- | (+) | | |
| CAG- | | | |
| 1- | | | |
| 7 | | | |
| Homo_ | chr16: | GTCAGGATGGCCGAGCGGTCTAAGGCGCTG | 416 |
| sapiens_ | 57299951- | CGTTCAGGTCGCAGTCTCCCCTGGAGGCGT | |
| tRNA- | 57300033 | GGGTTCGAATCCCACTTCTGACA | |
| Leu- | (+) | | |
| CAG- | | | |
| 2- | | | |
| 1 | | | |
| Homo_ | chr16: | GTCAGGATGGCCGAGCGGTCTAAGGCGCTG | 417 |
| sapiens_ | 57300480- | CGTTCAGGTCGCAGTCTCCCCTGGAGGCGT | |
| tRNA- | 57300562 | GGGTTCGAATCCCACTTCTGACA | |
| Leu- | (−) | | |
| CAG- | | | |
| 2- | | | |
| 2 | | | |
| Homo_ | chr6: | ACCAGGATGGCCGAGTGGTTAAGGCGTTGG | 418 |
| sapiens_ | 144216547- | ACTTAAGATCCAATGGACATATGTCCGCGT | |
| tRNA- | 144216629 | GGGTTCGAACCCCACTCCTGGTA | |
| Leu- | (+) | | |
| TAA- | | | |
| 1- | | | |
| 1 | | | |
| Homo_ | chr6: | ACCGGGATGGCCGAGTGGTTAAGGCGTTGG | 419 |
| sapiens_ | 27721119- | ACTTAAGATCCAATGGGCTGGTGCCCGCGT | |
| tRNA- | 27721201 | GGGTTCGAACCCCACTCTCGGTA | |
| Leu- | (−) | | |
| TAA- | | | |
| 2- | | | |
| 1 | | | |
| Homo_ | chr11: | ACCAGAATGGCCGAGTGGTTAAGGCGTTGG | 420 |
| sapiens_ | 59551755- | ACTTAAGATCCAATGGATTCATATCCGCGT | |
| tRNA- | 59551837 | GGGTTCGAACCCCACTTCTGGTA | |
| Leu- | (+) | | |
| TAA- | | | |
| 3- | | | |
| 1 | | | |
| Homo_ | chr6: | ACCGGGATGGCTGAGTGGTTAAGGCGTTGG | 421 |
| sapiens_ | 27230555- | ACTTAAGATCCAATGGACAGGTGTCCGCGT | |
| tRNA- | 27230637 | GGGTTCGAGCCCCACTCCCGGTA | |
| Leu- | (−) | | |
| TAA- | | | |
| 4- | | | |
| 1 | | | |
| Homo_ | chr17: | GGTAGCGTGGCCGAGCGGTCTAAGGCGCTG | 422 |
| sapiens_ | 8120314- | GATTTAGGCTCCAGTCTCTTCGGAGGCGTG | |
| tRNA- | 8120395 | GGTTCGAATCCCACCGCTGCCA | |
| Leu- | (−) | | |
| TAG- | | | |
| 1- | | | |
| 1 | | | |
| Homo_ | chr14: | GGTAGTGTGGCCGAGCGGTCTAAGGCGCTG | 423 |
| sapiens_ | 20625370- | GATTTAGGCTCCAGTCTCTTCGGGGGCGTG | |
| tRNA- | 20625451 | GGTTCGAATCCCACCACTGCCA | |
| Leu- | (+) | | |
| TAG- | | | |
| 2- | | | |
| 1 | | | |
| Homo_ | chr16: | GGTAGCGTGGCCGAGTGGTCTAAGGCGCTG | 424 |
| sapiens_ | 22195711- | GATTTAGGCTCCAGTCATTTCGATGGCGTG | |
| tRNA- | 22195792 | GGTTCGAATCCCACCGCTGCCA | |
| Leu- | (−) | | |
| TAG- | | | |
| 3- | | | |
| 1 | | | |
| Homo_ | chr14: | GCCCGGCTAGCTCAGTCGGTAGAGCATGGG | 425 |
| sapiens_ | 58239895- | ACTCTTAATCCCAGGGTCGTGGGTTCGAGC | |
| tRNA- | 58239967 | CCCACGTTGGGCG | |
| Lys- | (−) | | |
| CTT- | | | |
| 1- | | | |
| 1 | | | |
| Homo_ | chr15: | GCCCGGCTAGCTCAGTCGGTAGAGCATGGG | 426 |
| sapiens_ | 78860562- | ACTCTTAATCCCAGGGTCGTGGGTTCGAGC | |
| tRNA- | 78860634 | CCCACGTTGGGCG | |
| Lys- | (+) | | |
| CTT- | | | |
| 1- | | | |
| 2 | | | |
| Homo_ | chr19: | GCCCAGCTAGCTCAGTCGGTAGAGCATAAG | 427 |
| sapiens_ | 35575848- | ACTCTTAATCTCAGGGTTGTGGATTCGTGC | |
| tRNA- | 35575920 | CCCATGCTGGGTG | |
| Lys- | (+) | | |
| CTT- | | | |
| 10- | | | |
| 1 | | | |
| Homo_ | chr19: | GCAGCTAGCTCAGTCGGTAGAGCATGAGAC | 428 |
| sapiens_ | 51922140- | TCTTAATCTCAGGGTCATGGGTTCGTGCCC | |
| tRNA- | 51922213 | CATGTTGGGTGCCA | |
| Lys- | (−) | | |
| CTT- | | | |
| 11- | | | |
| 1 | | | |
| Homo_ | chr1: | GCCCGGCTAGCTCAGTCGGTAGAGCATGAG | 429 |
| sapiens_ | 146039401- | ACTCTTAATCTCAGGGTCGTGGGTTCGAGC | |
| tRNA- | 146039473 | CCCACGTTGGGCG | |
| Lys- | (+) | | |
| CTT- | | | |
| 2- | | | |
| 1 | | | |
| Homo_ | chr5: | GCCCGGCTAGCTCAGTCGGTAGAGCATGAG | 430 |
| sapiens_ | 181207755- | ACTCTTAATCTCAGGGTCGTGGGTTCGAGC | |
| tRNA- | 181207827 | CCCACGTTGGGCG | |
| Lys- | (+) | | |
| CTT- | | | |
| 2- | | | |
| 2 | | | |
| Homo_ | chr5: | GCCCGGCTAGCTCAGTCGGTAGAGCATGAG | 431 |
| sapiens_ | 181221979- | ACTCTTAATCTCAGGGTCGTGGGTTCGAGC | |
| tRNA- | 181222051 | CCCACGTTGGGCG | |
| Lys- | (−) | | |
| CTT- | | | |
| 2- | | | |
| 3 | | | |
| Homo_ | chr6: | GCCCGGCTAGCTCAGTCGGTAGAGCATGAG | 432 |
| sapiens_ | 26556546- | ACTCTTAATCTCAGGGTCGTGGGTTCGAGC | |
| tRNA- | 26556618 | CCCACGTTGGGCG | |
| Lys- | (+) | | |
| CTT- | | | |
| 2- | | | |
| 4 | | | |
| Homo_ | chr16: | GCCCGGCTAGCTCAGTCGGTAGAGCATGAG | 433 |
| sapiens_ | 3175691- | ACTCTTAATCTCAGGGTCGTGGGTTCGAGC | |
| tRNA- | 3175763 | CCCACGTTGGGCG | |
| Lys- | (+) | | |
| CTT- | | | |
| 2- | | | |
| 5 | | | |
| Homo_ | chr16: | GCCCGGCTAGCTCAGTCGGTAGAGCATGAG | 434 |
| sapiens_ | 3157405- | ACCCTTAATCTCAGGGTCGTGGGTTCGAGC | |
| tRNA- | 3157477 | CCCACGTTGGGCG | |
| Lys- | (−) | | |
| CTT- | | | |
| 3- | | | |
| 1 | | | |
| Homo_ | chr16: | GCCCGGCTAGCTCAGTCGGTAGAGCATGGG | 435 |
| sapiens_ | 3191501- | ACTCTTAATCTCAGGGTCGTGGGTTCGAGC | |
| tRNA- | 3191573 | CCCACGTTGGGCG | |
| Lys- | (+) | | |
| CTT- | | | |
| 4- | | | |
| 1 | | | |
| Homo_ | chr16: | GCCCGGCTAGCTCAGTCGATAGAGCATGAG | 436 |
| sapiens_ | 3180554- | ACTCTTAATCTCAGGGTCGTGGGTTCGAGC | |
| tRNA- | 3180626 | CGCACGTTGGGCG | |
| Lys- | (−) | | |
| CTT- | | | |
| 5- | | | |
| 1 | | | |
| Homo_ | chr1: | GCCCAGCTAGCTCAGTCGGTAGAGCATGAG | 437 |
| sapiens_ | 54957869- | ACTCTTAATCTCAGGGTCATGGGTTTGAGC | |
| tRNA- | 54957941 | CCCACGTTTGGTG | |
| Lys- | (−) | | |
| CTT- | | | |
| 7- | | | |
| 1 | | | |
| Homo_ | chr16: | GCCTGGCTAGCTCAGTCGGCAAAGCATGAG | 438 |
| sapiens_ | 3164938- | ACTCTTAATCTCAGGGTCGTGGGCTCGAGC | |
| tRNA- | 3165010 | TCCATGTTGGGCG | |
| Lys- | (+) | | |
| CTT- | | | |
| 8- | | | |
| 1 | | | |
| Homo_ | chr5: | GCCCGACTACCTCAGTCGGTGGAGCATGGG | 439 |
| sapiens_ | 26198430- | ACTCTTCATCCCAGGGTTGTGGGTTCGAGC | |
| tRNA- | 26198502 | CCCACATTGGGCA | |
| Lys- | (−). | | |
| CTT- | | | |
| 9- | | | |
| 1 | | | |
| Homo_ | chr16: | GCCTGGATAGCTCAGTTGGTAGAGCATCAG | 440 |
| sapiens_ | 73478317- | ACTTTTAATCTGAGGGTCCAGGGTTCAAGT | |
| tRNA- | 73478389 | CCCTGTTCAGGCA | |
| Lys- | (−) | | |
| TTT- | | | |
| 1- | | | |
| 1 | | | |
| Homo_ | chr12: | ACCCAGATAGCTCAGTCAGTAGAGCATCAG | 441 |
| sapiens_ | 27690373- | ACTTTTAATCTGAGGGTCCAAGGTTCATGT | |
| tRNA- | 27690445 | CCCTTTTTGGGTG | |
| Lys- | (+) | | |
| TTT- | | | |
| 11- | | | |
| 1 | | | |
| Homo_ | chr11: | GCCTGGATAGCTCAGTTGGTAGAGCATCAG | 442 |
| sapiens_ | 122559947- | ACTTTTAATCTGAGGGTCCAGGGTTCAAGT | |
| tRNA- | 122560019 | CCCTGTTCAGGCG | |
| Lys- | (+) | | |
| TTT- | | | |
| 2- | | | |
| 1 | | | |
| Homo_ | chr1: | GCCCGGATAGCTCAGTCGGTAGAGCATCAG | 443 |
| sapiens_ | 204506527- | ACTTTTAATCTGAGGGTCCAGGGTTCAAGT | |
| tRNA- | 204506599 | CCCTGTTCGGGCG | |
| Lys- | (+) | | |
| TTT- | | | |
| 3- | | | |
| 1 | | | |
| Homo_ | chr1: | GCCCGGATAGCTCAGTCGGTAGAGCATCAG | 444 |
| sapiens_ | 204507030- | ACTTTTAATCTGAGGGTCCAGGGTTCAAGT | |
| tRNA- | 204507102 | CCCTGTTCGGGCG | |
| Lys- | (−) | | |
| TTT- | | | |
| 3- | | | |
| 2 | | | |
| Homo_ | chr6: | GCCCGGATAGCTCAGTCGGTAGAGCATCAG | 445 |
| sapiens_ | 28951029- | ACTTTTAATCTGAGGGTCCAGGGTTCAAGT | |
| tRNA- | 28951101 | CCCTGTTCGGGCG | |
| Lys- | (+) | | |
| TTT- | | | |
| 3- | | | |
| 3 | | | |
| Homo_ | chr11: | GCCCGGATAGCTCAGTCGGTAGAGCATCAG | 446 |
| sapiens_ | 59560335- | ACTTTTAATCTGAGGGTCCAGGGTTCAAGT | |
| tRNA- | 59560407 | CCCTGTTCGGGCG | |
| Lys- | (−) | | |
| TTT- | | | |
| 3- | | | |
| 4 | | | |
| Homo_ | chr17: | GCCCGGATAGCTCAGTCGGTAGAGCATCAG | 447 |
| sapiens_ | 8119155- | ACTTTTAATCTGAGGGTCCAGGGTTCAAGT | |
| tRNA- | 8119227 | CCCTGTTCGGGCG | |
| Lys- | (+) | | |
| TTT- | | | |
| 3- | | | |
| 5 | | | |
| Homo_ | chr6: | GCCTGGATAGCTCAGTCGGTAGAGCATCAG | 448 |
| sapiens_ | 27591814- | ACTTTTAATCTGAGGGTCCAGGGTTCAAGT | |
| tRNA- | 27591886 | CCCTGTTCAGGCG | |
| Lys- | (−) | | |
| TTT- | | | |
| 4- | | | |
| 1 | | | |
| Homo_ | chr11: | GCCCGGATAGCTCAGTCGGTAGAGCATCAG | 449 |
| sapiens_ | 59556429- | ACTTTTAATCTGAGGGTCCGGGGTTCAAGT | |
| tRNA- | 59556501 | CCCTGTTCGGGCG | |
| Lys- | (+) | | |
| TTT- | | | |
| 5- | | | |
| 1 | | | |
| Homo_ | chr6: | GCCTGGGTAGCTCAGTCGGTAGAGCATCAG | 450 |
| sapiens_ | 27334990- | ACTTTTAATCTGAGGGTCCAGGGTTCAAGT | |
| tRNA- | 27335062 | CCCTGTCCAGGCG | |
| Lys- | (−) | | |
| TTT- | | | |
| 6- | | | |
| 1 | | | |
| Homo_ | chr6: | GCCTGGATAGCTCAGTTGGTAGAACATCAG | 451 |
| sapiens_ | 28747744- | ACTTTTAATCTGACGGTGCAGGGTTCAAGT | |
| tRNA- | 28747816 | CCCTGTTCAGGCG | |
| Lys- | (+) | | |
| TTT- | | | |
| 7- | | | |
| 1 | | | |
| Homo_ | chr8: | GCCTCGTTAGCGCAGTAGGTAGCGCGTCAG | 452 |
| sapiens_ | 123157230- | TCTCATAATCTGAAGGTCGTGAGTTCGATC | |
| tRNA- | 123157302 | CTCACACGGGGCA | |
| Met- | (−) | | |
| CAT- | | | |
| 1- | | | |
| 1 | | | |
| Homo_ | chr16: | GCCCTCTTAGCGCAGTGGGCAGCGCGTCAG | 453 |
| sapiens_ | 71426493- | TCTCATAATCTGAAGGTCCTGAGTTCGAGC | |
| tRNA- | 71426565 | CTCAGAGAGGGCA | |
| Met- | (+) | | |
| CAT- | | | |
| 2- | | | |
| 1 | | | |
| Homo_ | chr6: | GCCTCCTTAGCGCAGTAGGCAGCGCGTCAG | 454 |
| sapiens_ | 28944575- | TCTCATAATCTGAAGGTCCTGAGTTCGAAC | |
| tRNA- | 28944647 | CTCAGAGGGGGCA | |
| Met- | (+) | | |
| CAT- | | | |
| 3- | | | |
| 1 | | | |
| Homo_ | chr6: | GCCTCCTTAGCGCAGTAGGCAGCGCGTCAG | 455 |
| sapiens_ | 28953265- | TCTCATAATCTGAAGGTCCTGAGTTCGAAC | |
| tRNA- | 28953337 | CTCAGAGGGGGCA | |
| Met- | (−) | | |
| CAT- | | | |
| 3- | | | |
| 2 | | | |
| Homo_ | chr6: | GCCCTCTTAGCGCAGCGGGCAGCGCGTCAG | 456 |
| sapiens_ | 26735370- | TCTCATAATCTGAAGGTCCTGAGTTCGAGC | |
| tRNA- | 26735442 | CTCAGAGAGGGCA | |
| Met- | (−) | | |
| CAT- | | | |
| 4- | | | |
| 1 | | | |
| Homo_ | chr6: | GCCCTCTTAGCGCAGCGGGCAGCGCGTCAG | 457 |
| sapiens_ | 26743263- | TCTCATAATCTGAAGGTCCTGAGTTCGAGC | |
| tRNA- | 26743335 | CTCAGAGAGGGCA | |
| Met- | (+) | | |
| CAT- | | | |
| 4- | | | |
| 2 | | | |
| Homo_ | chr6: | GCCCTCTTAGCGCAGCGGGCAGCGCGTCAG | 458 |
| sapiens_ | 26766234- | TCTCATAATCTGAAGGTCCTGAGTTCGAGC | |
| tRNA- | 26766306 | CTCAGAGAGGGCA | |
| Met- | | | |
| CAT- | | | |
| 4- | | | |
| 3 | | | |
| Homo_ | chr6: | GCCCTCTTAGCGCAGCTGGCAGCGCGTCAG | 459 |
| sapiens_ | 26701483- | TCTCATAATCTGAAGGTCCTGAGTTCAAGC | |
| tRNA- | 26701555 | CTCAGAGAGGGCA | |
| Met- | (+) | | |
| CAT- | | | |
| 5- | | | |
| 1 | | | |
| Homo_ | chr6: | GCCCTCTTAGCGCAGCTGGCAGCGCGTCAG | 460 |
| sapiens_ | 26800113- | TCTCATAATCTGAAGGTCCTGAGTTCAAGC | |
| tRNA- | 26800185 | CTCAGAGAGGGCA | |
| Met- | (−) | | |
| CAT- | | | |
| 5- | | | |
| 2 | | | |
| Homo_ | chr16: | GCCTCGTTAGCGCAGTAGGCAGCGCGTCAG | 461 |
| sapiens_ | 87384022- | TCTCATAATCTGAAGGTCGTGAGTTCGAGC | |
| tRNA- | 87384094 | CTCACACGGGGCA | |
| Met- | (−) | | |
| CAT- | | | |
| 6- | | | |
| 1 | | | |
| Homo_ | chr6: | GCCCTCTTAGTGCAGCTGGCAGCGCGTCAG | 462 |
| sapiens_ | 57842214- | TTTCATAATCTGAAAGTCCTGAGTTCAAGC | |
| tRNA- | 57842286 | CTCAGAGAGGGCA | |
| Met- | (−) | | |
| CAT- | | | |
| 7- | | | |
| 1 | | | |
| Homo_ | chr6: | GCCGAAATAGCTCAGTTGGGAGAGCGTTAG | 463 |
| sapiens_ | 28790722- | ACTGAAGATCTAAAGGTCCCTGGTTCGATC | |
| tRNA- | 28790794 | CCGGGTTTCGGCA | |
| Phe- | (−) | | |
| GAA- | | | |
| 1- | | | |
| 1 | | | |
| Homo_ | chr6: | GCCGAAATAGCTCAGTTGGGAGAGCGTTAG | 464 |
| sapiens_ | 28981672- | ACTGAAGATCTAAAGGTCCCTGGTTCGATC | |
| tRNA- | 28981744 | CCGGGTTTCGGCA | |
| Phe- | (−) | | |
| GAA- | | | |
| 1- | | | |
| 2 | | | |
| Homo_ | chr11: | GCCGAAATAGCTCAGTTGGGAGAGCGTTAG | 465 |
| sapiens_ | 59557497- | ACTGAAGATCTAAAGGTCCCTGGTTCGATC | |
| tRNA- | 59557569 | CCGGGTTTCGGCA | |
| Phe- | (−) | | |
| GAA- | | | |
| 1- | | | |
| 3 | | | |
| Homo_ | chr12: | GCCGAAATAGCTCAGTTGGGAGAGCGTTAG | 466 |
| sapiens_ | 124927843- | ACTGAAGATCTAAAGGTCCCTGGTTCGATC | |
| tRNA- | 124927915 | CCGGGTTTCGGCA | |
| Phe- | (−) | | |
| GAA- | | | |
| 1- | | | |
| 4 | | | |
| Homo_ | chr13: | GCCGAAATAGCTCAGTTGGGAGAGCGTTAG | 467 |
| sapiens_ | 94549650- | ACTGAAGATCTAAAGGTCCCTGGTTCGATC | |
| tRNA- | 94549722 | CCGGGTTTCGGCA | |
| Phe- | (−) | | |
| GAA- | | | |
| 1- | | | |
| 5 | | | |
| Homo_ | chr19: | GCCGAAATAGCTCAGTTGGGAGAGCGTTAG | 468 |
| sapiens_ | 1383362- | ACTGAAGATCTAAAGGTCCCTGGTTCGATC | |
| tRNA- | 1383434 | CCGGGTTTCGGCA | |
| Phe- | (−) | | |
| GAA- | | | |
| 1- | | | |
| 6 | | | |
| Homo_ | chr11: | GCCGAAATAGCTCAGTTGGGAGAGCGTTAG | 469 |
| sapiens_ | 59566380- | ACTGAAGATCTAAAGGTCCCTGGTTCAATC | |
| tRNA- | 59566452 | CCGGGTTTCGGCA | |
| Phe- | (−) | | |
| GAA- | | | |
| 2- | | | |
| 1 | | | |
| Homo_ | chr6: | GCCGAGATAGCTCAGTTGGGAGAGCGTTAG | 470 |
| sapiens_ | 28807833- | ACTGAAGATCTAAAGGTCCCTGGTTCAATC | |
| tRNA- | 28807905 | CCGGGTTTCGGCA | |
| Phe- | (−) | | |
| GAA- | | | |
| 3- | | | |
| 1 | | | |
| Homo_ | chr6: | GCCGAAATAGCTCAGTTGGGAGAGCGTTAG | 471 |
| sapiens_ | 28823316- | ACCGAAGATCTTAAAGGTCCCTGGTTCAAT | |
| tRNA- | 28823389 | CCCGGGTTTCGGCA | |
| Phe- | (−) | | |
| GAA- | | | |
| 4- | | | |
| 1 | | | |
| Homo_ | chr6: | GCTGAAATAGCTCAGTTGGGAGAGCGTTAG | 472 |
| sapiens_ | 28763597- | ACTGAAGATCTTAAAGTTCCCTGGTTCAAC | |
| tRNA- | 28763670 | CCTGGGTTTCAGCC | |
| Phe- | (−) | | |
| GAA- | | | |
| 6- | | | |
| 1 | | | |
| Homo_ | chr16: | GGCTCGTTGGTCTAGGGGTATGATTCTCGC | 473 |
| sapiens_ | 3191989- | TTAGGATGCGAGAGGTCCCGGGTTCAAATC | |
| tRNA- | 3192060 | CCGGACGAGCCC | |
| Pro- | (+) | | |
| AGG- | | | |
| 1- | | | |
| 1 | | | |
| Homo_ | chr1: | GGCTCGTTGGTCTAGGGGTATGATTCTCGC | 474 |
| sapiens_ | 167715488- | TTAGGGTGCGAGAGGTCCCGGGTTCAAATC | |
| tRNA- | 167715559 | CCGGACGAGCCC | |
| Pro- | (−) | | |
| AGG- | | | |
| 2- | | | |
| 1 | | | |
| Homo_ | chr6: | GGCTCGTTGGTCTAGGGGTATGATTCTCGC | 475 |
| sapiens_ | 26555270- | TTAGGGTGCGAGAGGTCCCGGGTTCAAATC | |
| tRNA- | 26555341 | CCGGACGAGCCC | |
| Pro- | (+) | | |
| AGG- | | | |
| 2- | | | |
| 2 | | | |
| Homo_ | chr7: | GGCTCGTTGGTCTAGGGGTATGATTCTCGC | 476 |
| sapiens_ | 128783450- | TTAGGGTGCGAGAGGTCCCGGGTTCAAATC | |
| tRNA- | 128783521 | CCGGACGAGCCC | |
| Pro- | (+) | | |
| AGG- | | | |
| 2- | | | |
| 3 | | | |
| Homo_ | chr11: | GGCTCGTTGGTCTAGGGGTATGATTCTCGC | 477 |
| sapiens_ | 76235513- | TTAGGGTGCGAGAGGTCCCGGGTTCAAATC | |
| tRNA- | 76235584 | CCGGACGAGCCC | |
| Pro- | (+) | | |
| AGG- | | | |
| 2- | | | |
| 4 | | | |
| Homo_ | chr14: | GGCTCGTTGGTCTAGGGGTATGATTCTCGC | 478 |
| sapiens_ | 20609336- | TTAGGGTGCGAGAGGTCCCGGGTTCAAATC | |
| tRNA- | 20609407 | CCGGACGAGCCC | |
| Pro- | (−) | | |
| AGG- | | | |
| 2- | | | |
| 5 | | | |
| Homo_ | chr14: | GGCTCGTTGGTCTAGGGGTATGATTCTCGC | 479 |
| sapiens_ | 20613401- | TTAGGGTGCGAGAGGTCCCGGGTTCAAATC | |
| tRNA- | 20613472 | CCGGACGAGCCC | |
| Pro- | (−) | | |
| AGG- | | | |
| 2- | | | |
| 6 | | | |
| Homo_ | chr16: | GGCTCGTTGGTCTAGGGGTATGATTCTCGC | 480 |
| sapiens_ | 3182635- | TTAGGGTGCGAGAGGTCCCGGGTTCAAATC | |
| tRNA- | 3182706 | CCGGACGAGCCC | |
| Pro- | | | |
| AGG- | | | |
| 2- | | | |
| 7 | | | |
| Homo_ | chr16: | GGCTCGTTGGTCTAGGGGTATGATTCTCGC | 481 |
| sapiens_ | 3189634- | TTAGGGTGCGAGAGGTCCCGGGTTCAAATC | |
| tRNA- | 3189705 | CCGGACGAGCCC | |
| Pro- | | | |
| AGG- | | | |
| 2- | | | |
| 8 | | | |
| Homo_ | chr1: | GGCTCGTTGGTCTAGGGGTATGATTCTCGC | 482 |
| sapiens_ | 167714725- | TTCGGGTGCGAGAGGTCCCGGGTTCAAATC | |
| tRNA- | 167714796 | CCGGACGAGCCC | |
| Pro- | (+) | | |
| CGG- | | | |
| 1- | | | |
| 1 | | | |
| Homo_ | chr16: | GGCTCGTTGGTCTAGGGGTATGATTCTCGC | 483 |
| sapiens_ | 3172048- | TTCGGGTGCGAGAGGTCCCGGGTTCAAATC | |
| tRNA- | 3172119 | CCGGACGAGCCC | |
| Pro- | | | |
| CGG- | | | |
| 1- | | | |
| 2 | | | |
| Homo_ | chr17: | GGCTCGTTGGTCTAGGGGTATGATTCTCGC | 484 |
| sapiens_ | 8222833- | TTCGGGTGCGAGAGGTCCCGGGTTCAAATC | |
| tRNA- | 8222904 | CCGGACGAGCCC | |
| Pro- | (−) | | |
| CGG- | | | |
| 1- | | | |
| 3 | | | |
| Homo_ | chr6: | GGCTCGTTGGTCTAGGGGTATGATTCTCGC | 485 |
| sapiens_ | 27091742- | TTCGGGTGTGAGAGGTCCCGGGTTCAAATC | |
| tRNA- | 27091813+) | CCGGACGAGCCC | |
| Pro- | | | |
| CGG- | | | |
| 2- | | | |
| 1 | | | |
| Homo_ | chr14: | GGCTCGTTGGTCTAGTGGTATGATTCTCGC | 486 |
| sapiens_ | 20633006- | TTTGGGTGCGAGAGGTCCCGGGTTCAAATC | |
| tRNA- | 20633077 | CCGGACGAGCCC | |
| Pro- | (+) | | |
| TGG- | | | |
| 1- | | | |
| 1 | | | |
| Homo_ | chr11: | GGCTCGTTGGTCTAGGGGTATGATTCTCGG | 487 |
| sapiens_ | 76235825- | TTTGGGTCCGAGAGGTCCCGGGTTCAAATC | |
| tRNA- | 76235896 | CCGGACGAGCCC | |
| Pro- | (−) | | |
| TGG- | | | |
| 2- | | | |
| 1 | | | |
| Homo_ | chr5: | GGCTCGTTGGTCTAGGGGTATGATTCTCGC | 488 |
| sapiens_ | 181188854- | TTTGGGTGCGAGAGGTCCCGGGTTCAAATC | |
| tRNA- | 181188925 | CCGGACGAGCCC | |
| Pro- | (−) | | |
| TGG- | | | |
| 3- | | | |
| 1 | | | |
| Homo_ | chr14: | GGCTCGTTGGTCTAGGGGTATGATTCTCGC | 489 |
| sapiens_ | 20684016- | TTTGGGTGCGAGAGGTCCCGGGTTCAAATC | |
| tRNA- | 20684087 | CCGGACGAGCCC | |
| Pro- | (+) | | |
| TGG- | | | |
| 3- | | | |
| 2 | | | |
| Homo_ | chr16: | GGCTCGTTGGTCTAGGGGTATGATTCTCGC | 490 |
| sapiens_ | 3158922- | TTTGGGTGCGAGAGGTCCCGGGTTCAAATC | |
| tRNA- | 3158993 | CCGGACGAGCCC | |
| Pro- | (+) | | |
| TGG- | | | |
| 3- | | | |
| 3 | | | |
| Homo_ | chr16: | GGCTCGTTGGTCTAGGGGTATGATTCTCGC | 491 |
| sapiens_ | 3184133- | TTTGGGTGCGAGAGGTCCCGGGTTCAAATC | |
| tRNA- | 3184204 | CCGGACGAGCCC | |
| Pro- | (−) | | |
| TGG- | | | |
| 3- | | | |
| 4 | | | |
| Homo_ | chr16: | GGCTCGTTGGTCTAGGGGTATGATTCTCGC | 492 |
| sapiens_ | 3188094- | TTTGGGTGCGAGAGGTCCCGGGTTCAAATC | |
| tRNA- | 3188165 | CCGGACGAGCCC | |
| Pro- | (+) | | |
| TGG- | | | |
| 3- | | | |
| 5 | | | |
| Homo_ | chr19: | GCCCGGATGATCCTCAGTGGTCTGGGGTGC | 493 |
| sapiens_ | 45478601- | AGGCTTCAAACCTGTAGCTGTCTAGCGACA | |
| tRNA- | 45478687 | GAGTGGTTCAATTCCACCTTTCGGGCG | |
| SeC- | (−) | | |
| TCA- | | | |
| 1- | | | |
| 1 | | | |
| Homo_ | chr6: | GTAGTCGTGGCCGAGTGGTTAAGGCGATGG | 494 |
| sapiens_ | 27541775- | ACTAGAAATCCATTGGGGTTTCCCCGCGCA | |
| tRNA- | 27541856 | GGTTCGAATCCTGCCGACTACG | |
| Ser- | (−) | | |
| AGA- | | | |
| 1- | | | |
| 1 | | | |
| Homo_ | chr6: | GTAGTCGTGGCCGAGTGGTTAAGGCGATGG | 495 |
| sapiens_ | 26327589- | ACTAGAAATCCATTGGGGTCTCCCCGCGCA | |
| tRNA- | 26327670 | GGTTCGAATCCTGCCGACTACG | |
| Ser- | (+) | | |
| AGA- | | | |
| 2- | | | |
| 1 | | | |
| Homo_ | chr6: | GTAGTCGTGGCCGAGTGGTTAAGGCGATGG | 496 |
| sapiens_ | 27478812- | ACTAGAAATCCATTGGGGTCTCCCCGCGCA | |
| tRNA- | 27478893+) | GGTTCGAATCCTGCCGACTACG | |
| Ser- | | | |
| AGA- | | | |
| 2- | | | |
| 2 | | | |
| Homo_ | chr6: | GTAGTCGTGGCCGAGTGGTTAAGGCGATGG | 497 |
| sapiens_ | 27495814- | ACTAGAAATCCATTGGGGTCTCCCCGCGCA | |
| tRNA- | 27495895 | GGTTCGAATCCTGCCGACTACG | |
| Ser- | (+) | | |
| AGA- | | | |
| 2- | | | |
| 3 | | | |
| Homo_ | chr6: | GTAGTCGTGGCCGAGTGGTTAAGGCGATGG | 498 |
| sapiens_ | 27503039- | ACTAGAAATCCATTGGGGTCTCCCCGCGCA | |
| tRNA- | 27503120 | GGTTCGAATCCTGCCGACTACG | |
| Ser- | (+) | | |
| AGA- | | | |
| 2- | | | |
| 4 | | | |
| Homo_ | chr8: | GTAGTCGTGGCCGAGTGGTTAAGGCGATGG | 499 |
| sapiens_ | 95269657- | ACTAGAAATCCATTGGGGTCTCCCCGCGCA | |
| tRNA- | 95269738 | GGTTCGAATCCTGCCGACTACG | |
| Ser- | (−) | | |
| AGA- | | | |
| 2- | | | |
| 5 | | | |
| Homo_ | chr17: | GTAGTCGTGGCCGAGTGGTTAAGGCGATGG | 500 |
| sapiens_ | 8226610- | ACTAGAAATCCATTGGGGTCTCCCCGCGCA | |
| tRNA- | 8226691 | GGTTCGAATCCTGCCGACTACG | |
| Ser- | (−) | | |
| AGA- | | | |
| 2- | | | |
| 6 | | | |
| Homo_ | chr6: | GTAGTCGTGGCCGAGTGGTTAAGGCGATGG | 501 |
| sapiens_ | 27532208- | ACTAGAAATCCATTGGGGTTTCCCCACGCA | |
| tRNA- | 27532289 | GGTTCGAATCCTGCCGACTACG | |
| Ser- | (+) | | |
| AGA- | | | |
| 3- | | | |
| 1 | | | |
| Homo_ | chr6: | GTAGTCGTGGCCGAGTGGTTAAGGTGATGG | 502 |
| sapiens_ | 27553413- | ACTAGAAACCCATTGGGGTCTCCCCGCGCA | |
| tRNA- | 27553494 | GGTTCGAATCCTGCCGACTACG | |
| Ser- | (−) | | |
| AGA- | | | |
| 4- | | | |
| 1 | | | |
| Homo_ | chr17: | GCTGTGATGGCCGAGTGGTTAAGGCGTTGG | 503 |
| sapiens_ | 8138881- | ACTCGAAATCCAATGGGGTCTCCCCGCGCA | |
| tRNA- | 8138962 | GGTTCGAATCCTGCTCACAGCG | |
| Ser- | (−) | | |
| CGA- | | | |
| 1- | | | |
| 1 | | | |
| Homo_ | chr6: | GCTGTGATGGCCGAGTGGTTAAGGCGTTGG | 504 |
| sapiens_ | 27209849- | ACTCGAAATCCAATGGGGTCTCCCCGCGCA | |
| tRNA- | 27209930 | GGTTCAAATCCTGCTCACAGCG | |
| Ser- | (+) | | |
| CGA- | | | |
| 2- | | | |
| 1 | | | |
| Homo_ | chr6: | GCTGTGATGGCCGAGTGGTTAAGGTGTTGG | 505 |
| sapiens_ | 27672450- | ACTCGAAATCCAATGGGGGTTCCCCGCGCA | |
| tRNA- | 27672531 | GGTTCAAATCCTGCTCACAGCG | |
| Ser- | (−) | | |
| CGA- | | | |
| 3- | | | |
| 1 | | | |
| Homo_ | chr12: | GTCACGGTGGCCGAGTGGTTAAGGCGTTGG | 506 |
| sapiens_ | 56190364- | ACTCGAAATCCAATGGGGTTTCCCCGCACA | |
| tRNA- | 56190445 | GGTTCGAATCCTGTTCGTGACG | |
| Ser- | (+) | | |
| CGA- | | | |
| 4- | | | |
| 1 | | | |
| Homo_ | chr6: | GACGAGGTGGCCGAGTGGTTAAGGCGATGG | 507 |
| sapiens_ | 27097306- | ACTGCTAATCCATTGTGCTCTGCACGCGTG | |
| tRNA- | 27097387 | GGTTCGAATCCCACCCTCGTCG | |
| Ser- | (+) | | |
| GCT- | | | |
| 1- | | | |
| 1 | | | |
| Homo_ | chr6: | GACGAGGTGGCCGAGTGGTTAAGGCGATGG | 508 |
| sapiens_ | 27297996- | ACTGCTAATCCATTGTGCTCTGCACGCGTG | |
| tRNA- | 27298077 | GGTTCGAATCCCACCTTCGTCG | |
| Ser- | (+) | | |
| GCT- | | | |
| 2- | | | |
| 1 | | | |
| Homo_ | chr11: | GACGAGGTGGCCGAGTGGTTAAGGCGATGG | 509 |
| sapiens_ | 66348120- | ACTGCTAATCCATTGTGCTTTGCACGCGTG | |
| tRNA- | 66348201 | GGTTCGAATCCCATCCTCGTCG | |
| Ser- | (+) | | |
| GCT- | | | |
| 3- | | | |
| 1 | | | |
| Homo_ | chr6: | GACGAGGTGGCCGAGTGGTTAAGGCGATGG | 510 |
| sapiens_ | 28597340- | ACTGCTAATCCATTGTGCTCTGCACGCGTG | |
| tRNA- | 28597421 | GGTTCGAATCCCATCCTCGTCG | |
| Ser- | (−) | | |
| GCT- | | | |
| 4- | | | |
| 1 | | | |
| Homo_ | chr15: | GACGAGGTGGCCGAGTGGTTAAGGCGATGG | 511 |
| sapiens_ | 40593825- | ACTGCTAATCCATTGTGCTCTGCACGCGTG | |
| tRNA- | 40593906 | GGTTCGAATCCCATCCTCGTCG | |
| Ser- | (−) | | |
| GCT- | | | |
| 4- | | | |
| 2 | | | |
| Homo_ | chr17: | GACGAGGTGGCCGAGTGGTTAAGGCGATGG | 512 |
| sapiens_ | 8186866- | ACTGCTAATCCATTGTGCTCTGCACGCGTG | |
| tRNA- | 8186947 | GGTTCGAATCCCATCCTCGTCG | |
| Ser- | (+) | | |
| GCT- | | | |
| 4- | | | |
| 3 | | | |
| Homo_ | chr6: | GACGAGGTGGCCGAGTGGTTAAGGCGATGG | 513 |
| sapiens_ | 28213037- | ACTGCTAATCCATTGTGCTCTGCACACGTG | |
| tRNA- | 28213118 | GGTTCGAATCCCATCCTCGTCG | |
| Ser- | (+) | | |
| GCT- | | | |
| 5- | | | |
| 1 | | | |
| Homo_ | chr6: | GGAGAGGCCTGGCCGAGTGGTTAAGGCGAT | 514 |
| sapiens_ | 26305490- | GGACTGCTAATCCATTGTGCTCTGCACGCG | |
| tRNA- | 26305573 | TGGGTTCGAATCCCATCCTCGTCG | |
| Ser- | (−) | | |
| GCT- | | | |
| 6- | | | |
| 1 | | | |
| Homo_ | chr10: | GCAGCGATGGCCGAGTGGTTAAGGCGTTGG | 515 |
| sapiens_ | 67764503- | ACTTGAAATCCAATGGGGTCTCCCCGCGCA | |
| tRNA- | 67764584 | GGTTCGAACCCTGCTCGCTGCG | |
| Ser- | (+) | | |
| TGA- | | | |
| 1- | | | |
| 1 | | | |
| Homo_ | chr6: | GTAGTCGTGGCCGAGTGGTTAAGGCGATGG | 516 |
| sapiens_ | 27545689- | ACTTGAAATCCATTGGGGTTTCCCCGCGCA | |
| tRNA- | 27545770 | GGTTCGAATCCTGCCGACTACG | |
| Ser- | (+) | | |
| TGA- | | | |
| 2- | | | |
| 1 | | | |
| Homo_ | chr6: | GTAGTCGTGGCCGAGTGGTTAAGGCGATGG | 517 |
| sapiens_ | 26312596- | ACTTGAAATCCATTGGGGTCTCCCCGCGCA | |
| tRNA- | 26312677 | GGTTCGAATCCTGCCGACTACG | |
| Ser- | (−) | | |
| TGA- | | | |
| 3- | | | |
| 1 | | | |
| Homo_ | chr6: | GTAGTCGTGGCCGAGTGGTTAAGGCGATGG | 518 |
| sapiens_ | 27505828- | ACTTGAAATCCATTGGGGTTTCCCCGCGCA | |
| tRNA- | 27505909 | GGTTCGAATCCTGTCGGCTACG | |
| Ser- | (−) | | |
| TGA- | | | |
| 4- | | | |
| 1 | | | |
| Homo_ | chr17: | GGCGCCGTGGCTTAGTTGGTTAAAGCGCCT | 519 |
| sapiens_ | 8187160- | GTCTAGTAAACAGGAGATCCTGGGTTCGAA | |
| tRNA- | 8187233 | TCCCAGCGGTGCCT | |
| Thr- | | | |
| AGT- | | | |
| 1- | | | |
| 1 | | | |
| Homo_ | chr17: | GGCGCCGTGGCTTAGTTGGTTAAAGCGCCT | 520 |
| sapiens_ | 8226235- | GTCTAGTAAACAGGAGATCCTGGGTTCGAA | |
| tRNA- | 8226308 | TCCCAGCGGTGCCT | |
| Thr- | (−) | | |
| AGT- | | | |
| 1- | | | |
| 2 | | | |
| Homo_ | chr19: | GGCGCCGTGGCTTAGTTGGTTAAAGCGCCT | 521 |
| sapiens_ | 33177057- | GTCTAGTAAACAGGAGATCCTGGGTTCGAA | |
| tRNA- | 33177130 | TCCCAGCGGTGCCT | |
| Thr- | (+) | | |
| AGT- | | | |
| 1- | | | |
| 3 | | | |
| Homo_ | chr6: | GGCTCCGTGGCTTAGCTGGTTAAAGCGCCT | 522 |
| sapiens_ | 26532917- | GTCTAGTAAACAGGAGATCCTGGGTTCGAA | |
| tRNA- | 26532990 | TCCCAGCGGGGCCT | |
| Thr- | (−) | | |
| AGT- | | | |
| 2- | | | |
| 1 | | | |
| Homo_ | chr6: | GGCTCCGTGGCTTAGCTGGTTAAAGCGCCT | 523 |
| sapiens_ | 27684695- | GTCTAGTAAACAGGAGATCCTGGGTTCGAA | |
| tRNA- | 27684768 | TCCCAGCGGGGCCT | |
| Thr- | (−) | | |
| AGT- | | | |
| 2- | | | |
| 2 | | | |
| Homo_ | chr6: | GGCTCCGTAGCTTAGTTGGTTAAAGCGCCT | 524 |
| sapiens_ | 28726018- | GTCTAGTAAACAGGAGATCCTGGGTTCGAC | |
| tRNA- | 28726091 | TCCCAGCGGGGCCT | |
| Thr- | (+) | | |
| AGT- | | | |
| 3- | | | |
| 1 | | | |
| Homo_ | chr6: | GGCTTCGTGGCTTAGCTGGTTAAAGCGCCT | 525 |
| sapiens_ | 27726694- | GTCTAGTAAACAGGAGATCCTGGGTTCGAA | |
| tRNA- | 27726767 | TCCCAGCGAGGCCT | |
| Thr- | (+) | | |
| AGT- | | | |
| 4- | | | |
| 1 | | | |
| Homo_ | chr17: | GGCGCCGTGGCTTAGCTGGTTAAAGCGCCT | 526 |
| sapiens_ | 8139452- | GTCTAGTAAACAGGAGATCCTGGGTTCGAA | |
| tRNA- | 8139525 | TCCCAGCGGTGCCT | |
| Thr- | (−) | | |
| AGT- | | | |
| 5- | | | |
| 1 | | | |
| Homo_ | chr6: | GGCCCTGTGGCTTAGCTGGTCAAAGCGCCT | 527 |
| sapiens_ | 27162271- | GTCTAGTAAACAGGAGATCCTGGGTTCGAA | |
| tRNA- | 27162344 | TCCCAGCGGGGCCT | |
| Thr- | | | |
| AGT- | | | |
| 6- | | | |
| 1 | | | |
| Homo_ | chr6: | GGCTCTATGGCTTAGTTGGTTAAAGCGCCT | 528 |
| sapiens_ | 28488993- | GTCTCGTAAACAGGAGATCCTGGGTTCGAC | |
| tRNA- | 28489066 | TCCCAGTGGGGCCT | |
| Thr- | (−) | | |
| CGT- | | | |
| 1- | | | |
| 1 | | | |
| Homo_ | chr16: | GGCGCGGTGGCCAAGTGGTAAGGCGTCGGT | 529 |
| sapiens_ | 14285893- | CTCGTAAACCGAAGATCACGGGTTCGAACC | |
| tRNA- | 14285964 | CCGTCCGTGCCT | |
| Thr- | (+) | | |
| CGT- | | | |
| 2- | | | |
| 1 | | | |
| Homo_ | chr6: | GGCTCTGTGGCTTAGTTGGCTAAAGCGCCT | 530 |
| sapiens_ | 28648207- | GTCTCGTAAACAGGAGATCCTGGGTTCGAA | |
| tRNA- | 28648280 | TCCCAGCGGGGCCT | |
| Thr- | (−) | | |
| CGT- | | | |
| 3- | | | |
| 1 | | | |
| Homo_ | chr17: | GGCGCGGTGGCCAAGTGGTAAGGCGTCGGT | 531 |
| sapiens_ | 31550074- | CTCGTAAACCGAAGATCGCGGGTTCGAACC | |
| tRNA- | 31550145 | CCGTCCGTGCCT | |
| Thr- | (+) | | |
| CGT- | | | |
| 4- | | | |
| 1 | | | |
| Homo_ | chr6: | GGCCCTGTAGCTCAGCGGTTGGAGCGCTGG | 532 |
| sapiens_ | 27618356- | TCTCGTAAACCTAGGGGTCGTGAGTTCAAA | |
| tRNA- | 27618429 | TCTCACCAGGGCCT | |
| Thr- | (+) | | |
| CGT- | | | |
| 5- | | | |
| 1 | | | |
| Homo_ | chr6: | GGCTCTATGGCTTAGTTGGTTAAAGCGCCT | 533 |
| sapiens_ | 28474552- | GTCTTGTAAACAGGAGATCCTGGGTTCGAA | |
| tRNA- | 28474625 | TCCCAGTAGAGCCT | |
| Thr- | (−) | | |
| TGT- | | | |
| 1- | | | |
| 1 | | | |
| Homo_ | chr1: | GGCTCCATAGCTCAGTGGTTAGAGCACTGG | 534 |
| sapiens_ | 222465005- | TCTTGTAAACCAGGGGTCGCGAGTTCGATC | |
| tRNA- | 222465077 | CTCGCTGGGGCCT | |
| Thr- | (+) | | |
| TGT- | | | |
| 2- | | | |
| 1 | | | |
| Homo_ | chr14: | GGCTCCATAGCTCAGGGGTTAGAGCGCTGG | 535 |
| sapiens_ | 20613790- | TCTTGTAAACCAGGGGTCGCGAGTTCAATT | |
| tRNA- | 20613862 | CTCGCTGGGGCCT | |
| Thr- | (−) | | |
| TGT- | | | |
| 3- | | | |
| 1 | | | |
| Homo_ | chr14: | GGCTCCATAGCTCAGGGGTTAGAGCACTGG | 536 |
| sapiens_ | 20631160- | TCTTGTAAACCAGGGGTCGCGAGTTCAAAT | |
| tRNA- | 20631232 | CTCGCTGGGGCCT | |
| Thr- | (−) | | |
| TGT- | | | |
| 4- | | | |
| 1 | | | |
| Homo_ | chr14: | GGCCCTATAGCTCAGGGGTTAGAGCACTGG | 537 |
| sapiens_ | 20681690- | TCTTGTAAACCAGGGGTCGCGAGTTCAAAT | |
| tRNA- | 20681762 | CTCGCTGGGGCCT | |
| Thr- | (+) | | |
| TGT- | | | |
| 5- | | | |
| 1 | | | |
| Homo_ | chr5: | GGCTCCATAGCTCAGGGGTTAGAGCACTGG | 538 |
| sapiens_ | 181191687- | TCTTGTAAACCAGGGTCGCGAGTTCAAATC | |
| tRNA- | 181191758 | TCGCTGGGGCCT | |
| Thr- | (−) | | |
| TGT- | | | |
| 6- | | | |
| 1 | | | |
| Homo_ | chr17: | GGCCTCGTGGCGCAACGGTAGCGCGTCTGA | 539 |
| sapiens_ | 8220869- | CTCCAGATCAGAAGGTTGCGTGTTCAAATC | |
| tRNA- | 8220940 | ACGTCGGGGTCA | |
| Trp- | (−) | | |
| CCA- | | | |
| 1- | | | |
| 1 | | | |
| Homo_ | chr17: | GACCTCGTGGCGCAATGGTAGCGCGTCTGA | 540 |
| sapiens_ | 19508181- | CTCCAGATCAGAAGGTTGCGTGTTCAAGTC | |
| tRNA- | 19508252 | ACGTCGGGGTCA | |
| Trp- | (+) | | |
| CCA- | | | |
| 2- | | | |
| 1 | | | |
| Homo_ | chr6: | GACCTCGTGGCGCAACGGTAGCGCGTCTGA | 541 |
| sapiens_ | 26319102- | CTCCAGATCAGAAGGTTGCGTGTTCAAATC | |
| tRNA- | 26319173 | ACGTCGGGGTCA | |
| Trp- | (−) | | |
| CCA- | | | |
| 3- | | | |
| 1 | | | |
| Homo_ | chr6: | GACCTCGTGGCGCAACGGTAGCGCGTCTGA | 542 |
| sapiens_ | 26331444- | CTCCAGATCAGAAGGTTGCGTGTTCAAATC | |
| tRNA- | 26331515 | ACGTCGGGGTCA | |
| Trp- | (−) | | |
| CCA- | | | |
| 3- | | | |
| 2 | | | |
| Homo_ | chr17: | GACCTCGTGGCGCAACGGTAGCGCGTCTGA | 543 |
| sapiens_ | 8186358- | CTCCAGATCAGAAGGTTGCGTGTTCAAATC | |
| tRNA- | 8186429 | ACGTCGGGGTCA | |
| Trp- | (+) | | |
| CCA- | | | |
| 3- | | | |
| 3 | | | |
| Homo_ | chr12: | GACCTCGTGGCGCAACGGTAGCGCGTCTGA | 544 |
| sapiens_ | 98504252- | CTCCAGATCAGAAGGCTGCGTGTTCGAATC | |
| tRNA- | 98504323 | ACGTCGGGGTCA | |
| Trp- | (+) | | |
| CCA- | | | |
| 4- | | | |
| 1 | | | |
| Homo_ | chr7: | GACCTCGTGGCGCAACGGCAGCGCGTCTGA | 545 |
| sapiens_ | 99469684- | CTCCAGATCAGAAGGTTGCGTGTTCAAATC | |
| tRNA- | 99469755 | ACGTCGGGGTCA | |
| Trp- | (+) | | |
| CCA- | | | |
| 5- | | | |
| 1 | | | |
| Homo_ | chr2: | CCTTCAATAGTTCAGCTGGTAGAGCAGAGG | 546 |
| sapiens_ | 218245826- | ACTATAGCTACTTCCTCAGTAGGAGACGTC | |
| tRNA- | 218245918 | CTTAGGTTGCTGGTTCGATTCCAGCTTGAA | |
| Tyr- | (+) | GGA | |
| ATA- | | | |
| 1- | | | |
| 1 | | | |
| Homo_ | chr6: | CCTTCGATAGCTCAGTTGGTAGAGCGGAGG | 547 |
| sapiens_ | 26568858- | ACTGTAGTTGGCTGTGTCCTTAGACATCCT | |
| tRNA- | 26568948 | TAGGTCGCTGGTTCGAATCCGGCTCGAAGG | |
| Tyr- | (+) | A | |
| GTA- | | | |
| 1- | | | |
| 1 | | | |
| Homo_ | chr2: | CCTTCGATAGCTCAGTTGGTAGAGCGGAGG | 548 |
| sapiens_ | 27050782- | ACTGTAGTGGATAGGGCGTGGCAATCCTTA | |
| tRNA- | 27050870 | GGTCGCTGGTTCGATTCCGGCTCGAAGGA | |
| Tyr- | (+) | | |
| GTA- | | | |
| 2- | | | |
| 1 | | | |
| Homo_ | chr6: | CCTTCGATAGCTCAGTTGGTAGAGCGGAGG | 549 |
| sapiens_ | 26577104- | ACTGTAGGCTCATTAAGCAAGGTATCCTTA | |
| tRNA- | 26577192 | GGTCGCTGGTTCGAATCCGGCTCGGAGGA | |
| Tyr- | (+) | | |
| GTA- | | | |
| 3- | | | |
| 1 | | | |
| Homo_ | chr14: | CCTTCGATAGCTCAGCTGGTAGAGCGGAGG | 550 |
| sapiens_ | 20657464- | ACTGTAGATTGTATAGACATTTGCGGACAT | |
| tRNA- | 20657557 | CCTTAGGTCGCTGGTTCGATTCCAGCTCGA | |
| Tyr- | (−) | AGGA | |
| GTA- | | | |
| 4- | | | |
| 1 | | | |
| Homo_ | chr8: | CCTTCGATAGCTCAGCTGGTAGAGCGGAGG | 551 |
| sapiens_ | 66113367- | ACTGTAGCTACTTCCTCAGCAGGAGACATC | |
| tRNA- | 66113459+) | CTTAGGTCGCTGGTTCGATTCCGGCTCGAA | |
| Tyr- | | GGA | |
| GTA- | | | |
| 5- | | | |
| 1 | | | |
| Homo_ | chr8: | CCTTCGATAGCTCAGCTGGTAGAGCGGAGG | 552 |
| sapiens_ | 66113988- | ACTGTAGGCGCGCGCCCGTGGCCATCCTTA | |
| tRNA- | 66114076 | GGTCGCTGGTTCGATTCCGGCTCGAAGGA | |
| Tyr- | | | |
| GTA- | | | |
| 5- | | | |
| 2 | | | |
| Homo_ | chr14: | CCTTCGATAGCTCAGCTGGTAGAGCGGAGG | 553 |
| sapiens_ | 20653099- | ACTGTAGCCTGTAGAAACATTTGTGGACAT | |
| tRNA- | 20653192 | CCTTAGGTCGCTGGTTCGATTCCGGCTCGA | |
| Tyr- | (−) | AGGA | |
| GTA- | | | |
| 5- | | | |
| 3 | | | |
| Homo_ | chr14: | CCTTCGATAGCTCAGCTGGTAGAGCGGAGG | 554 |
| sapiens_ | 20663192- | ACTGTAGATTGTACAGACATTTGCGGACAT | |
| tRNA- | 206632850 | CCTTAGGTCGCTGGTTCGATTCCGGCTCGA | |
| Tyr- | | AGGA | |
| GTA- | | | |
| 5- | | | |
| 4 | | | |
| Homo_ | chr14: | CCTTCGATAGCTCAGCTGGTAGAGCGGAGG | 555 |
| sapiens_ | 20683273- | ACTGTAGTACTTAATGTGTGGTCATCCTTA | |
| tRNA- | 20683361 | GGTCGCTGGTTCGATTCCGGCTCGAAGGA | |
| Tyr- | | | |
| GTA- | | | |
| 5- | | | |
| 5 | | | |
| Homo_ | chr6: | CCTTCGATAGCTCAGCTGGTAGAGCGGAGG | 556 |
| sapiens_ | 26594874- | ACTGTAGGGGTTTGAATGTGGTCATCCTTA | |
| tRNA- | 26594962 | GGTCGCTGGTTCGAATCCGGCTCGGAGGA | |
| Tyr- | (+) | | |
| GTA- | | | |
| 6- | | | |
| 1 | | | |
| Homo_ | chr14: | CCTTCGATAGCTCAGCTGGTAGAGCGGAGG | 557 |
| sapiens_ | 20659958- | ACTGTAGACTGCGGAAACGTTTGTGGACAT | |
| tRNA- | 20660051 | CCTTAGGTCGCTGGTTCAATTCCGGCTCGA | |
| Tyr- | (−) | AGGA | |
| GTA- | | | |
| 7- | | | |
| 1 | | | |
| Homo_ | chr6: | CTTTCGATAGCTCAGTTGGTAGAGCGGAGG | 558 |
| sapiens_ | 26575570- | ACTGTAGGTTCATTAAACTAAGGCATCCTT | |
| tRNA- | 26575659 | AGGTCGCTGGTTCGAATCCGGCTCGAAGGA | |
| Tyr- | | | |
| GTA- | | | |
| 8- | | | |
| 1 | | | |
| Homo_ | chr8: | TCTTCAATAGCTCAGCTGGTAGAGCGGAGG | 559 |
| sapiens_ | 65697297- | ACTGTAGGTGCACGCCCGTGGCCATTCTTA | |
| tRNA- | 65697384 | GGTGCTGGTTTGATTCCGACTTGGAGAG | |
| Tyr- | (−) | | |
| GTA- | | | |
| 9- | | | |
| 1 | | | |
| Homo_ | chr3: | GTTTCCGTAGTGTAGTGGTTATCACGTTCG | 560 |
| sapiens_ | 169772230- | CCTAACACGCGAAAGGTCCCCGGTTCGAAA | |
| tRNA- | 169772302 | CCGGGCGGAAACA | |
| Val- | (+) | | |
| AAC- | | | |
| 1- | | | |
| 1 | | | |
| Homo_ | chr5: | GTTTCCGTAGTGTAGTGGTTATCACGTTCG | 561 |
| sapiens_ | 181164154- | CCTAACACGCGAAAGGTCCCCGGTTCGAAA | |
| tRNA- | 181164226 | CCGGGCGGAAACA | |
| Val- | (+) | | |
| AAC- | | | |
| 1- | | | |
| 2 | | | |
| Homo_ | chr5: | GTTTCCGTAGTGTAGTGGTTATCACGTTCG | 562 |
| sapiens_ | 181169610- | CCTAACACGCGAAAGGTCCCCGGTTCGAAA | |
| tRNA- | 181169682 | CCGGGCGGAAACA | |
| Val- | (+) | | |
| AAC- | | | |
| 1- | | | |
| 3 | | | |
| Homo_ | chr5: | GTTTCCGTAGTGTAGTGGTTATCACGTTCG | 563 |
| sapiens_ | 181218270- | CCTAACACGCGAAAGGTCCCCGGTTCGAAA | |
| tRNA- | 181218342 | CCGGGCGGAAACA | |
| Val- | (−) | | |
| AAC- | | | |
| 1- | | | |
| 4 | | | |
| Homo_ | chr6: | GTTTCCGTAGTGTAGTGGTTATCACGTTCG | 564 |
| sapiens_ | 27753400- | CCTAACACGCGAAAGGTCCCCGGTTCGAAA | |
| tRNA- | 27753472 | CCGGGCGGAAACA | |
| Val- | (−) | | |
| AAC- | | | |
| 1- | | | |
| 5 | | | |
| Homo_ | chr5: | GTTTCCGTAGTGTAGTGGTCATCACGTTCG | 565 |
| sapiens_ | 181188416- | CCTAACACGCGAAAGGTCCCCGGTTCGAAA | |
| tRNA- | 181188488 | CCGGGCGGAAACA | |
| Val- | (−) | | |
| AAC- | | | |
| 2- | | | |
| 1 | | | |
| Homo_ | chr6: | GTTTCCGTAGTGTAGTGGTTATCACGTTCG | 566 |
| sapiens_ | 27650928- | CCTAACACGCGAAAGGTCCCTGGATCAAAA | |
| tRNA- | 27651000 | CCAGGCGGAAACA | |
| Val- | (−) | | |
| AAC- | | | |
| 3- | | | |
| 1 | | | |
| Homo_ | chr6: | GTTTCCGTAGTGTAGTGGTTATCACGTTCG | 567 |
| sapiens_ | 27681106- | CCTAACACGCGAAAGGTCCGCGGTTCGAAA | |
| tRNA- | 27681178 | CCGGGCGGAAACA | |
| Val- | (−) | | |
| AAC- | | | |
| 4- | | | |
| 1 | | | |
| Homo_ | chr6: | GTTTCCGTAGTGTAGTGGTTATCACGTTTG | 568 |
| sapiens_ | 27235509- | CCTAACACGCGAAAGGTCCCCGGTTCGAAA | |
| tRNA- | 27235581 | CCGGGCAGAAACA | |
| Val- | (+) | | |
| AAC- | | | |
| 5- | | | |
| 1 | | | |
| Homo_ | chr6: | GGGGGTGTAGCTCAGTGGTAGAGCGTATGC | 569 |
| sapiens_ | 28735429- | TTAACATTCATGAGGCTCTGGGTTCGATCC | |
| tRNA- | 28735500 | CCAGCACTTCCA | |
| Val- | (−) | | |
| AAC- | | | |
| 6- | | | |
| 1 | | | |
| Homo_ | chr1: | GTTTCCGTAGTGTAGTGGTTATCACGTTCG | 570 |
| sapiens_ | 161399700- | CCTCACACGCGAAAGGTCCCCGGTTCGAAA | |
| tRNA- | 161399772 | CCGGGCGGAAACA | |
| Val- | (−) | | |
| CAC- | | | |
| 1- | | | |
| 1 | | | |
| Homo_ | chr5: | GTTTCCGTAGTGTAGTGGTTATCACGTTCG | 571 |
| sapiens_ | 181097070- | CCTCACACGCGAAAGGTCCCCGGTTCGAAA | |
| tRNA- | 181097142 | CCGGGCGGAAACA | |
| Val- | (+) | | |
| CAC- | | | |
| 1- | | | |
| 2 | | | |
| Homo_ | chr5: | GTTTCCGTAGTGTAGTGGTTATCACGTTCG | 572 |
| sapiens_ | 181102253- | CCTCACACGCGAAAGGTCCCCGGTTCGAAA | |
| tRNA- | 181102325 | CCGGGCGGAAACA | |
| Val- | (−) | | |
| CAC- | | | |
| 1- | | | |
| 3 | | | |
| Homo_ | chr5: | GTTTCCGTAGTGTAGTGGTTATCACGTTCG | 573 |
| sapiens_ | 181173650- | CCTCACACGCGAAAGGTCCCCGGTTCGAAA | |
| tRNA- | 181173722 | CCGGGCGGAAACA | |
| Val- | (+) | | |
| CAC- | | | |
| 1- | | | |
| 4 | | | |
| Homo_ | chr5: | GTTTCCGTAGTGTAGTGGTTATCACGTTCG | 574 |
| sapiens_ | 181222395- | CCTCACACGCGAAAGGTCCCCGGTTCGAAA | |
| tRNA- | 181222467 | CCGGGCGGAAACA | |
| Val- | (−) | | |
| CAC- | | | |
| 1- | | | |
| 5 | | | |
| Homo_ | chr6: | GTTTCCGTAGTGTAGTGGTTATCACGTTCG | 575 |
| sapiens_ | 26538054- | CCTCACACGCGAAAGGTCCCCGGTTCGAAA | |
| tRNA- | 26538126 | CCGGGCGGAAACA | |
| Val- | (+) | | |
| CAC- | | | |
| 1- | | | |
| 6 | | | |
| Homo_ | chr1: | GTTTCCGTAGTGTAGTGGTTATCACGTTCG | 576 |
| sapiens_ | 149712552- | CCTCACACGCGAAAGGTCCCCGGTTCGAAA | |
| tRNA- | 149712624 | CCGGGCGGAAACA | |
| Val- | (−) | | |
| CAC- | | | |
| 1- | | | |
| 7 | | | |
| Homo_ | chr1: | GTTTCCGTAGTGTAGTGGTTATCATGTTCG | 577 |
| sapiens_ | 145157157- | CCTCACACGCGAAAGGTCCCCGGTTCGAAA | |
| tRNA- | 145157229 | CTGGATGGAAACA | |
| Val- | (+) | | |
| CAC- | | | |
| 14- | | | |
| 1 | | | |
| Homo_ | chr6: | GCTTCTGTAGTGTAGTGGTTATCACGTTCG | 578 |
| sapiens_ | 27280270- | CCTCACACGCGAAAGGTCCCCGGTTCGAAA | |
| tRNA- | 27280342 | CCGGGCAGAAGCA | |
| Val- | (−) | | |
| CAC- | | | |
| 2- | | | |
| 1 | | | |
| Homo_ | chr19: | GTTTCCGTAGTGTAGCGGTTATCACATTCG | 579 |
| sapiens_ | 4724635- | CCTCACACGCGAAAGGTCCCCGGTTCGATC | |
| tRNA- | 4724707 | CCGGGCGGAAACA | |
| Val- | (−) | | |
| CAC- | | | |
| 3- | | | |
| 1 | | | |
| Homo_ | chr1: | GTTTCCGTAGTGTAGTGGTTATCACGTTCG | 580 |
| sapiens_ | 143803994- | CCTCACACGCGAAAGGTCCCCGGTTCGAAA | |
| tRNA- | 143804066 | CTGGGCGGAAACA | |
| Val- | (−) | | |
| CAC- | | | |
| 4- | | | |
| 1 | | | |
| Homo_ | chr1: | GTTTCCGTAGTGTAGTGGTTATCACGTTCG | 581 |
| sapiens_ | 121020729- | CCTCACACGCGAAAGGTCCCCGGTTCGAAA | |
| tRNA- | 121020801 | CCGGGCGGAAACA | |
| Val- | (−) | | |
| CAC- | | | |
| 5- | | | |
| 1 | | | |
| Homo_ | chr6: | GTTTCCGTAGTGGAGTGGTTATCACGTTCG | 582 |
| sapiens_ | 27206088- | CCTCACACGCGAAAGGTCCCCGGTTTGAAA | |
| tRNA- | 27206160 | CCAGGCGGAAACA | |
| Val- | (−) | | |
| CAC- | | | |
| 6- | | | |
| 1 | | | |
| Homo_ | chr11: | GGTTCCATAGTGTAGTGGTTATCACGTCTG | 583 |
| sapiens_ | 59550629- | CTTTACACGCAGAAGGTCCTGGGTTCGAGC | |
| tRNA- | 59550701 | CCCAGTGGAACCA | |
| Val- | (−) | | |
| TAC- | | | |
| 1- | | | |
| 1 | | | |
| Homo_ | chrX: | GGTTCCATAGTGTAGTGGTTATCACGTCTG | 584 |
| sapiens_ | 18674909- | CTTTACACGCAGAAGGTCCTGGGTTCGAGC | |
| tRNA- | 18674981 | CCCAGTGGAACCA | |
| Val- | (−) | | |
| TAC- | | | |
| 1- | | | |
| 2 | | | |
| Homo_ | chr11: | GGTTCCATAGTGTAGCGGTTATCACGTCTG | 585 |
| sapiens_ | 59550987- | CTTTACACGCAGAAGGTCCTGGGTTCGAGC | |
| tRNA- | 59551059 | CCCAGTGGAACCA | |
| Val- | (−) | | |
| TAC- | | | |
| 2- | | | |
| 1 | | | |
| Homo_ | chr10: | GGTTCCATAGTGTAGTGGTTATCACATCTG | 586 |
| sapiens_ | 5853711- | CTTTACACGCAGAAGGTCCTGGGTTCAAGC | |
| tRNA- | 5853783 | CCCAGTGGAACCA | |
| Val- | (−) | | |
| TAC- | | | |
| 3- | | | |
| 1 | | | |
| Homo_ | chr6: | GTTTCCGTGGTGTAGTGGTTATCACATTCG | 587 |
| sapiens_ | 27290626- | CCTTACACGCGAAAGGTCCTCGGGTCGAAA | |
| tRNA- | 27290698 | CCGAGCGGAAACA | |
| Val- | (+) | | |
| TAC- | | | |
| 4- | | | |
| 1 | | | |
| Homo_ | chr1: | AGCAGAGTGGCGCAGCGGAAGCGTGCTGGG | 588 |
| sapiens_ | 153671250- | CCCATAACCCAGAGGTCGATGGATCGAAAC | |
| tRNA- | 153671321 | CATCCTCTGCTA | |
| iMet- | (+) | | |
| CAT- | | | |
| 1- | | | |
| 1 | | | |
| Homo_ | chr6: | AGCAGAGTGGCGCAGCGGAAGCGTGCTGGG | 589 |
| sapiens_ | 26286526- | CCCATAACCCAGAGGTCGATGGATCGAAAC | |
| tRNA- | 26286597 | CATCCTCTGCTA | |
| iMet- | (+) | | |
| CAT- | | | |
| 1- | | | |
| 2 | | | |
| Homo_ | chr6: | AGCAGAGTGGCGCAGCGGAAGCGTGCTGGG | 590 |
| sapiens_ | 26313124- | CCCATAACCCAGAGGTCGATGGATCGAAAC | |
| tRNA- | 26313195 | CATCCTCTGCTA | |
| iMet- | | | |
| CAT- | | | |
| 1- | | | |
| 3 | | | |
| Homo_ | chr6: | AGCAGAGTGGCGCAGCGGAAGCGTGCTGGG | 591 |
| sapiens_ | 26330301- | CCCATAACCCAGAGGTCGATGGATCGAAAC | |
| tRNA- | 26330372 | CATCCTCTGCTA | |
| iMet- | (−) | | |
| CAT- | | | |
| 1- | | | |
| 4 | | | |
| Homo_ | chr6: | AGCAGAGTGGCGCAGCGGAAGCGTGCTGGG | 592 |
| sapiens_ | 27332985- | CCCATAACCCAGAGGTCGATGGATCGAAAC | |
| tRNA- | 27333056 | CATCCTCTGCTA | |
| iMet- | (−) | | |
| CAT- | | | |
| 1- | | | |
| 5 | | | |
| Homo_ | chr6: | AGCAGAGTGGCGCAGCGGAAGCGTGCTGGG | 593 |
| sapiens_ | 27592821- | CCCATAACCCAGAGGTCGATGGATCGAAAC | |
| tRNA- | 27592892 | CATCCTCTGCTA | |
| iMet- | (−) | | |
| CAT- | | | |
| 1- | | | |
| 6 | | | |
| Homo_ | chr6: | AGCAGAGTGGCGCAGCGGAAGCGTGCTGGG | 594 |
| sapiens_ | 27902493- | CCCATAACCCAGAGGTCGATGGATCGAAAC | |
| tRNA- | 27902564 | CATCCTCTGCTA | |
| iMet- | (−) | | |
| CAT- | | | |
| 1- | | | |
| 7 | | | |
| Homo_ | chr17: | AGCAGAGTGGCGCAGCGGAAGCGTGCTGGG | 595 |
| sapiens_ | 82494721- | CCCATAACCCAGAGGTCGATGGATCGAAAC | |
| tRNA- | 82494792 | CATCCTCTGCTA | |
| iMet- | (−) | | |
| CAT- | | | |
| 1- | | | |
| 8 | | | |
| Homo_ | chr6: | AGCAGAGTGGCGCAGCGGAAGCGTGCTGGG | 596 |
| sapiens_ | 27777885- | CCCATAACCCAGAGGTCGATGGATCTAAAC | |
| tRNA- | 27777956 | CATCCTCTGCTA | |
| iMet- | (+) | | |
| CAT- | | | |
| 2- | | | |
| 1 | | | |

TABLE 2
Exemplary embodiments of possible human tRNA genes, relevant
protospacer sequences and the respective base editor capable of installing a
single transition mutation or single transversion mutation to convert the
endogenous tRNA anticodon into a nonsense suppressor anticodon.

| | | | SEQ ID |
|---|---|---|---|
| tRNA Target | Protospacer | Editor | NO: |
| Homo_sapiens_tRNA- | AAGTCAGACGCCTTATCCAT | CBE | 597 |
| Arg-TCG-1-1 | | | |
| Homo_sapiens_tRNA- | GAAGTCAGACGCCTTATCCA | CBE | 598 |
| Arg-TCG-1-1 | | | |
| Homo_sapiens_tRNA- | CGAAGTCAGACGCCTTATCC | CBE | 599 |
| Arg-TCG-1-1 | | | |
| Homo_sapiens_tRNA- | CCGAAGTCAGACGCCTTATC | CBE | 600 |
| Arg-TCG-1-1 | | | |
| Homo_sapiens_tRNA- | TCCGAAGTCAGACGCCTTAT | CBE | 601 |
| Arg-TCG-1-1 | | | |
| Homo_sapiens_tRNA- | ATCCGAAGTCAGACGCCTTA | CBE | 602 |
| Arg-TCG-1-1 | | | |
| Homo_sapiens_tRNA- | GATCCGAAGTCAGACGCCTT | CBE | 603 |
| Arg-TCG-1-1 | | | |
| Homo_sapiens_tRNA- | TGATCCGAAGTCAGACGCCT | CBE | 604 |
| Arg-TCG-1-1 | | | |
| Homo_sapiens_tRNA- | CTGATCCGAAGTCAGACGCC | CBE | 605 |
| Arg-TCG-1-1 | | | |
| Homo_sapiens_tRNA- | TCTGATCCGAAGTCAGACGC | CBE | 606 |
| Arg-TCG-1-1 | | | |
| Homo_sapiens_tRNA- | TTCTGATCCGAAGTCAGACG | CBE | 607 |
| Arg-TCG-1-1 | | | |
| Homo_sapiens_tRNA- | CTTCTGATCCGAAGTCAGAC | CBE | 608 |
| Arg-TCG-1-1 | | | |
| Homo_sapiens_tRNA- | TCTTCTGATCCGAAGTCAGA | CBE | 609 |
| Arg-TCG-1-1 | | | |
| Homo_sapiens_tRNA- | ATCTTCTGATCCGAAGTCAG | CBE | 610 |
| Arg-TCG-1-1 | | | |
| Homo_sapiens_tRNA- | AATCTTCTGATCCGAAGTCA | CBE | 611 |
| Arg-TCG-1-1 | | | |
| Homo_sapiens_tRNA- | CAATCTTCTGATCCGAAGTC | CBE | 612 |
| Arg-TCG-1-1 | | | |
| Homo_sapiens_tRNA- | GCAATCTTCTGATCCGAAGT | CBE | 613 |
| Arg-TCG-1-1 | | | |
| Homo_sapiens_tRNA- | TGCAATCTTCTGATCCGAAG | CBE | 614 |
| Arg-TCG-1-1 | | | |
| Homo_sapiens_tRNA- | AAGTCAGACGCCTTATCCAT | CBE | 615 |
| Arg-TCG-2-1 | | | |
| Homo_sapiens_tRNA- | GAAGTCAGACGCCTTATCCA | CBE | 616 |
| Arg-TCG-2-1 | | | |
| Homo_sapiens_tRNA- | CGAAGTCAGACGCCTTATCC | CBE | 617 |
| Arg-TCG-2-1 | | | |
| Homo_sapiens_tRNA- | CCGAAGTCAGACGCCTTATC | CBE | 618 |
| Arg-TCG-2-1 | | | |
| Homo_sapiens_tRNA- | TCCGAAGTCAGACGCCTTAT | CBE | 619 |
| Arg-TCG-2-1 | | | |
| Homo_sapiens_tRNA- | ATCCGAAGTCAGACGCCTTA | CBE | 620 |
| Arg-TCG-2-1 | | | |
| Homo_sapiens_tRNA- | GATCCGAAGTCAGACGCCTT | CBE | 621 |
| Arg-TCG-2-1 | | | |
| Homo_sapiens_tRNA- | TGATCCGAAGTCAGACGCCT | CBE | 622 |
| Arg-TCG-2-1 | | | |
| Homo_sapiens_tRNA- | CTGATCCGAAGTCAGACGCC | CBE | 623 |
| Arg-TCG-2-1 | | | |
| Homo_sapiens_tRNA- | TCTGATCCGAAGTCAGACGC | CBE | 624 |
| Arg-TCG-2-1 | | | |
| Homo_sapiens_tRNA- | TTCTGATCCGAAGTCAGACG | CBE | 625 |
| Arg-TCG-2-1 | | | |
| Homo_sapiens_tRNA- | CTTCTGATCCGAAGTCAGAC | CBE | 626 |
| Arg-TCG-2-1 | | | |
| Homo_sapiens_tRNA- | TCTTCTGATCCGAAGTCAGA | CBE | 627 |
| Arg-TCG-2-1 | | | |
| Homo_sapiens_tRNA- | ATCTTCTGATCCGAAGTCAG | CBE | 628 |
| Arg-TCG-2-1 | | | |
| Homo_sapiens_tRNA- | AATCTTCTGATCCGAAGTCA | CBE | 629 |
| Arg-TCG-2-1 | | | |
| Homo_sapiens_tRNA- | CAATCTTCTGATCCGAAGTC | CBE | 630 |
| Arg-TCG-2-1 | | | |
| Homo_sapiens_tRNA- | TCAATCTTCTGATCCGAAGT | CBE | 631 |
| Arg-TCG-2-1 | | | |
| Homo_sapiens_tRNA- | CTCAATCTTCTGATCCGAAG | CBE | 632 |
| Arg-TCG-2-1 | | | |
| Homo_sapiens_tRNA- | AAGTCAGACGCCTTATCCAT | CBE | 633 |
| Arg-TCG-3-1 | | | |
| Homo_sapiens_tRNA- | GAAGTCAGACGCCTTATCCA | CBE | 634 |
| Arg-TCG-3-1 | | | |
| Homo_sapiens_tRNA- | CGAAGTCAGACGCCTTATCC | CBE | 635 |
| Arg-TCG-3-1 | | | |
| Homo_sapiens_tRNA- | CCGAAGTCAGACGCCTTATC | CBE | 636 |
| Arg-TCG-3-1 | | | |
| Homo_sapiens_tRNA- | TCCGAAGTCAGACGCCTTAT | CBE | 637 |
| Arg-TCG-3-1 | | | |
| Homo_sapiens_tRNA- | ATCCGAAGTCAGACGCCTTA | CBE | 638 |
| Arg-TCG-3-1 | | | |
| Homo_sapiens_tRNA- | GATCCGAAGTCAGACGCCTT | CBE | 639 |
| Arg-TCG-3-1 | | | |
| Homo_sapiens_tRNA- | TGATCCGAAGTCAGACGCCT | CBE | 640 |
| Arg-TCG-3-1 | | | |
| Homo_sapiens_tRNA- | CTGATCCGAAGTCAGACGCC | CBE | 641 |
| Arg-TCG-3-1 | | | |
| Homo_sapiens_tRNA- | TCTGATCCGAAGTCAGACGC | CBE | 642 |
| Arg-TCG-3-1 | | | |
| Homo_sapiens_tRNA- | TTCTGATCCGAAGTCAGACG | CBE | 643 |
| Arg-TCG-3-1 | | | |
| Homo_sapiens_tRNA- | CTTCTGATCCGAAGTCAGAC | CBE | 644 |
| Arg-TCG-3-1 | | | |
| Homo_sapiens_tRNA- | TCTTCTGATCCGAAGTCAGA | CBE | 645 |
| Arg-TCG-3-1 | | | |
| Homo_sapiens_tRNA- | ATCTTCTGATCCGAAGTCAG | CBE | 646 |
| Arg-TCG-3-1 | | | |
| Homo_sapiens_tRNA- | AATCTTCTGATCCGAAGTCA | CBE | 647 |
| Arg-TCG-3-1 | | | |
| Homo_sapiens_tRNA- | CAATCTTCTGATCCGAAGTC | CBE | 648 |
| Arg-TCG-3-1 | | | |
| Homo_sapiens_tRNA- | TCAATCTTCTGATCCGAAGT | CBE | 649 |
| Arg-TCG-3-1 | | | |
| Homo_sapiens_tRNA- | CTCAATCTTCTGATCCGAAG | CBE | 650 |
| Arg-TCG-3-1 | | | |
| Homo_sapiens_tRNA- | AAGTCAGACGCCTTATCCAT | CBE | 651 |
| Arg-TCG-4-1 | | | |
| Homo_sapiens_tRNA- | GAAGTCAGACGCCTTATCCA | CBE | 652 |
| Arg-TCG-4-1 | | | |
| Homo_sapiens_tRNA- | CGAAGTCAGACGCCTTATCC | CBE | 653 |
| Arg-TCG-4-1 | | | |
| Homo_sapiens_tRNA- | CCGAAGTCAGACGCCTTATC | CBE | 654 |
| Arg-TCG-4-1 | | | |
| Homo_sapiens_tRNA- | TCCGAAGTCAGACGCCTTAT | CBE | 655 |
| Arg-TCG-4-1 | | | |
| Homo_sapiens_tRNA- | ATCCGAAGTCAGACGCCTTA | CBE | 656 |
| Arg-TCG-4-1 | | | |
| Homo_sapiens_tRNA- | GATCCGAAGTCAGACGCCTT | CBE | 657 |
| Arg-TCG-4-1 | | | |
| Homo_sapiens_tRNA- | TGATCCGAAGTCAGACGCCT | CBE | 658 |
| Arg-TCG-4-1 | | | |
| Homo_sapiens_tRNA- | CTGATCCGAAGTCAGACGCC | CBE | 659 |
| Arg-TCG-4-1 | | | |
| Homo_sapiens_tRNA- | TCTGATCCGAAGTCAGACGC | CBE | 660 |
| Arg-TCG-4-1 | | | |
| Homo_sapiens_tRNA- | TTCTGATCCGAAGTCAGACG | CBE | 661 |
| Arg-TCG-4-1 | | | |
| Homo_sapiens_tRNA- | CTTCTGATCCGAAGTCAGAC | CBE | 662 |
| Arg-TCG-4-1 | | | |
| Homo_sapiens_tRNA- | TCTTCTGATCCGAAGTCAGA | CBE | 663 |
| Arg-TCG-4-1 | | | |
| Homo_sapiens_tRNA- | ATCTTCTGATCCGAAGTCAG | CBE | 664 |
| Arg-TCG-4-1 | | | |
| Homo_sapiens_tRNA- | AATCTTCTGATCCGAAGTCA | CBE | 665 |
| Arg-TCG-4-1 | | | |
| Homo_sapiens_tRNA- | CAATCTTCTGATCCGAAGTC | CBE | 666 |
| Arg-TCG-4-1 | | | |
| Homo_sapiens_tRNA- | TCAATCTTCTGATCCGAAGT | CBE | 667 |
| Arg-TCG-4-1 | | | |
| Homo_sapiens_tRNA- | CTCAATCTTCTGATCCGAAG | CBE | 668 |
| Arg-TCG-4-1 | | | |
| Homo_sapiens_tRNA- | AAGTCAGACGCCTTATCCAT | CBE | 669 |
| Arg-TCG-5-1 | | | |
| Homo_sapiens_tRNA- | GAAGTCAGACGCCTTATCCA | CBE | 670 |
| Arg-TCG-5-1 | | | |
| Homo_sapiens_tRNA- | CGAAGTCAGACGCCTTATCC | CBE | 671 |
| Arg-TCG-5-1 | | | |
| Homo_sapiens_tRNA- | CCGAAGTCAGACGCCTTATC | CBE | 672 |
| Arg-TCG-5-1 | | | |
| Homo_sapiens_tRNA- | TCCGAAGTCAGACGCCTTAT | CBE | 673 |
| Arg-TCG-5-1 | | | |
| Homo_sapiens_tRNA- | ATCCGAAGTCAGACGCCTTA | CBE | 674 |
| Arg-TCG-5-1 | | | |
| Homo_sapiens_tRNA- | GATCCGAAGTCAGACGCCTT | CBE | 675 |
| Arg-TCG-5-1 | | | |
| Homo_sapiens_tRNA- | TGATCCGAAGTCAGACGCCT | CBE | 676 |
| Arg-TCG-5-1 | | | |
| Homo_sapiens_tRNA- | CTGATCCGAAGTCAGACGCC | CBE | 677 |
| Arg-TCG-5-1 | | | |
| Homo_sapiens_tRNA- | TCTGATCCGAAGTCAGACGC | CBE | 678 |
| Arg-TCG-5-1 | | | |
| Homo_sapiens_tRNA- | TTCTGATCCGAAGTCAGACG | CBE | 679 |
| Arg-TCG-5-1 | | | |
| Homo_sapiens_tRNA- | CTTCTGATCCGAAGTCAGAC | CBE | 680 |
| Arg-TCG-5-1 | | | |
| Homo_sapiens_tRNA- | TCTTCTGATCCGAAGTCAGA | CBE | 681 |
| Arg-TCG-5-1 | | | |
| Homo_sapiens_tRNA- | ATCTTCTGATCCGAAGTCAG | CBE | 682 |
| Arg-TCG-5-1 | | | |
| Homo_sapiens_tRNA- | AATCTTCTGATCCGAAGTCA | CBE | 683 |
| Arg-TCG-5-1 | | | |
| Homo_sapiens_tRNA- | CAATCTTCTGATCCGAAGTC | CBE | 684 |
| Arg-TCG-5-1 | | | |
| Homo_sapiens_tRNA- | TCAATCTTCTGATCCGAAGT | CBE | 685 |
| Arg-TCG-5-1 | | | |
| Homo_sapiens_tRNA- | CTCAATCTTCTGATCCGAAG | CBE | 686 |
| Arg-TCG-5-1 | | | |
| Homo_sapiens_tRNA- | AAGTCAGACGCCTTATCCAT | CBE | 687 |
| Arg-TCG-6-1 | | | |
| Homo_sapiens_tRNA- | GAAGTCAGACGCCTTATCCA | CBE | 688 |
| Arg-TCG-6-1 | | | |
| Homo_sapiens_tRNA- | CGAAGTCAGACGCCTTATCC | CBE | 689 |
| Arg-TCG-6-1 | | | |
| Homo_sapiens_tRNA- | CCGAAGTCAGACGCCTTATC | CBE | 690 |
| Arg-TCG-6-1 | | | |
| Homo_sapiens_tRNA- | TCCGAAGTCAGACGCCTTAT | CBE | 691 |
| Arg-TCG-6-1 | | | |
| Homo_sapiens_tRNA- | ATCCGAAGTCAGACGCCTTA | CBE | 692 |
| Arg-TCG-6-1 | | | |
| Homo_sapiens_tRNA- | GATCCGAAGTCAGACGCCTT | CBE | 693 |
| Arg-TCG-6-1 | | | |
| Homo_sapiens_tRNA- | TGATCCGAAGTCAGACGCCT | CBE | 694 |
| Arg-TCG-6-1 | | | |
| Homo_sapiens_tRNA- | TTGATCCGAAGTCAGACGCC | CBE | 695 |
| Arg-TCG-6-1 | | | |
| Homo_sapiens_tRNA- | TTTGATCCGAAGTCAGACGC | CBE | 696 |
| Arg-TCG-6-1 | | | |
| Homo_sapiens_tRNA- | TTTTGATCCGAAGTCAGACG | CBE | 697 |
| Arg-TCG-6-1 | | | |
| Homo_sapiens_tRNA- | CTTTTGATCCGAAGTCAGAC | CBE | 698 |
| Arg-TCG-6-1 | | | |
| Homo_sapiens_tRNA- | TCTTTTGATCCGAAGTCAGA | CBE | 699 |
| Arg-TCG-6-1 | | | |
| Homo_sapiens_tRNA- | ATCTTTTGATCCGAAGTCAG | CBE | 700 |
| Arg-TCG-6-1 | | | |
| Homo_sapiens_tRNA- | AATCTTTTGATCCGAAGTCA | CBE | 701 |
| Arg-TCG-6-1 | | | |
| Homo_sapiens_tRNA- | CAATCTTTTGATCCGAAGTC | CBE | 702 |
| Arg-TCG-6-1 | | | |
| Homo_sapiens_tRNA- | GCAATCTTTTGATCCGAAGT | CBE | 703 |
| Arg-TCG-6-1 | | | |
| Homo_sapiens_tRNA- | TGCAATCTTTTGATCCGAAG | CBE | 704 |
| Arg-TCG-6-1 | | | |
| Homo_sapiens_tRNA- | GCAGTCAAATGCTCTACCAC | CABE | 705 |
| Cys-GCA-1-1 | | | |
| Homo_sapiens_tRNA- | TGCAGTCAAATGCTCTACCA | CABE | 706 |
| Cys-GCA-1-1 | | | |
| Homo_sapiens_tRNA- | CTGCAGTCAAATGCTCTACC | CABE | 707 |
| Cys-GCA-1-1 | | | |
| Homo_sapiens_tRNA- | TCTGCAGTCAAATGCTCTAC | CABE | 708 |
| Cys-GCA-1-1 | | | |
| Homo_sapiens_tRNA- | ATCTGCAGTCAAATGCTCTA | CABE | 709 |
| Cys-GCA-1-1 | | | |
| Homo_sapiens_tRNA- | GATCTGCAGTCAAATGCTCT | CABE | 710 |
| Cys-GCA-1-1 | | | |
| Homo_sapiens_tRNA- | TGATCTGCAGTCAAATGCTC | CABE | 711 |
| Cys-GCA-1-1 | | | |
| Homo_sapiens_tRNA- | TTGATCTGCAGTCAAATGCT | CABE | 712 |
| Cys-GCA-1-1 | | | |
| Homo_sapiens_tRNA- | CTTGATCTGCAGTCAAATGC | CABE | 713 |
| Cys-GCA-1-1 | | | |
| Homo_sapiens_tRNA- | TCTTGATCTGCAGTCAAATG | CABE | 714 |
| Cys-GCA-1-1 | | | |
| Homo_sapiens_tRNA- | CTCTTGATCTGCAGTCAAAT | CABE | 715 |
| Cys-GCA-1-1 | | | |
| Homo_sapiens_tRNA- | CCTCTTGATCTGCAGTCAAA | CABE | 716 |
| Cys-GCA-1-1 | | | |
| Homo_sapiens_tRNA- | ACCTCTTGATCTGCAGTCAA | CABE | 717 |
| Cys-GCA-1-1 | | | |
| Homo_sapiens_tRNA- | GACCTCTTGATCTGCAGTCA | CABE | 718 |
| Cys-GCA-1-1 | | | |
| Homo_sapiens_tRNA- | GGACCTCTTGATCTGCAGTC | CABE | 719 |
| Cys-GCA-1-1 | | | |
| Homo_sapiens_tRNA- | GGGACCTCTTGATCTGCAGT | CABE | 720 |
| Cys-GCA-1-1 | | | |
| Homo_sapiens_tRNA- | AGGGACCTCTTGATCTGCAG | CABE | 721 |
| Cys-GCA-1-1 | | | |
| Homo_sapiens_tRNA- | CAGGGACCTCTTGATCTGCA | CABE | 722 |
| Cys-GCA-1-1 | | | |
| Homo_sapiens_tRNA- | GCAGTCAAATGCTCTACCCC | CABE | 723 |
| Cys-GCA-10-1 | | | |
| Homo_sapiens_tRNA- | TGCAGTCAAATGCTCTACCC | CABE | 724 |
| Cys-GCA-10-1 | | | |
| Homo_sapiens_tRNA- | CTGCAGTCAAATGCTCTACC | CABE | 725 |
| Cys-GCA-10-1 | | | |
| Homo_sapiens_tRNA- | TCTGCAGTCAAATGCTCTAC | CABE | 726 |
| Cys-GCA-10-1 | | | |
| Homo_sapiens_tRNA- | ATCTGCAGTCAAATGCTCTA | CABE | 727 |
| Cys-GCA-10-1 | | | |
| Homo_sapiens_tRNA- | GATCTGCAGTCAAATGCTCT | CABE | 728 |
| Cys-GCA-10-1 | | | |
| Homo_sapiens_tRNA- | TGATCTGCAGTCAAATGCTC | CABE | 729 |
| Cys-GCA-10-1 | | | |
| Homo_sapiens_tRNA- | TTGATCTGCAGTCAAATGCT | CABE | 730 |
| Cys-GCA-10-1 | | | |
| Homo_sapiens_tRNA- | CTTGATCTGCAGTCAAATGC | CABE | 731 |
| Cys-GCA-10-1 | | | |
| Homo_sapiens_tRNA- | TCTTGATCTGCAGTCAAATG | CABE | 732 |
| Cys-GCA-10-1 | | | |
| Homo_sapiens_tRNA- | CTCTTGATCTGCAGTCAAAT | CABE | 733 |
| Cys-GCA-10-1 | | | |
| Homo_sapiens_tRNA- | CCTCTTGATCTGCAGTCAAA | CABE | 734 |
| Cys-GCA-10-1 | | | |
| Homo_sapiens_tRNA- | ACCTCTTGATCTGCAGTCAA | CABE | 735 |
| Cys-GCA-10-1 | | | |
| Homo_sapiens_tRNA- | GACCTCTTGATCTGCAGTCA | CABE | 736 |
| Cys-GCA-10-1 | | | |
| Homo_sapiens_tRNA- | GGACCTCTTGATCTGCAGTC | CABE | 737 |
| Cys-GCA-10-1 | | | |
| Homo_sapiens_tRNA- | GGGACCTCTTGATCTGCAGT | CABE | 738 |
| Cys-GCA-10-1 | | | |
| Homo_sapiens_tRNA- | AGGGACCTCTTGATCTGCAG | CABE | 739 |
| Cys-GCA-10-1 | | | |
| Homo_sapiens_tRNA- | CAGGGACCTCTTGATCTGCA | CABE | 740 |
| Cys-GCA-10-1 | | | |
| Homo_sapiens_tRNA- | GCAGTCAAATGCTCTACCGC | CABE | 741 |
| Cys-GCA-11-1 | | | |
| Homo_sapiens_tRNA- | TGCAGTCAAATGCTCTACCG | CABE | 742 |
| Cys-GCA-11-1 | | | |
| Homo_sapiens_tRNA- | CTGCAGTCAAATGCTCTACC | CABE | 743 |
| Cys-GCA-11-1 | | | |
| Homo_sapiens_tRNA- | TCTGCAGTCAAATGCTCTAC | CABE | 744 |
| Cys-GCA-11-1 | | | |
| Homo_sapiens_tRNA- | ATCTGCAGTCAAATGCTCTA | CABE | 745 |
| Cys-GCA-11-1 | | | |
| Homo_sapiens_tRNA- | GATCTGCAGTCAAATGCTCT | CABE | 746 |
| Cys-GCA-11-1 | | | |
| Homo_sapiens_tRNA- | TGATCTGCAGTCAAATGCTC | CABE | 747 |
| Cys-GCA-11-1 | | | |
| Homo_sapiens_tRNA- | TTGATCTGCAGTCAAATGCT | CABE | 748 |
| Cys-GCA-11-1 | | | |
| Homo_sapiens_tRNA- | CTTGATCTGCAGTCAAATGC | CABE | 749 |
| Cys-GCA-11-1 | | | |
| Homo_sapiens_tRNA- | TCTTGATCTGCAGTCAAATG | CABE | 750 |
| Cys-GCA-11-1 | | | |
| Homo_sapiens_tRNA- | CTCTTGATCTGCAGTCAAAT | CABE | 751 |
| Cys-GCA-11-1 | | | |
| Homo_sapiens_tRNA- | CCTCTTGATCTGCAGTCAAA | CABE | 752 |
| Cys-GCA-11-1 | | | |
| Homo_sapiens_tRNA- | ACCTCTTGATCTGCAGTCAA | CABE | 753 |
| Cys-GCA-11-1 | | | |
| Homo_sapiens_tRNA- | GACCTCTTGATCTGCAGTCA | CABE | 754 |
| Cys-GCA-11-1 | | | |
| Homo_sapiens_tRNA- | GGACCTCTTGATCTGCAGTC | CABE | 755 |
| Cys-GCA-11-1 | | | |
| Homo_sapiens_tRNA- | GGGACCTCTTGATCTGCAGT | CABE | 756 |
| Cys-GCA-11-1 | | | |
| Homo_sapiens_tRNA- | GGGGACCTCTTGATCTGCAG | CABE | 757 |
| Cys-GCA-11-1 | | | |
| Homo_sapiens_tRNA- | CGGGGACCTCTTGATCTGCA | CABE | 758 |
| Cys-GCA-11-1 | | | |
| Homo_sapiens_tRNA- | GCAGTCAAATGCTCTACCCC | CABE | 759 |
| Cys-GCA-12-1 | | | |
| Homo_sapiens_tRNA- | TGCAGTCAAATGCTCTACCC | CABE | 760 |
| Cys-GCA-12-1 | | | |
| Homo_sapiens_tRNA- | CTGCAGTCAAATGCTCTACC | CABE | 761 |
| Cys-GCA-12-1 | | | |
| Homo_sapiens_tRNA- | TCTGCAGTCAAATGCTCTAC | CABE | 762 |
| Cys-GCA-12-1 | | | |
| Homo_sapiens_tRNA- | ATCTGCAGTCAAATGCTCTA | CABE | 763 |
| Cys-GCA-12-1 | | | |
| Homo_sapiens_tRNA- | GATCTGCAGTCAAATGCTCT | CABE | 764 |
| Cys-GCA-12-1 | | | |
| Homo_sapiens_tRNA- | TGATCTGCAGTCAAATGCTC | CABE | 765 |
| Cys-GCA-12-1 | | | |
| Homo_sapiens_tRNA- | TTGATCTGCAGTCAAATGCT | CABE | 766 |
| Cys-GCA-12-1 | | | |
| Homo_sapiens_tRNA- | TTTGATCTGCAGTCAAATGC | CABE | 767 |
| Cys-GCA-12-1 | | | |
| Homo_sapiens_tRNA- | TTTTGATCTGCAGTCAAATG | CABE | 768 |
| Cys-GCA-12-1 | | | |
| Homo_sapiens_tRNA- | CTTTTGATCTGCAGTCAAAT | CABE | 769 |
| Cys-GCA-12-1 | | | |
| Homo_sapiens_tRNA- | CCTTTTGATCTGCAGTCAAA | CABE | 770 |
| Cys-GCA-12-1 | | | |
| Homo_sapiens_tRNA- | ACCTTTTGATCTGCAGTCAA | CABE | 771 |
| Cys-GCA-12-1 | | | |
| Homo_sapiens_tRNA- | GACCTTTTGATCTGCAGTCA | CABE | 772 |
| Cys-GCA-12-1 | | | |
| Homo_sapiens_tRNA- | GGACCTTTTGATCTGCAGTC | CABE | 773 |
| Cys-GCA-12-1 | | | |
| Homo_sapiens_tRNA- | GGGACCTTTTGATCTGCAGT | CABE | 774 |
| Cys-GCA-12-1 | | | |
| Homo_sapiens_tRNA- | AGGGACCTTTTGATCTGCAG | CABE | 775 |
| Cys-GCA-12-1 | | | |
| Homo_sapiens_tRNA- | CAGGGACCTTTTGATCTGCA | CABE | 776 |
| Cys-GCA-12-1 | | | |
| Homo_sapiens_tRNA- | GCAGTCAAATGCTCTACCCC | CABE | 777 |
| Cys-GCA-13-1 | | | |
| Homo_sapiens_tRNA- | TGCAGTCAAATGCTCTACCC | CABE | 778 |
| Cys-GCA-13-1 | | | |
| Homo_sapiens_tRNA- | CTGCAGTCAAATGCTCTACC | CABE | 779 |
| Cys-GCA-13-1 | | | |
| Homo_sapiens_tRNA- | TCTGCAGTCAAATGCTCTAC | CABE | 780 |
| Cys-GCA-13-1 | | | |
| Homo_sapiens_tRNA- | ATCTGCAGTCAAATGCTCTA | CABE | 781 |
| Cys-GCA-13-1 | | | |
| Homo_sapiens_tRNA- | GATCTGCAGTCAAATGCTCT | CABE | 782 |
| Cys-GCA-13-1 | | | |
| Homo_sapiens_tRNA- | TGATCTGCAGTCAAATGCTC | CABE | 783 |
| Cys-GCA-13-1 | | | |
| Homo_sapiens_tRNA- | TTGATCTGCAGTCAAATGCT | CABE | 784 |
| Cys-GCA-13-1 | | | |
| Homo_sapiens_tRNA- | CTTGATCTGCAGTCAAATGC | CABE | 785 |
| Cys-GCA-13-1 | | | |
| Homo_sapiens_tRNA- | TCTTGATCTGCAGTCAAATG | CABE | 786 |
| Cys-GCA-13-1 | | | |
| Homo_sapiens_tRNA- | CTCTTGATCTGCAGTCAAAT | CABE | 787 |
| Cys-GCA-13-1 | | | |
| Homo_sapiens_tRNA- | CCTCTTGATCTGCAGTCAAA | CABE | 788 |
| Cys-GCA-13-1 | | | |
| Homo_sapiens_tRNA- | ACCTCTTGATCTGCAGTCAA | CABE | 789 |
| Cys-GCA-13-1 | | | |
| Homo_sapiens_tRNA- | GACCTCTTGATCTGCAGTCA | CABE | 790 |
| Cys-GCA-13-1 | | | |
| Homo_sapiens_tRNA- | GGACCTCTTGATCTGCAGTC | CABE | 791 |
| Cys-GCA-13-1 | | | |
| Homo_sapiens_tRNA- | GGGACCTCTTGATCTGCAGT | CABE | 792 |
| Cys-GCA-13-1 | | | |
| Homo_sapiens_tRNA- | GGGGACCTCTTGATCTGCAG | CABE | 793 |
| Cys-GCA-13-1 | | | |
| Homo_sapiens_tRNA- | TGGGGACCTCTTGATCTGCA | CABE | 794 |
| Cys-GCA-13-1 | | | |
| Homo_sapiens_tRNA- | GCAGTCAAATGCTCTACCCC | CABE | 795 |
| Cys-GCA-14-1 | | | |
| Homo_sapiens_tRNA- | TGCAGTCAAATGCTCTACCC | CABE | 796 |
| Cys-GCA-14-1 | | | |
| Homo_sapiens_tRNA- | CTGCAGTCAAATGCTCTACC | CABE | 797 |
| Cys-GCA-14-1 | | | |
| Homo_sapiens_tRNA- | TCTGCAGTCAAATGCTCTAC | CABE | 798 |
| Cys-GCA-14-1 | | | |
| Homo_sapiens_tRNA- | ATCTGCAGTCAAATGCTCTA | CABE | 799 |
| Cys-GCA-14-1 | | | |
| Homo_sapiens_tRNA- | GATCTGCAGTCAAATGCTCT | CABE | 800 |
| Cys-GCA-14-1 | | | |
| Homo_sapiens_tRNA- | TGATCTGCAGTCAAATGCTC | CABE | 801 |
| Cys-GCA-14-1 | | | |
| Homo_sapiens_tRNA- | TTGATCTGCAGTCAAATGCT | CABE | 802 |
| Cys-GCA-14-1 | | | |
| Homo_sapiens_tRNA- | CTTGATCTGCAGTCAAATGC | CABE | 803 |
| Cys-GCA-14-1 | | | |
| Homo_sapiens_tRNA- | TCTTGATCTGCAGTCAAATG | CABE | 804 |
| Cys-GCA-14-1 | | | |
| Homo_sapiens_tRNA- | TTCTTGATCTGCAGTCAAAT | CABE | 805 |
| Cys-GCA-14-1 | | | |
| Homo_sapiens_tRNA- | CTTCTTGATCTGCAGTCAAA | CABE | 806 |
| Cys-GCA-14-1 | | | |
| Homo_sapiens_tRNA- | ACTTCTTGATCTGCAGTCAA | CABE | 807 |
| Cys-GCA-14-1 | | | |
| Homo_sapiens_tRNA- | GACTTCTTGATCTGCAGTCA | CABE | 808 |
| Cys-GCA-14-1 | | | |
| Homo_sapiens_tRNA- | GGACTTCTTGATCTGCAGTC | CABE | 809 |
| Cys-GCA-14-1 | | | |
| Homo_sapiens_tRNA- | GGGACTTCTTGATCTGCAGT | CABE | 810 |
| Cys-GCA-14-1 | | | |
| Homo_sapiens_tRNA- | GGGGACTTCTTGATCTGCAG | CABE | 811 |
| Cys-GCA-14-1 | | | |
| Homo_sapiens_tRNA- | CGGGGACTTCTTGATCTGCA | CABE | 812 |
| Cys-GCA-14-1 | | | |
| Homo_sapiens_tRNA- | GCAGTCAAATGCTCTACCCC | CABE | 813 |
| Cys-GCA-15-1 | | | |
| Homo_sapiens_tRNA- | TGCAGTCAAATGCTCTACCC | CABE | 814 |
| Cys-GCA-15-1 | | | |
| Homo_sapiens_tRNA- | CTGCAGTCAAATGCTCTACC | CABE | 815 |
| Cys-GCA-15-1 | | | |
| Homo_sapiens_tRNA- | TCTGCAGTCAAATGCTCTAC | CABE | 816 |
| Cys-GCA-15-1 | | | |
| Homo_sapiens_tRNA- | ATCTGCAGTCAAATGCTCTA | CABE | 817 |
| Cys-GCA-15-1 | | | |
| Homo_sapiens_tRNA- | GATCTGCAGTCAAATGCTCT | CABE | 818 |
| Cys-GCA-15-1 | | | |
| Homo_sapiens_tRNA- | TGATCTGCAGTCAAATGCTC | CABE | 819 |
| Cys-GCA-15-1 | | | |
| Homo_sapiens_tRNA- | TTGATCTGCAGTCAAATGCT | CABE | 820 |
| Cys-GCA-15-1 | | | |
| Homo_sapiens_tRNA- | CTTGATCTGCAGTCAAATGC | CABE | 821 |
| Cys-GCA-15-1 | | | |
| Homo_sapiens_tRNA- | TCTTGATCTGCAGTCAAATG | CABE | 822 |
| Cys-GCA-15-1 | | | |
| Homo_sapiens_tRNA- | CTCTTGATCTGCAGTCAAAT | CABE | 823 |
| Cys-GCA-15-1 | | | |
| Homo_sapiens_tRNA- | CCTCTTGATCTGCAGTCAAA | CABE | 824 |
| Cys-GCA-15-1 | | | |
| Homo_sapiens_tRNA- | ACCTCTTGATCTGCAGTCAA | CABE | 825 |
| Cys-GCA-15-1 | | | |
| Homo_sapiens_tRNA- | GACCTCTTGATCTGCAGTCA | CABE | 826 |
| Cys-GCA-15-1 | | | |
| Homo_sapiens_tRNA- | AGACCTCTTGATCTGCAGTC | CABE | 827 |
| Cys-GCA-15-1 | | | |
| Homo_sapiens_tRNA- | GAGACCTCTTGATCTGCAGT | CABE | 828 |
| Cys-GCA-15-1 | | | |
| Homo_sapiens_tRNA- | AGAGACCTCTTGATCTGCAG | CABE | 829 |
| Cys-GCA-15-1 | | | |
| Homo_sapiens_tRNA- | CAGAGACCTCTTGATCTGCA | CABE | 830 |
| Cys-GCA-15-1 | | | |
| Homo_sapiens_tRNA- | GCAGTCAAGTGCTCTACCCC | CABE | 831 |
| Cys-GCA-16-1 | | | |
| Homo_sapiens_tRNA- | TGCAGTCAAGTGCTCTACCC | CABE | 832 |
| Cys-GCA-16-1 | | | |
| Homo_sapiens_tRNA- | CTGCAGTCAAGTGCTCTACC | CABE | 833 |
| Cys-GCA-16-1 | | | |
| Homo_sapiens_tRNA- | TCTGCAGTCAAGTGCTCTAC | CABE | 834 |
| Cys-GCA-16-1 | | | |
| Homo_sapiens_tRNA- | ATCTGCAGTCAAGTGCTCTA | CABE | 835 |
| Cys-GCA-16-1 | | | |
| Homo_sapiens_tRNA- | GATCTGCAGTCAAGTGCTCT | CABE | 836 |
| Cys-GCA-16-1 | | | |
| Homo_sapiens_tRNA- | TGATCTGCAGTCAAGTGCTC | CABE | 837 |
| Cys-GCA-16-1 | | | |
| Homo_sapiens_tRNA- | TTGATCTGCAGTCAAGTGCT | CABE | 838 |
| Cys-GCA-16-1 | | | |
| Homo_sapiens_tRNA- | CTTGATCTGCAGTCAAGTGC | CABE | 839 |
| Cys-GCA-16-1 | | | |
| Homo_sapiens_tRNA- | TCTTGATCTGCAGTCAAGTG | CABE | 840 |
| Cys-GCA-16-1 | | | |
| Homo_sapiens_tRNA- | TTCTTGATCTGCAGTCAAGT | CABE | 841 |
| Cys-GCA-16-1 | | | |
| Homo_sapiens_tRNA- | CTTCTTGATCTGCAGTCAAG | CABE | 842 |
| Cys-GCA-16-1 | | | |
| Homo_sapiens_tRNA- | ACTTCTTGATCTGCAGTCAA | CABE | 843 |
| Cys-GCA-16-1 | | | |
| Homo_sapiens_tRNA- | GACTTCTTGATCTGCAGTCA | CABE | 844 |
| Cys-GCA-16-1 | | | |
| Homo_sapiens_tRNA- | GGACTTCTTGATCTGCAGTC | CABE | 845 |
| Cys-GCA-16-1 | | | |
| Homo_sapiens_tRNA- | AGGACTTCTTGATCTGCAGT | CABE | 846 |
| Cys-GCA-16-1 | | | |
| Homo_sapiens_tRNA- | AAGGACTTCTTGATCTGCAG | CABE | 847 |
| Cys-GCA-16-1 | | | |
| Homo_sapiens_tRNA- | CAAGGACTTCTTGATCTGCA | CABE | 848 |
| Cys-GCA-16-1 | | | |
| Homo_sapiens_tRNA- | GCAGTCAAATGCTCTACCCC | CABE | 849 |
| Cys-GCA-17-1 | | | |
| Homo_sapiens_tRNA- | TGCAGTCAAATGCTCTACCC | CABE | 850 |
| Cys-GCA-17-1 | | | |
| Homo_sapiens_tRNA- | CTGCAGTCAAATGCTCTACC | CABE | 851 |
| Cys-GCA-17-1 | | | |
| Homo_sapiens_tRNA- | TCTGCAGTCAAATGCTCTAC | CABE | 852 |
| Cys-GCA-17-1 | | | |
| Homo_sapiens_tRNA- | ATCTGCAGTCAAATGCTCTA | CABE | 853 |
| Cys-GCA-17-1 | | | |
| Homo_sapiens_tRNA- | GATCTGCAGTCAAATGCTCT | CABE | 854 |
| Cys-GCA-17-1 | | | |
| Homo_sapiens_tRNA- | TGATCTGCAGTCAAATGCTC | CABE | 855 |
| Cys-GCA-17-1 | | | |
| Homo_sapiens_tRNA- | TTGATCTGCAGTCAAATGCT | CABE | 856 |
| Cys-GCA-17-1 | | | |
| Homo_sapiens_tRNA- | CTTGATCTGCAGTCAAATGC | CABE | 857 |
| Cys-GCA-17-1 | | | |
| Homo_sapiens_tRNA- | TCTTGATCTGCAGTCAAATG | CABE | 858 |
| Cys-GCA-17-1 | | | |
| Homo_sapiens_tRNA- | CTCTTGATCTGCAGTCAAAT | CABE | 859 |
| Cys-GCA-17-1 | | | |
| Homo_sapiens_tRNA- | CCTCTTGATCTGCAGTCAAA | CABE | 860 |
| Cys-GCA-17-1 | | | |
| Homo_sapiens_tRNA- | ACCTCTTGATCTGCAGTCAA | CABE | 861 |
| Cys-GCA-17-1 | | | |
| Homo_sapiens_tRNA- | GACCTCTTGATCTGCAGTCA | CABE | 862 |
| Cys-GCA-17-1 | | | |
| Homo_sapiens_tRNA- | GGACCTCTTGATCTGCAGTC | CABE | 863 |
| Cys-GCA-17-1 | | | |
| Homo_sapiens_tRNA- | GGGACCTCTTGATCTGCAGT | CABE | 864 |
| Cys-GCA-17-1 | | | |
| Homo_sapiens_tRNA- | GGGGACCTCTTGATCTGCAG | CABE | 865 |
| Cys-GCA-17-1 | | | |
| Homo_sapiens_tRNA- | CGGGGACCTCTTGATCTGCA | CABE | 866 |
| Cys-GCA-17-1 | | | |
| Homo_sapiens_tRNA- | GCAGTCAAATGCTCTACCCC | CABE | 867 |
| Cys-GCA-18-1 | | | |
| Homo_sapiens_tRNA- | TGCAGTCAAATGCTCTACCC | CABE | 868 |
| Cys-GCA-18-1 | | | |
| Homo_sapiens_tRNA- | CTGCAGTCAAATGCTCTACC | CABE | 869 |
| Cys-GCA-18-1 | | | |
| Homo_sapiens_tRNA- | TCTGCAGTCAAATGCTCTAC | CABE | 870 |
| Cys-GCA-18-1 | | | |
| Homo_sapiens_tRNA- | ATCTGCAGTCAAATGCTCTA | CABE | 871 |
| Cys-GCA-18-1 | | | |
| Homo_sapiens_tRNA- | GATCTGCAGTCAAATGCTCT | CABE | 872 |
| Cys-GCA-18-1 | | | |
| Homo_sapiens_tRNA- | TGATCTGCAGTCAAATGCTC | CABE | 873 |
| Cys-GCA-18-1 | | | |
| Homo_sapiens_tRNA- | TTGATCTGCAGTCAAATGCT | CABE | 874 |
| Cys-GCA-18-1 | | | |
| Homo_sapiens_tRNA- | CTTGATCTGCAGTCAAATGC | CABE | 875 |
| Cys-GCA-18-1 | | | |
| Homo_sapiens_tRNA- | TCTTGATCTGCAGTCAAATG | CABE | 876 |
| Cys-GCA-18-1 | | | |
| Homo_sapiens_tRNA- | CTCTTGATCTGCAGTCAAAT | CABE | 877 |
| Cys-GCA-18-1 | | | |
| Homo_sapiens_tRNA- | CCTCTTGATCTGCAGTCAAA | CABE | 878 |
| Cys-GCA-18-1 | | | |
| Homo_sapiens_tRNA- | ACCTCTTGATCTGCAGTCAA | CABE | 879 |
| Cys-GCA-18-1 | | | |
| Homo_sapiens_tRNA- | GACCTCTTGATCTGCAGTCA | CABE | 880 |
| Cys-GCA-18-1 | | | |
| Homo_sapiens_tRNA- | GGACCTCTTGATCTGCAGTC | CABE | 881 |
| Cys-GCA-18-1 | | | |
| Homo_sapiens_tRNA- | GGGACCTCTTGATCTGCAGT | CABE | 882 |
| Cys-GCA-18-1 | | | |
| Homo_sapiens_tRNA- | AGGGACCTCTTGATCTGCAG | CABE | 883 |
| Cys-GCA-18-1 | | | |
| Homo_sapiens_tRNA- | CAGGGACCTCTTGATCTGCA | CABE | 884 |
| Cys-GCA-18-1 | | | |
| Homo_sapiens_tRNA- | GCAGTCAAATGCTCTACCCC | CABE | 885 |
| Cys-GCA-19-1 | | | |
| Homo_sapiens_tRNA- | TGCAGTCAAATGCTCTACCC | CABE | 886 |
| Cys-GCA-19-1 | | | |
| Homo_sapiens_tRNA- | TTGCAGTCAAATGCTCTACC | CABE | 887 |
| Cys-GCA-19-1 | | | |
| Homo_sapiens_tRNA- | TTTGCAGTCAAATGCTCTAC | CABE | 888 |
| Cys-GCA-19-1 | | | |
| Homo_sapiens_tRNA- | ATTTGCAGTCAAATGCTCTA | CABE | 889 |
| Cys-GCA-19-1 | | | |
| Homo_sapiens_tRNA- | GATTTGCAGTCAAATGCTCT | CABE | 890 |
| Cys-GCA-19-1 | | | |
| Homo_sapiens_tRNA- | TGATTTGCAGTCAAATGCTC | CABE | 891 |
| Cys-GCA-19-1 | | | |
| Homo_sapiens_tRNA- | TTGATTTGCAGTCAAATGCT | CABE | 892 |
| Cys-GCA-19-1 | | | |
| Homo_sapiens_tRNA- | CTTGATTTGCAGTCAAATGC | CABE | 893 |
| Cys-GCA-19-1 | | | |
| Homo_sapiens_tRNA- | TCTTGATTTGCAGTCAAATG | CABE | 894 |
| Cys-GCA-19-1 | | | |
| Homo_sapiens_tRNA- | CTCTTGATTTGCAGTCAAAT | CABE | 895 |
| Cys-GCA-19-1 | | | |
| Homo_sapiens_tRNA- | CCTCTTGATTTGCAGTCAAA | CABE | 896 |
| Cys-GCA-19-1 | | | |
| Homo_sapiens_tRNA- | ACCTCTTGATTTGCAGTCAA | CABE | 897 |
| Cys-GCA-19-1 | | | |
| Homo_sapiens_tRNA- | GACCTCTTGATTTGCAGTCA | CABE | 898 |
| Cys-GCA-19-1 | | | |
| Homo_sapiens_tRNA- | GGACCTCTTGATTTGCAGTC | CABE | 899 |
| Cys-GCA-19-1 | | | |
| Homo_sapiens_tRNA- | GGGACCTCTTGATTTGCAGT | CABE | 900 |
| Cys-GCA-19-1 | | | |
| Homo_sapiens_tRNA- | AGGGACCTCTTGATTTGCAG | CABE | 901 |
| Cys-GCA-19-1 | | | |
| Homo_sapiens_tRNA- | CAGGGACCTCTTGATTTGCA | CABE | 902 |
| Cys-GCA-19-1 | | | |
| Homo_sapiens_tRNA- | GCAGTCAAATGCTCTACCAC | CABE | 903 |
| Cys-GCA-2-1 | | | |
| Homo_sapiens_tRNA- | TGCAGTCAAATGCTCTACCA | CABE | 904 |
| Cys-GCA-2-1 | | | |
| Homo_sapiens_tRNA- | CTGCAGTCAAATGCTCTACC | CABE | 905 |
| Cys-GCA-2-1 | | | |
| Homo_sapiens_tRNA- | TCTGCAGTCAAATGCTCTAC | CABE | 906 |
| Cys-GCA-2-1 | | | |
| Homo_sapiens_tRNA- | ATCTGCAGTCAAATGCTCTA | CABE | 907 |
| Cys-GCA-2-1 | | | |
| Homo_sapiens_tRNA- | GATCTGCAGTCAAATGCTCT | CABE | 908 |
| Cys-GCA-2-1 | | | |
| Homo_sapiens_tRNA- | TGATCTGCAGTCAAATGCTC | CABE | 909 |
| Cys-GCA-2-1 | | | |
| Homo_sapiens_tRNA- | TTGATCTGCAGTCAAATGCT | CABE | 910 |
| Cys-GCA-2-1 | | | |
| Homo_sapiens_tRNA- | CTTGATCTGCAGTCAAATGC | CABE | 911 |
| Cys-GCA-2-1 | | | |
| Homo_sapiens_tRNA- | TCTTGATCTGCAGTCAAATG | CABE | 912 |
| Cys-GCA-2-1 | | | |
| Homo_sapiens_tRNA- | CTCTTGATCTGCAGTCAAAT | CABE | 913 |
| Cys-GCA-2-1 | | | |
| Homo_sapiens_tRNA- | CCTCTTGATCTGCAGTCAAA | CABE | 914 |
| Cys-GCA-2-1 | | | |
| Homo_sapiens_tRNA- | ACCTCTTGATCTGCAGTCAA | CABE | 915 |
| Cys-GCA-2-1 | | | |
| Homo_sapiens_tRNA- | GACCTCTTGATCTGCAGTCA | CABE | 916 |
| Cys-GCA-2-1 | | | |
| Homo_sapiens_tRNA- | GGACCTCTTGATCTGCAGTC | CABE | 917 |
| Cys-GCA-2-1 | | | |
| Homo_sapiens_tRNA- | GGGACCTCTTGATCTGCAGT | CABE | 918 |
| Cys-GCA-2-1 | | | |
| Homo_sapiens_tRNA- | GGGGACCTCTTGATCTGCAG | CABE | 919 |
| Cys-GCA-2-1 | | | |
| Homo_sapiens_tRNA- | CGGGGACCTCTTGATCTGCA | CABE | 920 |
| Cys-GCA-2-1 | | | |
| Homo_sapiens_tRNA- | GCAGTCAAATGCTCTACCAC | CABE | 921 |
| Cys-GCA-2-2 | | | |
| Homo_sapiens_tRNA- | TGCAGTCAAATGCTCTACCA | CABE | 922 |
| Cys-GCA-2-2 | | | |
| Homo_sapiens_tRNA- | CTGCAGTCAAATGCTCTACC | CABE | 923 |
| Cys-GCA-2-2 | | | |
| Homo_sapiens_tRNA- | TCTGCAGTCAAATGCTCTAC | CABE | 924 |
| Cys-GCA-2-2 | | | |
| Homo_sapiens_tRNA- | ATCTGCAGTCAAATGCTCTA | CABE | 925 |
| Cys-GCA-2-2 | | | |
| Homo_sapiens_tRNA- | GATCTGCAGTCAAATGCTCT | CABE | 926 |
| Cys-GCA-2-2 | | | |
| Homo_sapiens_tRNA- | TGATCTGCAGTCAAATGCTC | CABE | 927 |
| Cys-GCA-2-2 | | | |
| Homo_sapiens_tRNA- | TTGATCTGCAGTCAAATGCT | CABE | 928 |
| Cys-GCA-2-2 | | | |
| Homo_sapiens_tRNA- | CTTGATCTGCAGTCAAATGC | CABE | 929 |
| Cys-GCA-2-2 | | | |
| Homo_sapiens_tRNA- | TCTTGATCTGCAGTCAAATG | CABE | 930 |
| Cys-GCA-2-2 | | | |
| Homo_sapiens_tRNA- | CTCTTGATCTGCAGTCAAAT | CABE | 931 |
| Cys-GCA-2-2 | | | |
| Homo_sapiens_tRNA- | CCTCTTGATCTGCAGTCAAA | CABE | 932 |
| Cys-GCA-2-2 | | | |
| Homo_sapiens_tRNA- | ACCTCTTGATCTGCAGTCAA | CABE | 933 |
| Cys-GCA-2-2 | | | |
| Homo_sapiens_tRNA- | GACCTCTTGATCTGCAGTCA | CABE | 934 |
| Cys-GCA-2-2 | | | |
| Homo_sapiens_tRNA- | GGACCTCTTGATCTGCAGTC | CABE | 935 |
| Cys-GCA-2-2 | | | |
| Homo_sapiens_tRNA- | GGGACCTCTTGATCTGCAGT | CABE | 936 |
| Cys-GCA-2-2 | | | |
| Homo_sapiens_tRNA- | GGGGACCTCTTGATCTGCAG | CABE | 937 |
| Cys-GCA-2-2 | | | |
| Homo_sapiens_tRNA- | CGGGGACCTCTTGATCTGCA | CABE | 938 |
| Cys-GCA-2-2 | | | |
| Homo_sapiens_tRNA- | GCAGTCAAATGCTCTACCAC | CABE | 939 |
| Cys-GCA-2-3 | | | |
| Homo_sapiens_tRNA- | TGCAGTCAAATGCTCTACCA | CABE | 940 |
| Cys-GCA-2-3 | | | |
| Homo_sapiens_tRNA- | CTGCAGTCAAATGCTCTACC | CABE | 941 |
| Cys-GCA-2-3 | | | |
| Homo_sapiens_tRNA- | TCTGCAGTCAAATGCTCTAC | CABE | 942 |
| Cys-GCA-2-3 | | | |
| Homo_sapiens_tRNA- | ATCTGCAGTCAAATGCTCTA | CABE | 943 |
| Cys-GCA-2-3 | | | |
| Homo_sapiens_tRNA- | GATCTGCAGTCAAATGCTCT | CABE | 944 |
| Cys-GCA-2-3 | | | |
| Homo_sapiens_tRNA- | TGATCTGCAGTCAAATGCTC | CABE | 945 |
| Cys-GCA-2-3 | | | |
| Homo_sapiens_tRNA- | TTGATCTGCAGTCAAATGCT | CABE | 946 |
| Cys-GCA-2-3 | | | |
| Homo_sapiens_tRNA- | CTTGATCTGCAGTCAAATGC | CABE | 947 |
| Cys-GCA-2-3 | | | |
| Homo_sapiens_tRNA- | TCTTGATCTGCAGTCAAATG | CABE | 948 |
| Cys-GCA-2-3 | | | |
| Homo_sapiens_tRNA- | CTCTTGATCTGCAGTCAAAT | CABE | 949 |
| Cys-GCA-2-3 | | | |
| Homo_sapiens_tRNA- | CCTCTTGATCTGCAGTCAAA | CABE | 950 |
| Cys-GCA-2-3 | | | |
| Homo_sapiens_tRNA- | ACCTCTTGATCTGCAGTCAA | CABE | 951 |
| Cys-GCA-2-3 | | | |
| Homo_sapiens_tRNA- | GACCTCTTGATCTGCAGTCA | CABE | 952 |
| Cys-GCA-2-3 | | | |
| Homo_sapiens_tRNA- | GGACCTCTTGATCTGCAGTC | CABE | 953 |
| Cys-GCA-2-3 | | | |
| Homo_sapiens_tRNA- | GGGACCTCTTGATCTGCAGT | CABE | 954 |
| Cys-GCA-2-3 | | | |
| Homo_sapiens_tRNA- | GGGGACCTCTTGATCTGCAG | CABE | 955 |
| Cys-GCA-2-3 | | | |
| Homo_sapiens_tRNA- | CGGGGACCTCTTGATCTGCA | CABE | 956 |
| Cys-GCA-2-3 | | | |
| Homo_sapiens_tRNA- | GCAGTCAAATGCTCTACCAC | CABE | 957 |
| Cys-GCA-2-4 | | | |
| Homo_sapiens_tRNA- | TGCAGTCAAATGCTCTACCA | CABE | 958 |
| Cys-GCA-2-4 | | | |
| Homo_sapiens_tRNA- | CTGCAGTCAAATGCTCTACC | CABE | 959 |
| Cys-GCA-2-4 | | | |
| Homo_sapiens_tRNA- | TCTGCAGTCAAATGCTCTAC | CABE | 960 |
| Cys-GCA-2-4 | | | |
| Homo_sapiens_tRNA- | ATCTGCAGTCAAATGCTCTA | CABE | 961 |
| Cys-GCA-2-4 | | | |
| Homo_sapiens_tRNA- | GATCTGCAGTCAAATGCTCT | CABE | 962 |
| Cys-GCA-2-4 | | | |
| Homo_sapiens_tRNA- | TGATCTGCAGTCAAATGCTC | CABE | 963 |
| Cys-GCA-2-4 | | | |
| Homo_sapiens_tRNA- | TTGATCTGCAGTCAAATGCT | CABE | 964 |
| Cys-GCA-2-4 | | | |
| Homo_sapiens_tRNA- | CTTGATCTGCAGTCAAATGC | CABE | 965 |
| Cys-GCA-2-4 | | | |
| Homo_sapiens_tRNA- | TCTTGATCTGCAGTCAAATG | CABE | 966 |
| Cys-GCA-2-4 | | | |
| Homo_sapiens_tRNA- | CTCTTGATCTGCAGTCAAAT | CABE | 967 |
| Cys-GCA-2-4 | | | |
| Homo_sapiens_tRNA- | CCTCTTGATCTGCAGTCAAA | CABE | 968 |
| Cys-GCA-2-4 | | | |
| Homo_sapiens_tRNA- | ACCTCTTGATCTGCAGTCAA | CABE | 969 |
| Cys-GCA-2-4 | | | |
| Homo_sapiens_tRNA- | GACCTCTTGATCTGCAGTCA | CABE | 970 |
| Cys-GCA-2-4 | | | |
| Homo_sapiens_tRNA- | GGACCTCTTGATCTGCAGTC | CABE | 971 |
| Cys-GCA-2-4 | | | |
| Homo_sapiens_tRNA- | GGGACCTCTTGATCTGCAGT | CABE | 972 |
| Cys-GCA-2-4 | | | |
| Homo_sapiens_tRNA- | GGGGACCTCTTGATCTGCAG | CABE | 973 |
| Cys-GCA-2-4 | | | |
| Homo_sapiens_tRNA- | CGGGGACCTCTTGATCTGCA | CABE | 974 |
| Cys-GCA-2-4 | | | |
| Homo_sapiens_tRNA- | GCAGTCAAATGCTCTACCCC | CABE | 975 |
| Cys-GCA-20-1 | | | |
| Homo_sapiens_tRNA- | TGCAGTCAAATGCTCTACCC | CABE | 976 |
| Cys-GCA-20-1 | | | |
| Homo_sapiens_tRNA- | CTGCAGTCAAATGCTCTACC | CABE | 977 |
| Cys-GCA-20-1 | | | |
| Homo_sapiens_tRNA- | TCTGCAGTCAAATGCTCTAC | CABE | 978 |
| Cys-GCA-20-1 | | | |
| Homo_sapiens_tRNA- | ATCTGCAGTCAAATGCTCTA | CABE | 979 |
| Cys-GCA-20-1 | | | |
| Homo_sapiens_tRNA- | GATCTGCAGTCAAATGCTCT | CABE | 980 |
| Cys-GCA-20-1 | | | |
| Homo_sapiens_tRNA- | TGATCTGCAGTCAAATGCTC | CABE | 981 |
| Cys-GCA-20-1 | | | |
| Homo_sapiens_tRNA- | TTGATCTGCAGTCAAATGCT | CABE | 982 |
| Cys-GCA-20-1 | | | |
| Homo_sapiens_tRNA- | CTTGATCTGCAGTCAAATGC | CABE | 983 |
| Cys-GCA-20-1 | | | |
| Homo_sapiens_tRNA- | TCTTGATCTGCAGTCAAATG | CABE | 984 |
| Cys-GCA-20-1 | | | |
| Homo_sapiens_tRNA- | CTCTTGATCTGCAGTCAAAT | CABE | 985 |
| Cys-GCA-20-1 | | | |
| Homo_sapiens_tRNA- | CCTCTTGATCTGCAGTCAAA | CABE | 986 |
| Cys-GCA-20-1 | | | |
| Homo_sapiens_tRNA- | ACCTCTTGATCTGCAGTCAA | CABE | 987 |
| Cys-GCA-20-1 | | | |
| Homo_sapiens_tRNA- | GACCTCTTGATCTGCAGTCA | CABE | 988 |
| Cys-GCA-20-1 | | | |
| Homo_sapiens_tRNA- | GGACCTCTTGATCTGCAGTC | CABE | 989 |
| Cys-GCA-20-1 | | | |
| Homo_sapiens_tRNA- | GGGACCTCTTGATCTGCAGT | CABE | 990 |
| Cys-GCA-20-1 | | | |
| Homo_sapiens_tRNA- | GGGGACCTCTTGATCTGCAG | CABE | 991 |
| Cys-GCA-20-1 | | | |
| Homo_sapiens_tRNA- | TGGGGACCTCTTGATCTGCA | CABE | 992 |
| Cys-GCA-20-1 | | | |
| Homo_sapiens_tRNA- | GCAGTCAAATGCTCTACCTG | CABE | 993 |
| Cys-GCA-21-1 | | | |
| Homo_sapiens_tRNA- | TGCAGTCAAATGCTCTACCT | CABE | 994 |
| Cys-GCA-21-1 | | | |
| Homo_sapiens_tRNA- | CTGCAGTCAAATGCTCTACC | CABE | 995 |
| Cys-GCA-21-1 | | | |
| Homo_sapiens_tRNA- | TCTGCAGTCAAATGCTCTAC | CABE | 996 |
| Cys-GCA-21-1 | | | |
| Homo_sapiens_tRNA- | ATCTGCAGTCAAATGCTCTA | CABE | 997 |
| Cys-GCA-21-1 | | | |
| Homo_sapiens_tRNA- | GATCTGCAGTCAAATGCTCT | CABE | 998 |
| Cys-GCA-21-1 | | | |
| Homo_sapiens_tRNA- | TGATCTGCAGTCAAATGCTC | CABE | 999 |
| Cys-GCA-21-1 | | | |
| Homo_sapiens_tRNA- | TTGATCTGCAGTCAAATGCT | CABE | 1000 |
| Cys-GCA-21-1 | | | |
| Homo_sapiens_tRNA- | CTTGATCTGCAGTCAAATGC | CABE | 1001 |
| Cys-GCA-21-1 | | | |
| Homo_sapiens_tRNA- | TCTTGATCTGCAGTCAAATG | CABE | 1002 |
| Cys-GCA-21-1 | | | |
| Homo_sapiens_tRNA- | CTCTTGATCTGCAGTCAAAT | CABE | 1003 |
| Cys-GCA-21-1 | | | |
| Homo_sapiens_tRNA- | CCTCTTGATCTGCAGTCAAA | CABE | 1004 |
| Cys-GCA-21-1 | | | |
| Homo_sapiens_tRNA- | ACCTCTTGATCTGCAGTCAA | CABE | 1005 |
| Cys-GCA-21-1 | | | |
| Homo_sapiens_tRNA- | GACCTCTTGATCTGCAGTCA | CABE | 1006 |
| Cys-GCA-21-1 | | | |
| Homo_sapiens_tRNA- | GGACCTCTTGATCTGCAGTC | CABE | 1007 |
| Cys-GCA-21-1 | | | |
| Homo_sapiens_tRNA- | GGGACCTCTTGATCTGCAGT | CABE | 1008 |
| Cys-GCA-21-1 | | | |
| Homo_sapiens_tRNA- | GGGGACCTCTTGATCTGCAG | CABE | 1009 |
| Cys-GCA-21-1 | | | |
| Homo_sapiens_tRNA- | CGGGGACCTCTTGATCTGCA | CABE | 1010 |
| Cys-GCA-21-1 | | | |
| Homo_sapiens_tRNA- | GCAGTCAAATGCTCTACCCC | CABE | 1011 |
| Cys-GCA-22-1 | | | |
| Homo_sapiens_tRNA- | TGCAGTCAAATGCTCTACCC | CABE | 1012 |
| Cys-GCA-22-1 | | | |
| Homo_sapiens_tRNA- | CTGCAGTCAAATGCTCTACC | CABE | 1013 |
| Cys-GCA-22-1 | | | |
| Homo_sapiens_tRNA- | TCTGCAGTCAAATGCTCTAC | CABE | 1014 |
| Cys-GCA-22-1 | | | |
| Homo_sapiens_tRNA- | ATCTGCAGTCAAATGCTCTA | CABE | 1015 |
| Cys-GCA-22-1 | | | |
| Homo_sapiens_tRNA- | GATCTGCAGTCAAATGCTCT | CABE | 1016 |
| Cys-GCA-22-1 | | | |
| Homo_sapiens_tRNA- | TGATCTGCAGTCAAATGCTC | CABE | 1017 |
| Cys-GCA-22-1 | | | |
| Homo_sapiens_tRNA- | TTGATCTGCAGTCAAATGCT | CABE | 1018 |
| Cys-GCA-22-1 | | | |
| Homo_sapiens_tRNA- | CTTGATCTGCAGTCAAATGC | CABE | 1019 |
| Cys-GCA-22-1 | | | |
| Homo_sapiens_tRNA- | TCTTGATCTGCAGTCAAATG | CABE | 1020 |
| Cys-GCA-22-1 | | | |
| Homo_sapiens_tRNA- | CTCTTGATCTGCAGTCAAAT | CABE | 1021 |
| Cys-GCA-22-1 | | | |
| Homo_sapiens_tRNA- | CCTCTTGATCTGCAGTCAAA | CABE | 1022 |
| Cys-GCA-22-1 | | | |
| Homo_sapiens_tRNA- | ACCTCTTGATCTGCAGTCAA | CABE | 1023 |
| Cys-GCA-22-1 | | | |
| Homo_sapiens_tRNA- | GACCTCTTGATCTGCAGTCA | CABE | 1024 |
| Cys-GCA-22-1 | | | |
| Homo_sapiens_tRNA- | GGACCTCTTGATCTGCAGTC | CABE | 1025 |
| Cys-GCA-22-1 | | | |
| Homo_sapiens_tRNA- | GGGACCTCTTGATCTGCAGT | CABE | 1026 |
| Cys-GCA-22-1 | | | |
| Homo_sapiens_tRNA- | GGGGACCTCTTGATCTGCAG | CABE | 1027 |
| Cys-GCA-22-1 | | | |
| Homo_sapiens_tRNA- | TGGGGACCTCTTGATCTGCA | CABE | 1028 |
| Cys-GCA-22-1 | | | |
| Homo_sapiens_tRNA- | GCAGTCAAATGCTCTACCTG | CABE | 1029 |
| Cys-GCA-23-1 | | | |
| Homo_sapiens_tRNA- | TGCAGTCAAATGCTCTACCT | CABE | 1030 |
| Cys-GCA-23-1 | | | |
| Homo_sapiens_tRNA- | CTGCAGTCAAATGCTCTACC | CABE | 1031 |
| Cys-GCA-23-1 | | | |
| Homo_sapiens_tRNA- | TCTGCAGTCAAATGCTCTAC | CABE | 1032 |
| Cys-GCA-23-1 | | | |
| Homo_sapiens_tRNA- | ATCTGCAGTCAAATGCTCTA | CABE | 1033 |
| Cys-GCA-23-1 | | | |
| Homo_sapiens_tRNA- | GATCTGCAGTCAAATGCTCT | CABE | 1034 |
| Cys-GCA-23-1 | | | |
| Homo_sapiens_tRNA- | TGATCTGCAGTCAAATGCTC | CABE | 1035 |
| Cys-GCA-23-1 | | | |
| Homo_sapiens_tRNA- | TTGATCTGCAGTCAAATGCT | CABE | 1036 |
| Cys-GCA-23-1 | | | |
| Homo_sapiens_tRNA- | CTTGATCTGCAGTCAAATGC | CABE | 1037 |
| Cys-GCA-23-1 | | | |
| Homo_sapiens_tRNA- | TCTTGATCTGCAGTCAAATG | CABE | 1038 |
| Cys-GCA-23-1 | | | |
| Homo_sapiens_tRNA- | CTCTTGATCTGCAGTCAAAT | CABE | 1039 |
| Cys-GCA-23-1 | | | |
| Homo_sapiens_tRNA- | CCTCTTGATCTGCAGTCAAA | CABE | 1040 |
| Cys-GCA-23-1 | | | |
| Homo_sapiens_tRNA- | ACCTCTTGATCTGCAGTCAA | CABE | 1041 |
| Cys-GCA-23-1 | | | |
| Homo_sapiens_tRNA- | GACCTCTTGATCTGCAGTCA | CABE | 1042 |
| Cys-GCA-23-1 | | | |
| Homo_sapiens_tRNA- | GGACCTCTTGATCTGCAGTC | CABE | 1043 |
| Cys-GCA-23-1 | | | |
| Homo_sapiens_tRNA- | GGGACCTCTTGATCTGCAGT | CABE | 1044 |
| Cys-GCA-23-1 | | | |
| Homo_sapiens_tRNA- | GGGGACCTCTTGATCTGCAG | CABE | 1045 |
| Cys-GCA-23-1 | | | |
| Homo_sapiens_tRNA- | CGGGGACCTCTTGATCTGCA | CABE | 1046 |
| Cys-GCA-23-1 | | | |
| Homo_sapiens_tRNA- | GCAGTCAAGTGCTCTACCCC | CABE | 1047 |
| Cys-GCA-3-1 | | | |
| Homo_sapiens_tRNA- | TGCAGTCAAGTGCTCTACCC | CABE | 1048 |
| Cys-GCA-3-1 | | | |
| Homo_sapiens_tRNA- | CTGCAGTCAAGTGCTCTACC | CABE | 1049 |
| Cys-GCA-3-1 | | | |
| Homo_sapiens_tRNA- | TCTGCAGTCAAGTGCTCTAC | CABE | 1050 |
| Cys-GCA-3-1 | | | |
| Homo_sapiens_tRNA- | ATCTGCAGTCAAGTGCTCTA | CABE | 1051 |
| Cys-GCA-3-1 | | | |
| Homo_sapiens_tRNA- | GATCTGCAGTCAAGTGCTCT | CABE | 1052 |
| Cys-GCA-3-1 | | | |
| Homo_sapiens_tRNA- | TGATCTGCAGTCAAGTGCTC | CABE | 1053 |
| Cys-GCA-3-1 | | | |
| Homo_sapiens_tRNA- | TTGATCTGCAGTCAAGTGCT | CABE | 1054 |
| Cys-GCA-3-1 | | | |
| Homo_sapiens_tRNA- | CTTGATCTGCAGTCAAGTGC | CABE | 1055 |
| Cys-GCA-3-1 | | | |
| Homo_sapiens_tRNA- | TCTTGATCTGCAGTCAAGTG | CABE | 1056 |
| Cys-GCA-3-1 | | | |
| Homo_sapiens_tRNA- | CTCTTGATCTGCAGTCAAGT | CABE | 1057 |
| Cys-GCA-3-1 | | | |
| Homo_sapiens_tRNA- | CCTCTTGATCTGCAGTCAAG | CABE | 1058 |
| Cys-GCA-3-1 | | | |
| Homo_sapiens_tRNA- | ACCTCTTGATCTGCAGTCAA | CABE | 1059 |
| Cys-GCA-3-1 | | | |
| Homo_sapiens_tRNA- | GACCTCTTGATCTGCAGTCA | CABE | 1060 |
| Cys-GCA-3-1 | | | |
| Homo_sapiens_tRNA- | GGACCTCTTGATCTGCAGTC | CABE | 1061 |
| Cys-GCA-3-1 | | | |
| Homo_sapiens_tRNA- | GGGACCTCTTGATCTGCAGT | CABE | 1062 |
| Cys-GCA-3-1 | | | |
| Homo_sapiens_tRNA- | AGGGACCTCTTGATCTGCAG | CABE | 1063 |
| Cys-GCA-3-1 | | | |
| Homo_sapiens_tRNA- | CAGGGACCTCTTGATCTGCA | CABE | 1064 |
| Cys-GCA-3-1 | | | |
| Homo_sapiens_tRNA- | GCAGTCAAATGCTCTACCAC | CABE | 1065 |
| Cys-GCA-4-1 | | | |
| Homo_sapiens_tRNA- | TGCAGTCAAATGCTCTACCA | CABE | 1066 |
| Cys-GCA-4-1 | | | |
| Homo_sapiens_tRNA- | CTGCAGTCAAATGCTCTACC | CABE | 1067 |
| Cys-GCA-4-1 | | | |
| Homo_sapiens_tRNA- | TCTGCAGTCAAATGCTCTAC | CABE | 1068 |
| Cys-GCA-4-1 | | | |
| Homo_sapiens_tRNA- | ATCTGCAGTCAAATGCTCTA | CABE | 1069 |
| Cys-GCA-4-1 | | | |
| Homo_sapiens_tRNA- | GATCTGCAGTCAAATGCTCT | CABE | 1070 |
| Cys-GCA-4-1 | | | |
| Homo_sapiens_tRNA- | TGATCTGCAGTCAAATGCTC | CABE | 1071 |
| Cys-GCA-4-1 | | | |
| Homo_sapiens_tRNA- | TTGATCTGCAGTCAAATGCT | CABE | 1072 |
| Cys-GCA-4-1 | | | |
| Homo_sapiens_tRNA- | CTTGATCTGCAGTCAAATGC | CABE | 1073 |
| Cys-GCA-4-1 | | | |
| Homo_sapiens_tRNA- | TCTTGATCTGCAGTCAAATG | CABE | 1074 |
| Cys-GCA-4-1 | | | |
| Homo_sapiens_tRNA- | CTCTTGATCTGCAGTCAAAT | CABE | 1075 |
| Cys-GCA-4-1 | | | |
| Homo_sapiens_tRNA- | CCTCTTGATCTGCAGTCAAA | CABE | 1076 |
| Cys-GCA-4-1 | | | |
| Homo_sapiens_tRNA- | ACCTCTTGATCTGCAGTCAA | CABE | 1077 |
| Cys-GCA-4-1 | | | |
| Homo_sapiens_tRNA- | GACCTCTTGATCTGCAGTCA | CABE | 1078 |
| Cys-GCA-4-1 | | | |
| Homo_sapiens_tRNA- | GGACCTCTTGATCTGCAGTC | CABE | 1079 |
| Cys-GCA-4-1 | | | |
| Homo_sapiens_tRNA- | GGGACCTCTTGATCTGCAGT | CABE | 1080 |
| Cys-GCA-4-1 | | | |
| Homo_sapiens_tRNA- | AGGGACCTCTTGATCTGCAG | CABE | 1081 |
| Cys-GCA-4-1 | | | |
| Homo_sapiens_tRNA- | CAGGGACCTCTTGATCTGCA | CABE | 1082 |
| Cys-GCA-4-1 | | | |
| Homo_sapiens_tRNA- | CAGTCAAATGCTCTACCCAC | CABE | 1083 |
| Cys-GCA-5-1 | | | |
| Homo_sapiens_tRNA- | GCAGTCAAATGCTCTACCCA | CABE | 1084 |
| Cys-GCA-5-1 | | | |
| Homo_sapiens_tRNA- | TGCAGTCAAATGCTCTACCC | CABE | 1085 |
| Cys-GCA-5-1 | | | |
| Homo_sapiens_tRNA- | CTGCAGTCAAATGCTCTACC | CABE | 1086 |
| Cys-GCA-5-1 | | | |
| Homo_sapiens_tRNA- | TCTGCAGTCAAATGCTCTAC | CABE | 1087 |
| Cys-GCA-5-1 | | | |
| Homo_sapiens_tRNA- | ATCTGCAGTCAAATGCTCTA | CABE | 1088 |
| Cys-GCA-5-1 | | | |
| Homo_sapiens_tRNA- | GATCTGCAGTCAAATGCTCT | CABE | 1089 |
| Cys-GCA-5-1 | | | |
| Homo_sapiens_tRNA- | TGATCTGCAGTCAAATGCTC | CABE | 1090 |
| Cys-GCA-5-1 | | | |
| Homo_sapiens_tRNA- | TTGATCTGCAGTCAAATGCT | CABE | 1091 |
| Cys-GCA-5-1 | | | |
| Homo_sapiens_tRNA- | CTTGATCTGCAGTCAAATGC | CABE | 1092 |
| Cys-GCA-5-1 | | | |
| Homo_sapiens_tRNA- | TCTTGATCTGCAGTCAAATG | CABE | 1093 |
| Cys-GCA-5-1 | | | |
| Homo_sapiens_tRNA- | CTCTTGATCTGCAGTCAAAT | CABE | 1094 |
| Cys-GCA-5-1 | | | |
| Homo_sapiens_tRNA- | CCTCTTGATCTGCAGTCAAA | CABE | 1095 |
| Cys-GCA-5-1 | | | |
| Homo_sapiens_tRNA- | ACCTCTTGATCTGCAGTCAA | CABE | 1096 |
| Cys-GCA-5-1 | | | |
| Homo_sapiens_tRNA- | GACCTCTTGATCTGCAGTCA | CABE | 1097 |
| Cys-GCA-5-1 | | | |
| Homo_sapiens_tRNA- | GGACCTCTTGATCTGCAGTC | CABE | 1098 |
| Cys-GCA-5-1 | | | |
| Homo_sapiens_tRNA- | GGGACCTCTTGATCTGCAGT | CABE | 1099 |
| Cys-GCA-5-1 | | | |
| Homo_sapiens_tRNA- | GGGGACCTCTTGATCTGCAG | CABE | 1100 |
| Cys-GCA-5-1 | | | |
| Homo_sapiens_tRNA- | GCAGTCAAATGCTCTACCAC | CABE | 1101 |
| Cys-GCA-6-1 | | | |
| Homo_sapiens_tRNA- | TGCAGTCAAATGCTCTACCA | CABE | 1102 |
| Cys-GCA-6-1 | | | |
| Homo_sapiens_tRNA- | CTGCAGTCAAATGCTCTACC | CABE | 1103 |
| Cys-GCA-6-1 | | | |
| Homo_sapiens_tRNA- | TCTGCAGTCAAATGCTCTAC | CABE | 1104 |
| Cys-GCA-6-1 | | | |
| Homo_sapiens_tRNA- | ATCTGCAGTCAAATGCTCTA | CABE | 1105 |
| Cys-GCA-6-1 | | | |
| Homo_sapiens_tRNA- | GATCTGCAGTCAAATGCTCT | CABE | 1106 |
| Cys-GCA-6-1 | | | |
| Homo_sapiens_tRNA- | TGATCTGCAGTCAAATGCTC | CABE | 1107 |
| Cys-GCA-6-1 | | | |
| Homo_sapiens_tRNA- | TTGATCTGCAGTCAAATGCT | CABE | 1108 |
| Cys-GCA-6-1 | | | |
| Homo_sapiens_tRNA- | CTTGATCTGCAGTCAAATGC | CABE | 1109 |
| Cys-GCA-6-1 | | | |
| Homo_sapiens_tRNA- | TCTTGATCTGCAGTCAAATG | CABE | 1110 |
| Cys-GCA-6-1 | | | |
| Homo_sapiens_tRNA- | CTCTTGATCTGCAGTCAAAT | CABE | 1111 |
| Cys-GCA-6-1 | | | |
| Homo_sapiens_tRNA- | CCTCTTGATCTGCAGTCAAA | CABE | 1112 |
| Cys-GCA-6-1 | | | |
| Homo_sapiens_tRNA- | ACCTCTTGATCTGCAGTCAA | CABE | 1113 |
| Cys-GCA-6-1 | | | |
| Homo_sapiens_tRNA- | GACCTCTTGATCTGCAGTCA | CABE | 1114 |
| Cys-GCA-6-1 | | | |
| Homo_sapiens_tRNA- | GGACCTCTTGATCTGCAGTC | CABE | 1115 |
| Cys-GCA-6-1 | | | |
| Homo_sapiens_tRNA- | GGGACCTCTTGATCTGCAGT | CABE | 1116 |
| Cys-GCA-6-1 | | | |
| Homo_sapiens_tRNA- | AGGGACCTCTTGATCTGCAG | CABE | 1117 |
| Cys-GCA-6-1 | | | |
| Homo_sapiens_tRNA- | CAGGGACCTCTTGATCTGCA | CABE | 1118 |
| Cys-GCA-6-1 | | | |
| Homo_sapiens_tRNA- | CAGTCAAATGCTCTACCACC | CABE | 1119 |
| Cys-GCA-7-1 | | | |
| Homo_sapiens_tRNA- | GCAGTCAAATGCTCTACCAC | CABE | 1120 |
| Cys-GCA-7-1 | | | |
| Homo_sapiens_tRNA- | TGCAGTCAAATGCTCTACCA | CABE | 1121 |
| Cys-GCA-7-1 | | | |
| Homo_sapiens_tRNA- | CTGCAGTCAAATGCTCTACC | CABE | 1122 |
| Cys-GCA-7-1 | | | |
| Homo_sapiens_tRNA- | TCTGCAGTCAAATGCTCTAC | CABE | 1123 |
| Cys-GCA-7-1 | | | |
| Homo_sapiens_tRNA- | ATCTGCAGTCAAATGCTCTA | CABE | 1124 |
| Cys-GCA-7-1 | | | |
| Homo_sapiens_tRNA- | GATCTGCAGTCAAATGCTCT | CABE | 1125 |
| Cys-GCA-7-1 | | | |
| Homo_sapiens_tRNA- | TGATCTGCAGTCAAATGCTC | CABE | 1126 |
| Cys-GCA-7-1 | | | |
| Homo_sapiens_tRNA- | TTGATCTGCAGTCAAATGCT | CABE | 1127 |
| Cys-GCA-7-1 | | | |
| Homo_sapiens_tRNA- | CTTGATCTGCAGTCAAATGC | CABE | 1128 |
| Cys-GCA-7-1 | | | |
| Homo_sapiens_tRNA- | TCTTGATCTGCAGTCAAATG | CABE | 1129 |
| Cys-GCA-7-1 | | | |
| Homo_sapiens_tRNA- | CTCTTGATCTGCAGTCAAAT | CABE | 1130 |
| Cys-GCA-7-1 | | | |
| Homo_sapiens_tRNA- | CCTCTTGATCTGCAGTCAAA | CABE | 1131 |
| Cys-GCA-7-1 | | | |
| Homo_sapiens_tRNA- | ACCTCTTGATCTGCAGTCAA | CABE | 1132 |
| Cys-GCA-7-1 | | | |
| Homo_sapiens_tRNA- | GACCTCTTGATCTGCAGTCA | CABE | 1133 |
| Cys-GCA-7-1 | | | |
| Homo_sapiens_tRNA- | GGACCTCTTGATCTGCAGTC | CABE | 1134 |
| Cys-GCA-7-1 | | | |
| Homo_sapiens_tRNA- | GGGACCTCTTGATCTGCAGT | CABE | 1135 |
| Cys-GCA-7-1 | | | |
| Homo_sapiens_tRNA- | GGGGACCTCTTGATCTGCAG | CABE | 1136 |
| Cys-GCA-7-1 | | | |
| Homo_sapiens_tRNA- | GCAGTCAAATGCTCTACCCC | CABE | 1137 |
| Cys-GCA-8-1 | | | |
| Homo_sapiens_tRNA- | TGCAGTCAAATGCTCTACCC | CABE | 1138 |
| Cys-GCA-8-1 | | | |
| Homo_sapiens_tRNA- | CTGCAGTCAAATGCTCTACC | CABE | 1139 |
| Cys-GCA-8-1 | | | |
| Homo_sapiens_tRNA- | TCTGCAGTCAAATGCTCTAC | CABE | 1140 |
| Cys-GCA-8-1 | | | |
| Homo_sapiens_tRNA- | ATCTGCAGTCAAATGCTCTA | CABE | 1141 |
| Cys-GCA-8-1 | | | |
| Homo_sapiens_tRNA- | GATCTGCAGTCAAATGCTCT | CABE | 1142 |
| Cys-GCA-8-1 | | | |
| Homo_sapiens_tRNA- | TGATCTGCAGTCAAATGCTC | CABE | 1143 |
| Cys-GCA-8-1 | | | |
| Homo_sapiens_tRNA- | TTGATCTGCAGTCAAATGCT | CABE | 1144 |
| Cys-GCA-8-1 | | | |
| Homo_sapiens_tRNA- | CTTGATCTGCAGTCAAATGC | CABE | 1145 |
| Cys-GCA-8-1 | | | |
| Homo_sapiens_tRNA- | TCTTGATCTGCAGTCAAATG | CABE | 1146 |
| Cys-GCA-8-1 | | | |
| Homo_sapiens_tRNA- | CTCTTGATCTGCAGTCAAAT | CABE | 1147 |
| Cys-GCA-8-1 | | | |
| Homo_sapiens_tRNA- | CCTCTTGATCTGCAGTCAAA | CABE | 1148 |
| Cys-GCA-8-1 | | | |
| Homo_sapiens_tRNA- | ACCTCTTGATCTGCAGTCAA | CABE | 1149 |
| Cys-GCA-8-1 | | | |
| Homo_sapiens_tRNA- | GACCTCTTGATCTGCAGTCA | CABE | 1150 |
| Cys-GCA-8-1 | | | |
| Homo_sapiens_tRNA- | GGACCTCTTGATCTGCAGTC | CABE | 1151 |
| Cys-GCA-8-1 | | | |
| Homo_sapiens_tRNA- | GGGACCTCTTGATCTGCAGT | CABE | 1152 |
| Cys-GCA-8-1 | | | |
| Homo_sapiens_tRNA- | GGGGACCTCTTGATCTGCAG | CABE | 1153 |
| Cys-GCA-8-1 | | | |
| Homo_sapiens_tRNA- | CGGGGACCTCTTGATCTGCA | CABE | 1154 |
| Cys-GCA-8-1 | | | |
| Homo_sapiens_tRNA- | GCAGTCAAATGCTCTACCCC | CABE | 1155 |
| Cys-GCA-9-1 | | | |
| Homo_sapiens_tRNA- | TGCAGTCAAATGCTCTACCC | CABE | 1156 |
| Cys-GCA-9-1 | | | |
| Homo_sapiens_tRNA- | CTGCAGTCAAATGCTCTACC | CABE | 1157 |
| Cys-GCA-9-1 | | | |
| Homo_sapiens_tRNA- | TCTGCAGTCAAATGCTCTAC | CABE | 1158 |
| Cys-GCA-9-1 | | | |
| Homo_sapiens_tRNA- | ATCTGCAGTCAAATGCTCTA | CABE | 1159 |
| Cys-GCA-9-1 | | | |
| Homo_sapiens_tRNA- | GATCTGCAGTCAAATGCTCT | CABE | 1160 |
| Cys-GCA-9-1 | | | |
| Homo_sapiens_tRNA- | TGATCTGCAGTCAAATGCTC | CABE | 1161 |
| Cys-GCA-9-1 | | | |
| Homo_sapiens_tRNA- | TTGATCTGCAGTCAAATGCT | CABE | 1162 |
| Cys-GCA-9-1 | | | |
| Homo_sapiens_tRNA- | CTTGATCTGCAGTCAAATGC | CABE | 1163 |
| Cys-GCA-9-1 | | | |
| Homo_sapiens_tRNA- | TCTTGATCTGCAGTCAAATG | CABE | 1164 |
| Cys-GCA-9-1 | | | |
| Homo_sapiens_tRNA- | CTCTTGATCTGCAGTCAAAT | CABE | 1165 |
| Cys-GCA-9-1 | | | |
| Homo_sapiens_tRNA- | CCTCTTGATCTGCAGTCAAA | CABE | 1166 |
| Cys-GCA-9-1 | | | |
| Homo_sapiens_tRNA- | ACCTCTTGATCTGCAGTCAA | CABE | 1167 |
| Cys-GCA-9-1 | | | |
| Homo_sapiens_tRNA- | GACCTCTTGATCTGCAGTCA | CABE | 1168 |
| Cys-GCA-9-1 | | | |
| Homo_sapiens_tRNA- | GGACCTCTTGATCTGCAGTC | CABE | 1169 |
| Cys-GCA-9-1 | | | |
| Homo_sapiens_tRNA- | GGGACCTCTTGATCTGCAGT | CABE | 1170 |
| Cys-GCA-9-1 | | | |
| Homo_sapiens_tRNA- | AGGGACCTCTTGATCTGCAG | CABE | 1171 |
| Cys-GCA-9-1 | | | |
| Homo_sapiens_tRNA- | CAGGGACCTCTTGATCTGCA | CABE | 1172 |
| Cys-GCA-9-1 | | | |
| Homo_sapiens_tRNA- | GCAGTCAAATGCTCTACCCC | CABE | 1173 |
| Cys-GCA-9-2 | | | |
| Homo_sapiens_tRNA- | TGCAGTCAAATGCTCTACCC | CABE | 1174 |
| Cys-GCA-9-2 | | | |
| Homo_sapiens_tRNA- | CTGCAGTCAAATGCTCTACC | CABE | 1175 |
| Cys-GCA-9-2 | | | |
| Homo_sapiens_tRNA- | TCTGCAGTCAAATGCTCTAC | CABE | 1176 |
| Cys-GCA-9-2 | | | |
| Homo_sapiens_tRNA- | ATCTGCAGTCAAATGCTCTA | CABE | 1177 |
| Cys-GCA-9-2 | | | |
| Homo_sapiens_tRNA- | GATCTGCAGTCAAATGCTCT | CABE | 1178 |
| Cys-GCA-9-2 | | | |
| Homo_sapiens_tRNA- | TGATCTGCAGTCAAATGCTC | CABE | 1179 |
| Cys-GCA-9-2 | | | |
| Homo_sapiens_tRNA- | TTGATCTGCAGTCAAATGCT | CABE | 1180 |
| Cys-GCA-9-2 | | | |
| Homo_sapiens_tRNA- | CTTGATCTGCAGTCAAATGC | CABE | 1181 |
| Cys-GCA-9-2 | | | |
| Homo_sapiens_tRNA- | TCTTGATCTGCAGTCAAATG | CABE | 1182 |
| Cys-GCA-9-2 | | | |
| Homo_sapiens_tRNA- | CTCTTGATCTGCAGTCAAAT | CABE | 1183 |
| Cys-GCA-9-2 | | | |
| Homo_sapiens_tRNA- | CCTCTTGATCTGCAGTCAAA | CABE | 1184 |
| Cys-GCA-9-2 | | | |
| Homo_sapiens_tRNA- | ACCTCTTGATCTGCAGTCAA | CABE | 1185 |
| Cys-GCA-9-2 | | | |
| Homo_sapiens_tRNA- | GACCTCTTGATCTGCAGTCA | CABE | 1186 |
| Cys-GCA-9-2 | | | |
| Homo_sapiens_tRNA- | GGACCTCTTGATCTGCAGTC | CABE | 1187 |
| Cys-GCA-9-2 | | | |
| Homo_sapiens_tRNA- | GGGACCTCTTGATCTGCAGT | CABE | 1188 |
| Cys-GCA-9-2 | | | |
| Homo_sapiens_tRNA- | AGGGACCTCTTGATCTGCAG | CABE | 1189 |
| Cys-GCA-9-2 | | | |
| Homo_sapiens_tRNA- | CAGGGACCTCTTGATCTGCA | CABE | 1190 |
| Cys-GCA-9-2 | | | |
| Homo_sapiens_tRNA- | GCAGTCAAATGCTCTACCCC | CABE | 1191 |
| Cys-GCA-9-3 | | | |
| Homo_sapiens_tRNA- | TGCAGTCAAATGCTCTACCC | CABE | 1192 |
| Cys-GCA-9-3 | | | |
| Homo_sapiens_tRNA- | CTGCAGTCAAATGCTCTACC | CABE | 1193 |
| Cys-GCA-9-3 | | | |
| Homo_sapiens_tRNA- | TCTGCAGTCAAATGCTCTAC | CABE | 1194 |
| Cys-GCA-9-3 | | | |
| Homo_sapiens_tRNA- | ATCTGCAGTCAAATGCTCTA | CABE | 1195 |
| Cys-GCA-9-3 | | | |
| Homo_sapiens_tRNA- | GATCTGCAGTCAAATGCTCT | CABE | 1196 |
| Cys-GCA-9-3 | | | |
| Homo_sapiens_tRNA- | TGATCTGCAGTCAAATGCTC | CABE | 1197 |
| Cys-GCA-9-3 | | | |
| Homo_sapiens_tRNA- | TTGATCTGCAGTCAAATGCT | CABE | 1198 |
| Cys-GCA-9-3 | | | |
| Homo_sapiens_tRNA- | CTTGATCTGCAGTCAAATGC | CABE | 1199 |
| Cys-GCA-9-3 | | | |
| Homo_sapiens_tRNA- | TCTTGATCTGCAGTCAAATG | CABE | 1200 |
| Cys-GCA-9-3 | | | |
| Homo_sapiens_tRNA- | CTCTTGATCTGCAGTCAAAT | CABE | 1201 |
| Cys-GCA-9-3 | | | |
| Homo_sapiens_tRNA- | CCTCTTGATCTGCAGTCAAA | CABE | 1202 |
| Cys-GCA-9-3 | | | |
| Homo_sapiens_tRNA- | ACCTCTTGATCTGCAGTCAA | CABE | 1203 |
| Cys-GCA-9-3 | | | |
| Homo_sapiens_tRNA- | GACCTCTTGATCTGCAGTCA | CABE | 1204 |
| Cys-GCA-9-3 | | | |
| Homo_sapiens_tRNA- | GGACCTCTTGATCTGCAGTC | CABE | 1205 |
| Cys-GCA-9-3 | | | |
| Homo_sapiens_tRNA- | GGGACCTCTTGATCTGCAGT | CABE | 1206 |
| Cys-GCA-9-3 | | | |
| Homo_sapiens_tRNA- | AGGGACCTCTTGATCTGCAG | CABE | 1207 |
| Cys-GCA-9-3 | | | |
| Homo_sapiens_tRNA- | CAGGGACCTCTTGATCTGCA | CABE | 1208 |
| Cys-GCA-9-3 | | | |
| Homo_sapiens_tRNA- | GCAGTCAAATGCTCTACCCC | CABE | 1209 |
| Cys-GCA-9-4 | | | |
| Homo_sapiens_tRNA- | TGCAGTCAAATGCTCTACCC | CABE | 1210 |
| Cys-GCA-9-4 | | | |
| Homo_sapiens_tRNA- | CTGCAGTCAAATGCTCTACC | CABE | 1211 |
| Cys-GCA-9-4 | | | |
| Homo_sapiens_tRNA- | TCTGCAGTCAAATGCTCTAC | CABE | 1212 |
| Cys-GCA-9-4 | | | |
| Homo_sapiens_tRNA- | ATCTGCAGTCAAATGCTCTA | CABE | 1213 |
| Cys-GCA-9-4 | | | |
| Homo_sapiens_tRNA- | GATCTGCAGTCAAATGCTCT | CABE | 1214 |
| Cys-GCA-9-4 | | | |
| Homo_sapiens_tRNA- | TGATCTGCAGTCAAATGCTC | CABE | 1215 |
| Cys-GCA-9-4 | | | |
| Homo_sapiens_tRNA- | TTGATCTGCAGTCAAATGCT | CABE | 1216 |
| Cys-GCA-9-4 | | | |
| Homo_sapiens_tRNA- | CTTGATCTGCAGTCAAATGC | CABE | 1217 |
| Cys-GCA-9-4 | | | |
| Homo_sapiens_tRNA- | TCTTGATCTGCAGTCAAATG | CABE | 1218 |
| Cys-GCA-9-4 | | | |
| Homo_sapiens_tRNA- | CTCTTGATCTGCAGTCAAAT | CABE | 1219 |
| Cys-GCA-9-4 | | | |
| Homo_sapiens_tRNA- | CCTCTTGATCTGCAGTCAAA | CABE | 1220 |
| Cys-GCA-9-4 | | | |
| Homo_sapiens_tRNA- | ACCTCTTGATCTGCAGTCAA | CABE | 1221 |
| Cys-GCA-9-4 | | | |
| Homo_sapiens_tRNA- | GACCTCTTGATCTGCAGTCA | CABE | 1222 |
| Cys-GCA-9-4 | | | |
| Homo_sapiens_tRNA- | GGACCTCTTGATCTGCAGTC | CABE | 1223 |
| Cys-GCA-9-4 | | | |
| Homo_sapiens_tRNA- | GGGACCTCTTGATCTGCAGT | CABE | 1224 |
| Cys-GCA-9-4 | | | |
| Homo_sapiens_tRNA- | AGGGACCTCTTGATCTGCAG | CABE | 1225 |
| Cys-GCA-9-4 | | | |
| Homo_sapiens_tRNA- | CAGGGACCTCTTGATCTGCA | CABE | 1226 |
| Cys-GCA-9-4 | | | |
| Homo_sapiens_tRNA- | GAGTCCAGAGTGCTAACCAT | CBE | 1227 |
| Gln-CTG-1-1 | | | |
| Homo_sapiens_tRNA- | AGAGTCCAGAGTGCTAACCA | CBE | 1228 |
| Gln-CTG-1-1 | | | |
| Homo_sapiens_tRNA- | CAGAGTCCAGAGTGCTAACC | CBE | 1229 |
| Gln-CTG-1-1 | | | |
| Homo_sapiens_tRNA- | TCAGAGTCCAGAGTGCTAAC | CBE | 1230 |
| Gln-CTG-1-1 | | | |
| Homo_sapiens_tRNA- | TTCAGAGTCCAGAGTGCTAA | CBE | 1231 |
| Gln-CTG-1-1 | | | |
| Homo_sapiens_tRNA- | ATTCAGAGTCCAGAGTGCTA | CBE | 1232 |
| Gln-CTG-1-1 | | | |
| Homo_sapiens_tRNA- | GATTCAGAGTCCAGAGTGCT | CBE | 1233 |
| Gln-CTG-1-1 | | | |
| Homo_sapiens_tRNA- | GGATTCAGAGTCCAGAGTGC | CBE | 1234 |
| Gln-CTG-1-1 | | | |
| Homo_sapiens_tRNA- | TGGATTCAGAGTCCAGAGTG | CBE | 1235 |
| Gln-CTG-1-1 | | | |
| Homo_sapiens_tRNA- | CTGGATTCAGAGTCCAGAGT | CBE | 1236 |
| Gln-CTG-1-1 | | | |
| Homo_sapiens_tRNA- | GCTGGATTCAGAGTCCAGAG | CBE | 1237 |
| Gln-CTG-1-1 | | | |
| Homo_sapiens_tRNA- | CGCTGGATTCAGAGTCCAGA | CBE | 1238 |
| Gln-CTG-1-1 | | | |
| Homo_sapiens_tRNA- | TCGCTGGATTCAGAGTCCAG | CBE | 1239 |
| Gln-CTG-1-1 | | | |
| Homo_sapiens_tRNA- | ATCGCTGGATTCAGAGTCCA | CBE | 1240 |
| Gln-CTG-1-1 | | | |
| Homo_sapiens_tRNA- | GATCGCTGGATTCAGAGTCC | CBE | 1241 |
| Gln-CTG-1-1 | | | |
| Homo_sapiens_tRNA- | GGATCGCTGGATTCAGAGTC | CBE | 1242 |
| Gln-CTG-1-1 | | | |
| Homo_sapiens_tRNA- | CGGATCGCTGGATTCAGAGT | CBE | 1243 |
| Gln-CTG-1-1 | | | |
| Homo_sapiens_tRNA- | TCGGATCGCTGGATTCAGAG | CBE | 1244 |
| Gln-CTG-1-1 | | | |
| Homo_sapiens_tRNA- | GAGTCCAGAGTGCTAACCAT | CBE | 1245 |
| Gln-CTG-1-2 | | | |
| Homo_sapiens_tRNA- | AGAGTCCAGAGTGCTAACCA | CBE | 1246 |
| Gln-CTG-1-2 | | | |
| Homo_sapiens_tRNA- | CAGAGTCCAGAGTGCTAACC | CBE | 1247 |
| Gln-CTG-1-2 | | | |
| Homo_sapiens_tRNA- | TCAGAGTCCAGAGTGCTAAC | CBE | 1248 |
| Gln-CTG-1-2 | | | |
| Homo_sapiens_tRNA- | TTCAGAGTCCAGAGTGCTAA | CBE | 1249 |
| Gln-CTG-1-2 | | | |
| Homo_sapiens_tRNA- | ATTCAGAGTCCAGAGTGCTA | CBE | 1250 |
| Gln-CTG-1-2 | | | |
| Homo_sapiens_tRNA- | GATTCAGAGTCCAGAGTGCT | CBE | 1251 |
| Gln-CTG-1-2 | | | |
| Homo_sapiens_tRNA- | GGATTCAGAGTCCAGAGTGC | CBE | 1252 |
| Gln-CTG-1-2 | | | |
| Homo_sapiens_tRNA- | TGGATTCAGAGTCCAGAGTG | CBE | 1253 |
| Gln-CTG-1-2 | | | |
| Homo_sapiens_tRNA- | CTGGATTCAGAGTCCAGAGT | CBE | 1254 |
| Gln-CTG-1-2 | | | |
| Homo_sapiens_tRNA- | GCTGGATTCAGAGTCCAGAG | CBE | 1255 |
| Gln-CTG-1-2 | | | |
| Homo_sapiens_tRNA- | CGCTGGATTCAGAGTCCAGA | CBE | 1256 |
| Gln-CTG-1-2 | | | |
| Homo_sapiens_tRNA- | TCGCTGGATTCAGAGTCCAG | CBE | 1257 |
| Gln-CTG-1-2 | | | |
| Homo_sapiens_tRNA- | ATCGCTGGATTCAGAGTCCA | CBE | 1258 |
| Gln-CTG-1-2 | | | |
| Homo_sapiens_tRNA- | GATCGCTGGATTCAGAGTCC | CBE | 1259 |
| Gln-CTG-1-2 | | | |
| Homo_sapiens_tRNA- | GGATCGCTGGATTCAGAGTC | CBE | 1260 |
| Gln-CTG-1-2 | | | |
| Homo_sapiens_tRNA- | CGGATCGCTGGATTCAGAGT | CBE | 1261 |
| Gln-CTG-1-2 | | | |
| Homo_sapiens_tRNA- | TCGGATCGCTGGATTCAGAG | CBE | 1262 |
| Gln-CTG-1-2 | | | |
| Homo_sapiens_tRNA- | GAGTCCAGAGTGCTAACCAT | CBE | 1263 |
| Gln-CTG-1-3 | | | |
| Homo_sapiens_tRNA- | AGAGTCCAGAGTGCTAACCA | CBE | 1264 |
| Gln-CTG-1-3 | | | |
| Homo_sapiens_tRNA- | CAGAGTCCAGAGTGCTAACC | CBE | 1265 |
| Gln-CTG-1-3 | | | |
| Homo_sapiens_tRNA- | TCAGAGTCCAGAGTGCTAAC | CBE | 1266 |
| Gln-CTG-1-3 | | | |
| Homo_sapiens_tRNA- | TTCAGAGTCCAGAGTGCTAA | CBE | 1267 |
| Gln-CTG-1-3 | | | |
| Homo_sapiens_tRNA- | ATTCAGAGTCCAGAGTGCTA | CBE | 1268 |
| Gln-CTG-1-3 | | | |
| Homo_sapiens_tRNA- | GATTCAGAGTCCAGAGTGCT | CBE | 1269 |
| Gln-CTG-1-3 | | | |
| Homo_sapiens_tRNA- | GGATTCAGAGTCCAGAGTGC | CBE | 1270 |
| Gln-CTG-1-3 | | | |
| Homo_sapiens_tRNA- | TGGATTCAGAGTCCAGAGTG | CBE | 1271 |
| Gln-CTG-1-3 | | | |
| Homo_sapiens_tRNA- | CTGGATTCAGAGTCCAGAGT | CBE | 1272 |
| Gln-CTG-1-3 | | | |
| Homo_sapiens_tRNA- | GCTGGATTCAGAGTCCAGAG | CBE | 1273 |
| Gln-CTG-1-3 | | | |
| Homo_sapiens_tRNA- | CGCTGGATTCAGAGTCCAGA | CBE | 1274 |
| Gln-CTG-1-3 | | | |
| Homo_sapiens_tRNA- | TCGCTGGATTCAGAGTCCAG | CBE | 1275 |
| Gln-CTG-1-3 | | | |
| Homo_sapiens_tRNA- | ATCGCTGGATTCAGAGTCCA | CBE | 1276 |
| Gln-CTG-1-3 | | | |
| Homo_sapiens_tRNA- | GATCGCTGGATTCAGAGTCC | CBE | 1277 |
| Gln-CTG-1-3 | | | |
| Homo_sapiens_tRNA- | GGATCGCTGGATTCAGAGTC | CBE | 1278 |
| Gln-CTG-1-3 | | | |
| Homo_sapiens_tRNA- | CGGATCGCTGGATTCAGAGT | CBE | 1279 |
| Gln-CTG-1-3 | | | |
| Homo_sapiens_tRNA- | TCGGATCGCTGGATTCAGAG | CBE | 1280 |
| Gln-CTG-1-3 | | | |
| Homo_sapiens_tRNA- | GAGTCCAGAGTGCTAACCAT | CBE | 1281 |
| Gln-CTG-1-4 | | | |
| Homo_sapiens_tRNA- | AGAGTCCAGAGTGCTAACCA | CBE | 1282 |
| Gln-CTG-1-4 | | | |
| Homo_sapiens_tRNA- | CAGAGTCCAGAGTGCTAACC | CBE | 1283 |
| Gln-CTG-1-4 | | | |
| Homo_sapiens_tRNA- | TCAGAGTCCAGAGTGCTAAC | CBE | 1284 |
| Gln-CTG-1-4 | | | |
| Homo_sapiens_tRNA- | TTCAGAGTCCAGAGTGCTAA | CBE | 1285 |
| Gln-CTG-1-4 | | | |
| Homo_sapiens_tRNA- | ATTCAGAGTCCAGAGTGCTA | CBE | 1286 |
| Gln-CTG-1-4 | | | |
| Homo_sapiens_tRNA- | GATTCAGAGTCCAGAGTGCT | CBE | 1287 |
| Gln-CTG-1-4 | | | |
| Homo_sapiens_tRNA- | GGATTCAGAGTCCAGAGTGC | CBE | 1288 |
| Gln-CTG-1-4 | | | |
| Homo_sapiens_tRNA- | TGGATTCAGAGTCCAGAGTG | CBE | 1289 |
| Gln-CTG-1-4 | | | |
| Homo_sapiens_tRNA- | CTGGATTCAGAGTCCAGAGT | CBE | 1290 |
| Gln-CTG-1-4 | | | |
| Homo_sapiens_tRNA- | GCTGGATTCAGAGTCCAGAG | CBE | 1291 |
| Gln-CTG-1-4 | | | |
| Homo_sapiens_tRNA- | CGCTGGATTCAGAGTCCAGA | CBE | 1292 |
| Gln-CTG-1-4 | | | |
| Homo_sapiens_tRNA- | TCGCTGGATTCAGAGTCCAG | CBE | 1293 |
| Gln-CTG-1-4 | | | |
| Homo_sapiens_tRNA- | ATCGCTGGATTCAGAGTCCA | CBE | 1294 |
| Gln-CTG-1-4 | | | |
| Homo_sapiens_tRNA- | GATCGCTGGATTCAGAGTCC | CBE | 1295 |
| Gln-CTG-1-4 | | | |
| Homo_sapiens_tRNA- | GGATCGCTGGATTCAGAGTC | CBE | 1296 |
| Gln-CTG-1-4 | | | |
| Homo_sapiens_tRNA- | CGGATCGCTGGATTCAGAGT | CBE | 1297 |
| Gln-CTG-1-4 | | | |
| Homo_sapiens_tRNA- | TCGGATCGCTGGATTCAGAG | CBE | 1298 |
| Gln-CTG-1-4 | | | |
| Homo_sapiens_tRNA- | GAGTCCAGAGTGCTAACCAT | CBE | 1299 |
| Gln-CTG-1-5 | | | |
| Homo_sapiens_tRNA- | AGAGTCCAGAGTGCTAACCA | CBE | 1300 |
| Gln-CTG-1-5 | | | |
| Homo_sapiens_tRNA- | CAGAGTCCAGAGTGCTAACC | CBE | 1301 |
| Gln-CTG-1-5 | | | |
| Homo_sapiens_tRNA- | TCAGAGTCCAGAGTGCTAAC | CBE | 1302 |
| Gln-CTG-1-5 | | | |
| Homo_sapiens_tRNA- | TTCAGAGTCCAGAGTGCTAA | CBE | 1303 |
| Gln-CTG-1-5 | | | |
| Homo_sapiens_tRNA- | ATTCAGAGTCCAGAGTGCTA | CBE | 1304 |
| Gln-CTG-1-5 | | | |
| Homo_sapiens_tRNA- | GATTCAGAGTCCAGAGTGCT | CBE | 1305 |
| Gln-CTG-1-5 | | | |
| Homo_sapiens_tRNA- | GGATTCAGAGTCCAGAGTGC | CBE | 1306 |
| Gln-CTG-1-5 | | | |
| Homo_sapiens_tRNA- | TGGATTCAGAGTCCAGAGTG | CBE | 1307 |
| Gln-CTG-1-5 | | | |
| Homo_sapiens_tRNA- | CTGGATTCAGAGTCCAGAGT | CBE | 1308 |
| Gln-CTG-1-5 | | | |
| Homo_sapiens_tRNA- | GCTGGATTCAGAGTCCAGAG | CBE | 1309 |
| Gln-CTG-1-5 | | | |
| Homo_sapiens_tRNA- | CGCTGGATTCAGAGTCCAGA | CBE | 1310 |
| Gln-CTG-1-5 | | | |
| Homo_sapiens_tRNA- | TCGCTGGATTCAGAGTCCAG | CBE | 1311 |
| Gln-CTG-1-5 | | | |
| Homo_sapiens_tRNA- | ATCGCTGGATTCAGAGTCCA | CBE | 1312 |
| Gln-CTG-1-5 | | | |
| Homo_sapiens_tRNA- | GATCGCTGGATTCAGAGTCC | CBE | 1313 |
| Gln-CTG-1-5 | | | |
| Homo_sapiens_tRNA- | GGATCGCTGGATTCAGAGTC | CBE | 1314 |
| Gln-CTG-1-5 | | | |
| Homo_sapiens_tRNA- | CGGATCGCTGGATTCAGAGT | CBE | 1315 |
| Gln-CTG-1-5 | | | |
| Homo_sapiens_tRNA- | TCGGATCGCTGGATTCAGAG | CBE | 1316 |
| Gln-CTG-1-5 | | | |
| Homo_sapiens_tRNA- | GAGTCCAGAGTGCTAACCAT | CBE | 1317 |
| Gln-CTG-2-1 | | | |
| Homo_sapiens_tRNA- | AGAGTCCAGAGTGCTAACCA | CBE | 1318 |
| Gln-CTG-2-1 | | | |
| Homo_sapiens_tRNA- | CAGAGTCCAGAGTGCTAACC | CBE | 1319 |
| Gln-CTG-2-1 | | | |
| Homo_sapiens_tRNA- | TCAGAGTCCAGAGTGCTAAC | CBE | 1320 |
| Gln-CTG-2-1 | | | |
| Homo_sapiens_tRNA- | TTCAGAGTCCAGAGTGCTAA | CBE | 1321 |
| Gln-CTG-2-1 | | | |
| Homo_sapiens_tRNA- | ATTCAGAGTCCAGAGTGCTA | CBE | 1322 |
| Gln-CTG-2-1 | | | |
| Homo_sapiens_tRNA- | GATTCAGAGTCCAGAGTGCT | CBE | 1323 |
| Gln-CTG-2-1 | | | |
| Homo_sapiens_tRNA- | GGATTCAGAGTCCAGAGTGC | CBE | 1324 |
| Gln-CTG-2-1 | | | |
| Homo_sapiens_tRNA- | TGGATTCAGAGTCCAGAGTG | CBE | 1325 |
| Gln-CTG-2-1 | | | |
| Homo_sapiens_tRNA- | CTGGATTCAGAGTCCAGAGT | CBE | 1326 |
| Gln-CTG-2-1 | | | |
| Homo_sapiens_tRNA- | GCTGGATTCAGAGTCCAGAG | CBE | 1327 |
| Gln-CTG-2-1 | | | |
| Homo_sapiens_tRNA- | CGCTGGATTCAGAGTCCAGA | CBE | 1328 |
| Gln-CTG-2-1 | | | |
| Homo_sapiens_tRNA- | TCGCTGGATTCAGAGTCCAG | CBE | 1329 |
| Gln-CTG-2-1 | | | |
| Homo_sapiens_tRNA- | ATCGCTGGATTCAGAGTCCA | CBE | 1330 |
| Gln-CTG-2-1 | | | |
| Homo_sapiens_tRNA- | GATCGCTGGATTCAGAGTCC | CBE | 1331 |
| Gln-CTG-2-1 | | | |
| Homo_sapiens_tRNA- | GGATCGCTGGATTCAGAGTC | CBE | 1332 |
| Gln-CTG-2-1 | | | |
| Homo_sapiens_tRNA- | CGGATCGCTGGATTCAGAGT | CBE | 1333 |
| Gln-CTG-2-1 | | | |
| Homo_sapiens_tRNA- | TCGGATCGCTGGATTCAGAG | CBE | 1334 |
| Gln-CTG-2-1 | | | |
| Homo_sapiens_tRNA- | GAGTCCAGAGTGCTCACCAT | CBE | 1335 |
| Gln-CTG-3-1 | | | |
| Homo_sapiens_tRNA- | AGAGTCCAGAGTGCTCACCA | CBE | 1336 |
| Gln-CTG-3-1 | | | |
| Homo_sapiens_tRNA- | CAGAGTCCAGAGTGCTCACC | CBE | 1337 |
| Gln-CTG-3-1 | | | |
| Homo_sapiens_tRNA- | TCAGAGTCCAGAGTGCTCAC | CBE | 1338 |
| Gln-CTG-3-1 | | | |
| Homo_sapiens_tRNA- | TTCAGAGTCCAGAGTGCTCA | CBE | 1339 |
| Gln-CTG-3-1 | | | |
| Homo_sapiens_tRNA- | ATTCAGAGTCCAGAGTGCTC | CBE | 1340 |
| Gln-CTG-3-1 | | | |
| Homo_sapiens_tRNA- | GATTCAGAGTCCAGAGTGCT | CBE | 1341 |
| Gln-CTG-3-1 | | | |
| Homo_sapiens_tRNA- | GGATTCAGAGTCCAGAGTGC | CBE | 1342 |
| Gln-CTG-3-1 | | | |
| Homo_sapiens_tRNA- | TGGATTCAGAGTCCAGAGTG | CBE | 1343 |
| Gln-CTG-3-1 | | | |
| Homo_sapiens_tRNA- | CTGGATTCAGAGTCCAGAGT | CBE | 1344 |
| Gln-CTG-3-1 | | | |
| Homo_sapiens_tRNA- | GCTGGATTCAGAGTCCAGAG | CBE | 1345 |
| Gln-CTG-3-1 | | | |
| Homo_sapiens_tRNA- | CGCTGGATTCAGAGTCCAGA | CBE | 1346 |
| Gln-CTG-3-1 | | | |
| Homo_sapiens_tRNA- | TCGCTGGATTCAGAGTCCAG | CBE | 1347 |
| Gln-CTG-3-1 | | | |
| Homo_sapiens_tRNA- | ATCGCTGGATTCAGAGTCCA | CBE | 1348 |
| Gln-CTG-3-1 | | | |
| Homo_sapiens_tRNA- | GATCGCTGGATTCAGAGTCC | CBE | 1349 |
| Gln-CTG-3-1 | | | |
| Homo_sapiens_tRNA- | GGATCGCTGGATTCAGAGTC | CBE | 1350 |
| Gln-CTG-3-1 | | | |
| Homo_sapiens_tRNA- | CGGATCGCTGGATTCAGAGT | CBE | 1351 |
| Gln-CTG-3-1 | | | |
| Homo_sapiens_tRNA- | TCGGATCGCTGGATTCAGAG | CBE | 1352 |
| Gln-CTG-3-1 | | | |
| Homo_sapiens_tRNA- | GAGTCCAGAGTGCTCACCAT | CBE | 1353 |
| Gln-CTG-3-2 | | | |
| Homo_sapiens_tRNA- | AGAGTCCAGAGTGCTCACCA | CBE | 1354 |
| Gln-CTG-3-2 | | | |
| Homo_sapiens_tRNA- | CAGAGTCCAGAGTGCTCACC | CBE | 1355 |
| Gln-CTG-3-2 | | | |
| Homo_sapiens_tRNA- | TCAGAGTCCAGAGTGCTCAC | CBE | 1356 |
| Gln-CTG-3-2 | | | |
| Homo_sapiens_tRNA- | TTCAGAGTCCAGAGTGCTCA | CBE | 1357 |
| Gln-CTG-3-2 | | | |
| Homo_sapiens_tRNA- | ATTCAGAGTCCAGAGTGCTC | CBE | 1358 |
| Gln-CTG-3-2 | | | |
| Homo_sapiens_tRNA- | GATTCAGAGTCCAGAGTGCT | CBE | 1359 |
| Gln-CTG-3-2 | | | |
| Homo_sapiens_tRNA- | GGATTCAGAGTCCAGAGTGC | CBE | 1360 |
| Gln-CTG-3-2 | | | |
| Homo_sapiens_tRNA- | TGGATTCAGAGTCCAGAGTG | CBE | 1361 |
| Gln-CTG-3-2 | | | |
| Homo_sapiens_tRNA- | CTGGATTCAGAGTCCAGAGT | CBE | 1362 |
| Gln-CTG-3-2 | | | |
| Homo_sapiens_tRNA- | GCTGGATTCAGAGTCCAGAG | CBE | 1363 |
| Gln-CTG-3-2 | | | |
| Homo_sapiens_tRNA- | CGCTGGATTCAGAGTCCAGA | CBE | 1364 |
| Gln-CTG-3-2 | | | |
| Homo_sapiens_tRNA- | TCGCTGGATTCAGAGTCCAG | CBE | 1365 |
| Gln-CTG-3-2 | | | |
| Homo_sapiens_tRNA- | ATCGCTGGATTCAGAGTCCA | CBE | 1366 |
| Gln-CTG-3-2 | | | |
| Homo_sapiens_tRNA- | GATCGCTGGATTCAGAGTCC | CBE | 1367 |
| Gln-CTG-3-2 | | | |
| Homo_sapiens_tRNA- | GGATCGCTGGATTCAGAGTC | CBE | 1368 |
| Gln-CTG-3-2 | | | |
| Homo_sapiens_tRNA- | CGGATCGCTGGATTCAGAGT | CBE | 1369 |
| Gln-CTG-3-2 | | | |
| Homo_sapiens_tRNA- | TCGGATCGCTGGATTCAGAG | CBE | 1370 |
| Gln-CTG-3-2 | | | |
| Homo_sapiens_tRNA- | GAGTCCAGAGTGCTTACCAT | CBE | 1371 |
| Gln-CTG-4-1 | | | |
| Homo_sapiens_tRNA- | AGAGTCCAGAGTGCTTACCA | CBE | 1372 |
| Gln-CTG-4-1 | | | |
| Homo_sapiens_tRNA- | CAGAGTCCAGAGTGCTTACC | CBE | 1373 |
| Gln-CTG-4-1 | | | |
| Homo_sapiens_tRNA- | TCAGAGTCCAGAGTGCTTAC | CBE | 1374 |
| Gln-CTG-4-1 | | | |
| Homo_sapiens_tRNA- | TTCAGAGTCCAGAGTGCTTA | CBE | 1375 |
| Gln-CTG-4-1 | | | |
| Homo_sapiens_tRNA- | ATTCAGAGTCCAGAGTGCTT | CBE | 1376 |
| Gln-CTG-4-1 | | | |
| Homo_sapiens_tRNA- | GATTCAGAGTCCAGAGTGCT | CBE | 1377 |
| Gln-CTG-4-1 | | | |
| Homo_sapiens_tRNA- | GGATTCAGAGTCCAGAGTGC | CBE | 1378 |
| Gln-CTG-4-1 | | | |
| Homo_sapiens_tRNA- | TGGATTCAGAGTCCAGAGTG | CBE | 1379 |
| Gln-CTG-4-1 | | | |
| Homo_sapiens_tRNA- | CTGGATTCAGAGTCCAGAGT | CBE | 1380 |
| Gln-CTG-4-1 | | | |
| Homo_sapiens_tRNA- | GCTGGATTCAGAGTCCAGAG | CBE | 1381 |
| Gln-CTG-4-1 | | | |
| Homo_sapiens_tRNA- | CGCTGGATTCAGAGTCCAGA | CBE | 1382 |
| Gln-CTG-4-1 | | | |
| Homo_sapiens_tRNA- | TCGCTGGATTCAGAGTCCAG | CBE | 1383 |
| Gln-CTG-4-1 | | | |
| Homo_sapiens_tRNA- | ATCGCTGGATTCAGAGTCCA | CBE | 1384 |
| Gln-CTG-4-1 | | | |
| Homo_sapiens_tRNA- | GATCGCTGGATTCAGAGTCC | CBE | 1385 |
| Gln-CTG-4-1 | | | |
| Homo_sapiens_tRNA- | GGATCGCTGGATTCAGAGTC | CBE | 1386 |
| Gln-CTG-4-1 | | | |
| Homo_sapiens_tRNA- | CGGATCGCTGGATTCAGAGT | CBE | 1387 |
| Gln-CTG-4-1 | | | |
| Homo_sapiens_tRNA- | TCGGATCGCTGGATTCAGAG | CBE | 1388 |
| Gln-CTG-4-1 | | | |
| Homo_sapiens_tRNA- | GAGTCCAGAGTGCTTACCAT | CBE | 1389 |
| Gln-CTG-4-2 | | | |
| Homo_sapiens_tRNA- | AGAGTCCAGAGTGCTTACCA | CBE | 1390 |
| Gln-CTG-4-2 | | | |
| Homo_sapiens_tRNA- | CAGAGTCCAGAGTGCTTACC | CBE | 1391 |
| Gln-CTG-4-2 | | | |
| Homo_sapiens_tRNA- | TCAGAGTCCAGAGTGCTTAC | CBE | 1392 |
| Gln-CTG-4-2 | | | |
| Homo_sapiens_tRNA- | TTCAGAGTCCAGAGTGCTTA | CBE | 1393 |
| Gln-CTG-4-2 | | | |
| Homo_sapiens_tRNA- | ATTCAGAGTCCAGAGTGCTT | CBE | 1394 |
| Gln-CTG-4-2 | | | |
| Homo_sapiens_tRNA- | GATTCAGAGTCCAGAGTGCT | CBE | 1395 |
| Gln-CTG-4-2 | | | |
| Homo_sapiens_tRNA- | GGATTCAGAGTCCAGAGTGC | CBE | 1396 |
| Gln-CTG-4-2 | | | |
| Homo_sapiens_tRNA- | TGGATTCAGAGTCCAGAGTG | CBE | 1397 |
| Gln-CTG-4-2 | | | |
| Homo_sapiens_tRNA- | CTGGATTCAGAGTCCAGAGT | CBE | 1398 |
| Gln-CTG-4-2 | | | |
| Homo_sapiens_tRNA- | GCTGGATTCAGAGTCCAGAG | CBE | 1399 |
| Gln-CTG-4-2 | | | |
| Homo_sapiens_tRNA- | CGCTGGATTCAGAGTCCAGA | CBE | 1400 |
| Gln-CTG-4-2 | | | |
| Homo_sapiens_tRNA- | TCGCTGGATTCAGAGTCCAG | CBE | 1401 |
| Gln-CTG-4-2 | | | |
| Homo_sapiens_tRNA- | ATCGCTGGATTCAGAGTCCA | CBE | 1402 |
| Gln-CTG-4-2 | | | |
| Homo_sapiens_tRNA- | GATCGCTGGATTCAGAGTCC | CBE | 1403 |
| Gln-CTG-4-2 | | | |
| Homo_sapiens_tRNA- | GGATCGCTGGATTCAGAGTC | CBE | 1404 |
| Gln-CTG-4-2 | | | |
| Homo_sapiens_tRNA- | CGGATCGCTGGATTCAGAGT | CBE | 1405 |
| Gln-CTG-4-2 | | | |
| Homo_sapiens_tRNA- | TCGGATCGCTGGATTCAGAG | CBE | 1406 |
| Gln-CTG-4-2 | | | |
| Homo_sapiens_tRNA- | GAGTCCAGAGTGCTAACCAT | CBE | 1407 |
| Gln-CTG-5-1 | | | |
| Homo_sapiens_tRNA- | AGAGTCCAGAGTGCTAACCA | CBE | 1408 |
| Gln-CTG-5-1 | | | |
| Homo_sapiens_tRNA- | CAGAGTCCAGAGTGCTAACC | CBE | 1409 |
| Gln-CTG-5-1 | | | |
| Homo_sapiens_tRNA- | TCAGAGTCCAGAGTGCTAAC | CBE | 1410 |
| Gln-CTG-5-1 | | | |
| Homo_sapiens_tRNA- | TTCAGAGTCCAGAGTGCTAA | CBE | 1411 |
| Gln-CTG-5-1 | | | |
| Homo_sapiens_tRNA- | ATTCAGAGTCCAGAGTGCTA | CBE | 1412 |
| Gln-CTG-5-1 | | | |
| Homo_sapiens_tRNA- | GATTCAGAGTCCAGAGTGCT | CBE | 1413 |
| Gln-CTG-5-1 | | | |
| Homo_sapiens_tRNA- | GGATTCAGAGTCCAGAGTGC | CBE | 1414 |
| Gln-CTG-5-1 | | | |
| Homo_sapiens_tRNA- | CGGATTCAGAGTCCAGAGTG | CBE | 1415 |
| Gln-CTG-5-1 | | | |
| Homo_sapiens_tRNA- | CCGGATTCAGAGTCCAGAGT | CBE | 1416 |
| Gln-CTG-5-1 | | | |
| Homo_sapiens_tRNA- | ACCGGATTCAGAGTCCAGAG | CBE | 1417 |
| Gln-CTG-5-1 | | | |
| Homo_sapiens_tRNA- | TACCGGATTCAGAGTCCAGA | CBE | 1418 |
| Gln-CTG-5-1 | | | |
| Homo_sapiens_tRNA- | TTACCGGATTCAGAGTCCAG | CBE | 1419 |
| Gln-CTG-5-1 | | | |
| Homo_sapiens_tRNA- | ATTACCGGATTCAGAGTCCA | CBE | 1420 |
| Gln-CTG-5-1 | | | |
| Homo_sapiens_tRNA- | GATTACCGGATTCAGAGTCC | CBE | 1421 |
| Gln-CTG-5-1 | | | |
| Homo_sapiens_tRNA- | GGATTACCGGATTCAGAGTC | CBE | 1422 |
| Gln-CTG-5-1 | | | |
| Homo_sapiens_tRNA- | CGGATTACCGGATTCAGAGT | CBE | 1423 |
| Gln-CTG-5-1 | | | |
| Homo_sapiens_tRNA- | TCGGATTACCGGATTCAGAG | CBE | 1424 |
| Gln-CTG-5-1 | | | |
| Homo_sapiens_tRNA- | GAGTCCAGAGTGCTGACCAT | CBE | 1425 |
| Gln-CTG-6-1 | | | |
| Homo_sapiens_tRNA- | AGAGTCCAGAGTGCTGACCA | CBE | 1426 |
| Gln-CTG-6-1 | | | |
| Homo_sapiens_tRNA- | CAGAGTCCAGAGTGCTGACC | CBE | 1427 |
| Gln-CTG-6-1 | | | |
| Homo_sapiens_tRNA- | TCAGAGTCCAGAGTGCTGAC | CBE | 1428 |
| Gln-CTG-6-1 | | | |
| Homo_sapiens_tRNA- | TTCAGAGTCCAGAGTGCTGA | CBE | 1429 |
| Gln-CTG-6-1 | | | |
| Homo_sapiens_tRNA- | ATTCAGAGTCCAGAGTGCTG | CBE | 1430 |
| Gln-CTG-6-1 | | | |
| Homo_sapiens_tRNA- | GATTCAGAGTCCAGAGTGCT | CBE | 1431 |
| Gln-CTG-6-1 | | | |
| Homo_sapiens_tRNA- | GGATTCAGAGTCCAGAGTGC | CBE | 1432 |
| Gln-CTG-6-1 | | | |
| Homo_sapiens_tRNA- | TGGATTCAGAGTCCAGAGTG | CBE | 1433 |
| Gln-CTG-6-1 | | | |
| Homo_sapiens_tRNA- | CTGGATTCAGAGTCCAGAGT | CBE | 1434 |
| Gln-CTG-6-1 | | | |
| Homo_sapiens_tRNA- | GCTGGATTCAGAGTCCAGAG | CBE | 1435 |
| Gln-CTG-6-1 | | | |
| Homo_sapiens_tRNA- | CGCTGGATTCAGAGTCCAGA | CBE | 1436 |
| Gln-CTG-6-1 | | | |
| Homo_sapiens_tRNA- | TCGCTGGATTCAGAGTCCAG | CBE | 1437 |
| Gln-CTG-6-1 | | | |
| Homo_sapiens_tRNA- | ATCGCTGGATTCAGAGTCCA | CBE | 1438 |
| Gln-CTG-6-1 | | | |
| Homo_sapiens_tRNA- | GATCGCTGGATTCAGAGTCC | CBE | 1439 |
| Gln-CTG-6-1 | | | |
| Homo_sapiens_tRNA- | GGATCGCTGGATTCAGAGTC | CBE | 1440 |
| Gln-CTG-6-1 | | | |
| Homo_sapiens_tRNA- | CGGATCGCTGGATTCAGAGT | CBE | 1441 |
| Gln-CTG-6-1 | | | |
| Homo_sapiens_tRNA- | TCGGATCGCTGGATTCAGAG | CBE | 1442 |
| Gln-CTG-6-1 | | | |
| Homo_sapiens_tRNA- | GAGTCCAGAGTGCTTACCAT | CBE | 1443 |
| Gln-CTG-7-1 | | | |
| Homo_sapiens_tRNA- | AGAGTCCAGAGTGCTTACCA | CBE | 1444 |
| Gln-CTG-7-1 | | | |
| Homo_sapiens_tRNA- | CAGAGTCCAGAGTGCTTACC | CBE | 1445 |
| Gln-CTG-7-1 | | | |
| Homo_sapiens_tRNA- | TCAGAGTCCAGAGTGCTTAC | CBE | 1446 |
| Gln-CTG-7-1 | | | |
| Homo_sapiens_tRNA- | TTCAGAGTCCAGAGTGCTTA | CBE | 1447 |
| Gln-CTG-7-1 | | | |
| Homo_sapiens_tRNA- | ATTCAGAGTCCAGAGTGCTT | CBE | 1448 |
| Gln-CTG-7-1 | | | |
| Homo_sapiens_tRNA- | GATTCAGAGTCCAGAGTGCT | CBE | 1449 |
| Gln-CTG-7-1 | | | |
| Homo_sapiens_tRNA- | GGATTCAGAGTCCAGAGTGC | CBE | 1450 |
| Gln-CTG-7-1 | | | |
| Homo_sapiens_tRNA- | TGGATTCAGAGTCCAGAGTG | CBE | 1451 |
| Gln-CTG-7-1 | | | |
| Homo_sapiens_tRNA- | CTGGATTCAGAGTCCAGAGT | CBE | 1452 |
| Gln-CTG-7-1 | | | |
| Homo_sapiens_tRNA- | GCTGGATTCAGAGTCCAGAG | CBE | 1453 |
| Gln-CTG-7-1 | | | |
| Homo_sapiens_tRNA- | GGCTGGATTCAGAGTCCAGA | CBE | 1454 |
| Gln-CTG-7-1 | | | |
| Homo_sapiens_tRNA- | TGGCTGGATTCAGAGTCCAG | CBE | 1455 |
| Gln-CTG-7-1 | | | |
| Homo_sapiens_tRNA- | ATGGCTGGATTCAGAGTCCA | CBE | 1456 |
| Gln-CTG-7-1 | | | |
| Homo_sapiens_tRNA- | GATGGCTGGATTCAGAGTCC | CBE | 1457 |
| Gln-CTG-7-1 | | | |
| Homo_sapiens_tRNA- | AGATGGCTGGATTCAGAGTC | CBE | 1458 |
| Gln-CTG-7-1 | | | |
| Homo_sapiens_tRNA- | CAGATGGCTGGATTCAGAGT | CBE | 1459 |
| Gln-CTG-7-1 | | | |
| Homo_sapiens_tRNA- | TCAGATGGCTGGATTCAGAG | CBE | 1460 |
| Gln-CTG-7-1 | | | |
| Homo_sapiens_tRNA- | AAGTCCAGAGTGCTAACCAT | CBE | 1461 |
| Gln-TTG-1-1 | | | |
| Homo_sapiens_tRNA- | AAAGTCCAGAGTGCTAACCA | CBE | 1462 |
| Gln-TTG-1-1 | | | |
| Homo_sapiens_tRNA- | CAAAGTCCAGAGTGCTAACC | CBE | 1463 |
| Gln-TTG-1-1 | | | |
| Homo_sapiens_tRNA- | TCAAAGTCCAGAGTGCTAAC | CBE | 1464 |
| Gln-TTG-1-1 | | | |
| Homo_sapiens_tRNA- | TTCAAAGTCCAGAGTGCTAA | CBE | 1465 |
| Gln-TTG-1-1 | | | |
| Homo_sapiens_tRNA- | ATTCAAAGTCCAGAGTGCTA | CBE | 1466 |
| Gln-TTG-1-1 | | | |
| Homo_sapiens_tRNA- | GATTCAAAGTCCAGAGTGCT | CBE | 1467 |
| Gln-TTG-1-1 | | | |
| Homo_sapiens_tRNA- | GGATTCAAAGTCCAGAGTGC | CBE | 1468 |
| Gln-TTG-1-1 | | | |
| Homo_sapiens_tRNA- | TGGATTCAAAGTCCAGAGTG | CBE | 1469 |
| Gln-TTG-1-1 | | | |
| Homo_sapiens_tRNA- | CTGGATTCAAAGTCCAGAGT | CBE | 1470 |
| Gln-TTG-1-1 | | | |
| Homo_sapiens_tRNA- | GCTGGATTCAAAGTCCAGAG | CBE | 1471 |
| Gln-TTG-1-1 | | | |
| Homo_sapiens_tRNA- | CGCTGGATTCAAAGTCCAGA | CBE | 1472 |
| Gln-TTG-1-1 | | | |
| Homo_sapiens_tRNA- | TCGCTGGATTCAAAGTCCAG | CBE | 1473 |
| Gln-TTG-1-1 | | | |
| Homo_sapiens_tRNA- | ATCGCTGGATTCAAAGTCCA | CBE | 1474 |
| Gln-TTG-1-1 | | | |
| Homo_sapiens_tRNA- | GATCGCTGGATTCAAAGTCC | CBE | 1475 |
| Gln-TTG-1-1 | | | |
| Homo_sapiens_tRNA- | GGATCGCTGGATTCAAAGTC | CBE | 1476 |
| Gln-TTG-1-1 | | | |
| Homo_sapiens_tRNA- | CGGATCGCTGGATTCAAAGT | CBE | 1477 |
| Gln-TTG-1-1 | | | |
| Homo_sapiens_tRNA- | TCGGATCGCTGGATTCAAAG | CBE | 1478 |
| Gln-TTG-1-1 | | | |
| Homo_sapiens_tRNA- | AAGTCCAGAGTGCTAACCAT | CBE | 1479 |
| Gln-TTG-2-1 | | | |
| Homo_sapiens_tRNA- | AAAGTCCAGAGTGCTAACCA | CBE | 1480 |
| Gln-TTG-2-1 | | | |
| Homo_sapiens_tRNA- | CAAAGTCCAGAGTGCTAACC | CBE | 1481 |
| Gln-TTG-2-1 | | | |
| Homo_sapiens_tRNA- | TCAAAGTCCAGAGTGCTAAC | CBE | 1482 |
| Gln-TTG-2-1 | | | |
| Homo_sapiens_tRNA- | TTCAAAGTCCAGAGTGCTAA | CBE | 1483 |
| Gln-TTG-2-1 | | | |
| Homo_sapiens_tRNA- | ATTCAAAGTCCAGAGTGCTA | CBE | 1484 |
| Gln-TTG-2-1 | | | |
| Homo_sapiens_tRNA- | GATTCAAAGTCCAGAGTGCT | CBE | 1485 |
| Gln-TTG-2-1 | | | |
| Homo_sapiens_tRNA- | GGATTCAAAGTCCAGAGTGC | CBE | 1486 |
| Gln-TTG-2-1 | | | |
| Homo_sapiens_tRNA- | TGGATTCAAAGTCCAGAGTG | CBE | 1487 |
| Gln-TTG-2-1 | | | |
| Homo_sapiens_tRNA- | CTGGATTCAAAGTCCAGAGT | CBE | 1488 |
| Gln-TTG-2-1 | | | |
| Homo_sapiens_tRNA- | GCTGGATTCAAAGTCCAGAG | CBE | 1489 |
| Gln-TTG-2-1 | | | |
| Homo_sapiens_tRNA- | TGCTGGATTCAAAGTCCAGA | CBE | 1490 |
| Gln-TTG-2-1 | | | |
| Homo_sapiens_tRNA- | TTGCTGGATTCAAAGTCCAG | CBE | 1491 |
| Gln-TTG-2-1 | | | |
| Homo_sapiens_tRNA- | ATTGCTGGATTCAAAGTCCA | CBE | 1492 |
| Gln-TTG-2-1 | | | |
| Homo_sapiens_tRNA- | GATTGCTGGATTCAAAGTCC | CBE | 1493 |
| Gln-TTG-2-1 | | | |
| Homo_sapiens_tRNA- | GGATTGCTGGATTCAAAGTC | CBE | 1494 |
| Gln-TTG-2-1 | | | |
| Homo_sapiens_tRNA- | CGGATTGCTGGATTCAAAGT | CBE | 1495 |
| Gln-TTG-2-1 | | | |
| Homo_sapiens_tRNA- | TCGGATTGCTGGATTCAAAG | CBE | 1496 |
| Gln-TTG-2-1 | | | |
| Homo_sapiens_tRNA- | AAGTCCAGAGTGCTAACCAT | CBE | 1497 |
| Gln-TTG-3-1 | | | |
| Homo_sapiens_tRNA- | AAAGTCCAGAGTGCTAACCA | CBE | 1498 |
| Gln-TTG-3-1 | | | |
| Homo_sapiens_tRNA- | CAAAGTCCAGAGTGCTAACC | CBE | 1499 |
| Gln-TTG-3-1 | | | |
| Homo_sapiens_tRNA- | TCAAAGTCCAGAGTGCTAAC | CBE | 1500 |
| Gln-TTG-3-1 | | | |
| Homo_sapiens_tRNA- | TTCAAAGTCCAGAGTGCTAA | CBE | 1501 |
| Gln-TTG-3-1 | | | |
| Homo_sapiens_tRNA- | ATTCAAAGTCCAGAGTGCTA | CBE | 1502 |
| Gln-TTG-3-1 | | | |
| Homo_sapiens_tRNA- | GATTCAAAGTCCAGAGTGCT | CBE | 1503 |
| Gln-TTG-3-1 | | | |
| Homo_sapiens_tRNA- | GGATTCAAAGTCCAGAGTGC | CBE | 1504 |
| Gln-TTG-3-1 | | | |
| Homo_sapiens_tRNA- | TGGATTCAAAGTCCAGAGTG | CBE | 1505 |
| Gln-TTG-3-1 | | | |
| Homo_sapiens_tRNA- | CTGGATTCAAAGTCCAGAGT | CBE | 1506 |
| Gln-TTG-3-1 | | | |
| Homo_sapiens_tRNA- | GCTGGATTCAAAGTCCAGAG | CBE | 1507 |
| Gln-TTG-3-1 | | | |
| Homo_sapiens_tRNA- | CGCTGGATTCAAAGTCCAGA | CBE | 1508 |
| Gln-TTG-3-1 | | | |
| Homo_sapiens_tRNA- | TCGCTGGATTCAAAGTCCAG | CBE | 1509 |
| Gln-TTG-3-1 | | | |
| Homo_sapiens_tRNA- | ATCGCTGGATTCAAAGTCCA | CBE | 1510 |
| Gln-TTG-3-1 | | | |
| Homo_sapiens_tRNA- | GATCGCTGGATTCAAAGTCC | CBE | 1511 |
| Gln-TTG-3-1 | | | |
| Homo_sapiens_tRNA- | GGATCGCTGGATTCAAAGTC | CBE | 1512 |
| Gln-TTG-3-1 | | | |
| Homo_sapiens_tRNA- | CGGATCGCTGGATTCAAAGT | CBE | 1513 |
| Gln-TTG-3-1 | | | |
| Homo_sapiens_tRNA- | TCGGATCGCTGGATTCAAAG | CBE | 1514 |
| Gln-TTG-3-1 | | | |
| Homo_sapiens_tRNA- | AAGTCCAGAGTGCTAACCAT | CBE | 1515 |
| Gln-TTG-3-2 | | | |
| Homo_sapiens_tRNA- | AAAGTCCAGAGTGCTAACCA | CBE | 1516 |
| Gln-TTG-3-2 | | | |
| Homo_sapiens_tRNA- | CAAAGTCCAGAGTGCTAACC | CBE | 1517 |
| Gln-TTG-3-2 | | | |
| Homo_sapiens_tRNA- | TCAAAGTCCAGAGTGCTAAC | CBE | 1518 |
| Gln-TTG-3-2 | | | |
| Homo_sapiens_tRNA- | TTCAAAGTCCAGAGTGCTAA | CBE | 1519 |
| Gln-TTG-3-2 | | | |
| Homo_sapiens_tRNA- | ATTCAAAGTCCAGAGTGCTA | CBE | 1520 |
| Gln-TTG-3-2 | | | |
| Homo_sapiens_tRNA- | GATTCAAAGTCCAGAGTGCT | CBE | 1521 |
| Gln-TTG-3-2 | | | |
| Homo_sapiens_tRNA- | GGATTCAAAGTCCAGAGTGC | CBE | 1522 |
| Gln-TTG-3-2 | | | |
| Homo_sapiens_tRNA- | TGGATTCAAAGTCCAGAGTG | CBE | 1523 |
| Gln-TTG-3-2 | | | |
| Homo_sapiens_tRNA- | CTGGATTCAAAGTCCAGAGT | CBE | 1524 |
| Gln-TTG-3-2 | | | |
| Homo_sapiens_tRNA- | GCTGGATTCAAAGTCCAGAG | CBE | 1525 |
| Gln-TTG-3-2 | | | |
| Homo_sapiens_tRNA- | CGCTGGATTCAAAGTCCAGA | CBE | 1526 |
| Gln-TTG-3-2 | | | |
| Homo_sapiens_tRNA- | TCGCTGGATTCAAAGTCCAG | CBE | 1527 |
| Gln-TTG-3-2 | | | |
| Homo_sapiens_tRNA- | ATCGCTGGATTCAAAGTCCA | CBE | 1528 |
| Gln-TTG-3-2 | | | |
| Homo_sapiens_tRNA- | GATCGCTGGATTCAAAGTCC | CBE | 1529 |
| Gln-TTG-3-2 | | | |
| Homo_sapiens_tRNA- | GGATCGCTGGATTCAAAGTC | CBE | 1530 |
| Gln-TTG-3-2 | | | |
| Homo_sapiens_tRNA- | CGGATCGCTGGATTCAAAGT | CBE | 1531 |
| Gln-TTG-3-2 | | | |
| Homo_sapiens_tRNA- | TCGGATCGCTGGATTCAAAG | CBE | 1532 |
| Gln-TTG-3-2 | | | |
| Homo_sapiens_tRNA- | AAGTCCAGAGTGCTAACCAT | CBE | 1533 |
| Gln-TTG-3-3 | | | |
| Homo_sapiens_tRNA- | AAAGTCCAGAGTGCTAACCA | CBE | 1534 |
| Gln-TTG-3-3 | | | |
| Homo_sapiens_tRNA- | CAAAGTCCAGAGTGCTAACC | CBE | 1535 |
| Gln-TTG-3-3 | | | |
| Homo_sapiens_tRNA- | TCAAAGTCCAGAGTGCTAAC | CBE | 1536 |
| Gln-TTG-3-3 | | | |
| Homo_sapiens_tRNA- | TTCAAAGTCCAGAGTGCTAA | CBE | 1537 |
| Gln-TTG-3-3 | | | |
| Homo_sapiens_tRNA- | ATTCAAAGTCCAGAGTGCTA | CBE | 1538 |
| Gln-TTG-3-3 | | | |
| Homo_sapiens_tRNA- | GATTCAAAGTCCAGAGTGCT | CBE | 1539 |
| Gln-TTG-3-3 | | | |
| Homo_sapiens_tRNA- | GGATTCAAAGTCCAGAGTGC | CBE | 1540 |
| Gln-TTG-3-3 | | | |
| Homo_sapiens_tRNA- | TGGATTCAAAGTCCAGAGTG | CBE | 1541 |
| Gln-TTG-3-3 | | | |
| Homo_sapiens_tRNA- | CTGGATTCAAAGTCCAGAGT | CBE | 1542 |
| Gln-TTG-3-3 | | | |
| Homo_sapiens_tRNA- | GCTGGATTCAAAGTCCAGAG | CBE | 1543 |
| Gln-TTG-3-3 | | | |
| Homo_sapiens_tRNA- | CGCTGGATTCAAAGTCCAGA | CBE | 1544 |
| Gln-TTG-3-3 | | | |
| Homo_sapiens_tRNA- | TCGCTGGATTCAAAGTCCAG | CBE | 1545 |
| Gln-TTG-3-3 | | | |
| Homo_sapiens_tRNA- | ATCGCTGGATTCAAAGTCCA | CBE | 1546 |
| Gln-TTG-3-3 | | | |
| Homo_sapiens_tRNA- | GATCGCTGGATTCAAAGTCC | CBE | 1547 |
| Gln-TTG-3-3 | | | |
| Homo_sapiens_tRNA- | GGATCGCTGGATTCAAAGTC | CBE | 1548 |
| Gln-TTG-3-3 | | | |
| Homo_sapiens_tRNA- | CGGATCGCTGGATTCAAAGT | CBE | 1549 |
| Gln-TTG-3-3 | | | |
| Homo_sapiens_tRNA- | TCGGATCGCTGGATTCAAAG | CBE | 1550 |
| Gln-TTG-3-3 | | | |
| Homo_sapiens_tRNA- | AAGCCCAGAGTGCTAACCAT | CBE | 1551 |
| Gln-TTG-4-1 | | | |
| Homo_sapiens_tRNA- | AAAGCCCAGAGTGCTAACCA | CBE | 1552 |
| Gln-TTG-4-1 | | | |
| Homo_sapiens_tRNA- | CAAAGCCCAGAGTGCTAACC | CBE | 1553 |
| Gln-TTG-4-1 | | | |
| Homo_sapiens_tRNA- | TCAAAGCCCAGAGTGCTAAC | CBE | 1554 |
| Gln-TTG-4-1 | | | |
| Homo_sapiens_tRNA- | TTCAAAGCCCAGAGTGCTAA | CBE | 1555 |
| Gln-TTG-4-1 | | | |
| Homo_sapiens_tRNA- | ATTCAAAGCCCAGAGTGCTA | CBE | 1556 |
| Gln-TTG-4-1 | | | |
| Homo_sapiens_tRNA- | GATTCAAAGCCCAGAGTGCT | CBE | 1557 |
| Gln-TTG-4-1 | | | |
| Homo_sapiens_tRNA- | GGATTCAAAGCCCAGAGTGC | CBE | 1558 |
| Gln-TTG-4-1 | | | |
| Homo_sapiens_tRNA- | TGGATTCAAAGCCCAGAGTG | CBE | 1559 |
| Gln-TTG-4-1 | | | |
| Homo_sapiens_tRNA- | CTGGATTCAAAGCCCAGAGT | CBE | 1560 |
| Gln-TTG-4-1 | | | |
| Homo_sapiens_tRNA- | GCTGGATTCAAAGCCCAGAG | CBE | 1561 |
| Gln-TTG-4-1 | | | |
| Homo_sapiens_tRNA- | TGCTGGATTCAAAGCCCAGA | CBE | 1562 |
| Gln-TTG-4-1 | | | |
| Homo_sapiens_tRNA- | TTGCTGGATTCAAAGCCCAG | CBE | 1563 |
| Gln-TTG-4-1 | | | |
| Homo_sapiens_tRNA- | ATTGCTGGATTCAAAGCCCA | CBE | 1564 |
| Gln-TTG-4-1 | | | |
| Homo_sapiens_tRNA- | GATTGCTGGATTCAAAGCCC | CBE | 1565 |
| Gln-TTG-4-1 | | | |
| Homo_sapiens_tRNA- | GGATTGCTGGATTCAAAGCC | CBE | 1566 |
| Gln-TTG-4-1 | | | |
| Homo_sapiens_tRNA- | CGGATTGCTGGATTCAAAGC | CBE | 1567 |
| Gln-TTG-4-1 | | | |
| Homo_sapiens_tRNA- | TCGGATTGCTGGATTCAAAG | CBE | 1568 |
| Gln-TTG-4-1 | | | |
| Homo_sapiens_tRNA- | GTGGTTAGGATTCGGCGCTC | CABE | 1569 |
| Glu-CTC-1-1 | | | |
| Homo_sapiens_tRNA- | TGGTTAGGATTCGGCGCTCT | CABE | 1570 |
| Glu-CTC-1-1 | | | |
| Homo_sapiens_tRNA- | GGTTAGGATTCGGCGCTCTC | CABE | 1571 |
| Glu-CTC-1-1 | | | |
| Homo_sapiens_tRNA- | GTTAGGATTCGGCGCTCTCA | CABE | 1572 |
| Glu-CTC-1-1 | | | |
| Homo_sapiens_tRNA- | TTAGGATTCGGCGCTCTCAC | CABE | 1573 |
| Glu-CTC-1-1 | | | |
| Homo_sapiens_tRNA- | TAGGATTCGGCGCTCTCACC | CABE | 1574 |
| Glu-CTC-1-1 | | | |
| Homo_sapiens_tRNA- | AGGATTCGGCGCTCTCACCG | CABE | 1575 |
| Glu-CTC-1-1 | | | |
| Homo_sapiens_tRNA- | GGATTCGGCGCTCTCACCGC | CABE | 1576 |
| Glu-CTC-1-1 | | | |
| Homo_sapiens_tRNA- | GATTCGGCGCTCTCACCGCC | CABE | 1577 |
| Glu-CTC-1-1 | | | |
| Homo_sapiens_tRNA- | ATTCGGCGCTCTCACCGCCG | CABE | 1578 |
| Glu-CTC-1-1 | | | |
| Homo_sapiens_tRNA- | TTCGGCGCTCTCACCGCCGC | CABE | 1579 |
| Glu-CTC-1-1 | | | |
| Homo_sapiens_tRNA- | TCGGCGCTCTCACCGCCGCG | CABE | 1580 |
| Glu-CTC-1-1 | | | |
| Homo_sapiens_tRNA- | CGGCGCTCTCACCGCCGCGG | CABE | 1581 |
| Glu-CTC-1-1 | | | |
| Homo_sapiens_tRNA- | GGCGCTCTCACCGCCGCGGC | CABE | 1582 |
| Glu-CTC-1-1 | | | |
| Homo_sapiens_tRNA- | GCGCTCTCACCGCCGCGGCC | CABE | 1583 |
| Glu-CTC-1-1 | | | |
| Homo_sapiens_tRNA- | CGCTCTCACCGCCGCGGCCC | CABE | 1584 |
| Glu-CTC-1-1 | | | |
| Homo_sapiens_tRNA- | GCTCTCACCGCCGCGGCCCG | CABE | 1585 |
| Glu-CTC-1-1 | | | |
| Homo_sapiens_tRNA- | CTCTCACCGCCGCGGCCCGG | CABE | 1586 |
| Glu-CTC-1-1 | | | |
| Homo_sapiens_tRNA- | GTGGTTAGGATTCGGCGCTC | CABE | 1587 |
| Glu-CTC-1-2 | | | |
| Homo_sapiens_tRNA- | TGGTTAGGATTCGGCGCTCT | CABE | 1588 |
| Glu-CTC-1-2 | | | |
| Homo_sapiens_tRNA- | GGTTAGGATTCGGCGCTCTC | CABE | 1589 |
| Glu-CTC-1-2 | | | |
| Homo_sapiens_tRNA- | GTTAGGATTCGGCGCTCTCA | CABE | 1590 |
| Glu-CTC-1-2 | | | |
| Homo_sapiens_tRNA- | TTAGGATTCGGCGCTCTCAC | CABE | 1591 |
| Glu-CTC-1-2 | | | |
| Homo_sapiens_tRNA- | TAGGATTCGGCGCTCTCACC | CABE | 1592 |
| Glu-CTC-1-2 | | | |
| Homo_sapiens_tRNA- | AGGATTCGGCGCTCTCACCG | CABE | 1593 |
| Glu-CTC-1-2 | | | |
| Homo_sapiens_tRNA- | GGATTCGGCGCTCTCACCGC | CABE | 1594 |
| Glu-CTC-1-2 | | | |
| Homo_sapiens_tRNA- | GATTCGGCGCTCTCACCGCC | CABE | 1595 |
| Glu-CTC-1-2 | | | |
| Homo_sapiens_tRNA- | ATTCGGCGCTCTCACCGCCG | CABE | 1596 |
| Glu-CTC-1-2 | | | |
| Homo_sapiens_tRNA- | TTCGGCGCTCTCACCGCCGC | CABE | 1597 |
| Glu-CTC-1-2 | | | |
| Homo_sapiens_tRNA- | TCGGCGCTCTCACCGCCGCG | CABE | 1598 |
| Glu-CTC-1-2 | | | |
| Homo_sapiens_tRNA- | CGGCGCTCTCACCGCCGCGG | CABE | 1599 |
| Glu-CTC-1-2 | | | |
| Homo_sapiens_tRNA- | GGCGCTCTCACCGCCGCGGC | CABE | 1600 |
| Glu-CTC-1-2 | | | |
| Homo_sapiens_tRNA- | GCGCTCTCACCGCCGCGGCC | CABE | 1601 |
| Glu-CTC-1-2 | | | |
| Homo_sapiens_tRNA- | CGCTCTCACCGCCGCGGCCC | CABE | 1602 |
| Glu-CTC-1-2 | | | |
| Homo_sapiens_tRNA- | GCTCTCACCGCCGCGGCCCG | CABE | 1603 |
| Glu-CTC-1-2 | | | |
| Homo_sapiens_tRNA- | CTCTCACCGCCGCGGCCCGG | CABE | 1604 |
| Glu-CTC-1-2 | | | |
| Homo_sapiens_tRNA- | GTGGTTAGGATTCGGCGCTC | CABE | 1605 |
| Glu-CTC-1-3 | | | |
| Homo_sapiens_tRNA- | TGGTTAGGATTCGGCGCTCT | CABE | 1606 |
| Glu-CTC-1-3 | | | |
| Homo_sapiens_tRNA- | GGTTAGGATTCGGCGCTCTC | CABE | 1607 |
| Glu-CTC-1-3 | | | |
| Homo_sapiens_tRNA- | GTTAGGATTCGGCGCTCTCA | CABE | 1608 |
| Glu-CTC-1-3 | | | |
| Homo_sapiens_tRNA- | TTAGGATTCGGCGCTCTCAC | CABE | 1609 |
| Glu-CTC-1-3 | | | |
| Homo_sapiens_tRNA- | TAGGATTCGGCGCTCTCACC | CABE | 1610 |
| Glu-CTC-1-3 | | | |
| Homo_sapiens_tRNA- | AGGATTCGGCGCTCTCACCG | CABE | 1611 |
| Glu-CTC-1-3 | | | |
| Homo_sapiens_tRNA- | GGATTCGGCGCTCTCACCGC | CABE | 1612 |
| Glu-CTC-1-3 | | | |
| Homo_sapiens_tRNA- | GATTCGGCGCTCTCACCGCC | CABE | 1613 |
| Glu-CTC-1-3 | | | |
| Homo_sapiens_tRNA- | ATTCGGCGCTCTCACCGCCG | CABE | 1614 |
| Glu-CTC-1-3 | | | |
| Homo_sapiens_tRNA- | TTCGGCGCTCTCACCGCCGC | CABE | 1615 |
| Glu-CTC-1-3 | | | |
| Homo_sapiens_tRNA- | TCGGCGCTCTCACCGCCGCG | CABE | 1616 |
| Glu-CTC-1-3 | | | |
| Homo_sapiens_tRNA- | CGGCGCTCTCACCGCCGCGG | CABE | 1617 |
| Glu-CTC-1-3 | | | |
| Homo_sapiens_tRNA- | GGCGCTCTCACCGCCGCGGC | CABE | 1618 |
| Glu-CTC-1-3 | | | |
| Homo_sapiens_tRNA- | GCGCTCTCACCGCCGCGGCC | CABE | 1619 |
| Glu-CTC-1-3 | | | |
| Homo_sapiens_tRNA- | CGCTCTCACCGCCGCGGCCC | CABE | 1620 |
| Glu-CTC-1-3 | | | |
| Homo_sapiens_tRNA- | GCTCTCACCGCCGCGGCCCG | CABE | 1621 |
| Glu-CTC-1-3 | | | |
| Homo_sapiens_tRNA- | CTCTCACCGCCGCGGCCCGG | CABE | 1622 |
| Glu-CTC-1-3 | | | |
| Homo_sapiens_tRNA- | GTGGTTAGGATTCGGCGCTC | CABE | 1623 |
| Glu-CTC-1-4 | | | |
| Homo_sapiens_tRNA- | TGGTTAGGATTCGGCGCTCT | CABE | 1624 |
| Glu-CTC-1-4 | | | |
| Homo_sapiens_tRNA- | GGTTAGGATTCGGCGCTCTC | CABE | 1625 |
| Glu-CTC-1-4 | | | |
| Homo_sapiens_tRNA- | GTTAGGATTCGGCGCTCTCA | CABE | 1626 |
| Glu-CTC-1-4 | | | |
| Homo_sapiens_tRNA- | TTAGGATTCGGCGCTCTCAC | CABE | 1627 |
| Glu-CTC-1-4 | | | |
| Homo_sapiens_tRNA- | TAGGATTCGGCGCTCTCACC | CABE | 1628 |
| Glu-CTC-1-4 | | | |
| Homo_sapiens_tRNA- | AGGATTCGGCGCTCTCACCG | CABE | 1629 |
| Glu-CTC-1-4 | | | |
| Homo_sapiens_tRNA- | GGATTCGGCGCTCTCACCGC | CABE | 1630 |
| Glu-CTC-1-4 | | | |
| Homo_sapiens_tRNA- | GATTCGGCGCTCTCACCGCC | CABE | 1631 |
| Glu-CTC-1-4 | | | |
| Homo_sapiens_tRNA- | ATTCGGCGCTCTCACCGCCG | CABE | 1632 |
| Glu-CTC-1-4 | | | |
| Homo_sapiens_tRNA- | TTCGGCGCTCTCACCGCCGC | CABE | 1633 |
| Glu-CTC-1-4 | | | |
| Homo_sapiens_tRNA- | TCGGCGCTCTCACCGCCGCG | CABE | 1634 |
| Glu-CTC-1-4 | | | |
| Homo_sapiens_tRNA- | CGGCGCTCTCACCGCCGCGG | CABE | 1635 |
| Glu-CTC-1-4 | | | |
| Homo_sapiens_tRNA- | GGCGCTCTCACCGCCGCGGC | CABE | 1636 |
| Glu-CTC-1-4 | | | |
| Homo_sapiens_tRNA- | GCGCTCTCACCGCCGCGGCC | CABE | 1637 |
| Glu-CTC-1-4 | | | |
| Homo_sapiens_tRNA- | CGCTCTCACCGCCGCGGCCC | CABE | 1638 |
| Glu-CTC-1-4 | | | |
| Homo_sapiens_tRNA- | GCTCTCACCGCCGCGGCCCG | CABE | 1639 |
| Glu-CTC-1-4 | | | |
| Homo_sapiens_tRNA- | CTCTCACCGCCGCGGCCCGG | CABE | 1640 |
| Glu-CTC-1-4 | | | |
| Homo_sapiens_tRNA- | GTGGTTAGGATTCGGCGCTC | CABE | 1641 |
| Glu-CTC-1-5 | | | |
| Homo_sapiens_tRNA- | TGGTTAGGATTCGGCGCTCT | CABE | 1642 |
| Glu-CTC-1-5 | | | |
| Homo_sapiens_tRNA- | GGTTAGGATTCGGCGCTCTC | CABE | 1643 |
| Glu-CTC-1-5 | | | |
| Homo_sapiens_tRNA- | GTTAGGATTCGGCGCTCTCA | CABE | 1644 |
| Glu-CTC-1-5 | | | |
| Homo_sapiens_tRNA- | TTAGGATTCGGCGCTCTCAC | CABE | 1645 |
| Glu-CTC-1-5 | | | |
| Homo_sapiens_tRNA- | TAGGATTCGGCGCTCTCACC | CABE | 1646 |
| Glu-CTC-1-5 | | | |
| Homo_sapiens_tRNA- | AGGATTCGGCGCTCTCACCG | CABE | 1647 |
| Glu-CTC-1-5 | | | |
| Homo_sapiens_tRNA- | GGATTCGGCGCTCTCACCGC | CABE | 1648 |
| Glu-CTC-1-5 | | | |
| Homo_sapiens_tRNA- | GATTCGGCGCTCTCACCGCC | CABE | 1649 |
| Glu-CTC-1-5 | | | |
| Homo_sapiens_tRNA- | ATTCGGCGCTCTCACCGCCG | CABE | 1650 |
| Glu-CTC-1-5 | | | |
| Homo_sapiens_tRNA- | TTCGGCGCTCTCACCGCCGC | CABE | 1651 |
| Glu-CTC-1-5 | | | |
| Homo_sapiens_tRNA- | TCGGCGCTCTCACCGCCGCG | CABE | 1652 |
| Glu-CTC-1-5 | | | |
| Homo_sapiens_tRNA- | CGGCGCTCTCACCGCCGCGG | CABE | 1653 |
| Glu-CTC-1-5 | | | |
| Homo_sapiens_tRNA- | GGCGCTCTCACCGCCGCGGC | CABE | 1654 |
| Glu-CTC-1-5 | | | |
| Homo_sapiens_tRNA- | GCGCTCTCACCGCCGCGGCC | CABE | 1655 |
| Glu-CTC-1-5 | | | |
| Homo_sapiens_tRNA- | CGCTCTCACCGCCGCGGCCC | CABE | 1656 |
| Glu-CTC-1-5 | | | |
| Homo_sapiens_tRNA- | GCTCTCACCGCCGCGGCCCG | CABE | 1657 |
| Glu-CTC-1-5 | | | |
| Homo_sapiens_tRNA- | CTCTCACCGCCGCGGCCCGG | CABE | 1658 |
| Glu-CTC-1-5 | | | |
| Homo_sapiens_tRNA- | GTGGTTAGGATTCGGCGCTC | CABE | 1659 |
| Glu-CTC-1-6 | | | |
| Homo_sapiens_tRNA- | TGGTTAGGATTCGGCGCTCT | CABE | 1660 |
| Glu-CTC-1-6 | | | |
| Homo_sapiens_tRNA- | GGTTAGGATTCGGCGCTCTC | CABE | 1661 |
| Glu-CTC-1-6 | | | |
| Homo_sapiens_tRNA- | GTTAGGATTCGGCGCTCTCA | CABE | 1662 |
| Glu-CTC-1-6 | | | |
| Homo_sapiens_tRNA- | TTAGGATTCGGCGCTCTCAC | CABE | 1663 |
| Glu-CTC-1-6 | | | |
| Homo_sapiens_tRNA- | TAGGATTCGGCGCTCTCACC | CABE | 1664 |
| Glu-CTC-1-6 | | | |
| Homo_sapiens_tRNA- | AGGATTCGGCGCTCTCACCG | CABE | 1665 |
| Glu-CTC-1-6 | | | |
| Homo_sapiens_tRNA- | GGATTCGGCGCTCTCACCGC | CABE | 1666 |
| Glu-CTC-1-6 | | | |
| Homo_sapiens_tRNA- | GATTCGGCGCTCTCACCGCC | CABE | 1667 |
| Glu-CTC-1-6 | | | |
| Homo_sapiens_tRNA- | ATTCGGCGCTCTCACCGCCG | CABE | 1668 |
| Glu-CTC-1-6 | | | |
| Homo_sapiens_tRNA- | TTCGGCGCTCTCACCGCCGC | CABE | 1669 |
| Glu-CTC-1-6 | | | |
| Homo_sapiens_tRNA- | TCGGCGCTCTCACCGCCGCG | CABE | 1670 |
| Glu-CTC-1-6 | | | |
| Homo_sapiens_tRNA- | CGGCGCTCTCACCGCCGCGG | CABE | 1671 |
| Glu-CTC-1-6 | | | |
| Homo_sapiens_tRNA- | GGCGCTCTCACCGCCGCGGC | CABE | 1672 |
| Glu-CTC-1-6 | | | |
| Homo_sapiens_tRNA- | GCGCTCTCACCGCCGCGGCC | CABE | 1673 |
| Glu-CTC-1-6 | | | |
| Homo_sapiens_tRNA- | CGCTCTCACCGCCGCGGCCC | CABE | 1674 |
| Glu-CTC-1-6 | | | |
| Homo_sapiens_tRNA- | GCTCTCACCGCCGCGGCCCG | CABE | 1675 |
| Glu-CTC-1-6 | | | |
| Homo_sapiens_tRNA- | CTCTCACCGCCGCGGCCCGG | CABE | 1676 |
| Glu-CTC-1-6 | | | |
| Homo_sapiens_tRNA- | GTGGTTAGGATTCGGCGCTC | CABE | 1677 |
| Glu-CTC-1-7 | | | |
| Homo_sapiens_tRNA- | TGGTTAGGATTCGGCGCTCT | CABE | 1678 |
| Glu-CTC-1-7 | | | |
| Homo_sapiens_tRNA- | GGTTAGGATTCGGCGCTCTC | CABE | 1679 |
| Glu-CTC-1-7 | | | |
| Homo_sapiens_tRNA- | GTTAGGATTCGGCGCTCTCA | CABE | 1680 |
| Glu-CTC-1-7 | | | |
| Homo_sapiens_tRNA- | TTAGGATTCGGCGCTCTCAC | CABE | 1681 |
| Glu-CTC-1-7 | | | |
| Homo_sapiens_tRNA- | TAGGATTCGGCGCTCTCACC | CABE | 1682 |
| Glu-CTC-1-7 | | | |
| Homo_sapiens_tRNA- | AGGATTCGGCGCTCTCACCG | CABE | 1683 |
| Glu-CTC-1-7 | | | |
| Homo_sapiens_tRNA- | GGATTCGGCGCTCTCACCGC | CABE | 1684 |
| Glu-CTC-1-7 | | | |
| Homo_sapiens_tRNA- | GATTCGGCGCTCTCACCGCC | CABE | 1685 |
| Glu-CTC-1-7 | | | |
| Homo_sapiens_tRNA- | ATTCGGCGCTCTCACCGCCG | CABE | 1686 |
| Glu-CTC-1-7 | | | |
| Homo_sapiens_tRNA- | TTCGGCGCTCTCACCGCCGC | CABE | 1687 |
| Glu-CTC-1-7 | | | |
| Homo_sapiens_tRNA- | TCGGCGCTCTCACCGCCGCG | CABE | 1688 |
| Glu-CTC-1-7 | | | |
| Homo_sapiens_tRNA- | CGGCGCTCTCACCGCCGCGG | CABE | 1689 |
| Glu-CTC-1-7 | | | |
| Homo_sapiens_tRNA- | GGCGCTCTCACCGCCGCGGC | CABE | 1690 |
| Glu-CTC-1-7 | | | |
| Homo_sapiens_tRNA- | GCGCTCTCACCGCCGCGGCC | CABE | 1691 |
| Glu-CTC-1-7 | | | |
| Homo_sapiens_tRNA- | CGCTCTCACCGCCGCGGCCC | CABE | 1692 |
| Glu-CTC-1-7 | | | |
| Homo_sapiens_tRNA- | GCTCTCACCGCCGCGGCCCG | CABE | 1693 |
| Glu-CTC-1-7 | | | |
| Homo_sapiens_tRNA- | CTCTCACCGCCGCGGCCCGG | CABE | 1694 |
| Glu-CTC-1-7 | | | |
| Homo_sapiens_tRNA- | GTGGTTAGGATTCGGCGCTC | CABE | 1695 |
| Glu-CTC-2-1 | | | |
| Homo_sapiens_tRNA- | TGGTTAGGATTCGGCGCTCT | CABE | 1696 |
| Glu-CTC-2-1 | | | |
| Homo_sapiens_tRNA- | GGTTAGGATTCGGCGCTCTC | CABE | 1697 |
| Glu-CTC-2-1 | | | |
| Homo_sapiens_tRNA- | GTTAGGATTCGGCGCTCTCA | CABE | 1698 |
| Glu-CTC-2-1 | | | |
| Homo_sapiens_tRNA- | TTAGGATTCGGCGCTCTCAC | CABE | 1699 |
| Glu-CTC-2-1 | | | |
| Homo_sapiens_tRNA- | TAGGATTCGGCGCTCTCACC | CABE | 1700 |
| Glu-CTC-2-1 | | | |
| Homo_sapiens_tRNA- | AGGATTCGGCGCTCTCACCG | CABE | 1701 |
| Glu-CTC-2-1 | | | |
| Homo_sapiens_tRNA- | GGATTCGGCGCTCTCACCGC | CABE | 1702 |
| Glu-CTC-2-1 | | | |
| Homo_sapiens_tRNA- | GATTCGGCGCTCTCACCGCC | CABE | 1703 |
| Glu-CTC-2-1 | | | |
| Homo_sapiens_tRNA- | ATTCGGCGCTCTCACCGCCG | CABE | 1704 |
| Glu-CTC-2-1 | | | |
| Homo_sapiens_tRNA- | TTCGGCGCTCTCACCGCCGC | CABE | 1705 |
| Glu-CTC-2-1 | | | |
| Homo_sapiens_tRNA- | TCGGCGCTCTCACCGCCGCG | CABE | 1706 |
| Glu-CTC-2-1 | | | |
| Homo_sapiens_tRNA- | CGGCGCTCTCACCGCCGCGG | CABE | 1707 |
| Glu-CTC-2-1 | | | |
| Homo_sapiens_tRNA- | GGCGCTCTCACCGCCGCGGC | CABE | 1708 |
| Glu-CTC-2-1 | | | |
| Homo_sapiens_tRNA- | GCGCTCTCACCGCCGCGGCC | CABE | 1709 |
| Glu-CTC-2-1 | | | |
| Homo_sapiens_tRNA- | CGCTCTCACCGCCGCGGCCC | CABE | 1710 |
| Glu-CTC-2-1 | | | |
| Homo_sapiens_tRNA- | GCTCTCACCGCCGCGGCCCG | CABE | 1711 |
| Glu-CTC-2-1 | | | |
| Homo_sapiens_tRNA- | CTCTCACCGCCGCGGCCCGG | CABE | 1712 |
| Glu-CTC-2-1 | | | |
| Homo_sapiens_tRNA- | GCGGTTAGGATTCCTGGTTT | CABE | 1713 |
| Glu-TTC-1-1 | | | |
| Homo_sapiens_tRNA- | CGGTTAGGATTCCTGGTTTT | CABE | 1714 |
| Glu-TTC-1-1 | | | |
| Homo_sapiens_tRNA- | GGTTAGGATTCCTGGTTTTC | CABE | 1715 |
| Glu-TTC-1-1 | | | |
| Homo_sapiens_tRNA- | GTTAGGATTCCTGGTTTTCA | CABE | 1716 |
| Glu-TTC-1-1 | | | |
| Homo_sapiens_tRNA- | TTAGGATTCCTGGTTTTCAC | CABE | 1717 |
| Glu-TTC-1-1 | | | |
| Homo_sapiens_tRNA- | TAGGATTCCTGGTTTTCACC | CABE | 1718 |
| Glu-TTC-1-1 | | | |
| Homo_sapiens_tRNA- | AGGATTCCTGGTTTTCACCC | CABE | 1719 |
| Glu-TTC-1-1 | | | |
| Homo_sapiens_tRNA- | GGATTCCTGGTTTTCACCCA | CABE | 1720 |
| Glu-TTC-1-1 | | | |
| Homo_sapiens_tRNA- | GATTCCTGGTTTTCACCCAG | CABE | 1721 |
| Glu-TTC-1-1 | | | |
| Homo_sapiens_tRNA- | ATTCCTGGTTTTCACCCAGG | CABE | 1722 |
| Glu-TTC-1-1 | | | |
| Homo_sapiens_tRNA- | TTCCTGGTTTTCACCCAGGT | CABE | 1723 |
| Glu-TTC-1-1 | | | |
| Homo_sapiens_tRNA- | TCCTGGTTTTCACCCAGGTG | CABE | 1724 |
| Glu-TTC-1-1 | | | |
| Homo_sapiens_tRNA- | CCTGGTTTTCACCCAGGTGG | CABE | 1725 |
| Glu-TTC-1-1 | | | |
| Homo_sapiens_tRNA- | CTGGTTTTCACCCAGGTGGC | CABE | 1726 |
| Glu-TTC-1-1 | | | |
| Homo_sapiens_tRNA- | TGGTTTTCACCCAGGTGGCC | CABE | 1727 |
| Glu-TTC-1-1 | | | |
| Homo_sapiens_tRNA- | GGTTTTCACCCAGGTGGCCC | CABE | 1728 |
| Glu-TTC-1-1 | | | |
| Homo_sapiens_tRNA- | GTTTTCACCCAGGTGGCCCG | CABE | 1729 |
| Glu-TTC-1-1 | | | |
| Homo_sapiens_tRNA- | TTTTCACCCAGGTGGCCCGG | CABE | 1730 |
| Glu-TTC-1-1 | | | |
| Homo_sapiens_tRNA- | GCGGTTAGGATTCCTGGTTT | CABE | 1731 |
| Glu-TTC-1-2 | | | |
| Homo_sapiens_tRNA- | CGGTTAGGATTCCTGGTTTT | CABE | 1732 |
| Glu-TTC-1-2 | | | |
| Homo_sapiens_tRNA- | GGTTAGGATTCCTGGTTTTC | CABE | 1733 |
| Glu-TTC-1-2 | | | |
| Homo_sapiens_tRNA- | GTTAGGATTCCTGGTTTTCA | CABE | 1734 |
| Glu-TTC-1-2 | | | |
| Homo_sapiens_tRNA- | TTAGGATTCCTGGTTTTCAC | CABE | 1735 |
| Glu-TTC-1-2 | | | |
| Homo_sapiens_tRNA- | TAGGATTCCTGGTTTTCACC | CABE | 1736 |
| Glu-TTC-1-2 | | | |
| Homo_sapiens_tRNA- | AGGATTCCTGGTTTTCACCC | CABE | 1737 |
| Glu-TTC-1-2 | | | |
| Homo_sapiens_tRNA- | GGATTCCTGGTTTTCACCCA | CABE | 1738 |
| Glu-TTC-1-2 | | | |
| Homo_sapiens_tRNA- | GATTCCTGGTTTTCACCCAG | CABE | 1739 |
| Glu-TTC-1-2 | | | |
| Homo_sapiens_tRNA- | ATTCCTGGTTTTCACCCAGG | CABE | 1740 |
| Glu-TTC-1-2 | | | |
| Homo_sapiens_tRNA- | TTCCTGGTTTTCACCCAGGT | CABE | 1741 |
| Glu-TTC-1-2 | | | |
| Homo_sapiens_tRNA- | TCCTGGTTTTCACCCAGGTG | CABE | 1742 |
| Glu-TTC-1-2 | | | |
| Homo_sapiens_tRNA- | CCTGGTTTTCACCCAGGTGG | CABE | 1743 |
| Glu-TTC-1-2 | | | |
| Homo_sapiens_tRNA- | CTGGTTTTCACCCAGGTGGC | CABE | 1744 |
| Glu-TTC-1-2 | | | |
| Homo_sapiens_tRNA- | TGGTTTTCACCCAGGTGGCC | CABE | 1745 |
| Glu-TTC-1-2 | | | |
| Homo_sapiens_tRNA- | GGTTTTCACCCAGGTGGCCC | CABE | 1746 |
| Glu-TTC-1-2 | | | |
| Homo_sapiens_tRNA- | GTTTTCACCCAGGTGGCCCG | CABE | 1747 |
| Glu-TTC-1-2 | | | |
| Homo_sapiens_tRNA- | TTTTCACCCAGGTGGCCCGG | CABE | 1748 |
| Glu-TTC-1-2 | | | |
| Homo_sapiens_tRNA- | GCGGTTAGGATTCCTGGTTT | CABE | 1749 |
| Glu-TTC-2-1 | | | |
| Homo_sapiens_tRNA- | CGGTTAGGATTCCTGGTTTT | CABE | 1750 |
| Glu-TTC-2-1 | | | |
| Homo_sapiens_tRNA- | GGTTAGGATTCCTGGTTTTC | CABE | 1751 |
| Glu-TTC-2-1 | | | |
| Homo_sapiens_tRNA- | GTTAGGATTCCTGGTTTTCA | CABE | 1752 |
| Glu-TTC-2-1 | | | |
| Homo_sapiens_tRNA- | TTAGGATTCCTGGTTTTCAC | CABE | 1753 |
| Glu-TTC-2-1 | | | |
| Homo_sapiens_tRNA- | TAGGATTCCTGGTTTTCACC | CABE | 1754 |
| Glu-TTC-2-1 | | | |
| Homo_sapiens_tRNA- | AGGATTCCTGGTTTTCACCC | CABE | 1755 |
| Glu-TTC-2-1 | | | |
| Homo_sapiens_tRNA- | GGATTCCTGGTTTTCACCCA | CABE | 1756 |
| Glu-TTC-2-1 | | | |
| Homo_sapiens_tRNA- | GATTCCTGGTTTTCACCCAG | CABE | 1757 |
| Glu-TTC-2-1 | | | |
| Homo_sapiens_tRNA- | ATTCCTGGTTTTCACCCAGG | CABE | 1758 |
| Glu-TTC-2-1 | | | |
| Homo_sapiens_tRNA- | TTCCTGGTTTTCACCCAGGC | CABE | 1759 |
| Glu-TTC-2-1 | | | |
| Homo_sapiens_tRNA- | TCCTGGTTTTCACCCAGGCG | CABE | 1760 |
| Glu-TTC-2-1 | | | |
| Homo_sapiens_tRNA- | CCTGGTTTTCACCCAGGCGG | CABE | 1761 |
| Glu-TTC-2-1 | | | |
| Homo_sapiens_tRNA- | CTGGTTTTCACCCAGGCGGC | CABE | 1762 |
| Glu-TTC-2-1 | | | |
| Homo_sapiens_tRNA- | TGGTTTTCACCCAGGCGGCC | CABE | 1763 |
| Glu-TTC-2-1 | | | |
| Homo_sapiens_tRNA- | GGTTTTCACCCAGGCGGCCC | CABE | 1764 |
| Glu-TTC-2-1 | | | |
| Homo_sapiens_tRNA- | GTTTTCACCCAGGCGGCCCG | CABE | 1765 |
| Glu-TTC-2-1 | | | |
| Homo_sapiens_tRNA- | TTTTCACCCAGGCGGCCCGG | CABE | 1766 |
| Glu-TTC-2-1 | | | |
| Homo_sapiens_tRNA- | GCGGTTAGGATTCCTGGTTT | CABE | 1767 |
| Glu-TTC-2-2 | | | |
| Homo_sapiens_tRNA- | CGGTTAGGATTCCTGGTTTT | CABE | 1768 |
| Glu-TTC-2-2 | | | |
| Homo_sapiens_tRNA- | GGTTAGGATTCCTGGTTTTC | CABE | 1769 |
| Glu-TTC-2-2 | | | |
| Homo_sapiens_tRNA- | GTTAGGATTCCTGGTTTTCA | CABE | 1770 |
| Glu-TTC-2-2 | | | |
| Homo_sapiens_tRNA- | TTAGGATTCCTGGTTTTCAC | CABE | 1771 |
| Glu-TTC-2-2 | | | |
| Homo_sapiens_tRNA- | TAGGATTCCTGGTTTTCACC | CABE | 1772 |
| Glu-TTC-2-2 | | | |
| Homo_sapiens_tRNA- | AGGATTCCTGGTTTTCACCC | CABE | 1773 |
| Glu-TTC-2-2 | | | |
| Homo_sapiens_tRNA- | GGATTCCTGGTTTTCACCCA | CABE | 1774 |
| Glu-TTC-2-2 | | | |
| Homo_sapiens_tRNA- | GATTCCTGGTTTTCACCCAG | CABE | 1775 |
| Glu-TTC-2-2 | | | |
| Homo_sapiens_tRNA- | ATTCCTGGTTTTCACCCAGG | CABE | 1776 |
| Glu-TTC-2-2 | | | |
| Homo_sapiens_tRNA- | TTCCTGGTTTTCACCCAGGC | CABE | 1777 |
| Glu-TTC-2-2 | | | |
| Homo_sapiens_tRNA- | TCCTGGTTTTCACCCAGGCG | CABE | 1778 |
| Glu-TTC-2-2 | | | |
| Homo_sapiens_tRNA- | CCTGGTTTTCACCCAGGCGG | CABE | 1779 |
| Glu-TTC-2-2 | | | |
| Homo_sapiens_tRNA- | CTGGTTTTCACCCAGGCGGC | CABE | 1780 |
| Glu-TTC-2-2 | | | |
| Homo_sapiens_tRNA- | TGGTTTTCACCCAGGCGGCC | CABE | 1781 |
| Glu-TTC-2-2 | | | |
| Homo_sapiens_tRNA- | GGTTTTCACCCAGGCGGCCC | CABE | 1782 |
| Glu-TTC-2-2 | | | |
| Homo_sapiens_tRNA- | GTTTTCACCCAGGCGGCCCG | CABE | 1783 |
| Glu-TTC-2-2 | | | |
| Homo_sapiens_tRNA- | TTTTCACCCAGGCGGCCCGG | CABE | 1784 |
| Glu-TTC-2-2 | | | |
| Homo_sapiens_tRNA- | GTGGCTAGGATTCGGCGCTT | CABE | 1785 |
| Glu-TTC-3-1 | | | |
| Homo_sapiens_tRNA- | TGGCTAGGATTCGGCGCTTT | CABE | 1786 |
| Glu-TTC-3-1 | | | |
| Homo_sapiens_tRNA- | GGCTAGGATTCGGCGCTTTC | CABE | 1787 |
| Glu-TTC-3-1 | | | |
| Homo_sapiens_tRNA- | GCTAGGATTCGGCGCTTTCA | CABE | 1788 |
| Glu-TTC-3-1 | | | |
| Homo_sapiens_tRNA- | CTAGGATTCGGCGCTTTCAC | CABE | 1789 |
| Glu-TTC-3-1 | | | |
| Homo_sapiens_tRNA- | TAGGATTCGGCGCTTTCACC | CABE | 1790 |
| Glu-TTC-3-1 | | | |
| Homo_sapiens_tRNA- | AGGATTCGGCGCTTTCACCG | CABE | 1791 |
| Glu-TTC-3-1 | | | |
| Homo_sapiens_tRNA- | GGATTCGGCGCTTTCACCGC | CABE | 1792 |
| Glu-TTC-3-1 | | | |
| Homo_sapiens_tRNA- | GATTCGGCGCTTTCACCGCC | CABE | 1793 |
| Glu-TTC-3-1 | | | |
| Homo_sapiens_tRNA- | ATTCGGCGCTTTCACCGCCG | CABE | 1794 |
| Glu-TTC-3-1 | | | |
| Homo_sapiens_tRNA- | TTCGGCGCTTTCACCGCCGC | CABE | 1795 |
| Glu-TTC-3-1 | | | |
| Homo_sapiens_tRNA- | TCGGCGCTTTCACCGCCGCG | CABE | 1796 |
| Glu-TTC-3-1 | | | |
| Homo_sapiens_tRNA- | CGGCGCTTTCACCGCCGCGG | CABE | 1797 |
| Glu-TTC-3-1 | | | |
| Homo_sapiens_tRNA- | GGCGCTTTCACCGCCGCGGC | CABE | 1798 |
| Glu-TTC-3-1 | | | |
| Homo_sapiens_tRNA- | GCGCTTTCACCGCCGCGGCC | CABE | 1799 |
| Glu-TTC-3-1 | | | |
| Homo_sapiens_tRNA- | CGCTTTCACCGCCGCGGCCC | CABE | 1800 |
| Glu-TTC-3-1 | | | |
| Homo_sapiens_tRNA- | GCTTTCACCGCCGCGGCCCG | CABE | 1801 |
| Glu-TTC-3-1 | | | |
| Homo_sapiens_tRNA- | CTTTCACCGCCGCGGCCCGG | CABE | 1802 |
| Glu-TTC-3-1 | | | |
| Homo_sapiens_tRNA- | GTGGCTAGGATTCGGCGCTT | CABE | 1803 |
| Glu-TTC-4-1 | | | |
| Homo_sapiens_tRNA- | TGGCTAGGATTCGGCGCTTT | CABE | 1804 |
| Glu-TTC-4-1 | | | |
| Homo_sapiens_tRNA- | GGCTAGGATTCGGCGCTTTC | CABE | 1805 |
| Glu-TTC-4-1 | | | |
| Homo_sapiens_tRNA- | GCTAGGATTCGGCGCTTTCA | CABE | 1806 |
| Glu-TTC-4-1 | | | |
| Homo_sapiens_tRNA- | CTAGGATTCGGCGCTTTCAC | CABE | 1807 |
| Glu-TTC-4-1 | | | |
| Homo_sapiens_tRNA- | TAGGATTCGGCGCTTTCACC | CABE | 1808 |
| Glu-TTC-4-1 | | | |
| Homo_sapiens_tRNA- | AGGATTCGGCGCTTTCACCG | CABE | 1809 |
| Glu-TTC-4-1 | | | |
| Homo_sapiens_tRNA- | GGATTCGGCGCTTTCACCGC | CABE | 1810 |
| Glu-TTC-4-1 | | | |
| Homo_sapiens_tRNA- | GATTCGGCGCTTTCACCGCC | CABE | 1811 |
| Glu-TTC-4-1 | | | |
| Homo_sapiens_tRNA- | ATTCGGCGCTTTCACCGCCG | CABE | 1812 |
| Glu-TTC-4-1 | | | |
| Homo_sapiens_tRNA- | TTCGGCGCTTTCACCGCCGC | CABE | 1813 |
| Glu-TTC-4-1 | | | |
| Homo_sapiens_tRNA- | TCGGCGCTTTCACCGCCGCG | CABE | 1814 |
| Glu-TTC-4-1 | | | |
| Homo_sapiens_tRNA- | CGGCGCTTTCACCGCCGCGG | CABE | 1815 |
| Glu-TTC-4-1 | | | |
| Homo_sapiens_tRNA- | GGCGCTTTCACCGCCGCGGC | CABE | 1816 |
| Glu-TTC-4-1 | | | |
| Homo_sapiens_tRNA- | GCGCTTTCACCGCCGCGGCC | CABE | 1817 |
| Glu-TTC-4-1 | | | |
| Homo_sapiens_tRNA- | CGCTTTCACCGCCGCGGCCC | CABE | 1818 |
| Glu-TTC-4-1 | | | |
| Homo_sapiens_tRNA- | GCTTTCACCGCCGCGGCCCG | CABE | 1819 |
| Glu-TTC-4-1 | | | |
| Homo_sapiens_tRNA- | CTTTCACCGCCGCGGCCCGG | CABE | 1820 |
| Glu-TTC-4-1 | | | |
| Homo_sapiens_tRNA- | GTGGCTAGGATTCGGCGCTT | CABE | 1821 |
| Glu-TTC-4-2 | | | |
| Homo_sapiens_tRNA- | TGGCTAGGATTCGGCGCTTT | CABE | 1822 |
| Glu-TTC-4-2 | | | |
| Homo_sapiens_tRNA- | GGCTAGGATTCGGCGCTTTC | CABE | 1823 |
| Glu-TTC-4-2 | | | |
| Homo_sapiens_tRNA- | GCTAGGATTCGGCGCTTTCA | CABE | 1824 |
| Glu-TTC-4-2 | | | |
| Homo_sapiens_tRNA- | CTAGGATTCGGCGCTTTCAC | CABE | 1825 |
| Glu-TTC-4-2 | | | |
| Homo_sapiens_tRNA- | TAGGATTCGGCGCTTTCACC | CABE | 1826 |
| Glu-TTC-4-2 | | | |
| Homo_sapiens_tRNA- | AGGATTCGGCGCTTTCACCG | CABE | 1827 |
| Glu-TTC-4-2 | | | |
| Homo_sapiens_tRNA- | GGATTCGGCGCTTTCACCGC | CABE | 1828 |
| Glu-TTC-4-2 | | | |
| Homo_sapiens_tRNA- | GATTCGGCGCTTTCACCGCC | CABE | 1829 |
| Glu-TTC-4-2 | | | |
| Homo_sapiens_tRNA- | ATTCGGCGCTTTCACCGCCG | CABE | 1830 |
| Glu-TTC-4-2 | | | |
| Homo_sapiens_tRNA- | TTCGGCGCTTTCACCGCCGC | CABE | 1831 |
| Glu-TTC-4-2 | | | |
| Homo_sapiens_tRNA- | TCGGCGCTTTCACCGCCGCG | CABE | 1832 |
| Glu-TTC-4-2 | | | |
| Homo_sapiens_tRNA- | CGGCGCTTTCACCGCCGCGG | CABE | 1833 |
| Glu-TTC-4-2 | | | |
| Homo_sapiens_tRNA- | GGCGCTTTCACCGCCGCGGC | CABE | 1834 |
| Glu-TTC-4-2 | | | |
| Homo_sapiens_tRNA- | GCGCTTTCACCGCCGCGGCC | CABE | 1835 |
| Glu-TTC-4-2 | | | |
| Homo_sapiens_tRNA- | CGCTTTCACCGCCGCGGCCC | CABE | 1836 |
| Glu-TTC-4-2 | | | |
| Homo_sapiens_tRNA- | GCTTTCACCGCCGCGGCCCG | CABE | 1837 |
| Glu-TTC-4-2 | | | |
| Homo_sapiens_tRNA- | CTTTCACCGCCGCGGCCCGG | CABE | 1838 |
| Glu-TTC-4-2 | | | |
| Homo_sapiens_tRNA- | GTGGTTAGCATAGCTGCCTT | CABE | 1839 |
| Gly-TCC-1-1 | | | |
| Homo_sapiens_tRNA- | TGGTTAGCATAGCTGCCTTC | CABE | 1840 |
| Gly-TCC-1-1 | | | |
| Homo_sapiens_tRNA- | GGTTAGCATAGCTGCCTTCC | CABE | 1841 |
| Gly-TCC-1-1 | | | |
| Homo_sapiens_tRNA- | GTTAGCATAGCTGCCTTCCA | CABE | 1842 |
| Gly-TCC-1-1 | | | |
| Homo_sapiens_tRNA- | TTAGCATAGCTGCCTTCCAA | CABE | 1843 |
| Gly-TCC-1-1 | | | |
| Homo_sapiens_tRNA- | TAGCATAGCTGCCTTCCAAG | CABE | 1844 |
| Gly-TCC-1-1 | | | |
| Homo_sapiens_tRNA- | AGCATAGCTGCCTTCCAAGC | CABE | 1845 |
| Gly-TCC-1-1 | | | |
| Homo_sapiens_tRNA- | GCATAGCTGCCTTCCAAGCA | CABE | 1846 |
| Gly-TCC-1-1 | | | |
| Homo_sapiens_tRNA- | CATAGCTGCCTTCCAAGCAG | CABE | 1847 |
| Gly-TCC-1-1 | | | |
| Homo_sapiens_tRNA- | ATAGCTGCCTTCCAAGCAGT | CABE | 1848 |
| Gly-TCC-1-1 | | | |
| Homo_sapiens_tRNA- | TAGCTGCCTTCCAAGCAGTT | CABE | 1849 |
| Gly-TCC-1-1 | | | |
| Homo_sapiens_tRNA- | AGCTGCCTTCCAAGCAGTTG | CABE | 1850 |
| Gly-TCC-1-1 | | | |
| Homo_sapiens_tRNA- | GCTGCCTTCCAAGCAGTTGA | CABE | 1851 |
| Gly-TCC-1-1 | | | |
| Homo_sapiens_tRNA- | CTGCCTTCCAAGCAGTTGAC | CABE | 1852 |
| Gly-TCC-1-1 | | | |
| Homo_sapiens_tRNA- | TGCCTTCCAAGCAGTTGACC | CABE | 1853 |
| Gly-TCC-1-1 | | | |
| Homo_sapiens_tRNA- | GCCTTCCAAGCAGTTGACCC | CABE | 1854 |
| Gly-TCC-1-1 | | | |
| Homo_sapiens_tRNA- | CCTTCCAAGCAGTTGACCCG | CABE | 1855 |
| Gly-TCC-1-1 | | | |
| Homo_sapiens_tRNA- | CTTCCAAGCAGTTGACCCGG | CABE | 1856 |
| Gly-TCC-1-1 | | | |
| Homo_sapiens_tRNA- | GTGGTGAGCATAGCTGCCTT | CABE | 1857 |
| Gly-TCC-2-1 | | | |
| Homo_sapiens_tRNA- | TGGTGAGCATAGCTGCCTTC | CABE | 1858 |
| Gly-TCC-2-1 | | | |
| Homo_sapiens_tRNA- | GGTGAGCATAGCTGCCTTCC | CABE | 1859 |
| Gly-TCC-2-1 | | | |
| Homo_sapiens_tRNA- | GTGAGCATAGCTGCCTTCCA | CABE | 1860 |
| Gly-TCC-2-1 | | | |
| Homo_sapiens_tRNA- | TGAGCATAGCTGCCTTCCAA | CABE | 1861 |
| Gly-TCC-2-1 | | | |
| Homo_sapiens_tRNA- | GAGCATAGCTGCCTTCCAAG | CABE | 1862 |
| Gly-TCC-2-1 | | | |
| Homo_sapiens_tRNA- | AGCATAGCTGCCTTCCAAGC | CABE | 1863 |
| Gly-TCC-2-1 | | | |
| Homo_sapiens_tRNA- | GCATAGCTGCCTTCCAAGCA | CABE | 1864 |
| Gly-TCC-2-1 | | | |
| Homo_sapiens_tRNA- | CATAGCTGCCTTCCAAGCAG | CABE | 1865 |
| Gly-TCC-2-1 | | | |
| Homo_sapiens_tRNA- | ATAGCTGCCTTCCAAGCAGT | CABE | 1866 |
| Gly-TCC-2-1 | | | |
| Homo_sapiens_tRNA- | TAGCTGCCTTCCAAGCAGTT | CABE | 1867 |
| Gly-TCC-2-1 | | | |
| Homo_sapiens_tRNA- | AGCTGCCTTCCAAGCAGTTG | CABE | 1868 |
| Gly-TCC-2-1 | | | |
| Homo_sapiens_tRNA- | GCTGCCTTCCAAGCAGTTGA | CABE | 1869 |
| Gly-TCC-2-1 | | | |
| Homo_sapiens_tRNA- | CTGCCTTCCAAGCAGTTGAC | CABE | 1870 |
| Gly-TCC-2-1 | | | |
| Homo_sapiens_tRNA- | TGCCTTCCAAGCAGTTGACC | CABE | 1871 |
| Gly-TCC-2-1 | | | |
| Homo_sapiens_tRNA- | GCCTTCCAAGCAGTTGACCC | CABE | 1872 |
| Gly-TCC-2-1 | | | |
| Homo_sapiens_tRNA- | CCTTCCAAGCAGTTGACCCG | CABE | 1873 |
| Gly-TCC-2-1 | | | |
| Homo_sapiens_tRNA- | CTTCCAAGCAGTTGACCCGG | CABE | 1874 |
| Gly-TCC-2-1 | | | |
| Homo_sapiens_tRNA- | GTGGTGAGCATAGCTGCCTT | CABE | 1875 |
| Gly-TCC-2-2 | | | |
| Homo_sapiens_tRNA- | TGGTGAGCATAGCTGCCTTC | CABE | 1876 |
| Gly-TCC-2-2 | | | |
| Homo_sapiens_tRNA- | GGTGAGCATAGCTGCCTTCC | CABE | 1877 |
| Gly-TCC-2-2 | | | |
| Homo_sapiens_tRNA- | GTGAGCATAGCTGCCTTCCA | CABE | 1878 |
| Gly-TCC-2-2 | | | |
| Homo_sapiens_tRNA- | TGAGCATAGCTGCCTTCCAA | CABE | 1879 |
| Gly-TCC-2-2 | | | |
| Homo_sapiens_tRNA- | GAGCATAGCTGCCTTCCAAG | CABE | 1880 |
| Gly-TCC-2-2 | | | |
| Homo_sapiens_tRNA- | AGCATAGCTGCCTTCCAAGC | CABE | 1881 |
| Gly-TCC-2-2 | | | |
| Homo_sapiens_tRNA- | GCATAGCTGCCTTCCAAGCA | CABE | 1882 |
| Gly-TCC-2-2 | | | |
| Homo_sapiens_tRNA- | CATAGCTGCCTTCCAAGCAG | CABE | 1883 |
| Gly-TCC-2-2 | | | |
| Homo_sapiens_tRNA- | ATAGCTGCCTTCCAAGCAGT | CABE | 1884 |
| Gly-TCC-2-2 | | | |
| Homo_sapiens_tRNA- | TAGCTGCCTTCCAAGCAGTT | CABE | 1885 |
| Gly-TCC-2-2 | | | |
| Homo_sapiens_tRNA- | AGCTGCCTTCCAAGCAGTTG | CABE | 1886 |
| Gly-TCC-2-2 | | | |
| Homo_sapiens_tRNA- | GCTGCCTTCCAAGCAGTTGA | CABE | 1887 |
| Gly-TCC-2-2 | | | |
| Homo_sapiens_tRNA- | CTGCCTTCCAAGCAGTTGAC | CABE | 1888 |
| Gly-TCC-2-2 | | | |
| Homo_sapiens_tRNA- | TGCCTTCCAAGCAGTTGACC | CABE | 1889 |
| Gly-TCC-2-2 | | | |
| Homo_sapiens_tRNA- | GCCTTCCAAGCAGTTGACCC | CABE | 1890 |
| Gly-TCC-2-2 | | | |
| Homo_sapiens_tRNA- | CCTTCCAAGCAGTTGACCCG | CABE | 1891 |
| Gly-TCC-2-2 | | | |
| Homo_sapiens_tRNA- | CTTCCAAGCAGTTGACCCGG | CABE | 1892 |
| Gly-TCC-2-2 | | | |
| Homo_sapiens_tRNA- | GTGGTGAGCATAGCTGCCTT | CABE | 1893 |
| Gly-TCC-2-3 | | | |
| Homo_sapiens_tRNA- | TGGTGAGCATAGCTGCCTTC | CABE | 1894 |
| Gly-TCC-2-3 | | | |
| Homo_sapiens_tRNA- | GGTGAGCATAGCTGCCTTCC | CABE | 1895 |
| Gly-TCC-2-3 | | | |
| Homo_sapiens_tRNA- | GTGAGCATAGCTGCCTTCCA | CABE | 1896 |
| Gly-TCC-2-3 | | | |
| Homo_sapiens_tRNA- | TGAGCATAGCTGCCTTCCAA | CABE | 1897 |
| Gly-TCC-2-3 | | | |
| Homo_sapiens_tRNA- | GAGCATAGCTGCCTTCCAAG | CABE | 1898 |
| Gly-TCC-2-3 | | | |
| Homo_sapiens_tRNA- | AGCATAGCTGCCTTCCAAGC | CABE | 1899 |
| Gly-TCC-2-3 | | | |
| Homo_sapiens_tRNA- | GCATAGCTGCCTTCCAAGCA | CABE | 1900 |
| Gly-TCC-2-3 | | | |
| Homo_sapiens_tRNA- | CATAGCTGCCTTCCAAGCAG | CABE | 1901 |
| Gly-TCC-2-3 | | | |
| Homo_sapiens_tRNA- | ATAGCTGCCTTCCAAGCAGT | CABE | 1902 |
| Gly-TCC-2-3 | | | |
| Homo_sapiens_tRNA- | TAGCTGCCTTCCAAGCAGTT | CABE | 1903 |
| Gly-TCC-2-3 | | | |
| Homo_sapiens_tRNA- | AGCTGCCTTCCAAGCAGTTG | CABE | 1904 |
| Gly-TCC-2-3 | | | |
| Homo_sapiens_tRNA- | GCTGCCTTCCAAGCAGTTGA | CABE | 1905 |
| Gly-TCC-2-3 | | | |
| Homo_sapiens_tRNA- | CTGCCTTCCAAGCAGTTGAC | CABE | 1906 |
| Gly-TCC-2-3 | | | |
| Homo_sapiens_tRNA- | TGCCTTCCAAGCAGTTGACC | CABE | 1907 |
| Gly-TCC-2-3 | | | |
| Homo_sapiens_tRNA- | GCCTTCCAAGCAGTTGACCC | CABE | 1908 |
| Gly-TCC-2-3 | | | |
| Homo_sapiens_tRNA- | CCTTCCAAGCAGTTGACCCG | CABE | 1909 |
| Gly-TCC-2-3 | | | |
| Homo_sapiens_tRNA- | CTTCCAAGCAGTTGACCCGG | CABE | 1910 |
| Gly-TCC-2-3 | | | |
| Homo_sapiens_tRNA- | GTGGTGAGCATAGCTGCCTT | CABE | 1911 |
| Gly-TCC-2-4 | | | |
| Homo_sapiens_tRNA- | TGGTGAGCATAGCTGCCTTC | CABE | 1912 |
| Gly-TCC-2-4 | | | |
| Homo_sapiens_tRNA- | GGTGAGCATAGCTGCCTTCC | CABE | 1913 |
| Gly-TCC-2-4 | | | |
| Homo_sapiens_tRNA- | GTGAGCATAGCTGCCTTCCA | CABE | 1914 |
| Gly-TCC-2-4 | | | |
| Homo_sapiens_tRNA- | TGAGCATAGCTGCCTTCCAA | CABE | 1915 |
| Gly-TCC-2-4 | | | |
| Homo_sapiens_tRNA- | GAGCATAGCTGCCTTCCAAG | CABE | 1916 |
| Gly-TCC-2-4 | | | |
| Homo_sapiens_tRNA- | AGCATAGCTGCCTTCCAAGC | CABE | 1917 |
| Gly-TCC-2-4 | | | |
| Homo_sapiens_tRNA- | GCATAGCTGCCTTCCAAGCA | CABE | 1918 |
| Gly-TCC-2-4 | | | |
| Homo_sapiens_tRNA- | CATAGCTGCCTTCCAAGCAG | CABE | 1919 |
| Gly-TCC-2-4 | | | |
| Homo_sapiens_tRNA- | ATAGCTGCCTTCCAAGCAGT | CABE | 1920 |
| Gly-TCC-2-4 | | | |
| Homo_sapiens_tRNA- | TAGCTGCCTTCCAAGCAGTT | CABE | 1921 |
| Gly-TCC-2-4 | | | |
| Homo_sapiens_tRNA- | AGCTGCCTTCCAAGCAGTTG | CABE | 1922 |
| Gly-TCC-2-4 | | | |
| Homo_sapiens_tRNA- | GCTGCCTTCCAAGCAGTTGA | CABE | 1923 |
| Gly-TCC-2-4 | | | |
| Homo_sapiens_tRNA- | CTGCCTTCCAAGCAGTTGAC | CABE | 1924 |
| Gly-TCC-2-4 | | | |
| Homo_sapiens_tRNA- | TGCCTTCCAAGCAGTTGACC | CABE | 1925 |
| Gly-TCC-2-4 | | | |
| Homo_sapiens_tRNA- | GCCTTCCAAGCAGTTGACCC | CABE | 1926 |
| Gly-TCC-2-4 | | | |
| Homo_sapiens_tRNA- | CCTTCCAAGCAGTTGACCCG | CABE | 1927 |
| Gly-TCC-2-4 | | | |
| Homo_sapiens_tRNA- | CTTCCAAGCAGTTGACCCGG | CABE | 1928 |
| Gly-TCC-2-4 | | | |
| Homo_sapiens_tRNA- | GTGGTGAGCATAGCTGCCTT | CABE | 1929 |
| Gly-TCC-2-5 | | | |
| Homo_sapiens_tRNA- | TGGTGAGCATAGCTGCCTTC | CABE | 1930 |
| Gly-TCC-2-5 | | | |
| Homo_sapiens_tRNA- | GGTGAGCATAGCTGCCTTCC | CABE | 1931 |
| Gly-TCC-2-5 | | | |
| Homo_sapiens_tRNA- | GTGAGCATAGCTGCCTTCCA | CABE | 1932 |
| Gly-TCC-2-5 | | | |
| Homo_sapiens_tRNA- | TGAGCATAGCTGCCTTCCAA | CABE | 1933 |
| Gly-TCC-2-5 | | | |
| Homo_sapiens_tRNA- | GAGCATAGCTGCCTTCCAAG | CABE | 1934 |
| Gly-TCC-2-5 | | | |
| Homo_sapiens_tRNA- | AGCATAGCTGCCTTCCAAGC | CABE | 1935 |
| Gly-TCC-2-5 | | | |
| Homo_sapiens_tRNA- | GCATAGCTGCCTTCCAAGCA | CABE | 1936 |
| Gly-TCC-2-5 | | | |
| Homo_sapiens_tRNA- | CATAGCTGCCTTCCAAGCAG | CABE | 1937 |
| Gly-TCC-2-5 | | | |
| Homo_sapiens_tRNA- | ATAGCTGCCTTCCAAGCAGT | CABE | 1938 |
| Gly-TCC-2-5 | | | |
| Homo_sapiens_tRNA- | TAGCTGCCTTCCAAGCAGTT | CABE | 1939 |
| Gly-TCC-2-5 | | | |
| Homo_sapiens_tRNA- | AGCTGCCTTCCAAGCAGTTG | CABE | 1940 |
| Gly-TCC-2-5 | | | |
| Homo_sapiens_tRNA- | GCTGCCTTCCAAGCAGTTGA | CABE | 1941 |
| Gly-TCC-2-5 | | | |
| Homo_sapiens_tRNA- | CTGCCTTCCAAGCAGTTGAC | CABE | 1942 |
| Gly-TCC-2-5 | | | |
| Homo_sapiens_tRNA- | TGCCTTCCAAGCAGTTGACC | CABE | 1943 |
| Gly-TCC-2-5 | | | |
| Homo_sapiens_tRNA- | GCCTTCCAAGCAGTTGACCC | CABE | 1944 |
| Gly-TCC-2-5 | | | |
| Homo_sapiens_tRNA- | CCTTCCAAGCAGTTGACCCG | CABE | 1945 |
| Gly-TCC-2-5 | | | |
| Homo_sapiens_tRNA- | CTTCCAAGCAGTTGACCCGG | CABE | 1946 |
| Gly-TCC-2-5 | | | |
| Homo_sapiens_tRNA- | GTGGTGAGCATAGCTGCCTT | CABE | 1947 |
| Gly-TCC-2-6 | | | |
| Homo_sapiens_tRNA- | TGGTGAGCATAGCTGCCTTC | CABE | 1948 |
| Gly-TCC-2-6 | | | |
| Homo_sapiens_tRNA- | GGTGAGCATAGCTGCCTTCC | CABE | 1949 |
| Gly-TCC-2-6 | | | |
| Homo_sapiens_tRNA- | GTGAGCATAGCTGCCTTCCA | CABE | 1950 |
| Gly-TCC-2-6 | | | |
| Homo_sapiens_tRNA- | TGAGCATAGCTGCCTTCCAA | CABE | 1951 |
| Gly-TCC-2-6 | | | |
| Homo_sapiens_tRNA- | GAGCATAGCTGCCTTCCAAG | CABE | 1952 |
| Gly-TCC-2-6 | | | |
| Homo_sapiens_tRNA- | AGCATAGCTGCCTTCCAAGC | CABE | 1953 |
| Gly-TCC-2-6 | | | |
| Homo_sapiens_tRNA- | GCATAGCTGCCTTCCAAGCA | CABE | 1954 |
| Gly-TCC-2-6 | | | |
| Homo_sapiens_tRNA- | CATAGCTGCCTTCCAAGCAG | CABE | 1955 |
| Gly-TCC-2-6 | | | |
| Homo_sapiens_tRNA- | ATAGCTGCCTTCCAAGCAGT | CABE | 1956 |
| Gly-TCC-2-6 | | | |
| Homo_sapiens_tRNA- | TAGCTGCCTTCCAAGCAGTT | CABE | 1957 |
| Gly-TCC-2-6 | | | |
| Homo_sapiens_tRNA- | AGCTGCCTTCCAAGCAGTTG | CABE | 1958 |
| Gly-TCC-2-6 | | | |
| Homo_sapiens_tRNA- | GCTGCCTTCCAAGCAGTTGA | CABE | 1959 |
| Gly-TCC-2-6 | | | |
| Homo_sapiens_tRNA- | CTGCCTTCCAAGCAGTTGAC | CABE | 1960 |
| Gly-TCC-2-6 | | | |
| Homo_sapiens_tRNA- | TGCCTTCCAAGCAGTTGACC | CABE | 1961 |
| Gly-TCC-2-6 | | | |
| Homo_sapiens_tRNA- | GCCTTCCAAGCAGTTGACCC | CABE | 1962 |
| Gly-TCC-2-6 | | | |
| Homo_sapiens_tRNA- | CCTTCCAAGCAGTTGACCCG | CABE | 1963 |
| Gly-TCC-2-6 | | | |
| Homo_sapiens_tRNA- | CTTCCAAGCAGTTGACCCGG | CABE | 1964 |
| Gly-TCC-2-6 | | | |
| Homo_sapiens_tRNA- | GTGGTAAGCATAGCTGCCTT | CABE | 1965 |
| Gly-TCC-3-1 | | | |
| Homo_sapiens_tRNA- | TGGTAAGCATAGCTGCCTTC | CABE | 1966 |
| Gly-TCC-3-1 | | | |
| Homo_sapiens_tRNA- | GGTAAGCATAGCTGCCTTCC | CABE | 1967 |
| Gly-TCC-3-1 | | | |
| Homo_sapiens_tRNA- | GTAAGCATAGCTGCCTTCCA | CABE | 1968 |
| Gly-TCC-3-1 | | | |
| Homo_sapiens_tRNA- | TAAGCATAGCTGCCTTCCAA | CABE | 1969 |
| Gly-TCC-3-1 | | | |
| Homo_sapiens_tRNA- | AAGCATAGCTGCCTTCCAAG | CABE | 1970 |
| Gly-TCC-3-1 | | | |
| Homo_sapiens_tRNA- | AGCATAGCTGCCTTCCAAGC | CABE | 1971 |
| Gly-TCC-3-1 | | | |
| Homo_sapiens_tRNA- | GCATAGCTGCCTTCCAAGCA | CABE | 1972 |
| Gly-TCC-3-1 | | | |
| Homo_sapiens_tRNA- | CATAGCTGCCTTCCAAGCAG | CABE | 1973 |
| Gly-TCC-3-1 | | | |
| Homo_sapiens_tRNA- | ATAGCTGCCTTCCAAGCAGT | CABE | 1974 |
| Gly-TCC-3-1 | | | |
| Homo_sapiens_tRNA- | TAGCTGCCTTCCAAGCAGTT | CABE | 1975 |
| Gly-TCC-3-1 | | | |
| Homo_sapiens_tRNA- | AGCTGCCTTCCAAGCAGTTG | CABE | 1976 |
| Gly-TCC-3-1 | | | |
| Homo_sapiens_tRNA- | GCTGCCTTCCAAGCAGTTGA | CABE | 1977 |
| Gly-TCC-3-1 | | | |
| Homo_sapiens_tRNA- | CTGCCTTCCAAGCAGTTGAC | CABE | 1978 |
| Gly-TCC-3-1 | | | |
| Homo_sapiens_tRNA- | TGCCTTCCAAGCAGTTGACC | CABE | 1979 |
| Gly-TCC-3-1 | | | |
| Homo_sapiens_tRNA- | GCCTTCCAAGCAGTTGACCC | CABE | 1980 |
| Gly-TCC-3-1 | | | |
| Homo_sapiens_tRNA- | CCTTCCAAGCAGTTGACCCG | CABE | 1981 |
| Gly-TCC-3-1 | | | |
| Homo_sapiens_tRNA- | CTTCCAAGCAGTTGACCCGG | CABE | 1982 |
| Gly-TCC-3-1 | | | |
| Homo_sapiens_tRNA- | GTGGTGAGCATAGTTGCCTT | CABE | 1983 |
| Gly-TCC-4-1 | | | |
| Homo_sapiens_tRNA- | TGGTGAGCATAGTTGCCTTC | CABE | 1984 |
| Gly-TCC-4-1 | | | |
| Homo_sapiens_tRNA- | GGTGAGCATAGTTGCCTTCC | CABE | 1985 |
| Gly-TCC-4-1 | | | |
| Homo_sapiens_tRNA- | GTGAGCATAGTTGCCTTCCA | CABE | 1986 |
| Gly-TCC-4-1 | | | |
| Homo_sapiens_tRNA- | TGAGCATAGTTGCCTTCCAA | CABE | 1987 |
| Gly-TCC-4-1 | | | |
| Homo_sapiens_tRNA- | GAGCATAGTTGCCTTCCAAG | CABE | 1988 |
| Gly-TCC-4-1 | | | |
| Homo_sapiens_tRNA- | AGCATAGTTGCCTTCCAAGC | CABE | 1989 |
| Gly-TCC-4-1 | | | |
| Homo_sapiens_tRNA- | GCATAGTTGCCTTCCAAGCA | CABE | 1990 |
| Gly-TCC-4-1 | | | |
| Homo_sapiens_tRNA- | CATAGTTGCCTTCCAAGCAG | CABE | 1991 |
| Gly-TCC-4-1 | | | |
| Homo_sapiens_tRNA- | ATAGTTGCCTTCCAAGCAGT | CABE | 1992 |
| Gly-TCC-4-1 | | | |
| Homo_sapiens_tRNA- | TAGTTGCCTTCCAAGCAGTT | CABE | 1993 |
| Gly-TCC-4-1 | | | |
| Homo_sapiens_tRNA- | AGTTGCCTTCCAAGCAGTTG | CABE | 1994 |
| Gly-TCC-4-1 | | | |
| Homo_sapiens_tRNA- | GTTGCCTTCCAAGCAGTTGA | CABE | 1995 |
| Gly-TCC-4-1 | | | |
| Homo_sapiens_tRNA- | TTGCCTTCCAAGCAGTTGAC | CABE | 1996 |
| Gly-TCC-4-1 | | | |
| Homo_sapiens_tRNA- | TGCCTTCCAAGCAGTTGACC | CABE | 1997 |
| Gly-TCC-4-1 | | | |
| Homo_sapiens_tRNA- | GCCTTCCAAGCAGTTGACCC | CABE | 1998 |
| Gly-TCC-4-1 | | | |
| Homo_sapiens_tRNA- | CCTTCCAAGCAGTTGACCCG | CABE | 1999 |
| Gly-TCC-4-1 | | | |
| Homo_sapiens_tRNA- | CTTCCAAGCAGTTGACCCGG | CABE | 2000 |
| Gly-TCC-4-1 | | | |
| Homo_sapiens_tRNA- | GGTTAAGGCGTTGGACTTAA | ACBE | 2001 |
| Leu-TAA-1-1 | | | |
| Homo_sapiens_tRNA- | GTTAAGGCGTTGGACTTAAG | ACBE | 2002 |
| Leu-TAA-1-1 | | | |
| Homo_sapiens_tRNA- | TTAAGGCGTTGGACTTAAGA | ACBE | 2003 |
| Leu-TAA-1-1 | | | |
| Homo_sapiens_tRNA- | TAAGGCGTTGGACTTAAGAT | ACBE | 2004 |
| Leu-TAA-1-1 | | | |
| Homo_sapiens_tRNA- | AAGGCGTTGGACTTAAGATC | ACBE | 2005 |
| Leu-TAA-1-1 | | | |
| Homo_sapiens_tRNA- | AGGCGTTGGACTTAAGATCC | ACBE | 2006 |
| Leu-TAA-1-1 | | | |
| Homo_sapiens_tRNA- | GGCGTTGGACTTAAGATCCA | ACBE | 2007 |
| Leu-TAA-1-1 | | | |
| Homo_sapiens_tRNA- | GCGTTGGACTTAAGATCCAA | ACBE | 2008 |
| Leu-TAA-1-1 | | | |
| Homo_sapiens_tRNA- | CGTTGGACTTAAGATCCAAT | ACBE | 2009 |
| Leu-TAA-1-1 | | | |
| Homo_sapiens_tRNA- | GTTGGACTTAAGATCCAATG | ACBE | 2010 |
| Leu-TAA-1-1 | | | |
| Homo_sapiens_tRNA- | TTGGACTTAAGATCCAATGG | ACBE | 2011 |
| Leu-TAA-1-1 | | | |
| Homo_sapiens_tRNA- | TGGACTTAAGATCCAATGGA | ACBE | 2012 |
| Leu-TAA-1-1 | | | |
| Homo_sapiens_tRNA- | GGACTTAAGATCCAATGGAC | ACBE | 2013 |
| Leu-TAA-1-1 | | | |
| Homo_sapiens_tRNA- | GACTTAAGATCCAATGGACA | ACBE | 2014 |
| Leu-TAA-1-1 | | | |
| Homo_sapiens_tRNA- | GGTTAAGGCGTTGGACTTAA | ACBE | 2015 |
| Leu-TAA-2-1 | | | |
| Homo_sapiens_tRNA- | GTTAAGGCGTTGGACTTAAG | ACBE | 2016 |
| Leu-TAA-2-1 | | | |
| Homo_sapiens_tRNA- | TTAAGGCGTTGGACTTAAGA | ACBE | 2017 |
| Leu-TAA-2-1 | | | |
| Homo_sapiens_tRNA- | TAAGGCGTTGGACTTAAGAT | ACBE | 2018 |
| Leu-TAA-2-1 | | | |
| Homo_sapiens_tRNA- | AAGGCGTTGGACTTAAGATC | ACBE | 2019 |
| Leu-TAA-2-1 | | | |
| Homo_sapiens_tRNA- | AGGCGTTGGACTTAAGATCC | ACBE | 2020 |
| Leu-TAA-2-1 | | | |
| Homo_sapiens_tRNA- | GGCGTTGGACTTAAGATCCA | ACBE | 2021 |
| Leu-TAA-2-1 | | | |
| Homo_sapiens_tRNA- | GCGTTGGACTTAAGATCCAA | ACBE | 2022 |
| Leu-TAA-2-1 | | | |
| Homo_sapiens_tRNA- | CGTTGGACTTAAGATCCAAT | ACBE | 2023 |
| Leu-TAA-2-1 | | | |
| Homo_sapiens_tRNA- | GTTGGACTTAAGATCCAATG | ACBE | 2024 |
| Leu-TAA-2-1 | | | |
| Homo_sapiens_tRNA- | TTGGACTTAAGATCCAATGG | ACBE | 2025 |
| Leu-TAA-2-1 | | | |
| Homo_sapiens_tRNA- | TGGACTTAAGATCCAATGGG | ACBE | 2026 |
| Leu-TAA-2-1 | | | |
| Homo_sapiens_tRNA- | GGACTTAAGATCCAATGGGC | ACBE | 2027 |
| Leu-TAA-2-1 | | | |
| Homo_sapiens_tRNA- | GACTTAAGATCCAATGGGCT | ACBE | 2028 |
| Leu-TAA-2-1 | | | |
| Homo_sapiens_tRNA- | GGTTAAGGCGTTGGACTTAA | ACBE | 2029 |
| Leu-TAA-3-1 | | | |
| Homo_sapiens_tRNA- | GTTAAGGCGTTGGACTTAAG | ACBE | 2030 |
| Leu-TAA-3-1 | | | |
| Homo_sapiens_tRNA- | TTAAGGCGTTGGACTTAAGA | ACBE | 2031 |
| Leu-TAA-3-1 | | | |
| Homo_sapiens_tRNA- | TAAGGCGTTGGACTTAAGAT | ACBE | 2032 |
| Leu-TAA-3-1 | | | |
| Homo_sapiens_tRNA- | AAGGCGTTGGACTTAAGATC | ACBE | 2033 |
| Leu-TAA-3-1 | | | |
| Homo_sapiens_tRNA- | AGGCGTTGGACTTAAGATCC | ACBE | 2034 |
| Leu-TAA-3-1 | | | |
| Homo_sapiens_tRNA- | GGCGTTGGACTTAAGATCCA | ACBE | 2035 |
| Leu-TAA-3-1 | | | |
| Homo_sapiens_tRNA- | GCGTTGGACTTAAGATCCAA | ACBE | 2036 |
| Leu-TAA-3-1 | | | |
| Homo_sapiens_tRNA- | CGTTGGACTTAAGATCCAAT | ACBE | 2037 |
| Leu-TAA-3-1 | | | |
| Homo_sapiens_tRNA- | GTTGGACTTAAGATCCAATG | ACBE | 2038 |
| Leu-TAA-3-1 | | | |
| Homo_sapiens_tRNA- | TTGGACTTAAGATCCAATGG | ACBE | 2039 |
| Leu-TAA-3-1 | | | |
| Homo_sapiens_tRNA- | TGGACTTAAGATCCAATGGA | ACBE | 2040 |
| Leu-TAA-3-1 | | | |
| Homo_sapiens_tRNA- | GGACTTAAGATCCAATGGAT | ACBE | 2041 |
| Leu-TAA-3-1 | | | |
| Homo_sapiens_tRNA- | GACTTAAGATCCAATGGATT | ACBE | 2042 |
| Leu-TAA-3-1 | | | |
| Homo_sapiens_tRNA- | GGTTAAGGCGTTGGACTTAA | ACBE | 2043 |
| Leu-TAA-4-1 | | | |
| Homo_sapiens_tRNA- | GTTAAGGCGTTGGACTTAAG | ACBE | 2044 |
| Leu-TAA-4-1 | | | |
| Homo_sapiens_tRNA- | TTAAGGCGTTGGACTTAAGA | ACBE | 2045 |
| Leu-TAA-4-1 | | | |
| Homo_sapiens_tRNA- | TAAGGCGTTGGACTTAAGAT | ACBE | 2046 |
| Leu-TAA-4-1 | | | |
| Homo_sapiens_tRNA- | AAGGCGTTGGACTTAAGATC | ACBE | 2047 |
| Leu-TAA-4-1 | | | |
| Homo_sapiens_tRNA- | AGGCGTTGGACTTAAGATCC | ACBE | 2048 |
| Leu-TAA-4-1 | | | |
| Homo_sapiens_tRNA- | GGCGTTGGACTTAAGATCCA | ACBE | 2049 |
| Leu-TAA-4-1 | | | |
| Homo_sapiens_tRNA- | GCGTTGGACTTAAGATCCAA | ACBE | 2050 |
| Leu-TAA-4-1 | | | |
| Homo_sapiens_tRNA- | CGTTGGACTTAAGATCCAAT | ACBE | 2051 |
| Leu-TAA-4-1 | | | |
| Homo_sapiens_tRNA- | GTTGGACTTAAGATCCAATG | ACBE | 2052 |
| Leu-TAA-4-1 | | | |
| Homo_sapiens_tRNA- | TTGGACTTAAGATCCAATGG | ACBE | 2053 |
| Leu-TAA-4-1 | | | |
| Homo_sapiens_tRNA- | TGGACTTAAGATCCAATGGA | ACBE | 2054 |
| Leu-TAA-4-1 | | | |
| Homo_sapiens_tRNA- | GGACTTAAGATCCAATGGAC | ACBE | 2055 |
| Leu-TAA-4-1 | | | |
| Homo_sapiens_tRNA- | GACTTAAGATCCAATGGACA | ACBE | 2056 |
| Leu-TAA-4-1 | | | |
| Homo_sapiens_tRNA- | TCGAGTCCAACGCCTTAACC | CABE | 2057 |
| Ser-CGA-1-1 | | | |
| Homo_sapiens_tRNA- | TTCGAGTCCAACGCCTTAAC | CABE | 2058 |
| Ser-CGA-1-1 | | | |
| Homo_sapiens_tRNA- | TTTCGAGTCCAACGCCTTAA | CABE | 2059 |
| Ser-CGA-1-1 | | | |
| Homo_sapiens_tRNA- | ATTTCGAGTCCAACGCCTTA | CABE | 2060 |
| Ser-CGA-1-1 | | | |
| Homo_sapiens_tRNA- | GATTTCGAGTCCAACGCCTT | CABE | 2061 |
| Ser-CGA-1-1 | | | |
| Homo_sapiens_tRNA- | GGATTTCGAGTCCAACGCCT | CABE | 2062 |
| Ser-CGA-1-1 | | | |
| Homo_sapiens_tRNA- | TGGATTTCGAGTCCAACGCC | CABE | 2063 |
| Ser-CGA-1-1 | | | |
| Homo_sapiens_tRNA- | TTGGATTTCGAGTCCAACGC | CABE | 2064 |
| Ser-CGA-1-1 | | | |
| Homo_sapiens_tRNA- | ATTGGATTTCGAGTCCAACG | CABE | 2065 |
| Ser-CGA-1-1 | | | |
| Homo_sapiens_tRNA- | CATTGGATTTCGAGTCCAAC | CABE | 2066 |
| Ser-CGA-1-1 | | | |
| Homo_sapiens_tRNA- | CCATTGGATTTCGAGTCCAA | CABE | 2067 |
| Ser-CGA-1-1 | | | |
| Homo_sapiens_tRNA- | CCCATTGGATTTCGAGTCCA | CABE | 2068 |
| Ser-CGA-1-1 | | | |
| Homo_sapiens_tRNA- | CCCCATTGGATTTCGAGTCC | CABE | 2069 |
| Ser-CGA-1-1 | | | |
| Homo_sapiens_tRNA- | ACCCCATTGGATTTCGAGTC | CABE | 2070 |
| Ser-CGA-1-1 | | | |
| Homo_sapiens_tRNA- | TCGAGTCCAACGCCTTAACC | CABE | 2071 |
| Ser-CGA-2-1 | | | |
| Homo_sapiens_tRNA- | TTCGAGTCCAACGCCTTAAC | CABE | 2072 |
| Ser-CGA-2-1 | | | |
| Homo_sapiens_tRNA- | TTTCGAGTCCAACGCCTTAA | CABE | 2073 |
| Ser-CGA-2-1 | | | |
| Homo_sapiens_tRNA- | ATTTCGAGTCCAACGCCTTA | CABE | 2074 |
| Ser-CGA-2-1 | | | |
| Homo_sapiens_tRNA- | GATTTCGAGTCCAACGCCTT | CABE | 2075 |
| Ser-CGA-2-1 | | | |
| Homo_sapiens_tRNA- | GGATTTCGAGTCCAACGCCT | CABE | 2076 |
| Ser-CGA-2-1 | | | |
| Homo_sapiens_tRNA- | TGGATTTCGAGTCCAACGCC | CABE | 2077 |
| Ser-CGA-2-1 | | | |
| Homo_sapiens_tRNA- | TTGGATTTCGAGTCCAACGC | CABE | 2078 |
| Ser-CGA-2-1 | | | |
| Homo_sapiens_tRNA- | ATTGGATTTCGAGTCCAACG | CABE | 2079 |
| Ser-CGA-2-1 | | | |
| Homo_sapiens_tRNA- | CATTGGATTTCGAGTCCAAC | CABE | 2080 |
| Ser-CGA-2-1 | | | |
| Homo_sapiens_tRNA- | CCATTGGATTTCGAGTCCAA | CABE | 2081 |
| Ser-CGA-2-1 | | | |
| Homo_sapiens_tRNA- | CCCATTGGATTTCGAGTCCA | CABE | 2082 |
| Ser-CGA-2-1 | | | |
| Homo_sapiens_tRNA- | CCCCATTGGATTTCGAGTCC | CABE | 2083 |
| Ser-CGA-2-1 | | | |
| Homo_sapiens_tRNA- | ACCCCATTGGATTTCGAGTC | CABE | 2084 |
| Ser-CGA-2-1 | | | |
| Homo_sapiens_tRNA- | TCGAGTCCAACACCTTAACC | CABE | 2085 |
| Ser-CGA-3-1 | | | |
| Homo_sapiens_tRNA- | TTCGAGTCCAACACCTTAAC | CABE | 2086 |
| Ser-CGA-3-1 | | | |
| Homo_sapiens_tRNA- | TTTCGAGTCCAACACCTTAA | CABE | 2087 |
| Ser-CGA-3-1 | | | |
| Homo_sapiens_tRNA- | ATTTCGAGTCCAACACCTTA | CABE | 2088 |
| Ser-CGA-3-1 | | | |
| Homo_sapiens_tRNA- | GATTTCGAGTCCAACACCTT | CABE | 2089 |
| Ser-CGA-3-1 | | | |
| Homo_sapiens_tRNA- | GGATTTCGAGTCCAACACCT | CABE | 2090 |
| Ser-CGA-3-1 | | | |
| Homo_sapiens_tRNA- | TGGATTTCGAGTCCAACACC | CABE | 2091 |
| Ser-CGA-3-1 | | | |
| Homo_sapiens_tRNA- | TTGGATTTCGAGTCCAACAC | CABE | 2092 |
| Ser-CGA-3-1 | | | |
| Homo_sapiens_tRNA- | ATTGGATTTCGAGTCCAACA | CABE | 2093 |
| Ser-CGA-3-1 | | | |
| Homo_sapiens_tRNA- | CATTGGATTTCGAGTCCAAC | CABE | 2094 |
| Ser-CGA-3-1 | | | |
| Homo_sapiens_tRNA- | CCATTGGATTTCGAGTCCAA | CABE | 2095 |
| Ser-CGA-3-1 | | | |
| Homo_sapiens_tRNA- | CCCATTGGATTTCGAGTCCA | CABE | 2096 |
| Ser-CGA-3-1 | | | |
| Homo_sapiens_tRNA- | CCCCATTGGATTTCGAGTCC | CABE | 2097 |
| Ser-CGA-3-1 | | | |
| Homo_sapiens_tRNA- | CCCCCATTGGATTTCGAGTC | CABE | 2098 |
| Ser-CGA-3-1 | | | |
| Homo_sapiens_tRNA- | TCGAGTCCAACGCCTTAACC | CABE | 2099 |
| Ser-CGA-4-1 | | | |
| Homo_sapiens_tRNA- | TTCGAGTCCAACGCCTTAAC | CABE | 2100 |
| Ser-CGA-4-1 | | | |
| Homo_sapiens_tRNA- | TTTCGAGTCCAACGCCTTAA | CABE | 2101 |
| Ser-CGA-4-1 | | | |
| Homo_sapiens_tRNA- | ATTTCGAGTCCAACGCCTTA | CABE | 2102 |
| Ser-CGA-4-1 | | | |
| Homo_sapiens_tRNA- | GATTTCGAGTCCAACGCCTT | CABE | 2103 |
| Ser-CGA-4-1 | | | |
| Homo_sapiens_tRNA- | GGATTTCGAGTCCAACGCCT | CABE | 2104 |
| Ser-CGA-4-1 | | | |
| Homo_sapiens_tRNA- | TGGATTTCGAGTCCAACGCC | CABE | 2105 |
| Ser-CGA-4-1 | | | |
| Homo_sapiens_tRNA- | TTGGATTTCGAGTCCAACGC | CABE | 2106 |
| Ser-CGA-4-1 | | | |
| Homo_sapiens_tRNA- | ATTGGATTTCGAGTCCAACG | CABE | 2107 |
| Ser-CGA-4-1 | | | |
| Homo_sapiens_tRNA- | CATTGGATTTCGAGTCCAAC | CABE | 2108 |
| Ser-CGA-4-1 | | | |
| Homo_sapiens_tRNA- | CCATTGGATTTCGAGTCCAA | CABE | 2109 |
| Ser-CGA-4-1 | | | |
| Homo_sapiens_tRNA- | CCCATTGGATTTCGAGTCCA | CABE | 2110 |
| Ser-CGA-4-1 | | | |
| Homo_sapiens_tRNA- | CCCCATTGGATTTCGAGTCC | CABE | 2111 |
| Ser-CGA-4-1 | | | |
| Homo_sapiens_tRNA- | ACCCCATTGGATTTCGAGTC | CABE | 2112 |
| Ser-CGA-4-1 | | | |
| Homo_sapiens_tRNA- | TCAAGTCCAACGCCTTAACC | CABE or | 2113 |
| Ser-TGA-1-1 | | CGBE | |
| Homo_sapiens_tRNA- | TTCAAGTCCAACGCCTTAAC | CABE or | 2114 |
| Ser-TGA-1-1 | | CGBE | |
| Homo_sapiens_tRNA- | TTTCAAGTCCAACGCCTTAA | CABE or | 2115 |
| Ser-TGA-1-1 | | CGBE | |
| Homo_sapiens_tRNA- | ATTTCAAGTCCAACGCCTTA | CABE or | 2116 |
| Ser-TGA-1-1 | | CGBE | |
| Homo_sapiens_tRNA- | GATTTCAAGTCCAACGCCTT | CABE or | 2117 |
| Ser-TGA-1-1 | | CGBE | |
| Homo_sapiens_tRNA- | GGATTTCAAGTCCAACGCCT | CABE or | 2118 |
| Ser-TGA-1-1 | | CGBE | |
| Homo_sapiens_tRNA- | TGGATTTCAAGTCCAACGCC | CABE or | 2119 |
| Ser-TGA-1-1 | | CGBE | |
| Homo_sapiens_tRNA- | TTGGATTTCAAGTCCAACGC | CABE or | 2120 |
| Ser-TGA-1-1 | | CGBE | |
| Homo_sapiens_tRNA- | ATTGGATTTCAAGTCCAACG | CABE or | 2121 |
| Ser-TGA-1-1 | | CGBE | |
| Homo_sapiens_tRNA- | CATTGGATTTCAAGTCCAAC | CABE or | 2122 |
| Ser-TGA-1-1 | | CGBE | |
| Homo_sapiens_tRNA- | CCATTGGATTTCAAGTCCAA | CABE or | 2123 |
| Ser-TGA-1-1 | | CGBE | |
| Homo_sapiens_tRNA- | CCCATTGGATTTCAAGTCCA | CABE or | 2124 |
| Ser-TGA-1-1 | | CGBE | |
| Homo_sapiens_tRNA- | CCCCATTGGATTTCAAGTCC | CABE or | 2125 |
| Ser-TGA-1-1 | | CGBE | |
| Homo_sapiens_tRNA- | ACCCCATTGGATTTCAAGTC | CABE or | 2126 |
| Ser-TGA-1-1 | | CGBE | |
| Homo_sapiens_tRNA- | TCAAGTCCATCGCCTTAACC | CABE or | 2127 |
| Ser-TGA-2-1 | | CGBE | |
| Homo_sapiens_tRNA- | TTCAAGTCCATCGCCTTAAC | CABE or | 2128 |
| Ser-TGA-2-1 | | CGBE | |
| Homo_sapiens_tRNA- | TTTCAAGTCCATCGCCTTAA | CABE or | 2129 |
| Ser-TGA-2-1 | | CGBE | |
| Homo_sapiens_tRNA- | ATTTCAAGTCCATCGCCTTA | CABE or | 2130 |
| Ser-TGA-2-1 | | CGBE | |
| Homo_sapiens_tRNA- | GATTTCAAGTCCATCGCCTT | CABE or | 2131 |
| Ser-TGA-2-1 | | CGBE | |
| Homo_sapiens_tRNA- | GGATTTCAAGTCCATCGCCT | CABE or | 2132 |
| Ser-TGA-2-1 | | CGBE | |
| Homo_sapiens_tRNA- | TGGATTTCAAGTCCATCGCC | CABE or | 2133 |
| Ser-TGA-2-1 | | CGBE | |
| Homo_sapiens_tRNA- | ATGGATTTCAAGTCCATCGC | CABE or | 2134 |
| Ser-TGA-2-1 | | CGBE | |
| Homo_sapiens_tRNA- | AATGGATTTCAAGTCCATCG | CABE or | 2135 |
| Ser-TGA-2-1 | | CGBE | |
| Homo_sapiens_tRNA- | CAATGGATTTCAAGTCCATC | CABE or | 2136 |
| Ser-TGA-2-1 | | CGBE | |
| Homo_sapiens_tRNA- | CCAATGGATTTCAAGTCCAT | CABE or | 2137 |
| Ser-TGA-2-1 | | CGBE | |
| Homo_sapiens_tRNA- | CCCAATGGATTTCAAGTCCA | CABE or | 2138 |
| Ser-TGA-2-1 | | CGBE | |
| Homo_sapiens_tRNA- | CCCCAATGGATTTCAAGTCC | CABE or | 2139 |
| Ser-TGA-2-1 | | CGBE | |
| Homo_sapiens_tRNA- | ACCCCAATGGATTTCAAGTC | CABE or | 2140 |
| Ser-TGA-2-1 | | CGBE | |
| Homo_sapiens_tRNA- | TCAAGTCCATCGCCTTAACC | CABE or | 2141 |
| Ser-TGA-3-1 | | CGBE | |
| Homo_sapiens_tRNA- | TTCAAGTCCATCGCCTTAAC | CABE or | 2142 |
| Ser-TGA-3-1 | | CGBE | |
| Homo_sapiens_tRNA- | TTTCAAGTCCATCGCCTTAA | CABE or | 2143 |
| Ser-TGA-3-1 | | CGBE | |
| Homo_sapiens_tRNA- | ATTTCAAGTCCATCGCCTTA | CABE or | 2144 |
| Ser-TGA-3-1 | | CGBE | |
| Homo_sapiens_tRNA- | GATTTCAAGTCCATCGCCTT | CABE or | 2145 |
| Ser-TGA-3-1 | | CGBE | |
| Homo_sapiens_tRNA- | GGATTTCAAGTCCATCGCCT | CABE or | 2146 |
| Ser-TGA-3-1 | | CGBE | |
| Homo_sapiens_tRNA- | TGGATTTCAAGTCCATCGCC | CABE or | 2147 |
| Ser-TGA-3-1 | | CGBE | |
| Homo_sapiens_tRNA- | ATGGATTTCAAGTCCATCGC | CABE or | 2148 |
| Ser-TGA-3-1 | | CGBE | |
| Homo_sapiens_tRNA- | AATGGATTTCAAGTCCATCG | CABE or | 2149 |
| Ser-TGA-3-1 | | CGBE | |
| Homo_sapiens_tRNA- | CAATGGATTTCAAGTCCATC | CABE or | 2150 |
| Ser-TGA-3-1 | | CGBE | |
| Homo_sapiens_tRNA- | CCAATGGATTTCAAGTCCAT | CABE or | 2151 |
| Ser-TGA-3-1 | | CGBE | |
| Homo_sapiens_tRNA- | CCCAATGGATTTCAAGTCCA | CABE or | 2152 |
| Ser-TGA-3-1 | | CGBE | |
| Homo_sapiens_tRNA- | CCCCAATGGATTTCAAGTCC | CABE or | 2153 |
| Ser-TGA-3-1 | | CGBE | |
| Homo_sapiens_tRNA- | ACCCCAATGGATTTCAAGTC | CABE or | 2154 |
| Ser-TGA-3-1 | | CGBE | |
| Homo_sapiens_tRNA- | TCAAGTCCATCGCCTTAACC | CABE or | 2155 |
| Ser-TGA-4-1 | | CGBE | |
| Homo_sapiens_tRNA- | TTCAAGTCCATCGCCTTAAC | CABE or | 2156 |
| Ser-TGA-4-1 | | CGBE | |
| Homo_sapiens_tRNA- | TTTCAAGTCCATCGCCTTAA | CABE or | 2157 |
| Ser-TGA-4-1 | | CGBE | |
| Homo_sapiens_tRNA- | ATTTCAAGTCCATCGCCTTA | CABE or | 2158 |
| Ser-TGA-4-1 | | CGBE | |
| Homo_sapiens_tRNA- | GATTTCAAGTCCATCGCCTT | CABE or | 2159 |
| Ser-TGA-4-1 | | CGBE | |
| Homo_sapiens_tRNA- | GGATTTCAAGTCCATCGCCT | CABE or | 2160 |
| Ser-TGA-4-1 | | CGBE | |
| Homo_sapiens_tRNA- | TGGATTTCAAGTCCATCGCC | CABE or | 2161 |
| Ser-TGA-4-1 | | CGBE | |
| Homo_sapiens_tRNA- | ATGGATTTCAAGTCCATCGC | CABE or | 2162 |
| Ser-TGA-4-1 | | CGBE | |
| Homo_sapiens_tRNA- | AATGGATTTCAAGTCCATCG | CABE or | 2163 |
| Ser-TGA-4-1 | | CGBE | |
| Homo_sapiens_tRNA- | CAATGGATTTCAAGTCCATC | CABE or | 2164 |
| Ser-TGA-4-1 | | CGBE | |
| Homo_sapiens_tRNA- | CCAATGGATTTCAAGTCCAT | CABE or | 2165 |
| Ser-TGA-4-1 | | CGBE | |
| Homo_sapiens_tRNA- | CCCAATGGATTTCAAGTCCA | CABE or | 2166 |
| Ser-TGA-4-1 | | CGBE | |
| Homo_sapiens_tRNA- | CCCCAATGGATTTCAAGTCC | CABE or | 2167 |
| Ser-TGA-4-1 | | CGBE | |
| Homo_sapiens_tRNA- | ACCCCAATGGATTTCAAGTC | CABE or | 2168 |
| Ser-TGA-4-1 | | CGBE | |
| Homo_sapiens_tRNA- | ACGGTAGCGCGTCTGACTCC | CBE | 2169 |
| Trp-CCA-1-1 | | | |
| Homo_sapiens_tRNA- | CGGTAGCGCGTCTGACTCCA | CBE | 2170 |
| Trp-CCA-1-1 | | | |
| Homo_sapiens_tRNA- | GGTAGCGCGTCTGACTCCAG | CBE | 2171 |
| Trp-CCA-1-1 | | | |
| Homo_sapiens_tRNA- | GTAGCGCGTCTGACTCCAGA | CBE | 2172 |
| Trp-CCA-1-1 | | | |
| Homo_sapiens_tRNA- | TAGCGCGTCTGACTCCAGAT | CBE | 2173 |
| Trp-CCA-1-1 | | | |
| Homo_sapiens_tRNA- | AGCGCGTCTGACTCCAGATC | CBE | 2174 |
| Trp-CCA-1-1 | | | |
| Homo_sapiens_tRNA- | GCGCGTCTGACTCCAGATCA | CBE | 2175 |
| Trp-CCA-1-1 | | | |
| Homo_sapiens_tRNA- | CGCGTCTGACTCCAGATCAG | CBE | 2176 |
| Trp-CCA-1-1 | | | |
| Homo_sapiens_tRNA- | GCGTCTGACTCCAGATCAGA | CBE | 2177 |
| Trp-CCA-1-1 | | | |
| Homo_sapiens_tRNA- | CGTCTGACTCCAGATCAGAA | CBE | 2178 |
| Trp-CCA-1-1 | | | |
| Homo_sapiens_tRNA- | GTCTGACTCCAGATCAGAAG | CBE | 2179 |
| Trp-CCA-1-1 | | | |
| Homo_sapiens_tRNA- | TCTGACTCCAGATCAGAAGG | CBE | 2180 |
| Trp-CCA-1-1 | | | |
| Homo_sapiens_tRNA- | CTGACTCCAGATCAGAAGGT | CBE | 2181 |
| Trp-CCA-1-1 | | | |
| Homo_sapiens_tRNA- | TGACTCCAGATCAGAAGGTT | CBE | 2182 |
| Trp-CCA-1-1 | | | |
| Homo_sapiens_tRNA- | GACTCCAGATCAGAAGGTTG | CBE | 2183 |
| Trp-CCA-1-1 | | | |
| Homo_sapiens_tRNA- | ACTCCAGATCAGAAGGTTGC | CBE | 2184 |
| Trp-CCA-1-1 | | | |
| Homo_sapiens_tRNA- | CTCCAGATCAGAAGGTTGCG | CBE | 2185 |
| Trp-CCA-1-1 | | | |
| Homo_sapiens_tRNA- | TCCAGATCAGAAGGTTGCGT | CBE | 2186 |
| Trp-CCA-1-1 | | | |
| Homo_sapiens_tRNA- | ATGGTAGCGCGTCTGACTCC | CBE | 2187 |
| Trp-CCA-2-1 | | | |
| Homo_sapiens_tRNA- | TGGTAGCGCGTCTGACTCCA | CBE | 2188 |
| Trp-CCA-2-1 | | | |
| Homo_sapiens_tRNA- | GGTAGCGCGTCTGACTCCAG | CBE | 2189 |
| Trp-CCA-2-1 | | | |
| Homo_sapiens_tRNA- | GTAGCGCGTCTGACTCCAGA | CBE | 2190 |
| Trp-CCA-2-1 | | | |
| Homo_sapiens_tRNA- | TAGCGCGTCTGACTCCAGAT | CBE | 2191 |
| Trp-CCA-2-1 | | | |
| Homo_sapiens_tRNA- | AGCGCGTCTGACTCCAGATC | CBE | 2192 |
| Trp-CCA-2-1 | | | |
| Homo_sapiens_tRNA- | GCGCGTCTGACTCCAGATCA | CBE | 2193 |
| Trp-CCA-2-1 | | | |
| Homo_sapiens_tRNA- | CGCGTCTGACTCCAGATCAG | CBE | 2194 |
| Trp-CCA-2-1 | | | |
| Homo_sapiens_tRNA- | GCGTCTGACTCCAGATCAGA | CBE | 2195 |
| Trp-CCA-2-1 | | | |
| Homo_sapiens_tRNA- | CGTCTGACTCCAGATCAGAA | CBE | 2196 |
| Trp-CCA-2-1 | | | |
| Homo_sapiens_tRNA- | GTCTGACTCCAGATCAGAAG | CBE | 2197 |
| Trp-CCA-2-1 | | | |
| Homo_sapiens_tRNA- | TCTGACTCCAGATCAGAAGG | CBE | 2198 |
| Trp-CCA-2-1 | | | |
| Homo_sapiens_tRNA- | CTGACTCCAGATCAGAAGGT | CBE | 2199 |
| Trp-CCA-2-1 | | | |
| Homo_sapiens_tRNA- | TGACTCCAGATCAGAAGGTT | CBE | 2200 |
| Trp-CCA-2-1 | | | |
| Homo_sapiens_tRNA- | GACTCCAGATCAGAAGGTTG | CBE | 2201 |
| Trp-CCA-2-1 | | | |
| Homo_sapiens_tRNA- | ACTCCAGATCAGAAGGTTGC | CBE | 2202 |
| Trp-CCA-2-1 | | | |
| Homo_sapiens_tRNA- | CTCCAGATCAGAAGGTTGCG | CBE | 2203 |
| Trp-CCA-2-1 | | | |
| Homo_sapiens_tRNA- | TCCAGATCAGAAGGTTGCGT | CBE | 2204 |
| Trp-CCA-2-1 | | | |
| Homo_sapiens_tRNA- | ACGGTAGCGCGTCTGACTCC | CBE | 2205 |
| Trp-CCA-3-1 | | | |
| Homo_sapiens_tRNA- | CGGTAGCGCGTCTGACTCCA | CBE | 2206 |
| Trp-CCA-3-1 | | | |
| Homo_sapiens_tRNA- | GGTAGCGCGTCTGACTCCAG | CBE | 2207 |
| Trp-CCA-3-1 | | | |
| Homo_sapiens_tRNA- | GTAGCGCGTCTGACTCCAGA | CBE | 2208 |
| Trp-CCA-3-1 | | | |
| Homo_sapiens_tRNA- | TAGCGCGTCTGACTCCAGAT | CBE | 2209 |
| Trp-CCA-3-1 | | | |
| Homo_sapiens_tRNA- | AGCGCGTCTGACTCCAGATC | CBE | 2210 |
| Trp-CCA-3-1 | | | |
| Homo_sapiens_tRNA- | GCGCGTCTGACTCCAGATCA | CBE | 2211 |
| Trp-CCA-3-1 | | | |
| Homo_sapiens_tRNA- | CGCGTCTGACTCCAGATCAG | CBE | 2212 |
| Trp-CCA-3-1 | | | |
| Homo_sapiens_tRNA- | GCGTCTGACTCCAGATCAGA | CBE | 2213 |
| Trp-CCA-3-1 | | | |
| Homo_sapiens_tRNA- | CGTCTGACTCCAGATCAGAA | CBE | 2214 |
| Trp-CCA-3-1 | | | |
| Homo_sapiens_tRNA- | GTCTGACTCCAGATCAGAAG | CBE | 2215 |
| Trp-CCA-3-1 | | | |
| Homo_sapiens_tRNA- | TCTGACTCCAGATCAGAAGG | CBE | 2216 |
| Trp-CCA-3-1 | | | |
| Homo_sapiens_tRNA- | CTGACTCCAGATCAGAAGGT | CBE | 2217 |
| Trp-CCA-3-1 | | | |
| Homo_sapiens_tRNA- | TGACTCCAGATCAGAAGGTT | CBE | 2218 |
| Trp-CCA-3-1 | | | |
| Homo_sapiens_tRNA- | GACTCCAGATCAGAAGGTTG | CBE | 2219 |
| Trp-CCA-3-1 | | | |
| Homo_sapiens_tRNA- | ACTCCAGATCAGAAGGTTGC | CBE | 2220 |
| Trp-CCA-3-1 | | | |
| Homo_sapiens_tRNA- | CTCCAGATCAGAAGGTTGCG | CBE | 2221 |
| Trp-CCA-3-1 | | | |
| Homo_sapiens_tRNA- | TCCAGATCAGAAGGTTGCGT | CBE | 2222 |
| Trp-CCA-3-1 | | | |
| Homo_sapiens_tRNA- | ACGGTAGCGCGTCTGACTCC | CBE | 2223 |
| Trp-CCA-3-2 | | | |
| Homo_sapiens_tRNA- | CGGTAGCGCGTCTGACTCCA | CBE | 2224 |
| Trp-CCA-3-2 | | | |
| Homo_sapiens_tRNA- | GGTAGCGCGTCTGACTCCAG | CBE | 2225 |
| Trp-CCA-3-2 | | | |
| Homo_sapiens_tRNA- | GTAGCGCGTCTGACTCCAGA | CBE | 2226 |
| Trp-CCA-3-2 | | | |
| Homo_sapiens_tRNA- | TAGCGCGTCTGACTCCAGAT | CBE | 2227 |
| Trp-CCA-3-2 | | | |
| Homo_sapiens_tRNA- | AGCGCGTCTGACTCCAGATC | CBE | 2228 |
| Trp-CCA-3-2 | | | |
| Homo_sapiens_tRNA- | GCGCGTCTGACTCCAGATCA | CBE | 2229 |
| Trp-CCA-3-2 | | | |
| Homo_sapiens_tRNA- | CGCGTCTGACTCCAGATCAG | CBE | 2230 |
| Trp-CCA-3-2 | | | |
| Homo_sapiens_tRNA- | GCGTCTGACTCCAGATCAGA | CBE | 2231 |
| Trp-CCA-3-2 | | | |
| Homo_sapiens_tRNA- | CGTCTGACTCCAGATCAGAA | CBE | 2232 |
| Trp-CCA-3-2 | | | |
| Homo_sapiens_tRNA- | GTCTGACTCCAGATCAGAAG | CBE | 2233 |
| Trp-CCA-3-2 | | | |
| Homo_sapiens_tRNA- | TCTGACTCCAGATCAGAAGG | CBE | 2234 |
| Trp-CCA-3-2 | | | |
| Homo_sapiens_tRNA- | CTGACTCCAGATCAGAAGGT | CBE | 2235 |
| Trp-CCA-3-2 | | | |
| Homo_sapiens_tRNA- | TGACTCCAGATCAGAAGGTT | CBE | 2236 |
| Trp-CCA-3-2 | | | |
| Homo_sapiens_tRNA- | GACTCCAGATCAGAAGGTTG | CBE | 2237 |
| Trp-CCA-3-2 | | | |
| Homo_sapiens_tRNA- | ACTCCAGATCAGAAGGTTGC | CBE | 2238 |
| Trp-CCA-3-2 | | | |
| Homo_sapiens_tRNA- | CTCCAGATCAGAAGGTTGCG | CBE | 2239 |
| Trp-CCA-3-2 | | | |
| Homo_sapiens_tRNA- | TCCAGATCAGAAGGTTGCGT | CBE | 2240 |
| Trp-CCA-3-2 | | | |
| Homo_sapiens_tRNA- | ACGGTAGCGCGTCTGACTCC | CBE | 2241 |
| Trp-CCA-3-3 | | | |
| Homo_sapiens_tRNA- | CGGTAGCGCGTCTGACTCCA | CBE | 2242 |
| Trp-CCA-3-3 | | | |
| Homo_sapiens_tRNA- | GGTAGCGCGTCTGACTCCAG | CBE | 2243 |
| Trp-CCA-3-3 | | | |
| Homo_sapiens_tRNA- | GTAGCGCGTCTGACTCCAGA | CBE | 2244 |
| Trp-CCA-3-3 | | | |
| Homo_sapiens_tRNA- | TAGCGCGTCTGACTCCAGAT | CBE | 2245 |
| Trp-CCA-3-3 | | | |
| Homo_sapiens_tRNA- | AGCGCGTCTGACTCCAGATC | CBE | 2246 |
| Trp-CCA-3-3 | | | |
| Homo_sapiens_tRNA- | GCGCGTCTGACTCCAGATCA | CBE | 2247 |
| Trp-CCA-3-3 | | | |
| Homo_sapiens_tRNA- | CGCGTCTGACTCCAGATCAG | CBE | 2248 |
| Trp-CCA-3-3 | | | |
| Homo_sapiens_tRNA- | GCGTCTGACTCCAGATCAGA | CBE | 2249 |
| Trp-CCA-3-3 | | | |
| Homo_sapiens_tRNA- | CGTCTGACTCCAGATCAGAA | CBE | 2250 |
| Trp-CCA-3-3 | | | |
| Homo_sapiens_tRNA- | GTCTGACTCCAGATCAGAAG | CBE | 2251 |
| Trp-CCA-3-3 | | | |
| Homo_sapiens_tRNA- | TCTGACTCCAGATCAGAAGG | CBE | 2252 |
| Trp-CCA-3-3 | | | |
| Homo_sapiens_tRNA- | CTGACTCCAGATCAGAAGGT | CBE | 2253 |
| Trp-CCA-3-3 | | | |
| Homo_sapiens_tRNA- | TGACTCCAGATCAGAAGGTT | CBE | 2254 |
| Trp-CCA-3-3 | | | |
| Homo_sapiens_tRNA- | GACTCCAGATCAGAAGGTTG | CBE | 2255 |
| Trp-CCA-3-3 | | | |
| Homo_sapiens_tRNA- | ACTCCAGATCAGAAGGTTGC | CBE | 2256 |
| Trp-CCA-3-3 | | | |
| Homo_sapiens_tRNA- | CTCCAGATCAGAAGGTTGCG | CBE | 2257 |
| Trp-CCA-3-3 | | | |
| Homo_sapiens_tRNA- | TCCAGATCAGAAGGTTGCGT | CBE | 2258 |
| Trp-CCA-3-3 | | | |
| Homo_sapiens_tRNA- | ACGGTAGCGCGTCTGACTCC | CBE | 2259 |
| Trp-CCA-4-1 | | | |
| Homo_sapiens_tRNA- | CGGTAGCGCGTCTGACTCCA | CBE | 2260 |
| Trp-CCA-4-1 | | | |
| Homo_sapiens_tRNA- | GGTAGCGCGTCTGACTCCAG | CBE | 2261 |
| Trp-CCA-4-1 | | | |
| Homo_sapiens_tRNA- | GTAGCGCGTCTGACTCCAGA | CBE | 2262 |
| Trp-CCA-4-1 | | | |
| Homo_sapiens_tRNA- | TAGCGCGTCTGACTCCAGAT | CBE | 2263 |
| Trp-CCA-4-1 | | | |
| Homo_sapiens_tRNA- | AGCGCGTCTGACTCCAGATC | CBE | 2264 |
| Trp-CCA-4-1 | | | |
| Homo_sapiens_tRNA- | GCGCGTCTGACTCCAGATCA | CBE | 2265 |
| Trp-CCA-4-1 | | | |
| Homo_sapiens_tRNA- | CGCGTCTGACTCCAGATCAG | CBE | 2266 |
| Trp-CCA-4-1 | | | |
| Homo_sapiens_tRNA- | GCGTCTGACTCCAGATCAGA | CBE | 2267 |
| Trp-CCA-4-1 | | | |
| Homo_sapiens_tRNA- | CGTCTGACTCCAGATCAGAA | CBE | 2268 |
| Trp-CCA-4-1 | | | |
| Homo_sapiens_tRNA- | GTCTGACTCCAGATCAGAAG | CBE | 2269 |
| Trp-CCA-4-1 | | | |
| Homo_sapiens_tRNA- | TCTGACTCCAGATCAGAAGG | CBE | 2270 |
| Trp-CCA-4-1 | | | |
| Homo_sapiens_tRNA- | CTGACTCCAGATCAGAAGGC | CBE | 2271 |
| Trp-CCA-4-1 | | | |
| Homo_sapiens_tRNA- | TGACTCCAGATCAGAAGGCT | CBE | 2272 |
| Trp-CCA-4-1 | | | |
| Homo_sapiens_tRNA- | GACTCCAGATCAGAAGGCTG | CBE | 2273 |
| Trp-CCA-4-1 | | | |
| Homo_sapiens_tRNA- | ACTCCAGATCAGAAGGCTGC | CBE | 2274 |
| Trp-CCA-4-1 | | | |
| Homo_sapiens_tRNA- | CTCCAGATCAGAAGGCTGCG | CBE | 2275 |
| Trp-CCA-4-1 | | | |
| Homo_sapiens_tRNA- | TCCAGATCAGAAGGCTGCGT | CBE | 2276 |
| Trp-CCA-4-1 | | | |
| Homo_sapiens_tRNA- | ACGGCAGCGCGTCTGACTCC | CBE | 2277 |
| Trp-CCA-5-1 | | | |
| Homo_sapiens_tRNA- | CGGCAGCGCGTCTGACTCCA | CBE | 2278 |
| Trp-CCA-5-1 | | | |
| Homo_sapiens_tRNA- | GGCAGCGCGTCTGACTCCAG | CBE | 2279 |
| Trp-CCA-5-1 | | | |
| Homo_sapiens_tRNA- | GCAGCGCGTCTGACTCCAGA | CBE | 2280 |
| Trp-CCA-5-1 | | | |
| Homo_sapiens_tRNA- | CAGCGCGTCTGACTCCAGAT | CBE | 2281 |
| Trp-CCA-5-1 | | | |
| Homo_sapiens_tRNA- | AGCGCGTCTGACTCCAGATC | CBE | 2282 |
| Trp-CCA-5-1 | | | |
| Homo_sapiens_tRNA- | GCGCGTCTGACTCCAGATCA | CBE | 2283 |
| Trp-CCA-5-1 | | | |
| Homo_sapiens_tRNA- | CGCGTCTGACTCCAGATCAG | CBE | 2284 |
| Trp-CCA-5-1 | | | |
| Homo_sapiens_tRNA- | GCGTCTGACTCCAGATCAGA | CBE | 2285 |
| Trp-CCA-5-1 | | | |
| Homo_sapiens_tRNA- | CGTCTGACTCCAGATCAGAA | CBE | 2286 |
| Trp-CCA-5-1 | | | |
| Homo_sapiens_tRNA- | GTCTGACTCCAGATCAGAAG | CBE | 2287 |
| Trp-CCA-5-1 | | | |
| Homo_sapiens_tRNA- | TCTGACTCCAGATCAGAAGG | CBE | 2288 |
| Trp-CCA-5-1 | | | |
| Homo_sapiens_tRNA- | CTGACTCCAGATCAGAAGGT | CBE | 2289 |
| Trp-CCA-5-1 | | | |
| Homo_sapiens_tRNA- | TGACTCCAGATCAGAAGGTT | CBE | 2290 |
| Trp-CCA-5-1 | | | |
| Homo_sapiens_tRNA- | GACTCCAGATCAGAAGGTTG | CBE | 2291 |
| Trp-CCA-5-1 | | | |
| Homo_sapiens_tRNA- | ACTCCAGATCAGAAGGTTGC | CBE | 2292 |
| Trp-CCA-5-1 | | | |
| Homo_sapiens_tRNA- | CTCCAGATCAGAAGGTTGCG | CBE | 2293 |
| Trp-CCA-5-1 | | | |
| Homo_sapiens_tRNA- | TCCAGATCAGAAGGTTGCGT | CBE | 2294 |
| Trp-CCA-5-1 | | | |
| Homo_sapiens_tRNA- | TGGTAGAGCAGAGGACTATA | ACBE | 2295 |
| Tyr-ATA-1-1 | | | |
| Homo_sapiens_tRNA- | GGTAGAGCAGAGGACTATAG | ACBE | 2296 |
| Tyr-ATA-1-1 | | | |
| Homo_sapiens_tRNA- | GTAGAGCAGAGGACTATAGC | ACBE | 2297 |
| Tyr-ATA-1-1 | | | |
| Homo_sapiens_tRNA- | TAGAGCAGAGGACTATAGCT | ACBE | 2298 |
| Tyr-ATA-1-1 | | | |
| Homo_sapiens_tRNA- | AGAGCAGAGGACTATAGCTA | ACBE | 2299 |
| Tyr-ATA-1-1 | | | |
| Homo_sapiens_tRNA- | GAGCAGAGGACTATAGCTAC | ACBE | 2300 |
| Tyr-ATA-1-1 | | | |
| Homo_sapiens_tRNA- | AGCAGAGGACTATAGCTACT | ACBE | 2301 |
| Tyr-ATA-1-1 | | | |
| Homo_sapiens_tRNA- | GCAGAGGACTATAGCTACTT | ACBE | 2302 |
| Tyr-ATA-1-1 | | | |
| Homo_sapiens_tRNA- | CAGAGGACTATAGCTACTTC | ACBE | 2303 |
| Tyr-ATA-1-1 | | | |
| Homo_sapiens_tRNA- | AGAGGACTATAGCTACTTCC | ACBE | 2304 |
| Tyr-ATA-1-1 | | | |
| Homo_sapiens_tRNA- | GAGGACTATAGCTACTTCCT | ACBE | 2305 |
| Tyr-ATA-1-1 | | | |
| Homo_sapiens_tRNA- | AGGACTATAGCTACTTCCTC | ACBE | 2306 |
| Tyr-ATA-1-1 | | | |
| Homo_sapiens_tRNA- | GGACTATAGCTACTTCCTCA | ACBE | 2307 |
| Tyr-ATA-1-1 | | | |
| Homo_sapiens_tRNA- | GACTATAGCTACTTCCTCAG | ACBE | 2308 |
| Tyr-ATA-1-1 | | | |
| Homo_sapiens_tRNA- | TACAGTCCTCCGCTCTACCA | CABE | 2309 |
| Tyr-GTA-1-1 | | | |
| Homo_sapiens_tRNA- | CTACAGTCCTCCGCTCTACC | CABE | 2310 |
| Tyr-GTA-1-1 | | | |
| Homo_sapiens_tRNA- | ACTACAGTCCTCCGCTCTAC | CABE | 2311 |
| Tyr-GTA-1-1 | | | |
| Homo_sapiens_tRNA- | AACTACAGTCCTCCGCTCTA | CABE | 2312 |
| Tyr-GTA-1-1 | | | |
| Homo_sapiens_tRNA- | CAACTACAGTCCTCCGCTCT | CABE | 2313 |
| Tyr-GTA-1-1 | | | |
| Homo_sapiens_tRNA- | CCAACTACAGTCCTCCGCTC | CABE | 2314 |
| Tyr-GTA-1-1 | | | |
| Homo_sapiens_tRNA- | GCCAACTACAGTCCTCCGCT | CABE | 2315 |
| Tyr-GTA-1-1 | | | |
| Homo_sapiens_tRNA- | AGCCAACTACAGTCCTCCGC | CABE | 2316 |
| Tyr-GTA-1-1 | | | |
| Homo_sapiens_tRNA- | CAGCCAACTACAGTCCTCCG | CABE | 2317 |
| Tyr-GTA-1-1 | | | |
| Homo_sapiens_tRNA- | ACAGCCAACTACAGTCCTCC | CABE | 2318 |
| Tyr-GTA-1-1 | | | |
| Homo_sapiens_tRNA- | CACAGCCAACTACAGTCCTC | CABE | 2319 |
| Tyr-GTA-1-1 | | | |
| Homo_sapiens_tRNA- | ACACAGCCAACTACAGTCCT | CABE | 2320 |
| Tyr-GTA-1-1 | | | |
| Homo_sapiens_tRNA- | GACACAGCCAACTACAGTCC | CABE | 2321 |
| Tyr-GTA-1-1 | | | |
| Homo_sapiens_tRNA- | GGACACAGCCAACTACAGTC | CABE | 2322 |
| Tyr-GTA-1-1 | | | |
| Homo_sapiens_tRNA- | TACAGTCCTCCGCTCTACCA | CABE | 2323 |
| Tyr-GTA-2-1 | | | |
| Homo_sapiens_tRNA- | CTACAGTCCTCCGCTCTACC | CABE | 2324 |
| Tyr-GTA-2-1 | | | |
| Homo_sapiens_tRNA- | ACTACAGTCCTCCGCTCTAC | CABE | 2325 |
| Tyr-GTA-2-1 | | | |
| Homo_sapiens_tRNA- | CACTACAGTCCTCCGCTCTA | CABE | 2326 |
| Tyr-GTA-2-1 | | | |
| Homo_sapiens_tRNA- | CCACTACAGTCCTCCGCTCT | CABE | 2327 |
| Tyr-GTA-2-1 | | | |
| Homo_sapiens_tRNA- | TCCACTACAGTCCTCCGCTC | CABE | 2328 |
| Tyr-GTA-2-1 | | | |
| Homo_sapiens_tRNA- | ATCCACTACAGTCCTCCGCT | CABE | 2329 |
| Tyr-GTA-2-1 | | | |
| Homo_sapiens_tRNA- | TATCCACTACAGTCCTCCGC | CABE | 2330 |
| Tyr-GTA-2-1 | | | |
| Homo_sapiens_tRNA- | CTATCCACTACAGTCCTCCG | CABE | 2331 |
| Tyr-GTA-2-1 | | | |
| Homo_sapiens_tRNA- | CCTATCCACTACAGTCCTCC | CABE | 2332 |
| Tyr-GTA-2-1 | | | |
| Homo_sapiens_tRNA- | CCCTATCCACTACAGTCCTC | CABE | 2333 |
| Tyr-GTA-2-1 | | | |
| Homo_sapiens_tRNA- | GCCCTATCCACTACAGTCCT | CABE | 2334 |
| Tyr-GTA-2-1 | | | |
| Homo_sapiens_tRNA- | CGCCCTATCCACTACAGTCC | CABE | 2335 |
| Tyr-GTA-2-1 | | | |
| Homo_sapiens_tRNA- | ACGCCCTATCCACTACAGTC | CABE | 2336 |
| Tyr-GTA-2-1 | | | |
| Homo_sapiens_tRNA- | TACAGTCCTCCGCTCTACCA | CABE | 2337 |
| Tyr-GTA-3-1 | | | |
| Homo_sapiens_tRNA- | CTACAGTCCTCCGCTCTACC | CABE | 2338 |
| Tyr-GTA-3-1 | | | |
| Homo_sapiens_tRNA- | CCTACAGTCCTCCGCTCTAC | CABE | 2339 |
| Tyr-GTA-3-1 | | | |
| Homo_sapiens_tRNA- | GCCTACAGTCCTCCGCTCTA | CABE | 2340 |
| Tyr-GTA-3-1 | | | |
| Homo_sapiens_tRNA- | AGCCTACAGTCCTCCGCTCT | CABE | 2341 |
| Tyr-GTA-3-1 | | | |
| Homo_sapiens_tRNA- | GAGCCTACAGTCCTCCGCTC | CABE | 2342 |
| Tyr-GTA-3-1 | | | |
| Homo_sapiens_tRNA- | TGAGCCTACAGTCCTCCGCT | CABE | 2343 |
| Tyr-GTA-3-1 | | | |
| Homo_sapiens_tRNA- | ATGAGCCTACAGTCCTCCGC | CABE | 2344 |
| Tyr-GTA-3-1 | | | |
| Homo_sapiens_tRNA- | AATGAGCCTACAGTCCTCCG | CABE | 2345 |
| Tyr-GTA-3-1 | | | |
| Homo_sapiens_tRNA- | TAATGAGCCTACAGTCCTCC | CABE | 2346 |
| Tyr-GTA-3-1 | | | |
| Homo_sapiens_tRNA- | TTAATGAGCCTACAGTCCTC | CABE | 2347 |
| Tyr-GTA-3-1 | | | |
| Homo_sapiens_tRNA- | CTTAATGAGCCTACAGTCCT | CABE | 2348 |
| Tyr-GTA-3-1 | | | |
| Homo_sapiens_tRNA- | GCTTAATGAGCCTACAGTCC | CABE | 2349 |
| Tyr-GTA-3-1 | | | |
| Homo_sapiens_tRNA- | TGCTTAATGAGCCTACAGTC | CABE | 2350 |
| Tyr-GTA-3-1 | | | |
| Homo_sapiens_tRNA- | TACAGTCCTCCGCTCTACCA | CABE | 2351 |
| Tyr-GTA-4-1 | | | |
| Homo_sapiens_tRNA- | CTACAGTCCTCCGCTCTACC | CABE | 2352 |
| Tyr-GTA-4-1 | | | |
| Homo_sapiens_tRNA- | TCTACAGTCCTCCGCTCTAC | CABE | 2353 |
| Tyr-GTA-4-1 | | | |
| Homo_sapiens_tRNA- | ATCTACAGTCCTCCGCTCTA | CABE | 2354 |
| Tyr-GTA-4-1 | | | |
| Homo_sapiens_tRNA- | AATCTACAGTCCTCCGCTCT | CABE | 2355 |
| Tyr-GTA-4-1 | | | |
| Homo_sapiens_tRNA- | CAATCTACAGTCCTCCGCTC | CABE | 2356 |
| Tyr-GTA-4-1 | | | |
| Homo_sapiens_tRNA- | ACAATCTACAGTCCTCCGCT | CABE | 2357 |
| Tyr-GTA-4-1 | | | |
| Homo_sapiens_tRNA- | TACAATCTACAGTCCTCCGC | CABE | 2358 |
| Tyr-GTA-4-1 | | | |
| Homo_sapiens_tRNA- | ATACAATCTACAGTCCTCCG | CABE | 2359 |
| Tyr-GTA-4-1 | | | |
| Homo_sapiens_tRNA- | TATACAATCTACAGTCCTCC | CABE | 2360 |
| Tyr-GTA-4-1 | | | |
| Homo_sapiens_tRNA- | CTATACAATCTACAGTCCTC | CABE | 2361 |
| Tyr-GTA-4-1 | | | |
| Homo_sapiens_tRNA- | TCTATACAATCTACAGTCCT | CABE | 2362 |
| Tyr-GTA-4-1 | | | |
| Homo_sapiens_tRNA- | GTCTATACAATCTACAGTCC | CABE | 2363 |
| Tyr-GTA-4-1 | | | |
| Homo_sapiens_tRNA- | TGTCTATACAATCTACAGTC | CABE | 2364 |
| Tyr-GTA-4-1 | | | |
| Homo_sapiens_tRNA- | TACAGTCCTCCGCTCTACCA | CABE | 2365 |
| Tyr-GTA-5-1 | | | |
| Homo_sapiens_tRNA- | CTACAGTCCTCCGCTCTACC | CABE | 2366 |
| Tyr-GTA-5-1 | | | |
| Homo_sapiens_tRNA- | GCTACAGTCCTCCGCTCTAC | CABE | 2367 |
| Tyr-GTA-5-1 | | | |
| Homo_sapiens_tRNA- | AGCTACAGTCCTCCGCTCTA | CABE | 2368 |
| Tyr-GTA-5-1 | | | |
| Homo_sapiens_tRNA- | TAGCTACAGTCCTCCGCTCT | CABE | 2369 |
| Tyr-GTA-5-1 | | | |
| Homo_sapiens_tRNA- | GTAGCTACAGTCCTCCGCTC | CABE | 2370 |
| Tyr-GTA-5-1 | | | |
| Homo_sapiens_tRNA- | AGTAGCTACAGTCCTCCGCT | CABE | 2371 |
| Tyr-GTA-5-1 | | | |
| Homo_sapiens_tRNA- | AAGTAGCTACAGTCCTCCGC | CABE | 2372 |
| Tyr-GTA-5-1 | | | |
| Homo_sapiens_tRNA- | GAAGTAGCTACAGTCCTCCG | CABE | 2373 |
| Tyr-GTA-5-1 | | | |
| Homo_sapiens_tRNA- | GGAAGTAGCTACAGTCCTCC | CABE | 2374 |
| Tyr-GTA-5-1 | | | |
| Homo_sapiens_tRNA- | AGGAAGTAGCTACAGTCCTC | CABE | 2375 |
| Tyr-GTA-5-1 | | | |
| Homo_sapiens_tRNA- | GAGGAAGTAGCTACAGTCCT | CABE | 2376 |
| Tyr-GTA-5-1 | | | |
| Homo_sapiens_tRNA- | TGAGGAAGTAGCTACAGTCC | CABE | 2377 |
| Tyr-GTA-5-1 | | | |
| Homo_sapiens_tRNA- | CTGAGGAAGTAGCTACAGTC | CABE | 2378 |
| Tyr-GTA-5-1 | | | |
| Homo_sapiens_tRNA- | TACAGTCCTCCGCTCTACCA | CABE | 2379 |
| Tyr-GTA-5-2 | | | |
| Homo_sapiens_tRNA- | CTACAGTCCTCCGCTCTACC | CABE | 2380 |
| Tyr-GTA-5-2 | | | |
| Homo_sapiens_tRNA- | CCTACAGTCCTCCGCTCTAC | CABE | 2381 |
| Tyr-GTA-5-2 | | | |
| Homo_sapiens_tRNA- | GCCTACAGTCCTCCGCTCTA | CABE | 2382 |
| Tyr-GTA-5-2 | | | |
| Homo_sapiens_tRNA- | CGCCTACAGTCCTCCGCTCT | CABE | 2383 |
| Tyr-GTA-5-2 | | | |
| Homo_sapiens_tRNA- | GCGCCTACAGTCCTCCGCTC | CABE | 2384 |
| Tyr-GTA-5-2 | | | |
| Homo_sapiens_tRNA- | CGCGCCTACAGTCCTCCGCT | CABE | 2385 |
| Tyr-GTA-5-2 | | | |
| Homo_sapiens_tRNA- | GCGCGCCTACAGTCCTCCGC | CABE | 2386 |
| Tyr-GTA-5-2 | | | |
| Homo_sapiens_tRNA- | CGCGCGCCTACAGTCCTCCG | CABE | 2387 |
| Tyr-GTA-5-2 | | | |
| Homo_sapiens_tRNA- | GCGCGCGCCTACAGTCCTCC | CABE | 2388 |
| Tyr-GTA-5-2 | | | |
| Homo_sapiens_tRNA- | GGCGCGCGCCTACAGTCCTC | CABE | 2389 |
| Tyr-GTA-5-2 | | | |
| Homo_sapiens_tRNA- | GGGCGCGCGCCTACAGTCCT | CABE | 2390 |
| Tyr-GTA-5-2 | | | |
| Homo_sapiens_tRNA- | CGGGCGCGCGCCTACAGTCC | CABE | 2391 |
| Tyr-GTA-5-2 | | | |
| Homo_sapiens_tRNA- | ACGGGCGCGCGCCTACAGTC | CABE | 2392 |
| Tyr-GTA-5-2 | | | |
| Homo_sapiens_tRNA- | TACAGTCCTCCGCTCTACCA | CABE | 2393 |
| Tyr-GTA-5-3 | | | |
| Homo_sapiens_tRNA- | CTACAGTCCTCCGCTCTACC | CABE | 2394 |
| Tyr-GTA-5-3 | | | |
| Homo_sapiens_tRNA- | GCTACAGTCCTCCGCTCTAC | CABE | 2395 |
| Tyr-GTA-5-3 | | | |
| Homo_sapiens_tRNA- | GGCTACAGTCCTCCGCTCTA | CABE | 2396 |
| Tyr-GTA-5-3 | | | |
| Homo_sapiens_tRNA- | AGGCTACAGTCCTCCGCTCT | CABE | 2397 |
| Tyr-GTA-5-3 | | | |
| Homo_sapiens_tRNA- | CAGGCTACAGTCCTCCGCTC | CABE | 2398 |
| Tyr-GTA-5-3 | | | |
| Homo_sapiens_tRNA- | ACAGGCTACAGTCCTCCGCT | CABE | 2399 |
| Tyr-GTA-5-3 | | | |
| Homo_sapiens_tRNA- | TACAGGCTACAGTCCTCCGC | CABE | 2400 |
| Tyr-GTA-5-3 | | | |
| Homo_sapiens_tRNA- | CTACAGGCTACAGTCCTCCG | CABE | 2401 |
| Tyr-GTA-5-3 | | | |
| Homo_sapiens_tRNA- | TCTACAGGCTACAGTCCTCC | CABE | 2402 |
| Tyr-GTA-5-3 | | | |
| Homo_sapiens_tRNA- | TTCTACAGGCTACAGTCCTC | CABE | 2403 |
| Tyr-GTA-5-3 | | | |
| Homo_sapiens_tRNA- | TTTCTACAGGCTACAGTCCT | CABE | 2404 |
| Tyr-GTA-5-3 | | | |
| Homo_sapiens_tRNA- | GTTTCTACAGGCTACAGTCC | CABE | 2405 |
| Tyr-GTA-5-3 | | | |
| Homo_sapiens_tRNA- | TGTTTCTACAGGCTACAGTC | CABE | 2406 |
| Tyr-GTA-5-3 | | | |
| Homo_sapiens_tRNA- | TACAGTCCTCCGCTCTACCA | CABE | 2407 |
| Tyr-GTA-5-4 | | | |
| Homo_sapiens_tRNA- | CTACAGTCCTCCGCTCTACC | CABE | 2408 |
| Tyr-GTA-5-4 | | | |
| Homo_sapiens_tRNA- | TCTACAGTCCTCCGCTCTAC | CABE | 2409 |
| Tyr-GTA-5-4 | | | |
| Homo_sapiens_tRNA- | ATCTACAGTCCTCCGCTCTA | CABE | 2410 |
| Tyr-GTA-5-4 | | | |
| Homo_sapiens_tRNA- | AATCTACAGTCCTCCGCTCT | CABE | 2411 |
| Tyr-GTA-5-4 | | | |
| Homo_sapiens_tRNA- | CAATCTACAGTCCTCCGCTC | CABE | 2412 |
| Tyr-GTA-5-4 | | | |
| Homo_sapiens_tRNA- | ACAATCTACAGTCCTCCGCT | CABE | 2413 |
| Tyr-GTA-5-4 | | | |
| Homo_sapiens_tRNA- | TACAATCTACAGTCCTCCGC | CABE | 2414 |
| Tyr-GTA-5-4 | | | |
| Homo_sapiens_tRNA- | GTACAATCTACAGTCCTCCG | CABE | 2415 |
| Tyr-GTA-5-4 | | | |
| Homo_sapiens_tRNA- | TGTACAATCTACAGTCCTCC | CABE | 2416 |
| Tyr-GTA-5-4 | | | |
| Homo_sapiens_tRNA- | CTGTACAATCTACAGTCCTC | CABE | 2417 |
| Tyr-GTA-5-4 | | | |
| Homo_sapiens_tRNA- | TCTGTACAATCTACAGTCCT | CABE | 2418 |
| Tyr-GTA-5-4 | | | |
| Homo_sapiens_tRNA- | GTCTGTACAATCTACAGTCC | CABE | 2419 |
| Tyr-GTA-5-4 | | | |
| Homo_sapiens_tRNA- | TGTCTGTACAATCTACAGTC | CABE | 2420 |
| Tyr-GTA-5-4 | | | |
| Homo_sapiens_tRNA- | TACAGTCCTCCGCTCTACCA | CABE | 2421 |
| Tyr-GTA-5-5 | | | |
| Homo_sapiens_tRNA- | CTACAGTCCTCCGCTCTACC | CABE | 2422 |
| Tyr-GTA-5-5 | | | |
| Homo_sapiens_tRNA- | ACTACAGTCCTCCGCTCTAC | CABE | 2423 |
| Tyr-GTA-5-5 | | | |
| Homo_sapiens_tRNA- | TACTACAGTCCTCCGCTCTA | CABE | 2424 |
| Tyr-GTA-5-5 | | | |
| Homo_sapiens_tRNA- | GTACTACAGTCCTCCGCTCT | CABE | 2425 |
| Tyr-GTA-5-5 | | | |
| Homo_sapiens_tRNA- | AGTACTACAGTCCTCCGCTC | CABE | 2426 |
| Tyr-GTA-5-5 | | | |
| Homo_sapiens_tRNA- | AAGTACTACAGTCCTCCGCT | CABE | 2427 |
| Tyr-GTA-5-5 | | | |
| Homo_sapiens_tRNA- | TAAGTACTACAGTCCTCCGC | CABE | 2428 |
| Tyr-GTA-5-5 | | | |
| Homo_sapiens_tRNA- | TTAAGTACTACAGTCCTCCG | CABE | 2429 |
| Tyr-GTA-5-5 | | | |
| Homo_sapiens_tRNA- | ATTAAGTACTACAGTCCTCC | CABE | 2430 |
| Tyr-GTA-5-5 | | | |
| Homo_sapiens_tRNA- | CATTAAGTACTACAGTCCTC | CABE | 2431 |
| Tyr-GTA-5-5 | | | |
| Homo_sapiens_tRNA- | ACATTAAGTACTACAGTCCT | CABE | 2432 |
| Tyr-GTA-5-5 | | | |
| Homo_sapiens_tRNA- | CACATTAAGTACTACAGTCC | CABE | 2433 |
| Tyr-GTA-5-5 | | | |
| Homo_sapiens_tRNA- | ACACATTAAGTACTACAGTC | CABE | 2434 |
| Tyr-GTA-5-5 | | | |
| Homo_sapiens_tRNA- | TACAGTCCTCCGCTCTACCA | CABE | 2435 |
| Tyr-GTA-6-1 | | | |
| Homo_sapiens_tRNA- | CTACAGTCCTCCGCTCTACC | CABE | 2436 |
| Tyr-GTA-6-1 | | | |
| Homo_sapiens_tRNA- | CCTACAGTCCTCCGCTCTAC | CABE | 2437 |
| Tyr-GTA-6-1 | | | |
| Homo_sapiens_tRNA- | CCCTACAGTCCTCCGCTCTA | CABE | 2438 |
| Tyr-GTA-6-1 | | | |
| Homo_sapiens_tRNA- | CCCCTACAGTCCTCCGCTCT | CABE | 2439 |
| Tyr-GTA-6-1 | | | |
| Homo_sapiens_tRNA- | ACCCCTACAGTCCTCCGCTC | CABE | 2440 |
| Tyr-GTA-6-1 | | | |
| Homo_sapiens_tRNA- | AACCCCTACAGTCCTCCGCT | CABE | 2441 |
| Tyr-GTA-6-1 | | | |
| Homo_sapiens_tRNA- | AAACCCCTACAGTCCTCCGC | CABE | 2442 |
| Tyr-GTA-6-1 | | | |
| Homo_sapiens_tRNA- | CAAACCCCTACAGTCCTCCG | CABE | 2443 |
| Tyr-GTA-6-1 | | | |
| Homo_sapiens_tRNA- | TCAAACCCCTACAGTCCTCC | CABE | 2444 |
| Tyr-GTA-6-1 | | | |
| Homo_sapiens_tRNA- | TTCAAACCCCTACAGTCCTC | CABE | 2445 |
| Tyr-GTA-6-1 | | | |
| Homo_sapiens_tRNA- | ATTCAAACCCCTACAGTCCT | CABE | 2446 |
| Tyr-GTA-6-1 | | | |
| Homo_sapiens_tRNA- | CATTCAAACCCCTACAGTCC | CABE | 2447 |
| Tyr-GTA-6-1 | | | |
| Homo_sapiens_tRNA- | ACATTCAAACCCCTACAGTC | CABE | 2448 |
| Tyr-GTA-6-1 | | | |
| Homo_sapiens_tRNA- | TACAGTCCTCCGCTCTACCA | CABE | 2449 |
| Tyr-GTA-7-1 | | | |
| Homo_sapiens_tRNA- | CTACAGTCCTCCGCTCTACC | CABE | 2450 |
| Tyr-GTA-7-1 | | | |
| Homo_sapiens_tRNA- | TCTACAGTCCTCCGCTCTAC | CABE | 2451 |
| Tyr-GTA-7-1 | | | |
| Homo_sapiens_tRNA- | GTCTACAGTCCTCCGCTCTA | CABE | 2452 |
| Tyr-GTA-7-1 | | | |
| Homo_sapiens_tRNA- | AGTCTACAGTCCTCCGCTCT | CABE | 2453 |
| Tyr-GTA-7-1 | | | |
| Homo_sapiens_tRNA- | CAGTCTACAGTCCTCCGCTC | CABE | 2454 |
| Tyr-GTA-7-1 | | | |
| Homo_sapiens_tRNA- | GCAGTCTACAGTCCTCCGCT | CABE | 2455 |
| Tyr-GTA-7-1 | | | |
| Homo_sapiens_tRNA- | CGCAGTCTACAGTCCTCCGC | CABE | 2456 |
| Tyr-GTA-7-1 | | | |
| Homo_sapiens_tRNA- | CCGCAGTCTACAGTCCTCCG | CABE | 2457 |
| Tyr-GTA-7-1 | | | |
| Homo_sapiens_tRNA- | TCCGCAGTCTACAGTCCTCC | CABE | 2458 |
| Tyr-GTA-7-1 | | | |
| Homo_sapiens_tRNA- | TTCCGCAGTCTACAGTCCTC | CABE | 2459 |
| Tyr-GTA-7-1 | | | |
| Homo_sapiens_tRNA- | TTTCCGCAGTCTACAGTCCT | CABE | 2460 |
| Tyr-GTA-7-1 | | | |
| Homo_sapiens_tRNA- | GTTTCCGCAGTCTACAGTCC | CABE | 2461 |
| Tyr-GTA-7-1 | | | |
| Homo_sapiens_tRNA- | CGTTTCCGCAGTCTACAGTC | CABE | 2462 |
| Tyr-GTA-7-1 | | | |
| Homo_sapiens_tRNA- | TACAGTCCTCCGCTCTACCA | CABE | 2463 |
| Tyr-GTA-8-1 | | | |
| Homo_sapiens_tRNA- | CTACAGTCCTCCGCTCTACC | CABE | 2464 |
| Tyr-GTA-8-1 | | | |
| Homo_sapiens_tRNA- | CCTACAGTCCTCCGCTCTAC | CABE | 2465 |
| Tyr-GTA-8-1 | | | |
| Homo_sapiens_tRNA- | ACCTACAGTCCTCCGCTCTA | CABE | 2466 |
| Tyr-GTA-8-1 | | | |
| Homo_sapiens_tRNA- | AACCTACAGTCCTCCGCTCT | CABE | 2467 |
| Tyr-GTA-8-1 | | | |
| Homo_sapiens_tRNA- | GAACCTACAGTCCTCCGCTC | CABE | 2468 |
| Tyr-GTA-8-1 | | | |
| Homo_sapiens_tRNA- | TGAACCTACAGTCCTCCGCT | CABE | 2469 |
| Tyr-GTA-8-1 | | | |
| Homo_sapiens_tRNA- | ATGAACCTACAGTCCTCCGC | CABE | 2470 |
| Tyr-GTA-8-1 | | | |
| Homo_sapiens_tRNA- | AATGAACCTACAGTCCTCCG | CABE | 2471 |
| Tyr-GTA-8-1 | | | |
| Homo_sapiens_tRNA- | TAATGAACCTACAGTCCTCC | CABE | 2472 |
| Tyr-GTA-8-1 | | | |
| Homo_sapiens_tRNA- | TTAATGAACCTACAGTCCTC | CABE | 2473 |
| Tyr-GTA-8-1 | | | |
| Homo_sapiens_tRNA- | TTTAATGAACCTACAGTCCT | CABE | 2474 |
| Tyr-GTA-8-1 | | | |
| Homo_sapiens_tRNA- | GTTTAATGAACCTACAGTCC | CABE | 2475 |
| Tyr-GTA-8-1 | | | |
| Homo_sapiens_tRNA- | AGTTTAATGAACCTACAGTC | CABE | 2476 |
| Tyr-GTA-8-1 | | | |
| Homo_sapiens_tRNA- | TACAGTCCTCCGCTCTACCA | CABE | 2477 |
| Tyr-GTA-9-1 | | | |
| Homo_sapiens_tRNA- | CTACAGTCCTCCGCTCTACC | CABE | 2478 |
| Tyr-GTA-9-1 | | | |
| Homo_sapiens_tRNA- | CCTACAGTCCTCCGCTCTAC | CABE | 2479 |
| Tyr-GTA-9-1 | | | |
| Homo_sapiens_tRNA- | ACCTACAGTCCTCCGCTCTA | CABE | 2480 |
| Tyr-GTA-9-1 | | | |
| Homo_sapiens_tRNA- | CACCTACAGTCCTCCGCTCT | CABE | 2481 |
| Tyr-GTA-9-1 | | | |
| Homo_sapiens_tRNA- | GCACCTACAGTCCTCCGCTC | CABE | 2482 |
| Tyr-GTA-9-1 | | | |
| Homo_sapiens_tRNA- | TGCACCTACAGTCCTCCGCT | CABE | 2483 |
| Tyr-GTA-9-1 | | | |
| Homo_sapiens_tRNA- | GTGCACCTACAGTCCTCCGC | CABE | 2484 |
| Tyr-GTA-9-1 | | | |
| Homo_sapiens_tRNA- | CGTGCACCTACAGTCCTCCG | CABE | 2485 |
| Tyr-GTA-9-1 | | | |
| Homo_sapiens_tRNA- | GCGTGCACCTACAGTCCTCC | CABE | 2486 |
| Tyr-GTA-9-1 | | | |
| Homo_sapiens_tRNA- | GGCGTGCACCTACAGTCCTC | CABE | 2487 |
| Tyr-GTA-9-1 | | | |
| Homo_sapiens_tRNA- | GGGCGTGCACCTACAGTCCT | CABE | 2488 |
| Tyr-GTA-9-1 | | | |
| Homo_sapiens_tRNA- | CGGGCGTGCACCTACAGTCC | CABE | 2489 |
| Tyr-GTA-9-1 | | | |
| Homo_sapiens_tRNA- | ACGGGCGTGCACCTACAGTC | CABE | 2490 |
| Tyr-GTA-9-1 | | | |

napDNAbp Domain

In some embodiments, the base editors of the present disclosure comprises a (napDNAbp) domain. Any suitable napDNAbp domain known in the art may be used in the base editors described herein, such as those described in detail in United State Patent Application [[XXXX]] by David Liu, et al., filed on Jan. 11, 2021, which is incorporated herein by reference in its entirety. For example, in various embodiments, the napDNAbp may be any Class 2 CRISPR-Cas system, including any type II, type V, or type VI CRISPR-Cas enzyme. Given the rapid development of CRISPR-Cas as a tool for genome editing, there have been constant developments in the nomenclature used to describe and/or identify CRISPR-Cas enzymes, such as Cas9 and Cas9 orthologs. This application references CRISPR-Cas enzymes with nomenclature that may be old and/or new as described in U.S. Patent Application 63/136,194 (described elsewhere herein) or Makarova et al., The CRISPR Journal, Vol. 1, No. 5, 2018, which is incorporated herein by reference in its entirety.

Other napDNAbps are also possible in other embodiments. For example, in some embodiments, the napDNAbp comprises the canonical SpCas9, or any ortholog Cas9 protein, or any variant Cas9 protein—including any naturally occurring variant, mutant, or otherwise engineered version of Cas9—that is known or that may be made or evolved through a directed evolutionary or otherwise mutagenic process. In various embodiments, the Cas9 or Cas9 variants have a nickase activity, i.e., only cleave one strand of the target DNA sequence. In other embodiments, the Cas9 or Cas9 variants have inactive nucleases, i.e., are “dead” Cas9 proteins. Other variant Cas9 proteins that may be used are those having a smaller molecular weight than the canonical SpCas9 (e.g., for easier delivery) or having modified or rearranged primary amino acid structure (e.g., the circular permutant formats).

In various embodiments described herein, the base editors comprise a napDNAbp, such as a Cas9 protein. These proteins are “programmable” by way of their becoming complexed with a guide RNA (or a pegRNA, as the case may be), which guides the Cas9 protein to a target site on the DNA which possess a sequence that is complementary to the spacer portion of the gRNA (or pegRNA) and also which possesses the required PAM sequence. However, in certain embodiment envisioned here, the napDNAbp may be substituted with a different type of programmable protein, such as a zinc finger nuclease or a transcription activator-like effector nuclease (TALEN). See U.S. Ser. No. 12/965,590; U.S. Ser. No. 13/426,991 (U.S. Pat. No. 8,450,471); U.S. Ser. No. 13/427,040 (U.S. Pat. No. 8,440,431); U.S. Ser. No. 13/427,137 (U.S. Pat. No. 8,440,432); and U.S. Ser. No. 13/738,381, all of which are incorporated by reference herein in their entirety. In addition, TALENS are described in WO 2015/027134, U.S. Pat. No. 9,181,535, Boch et al., “Breaking the Code of DNA Binding Specificity of TAL-Type III Effectors”, Science, vol. 326, pp. 1509-1512 (2009), Bogdanove et al., TAL Effectors: Customizable Proteins for DNA Targeting, Science, vol. 333, pp. 1843-1846 (2011), Cade et al., “Highly efficient generation of heritable zebrafish gene mutations using homo- and heterodimeric TALENs”, Nucleic Acids Research, vol. 40, pp. 8001-8010 (2012), and Cermak et al., “Efficient design and assembly of custom TALEN and other TAL effector-based constructs for DNA targeting”, Nucleic Acids Research, vol. 39, No. 17, e82 (2011), each of which are incorporated herein by reference. See also, for example, in Carroll et al., “Genome Engineering with Zinc-Finger Nucleases,” Genetics, August 2011, Vol. 188: 773-782; Durai et al., “Zinc finger nucleases: custom-designed molecular scissors for genome engineering of plant and mammalian cells,” Nucleic Acids Res, 2005, Vol. 33: 5978-90; and Gaj et al., “ZFN, TALEN, and CRISPR/Cas-based methods for genome engineering,” Trends Biotechnol. 2013, Vol. 31: 397-405, each of which are incorporated herein by reference in their entireties.

### Transition and Transversion Base Editors

### Base Editing and Deaminase Domains

In some embodiments, the fusion proteins described herein comprise a deaminase domain (e.g., when the Cas proteins provided herein are being used in the context of a base editor). A deaminase domain may be a cytosine deaminase domain or an adenosine deaminase domain.

Base editor fusion proteins that convert a C to T, in some embodiments, comprise a cytosine deaminase. A “cytosine deaminase” refers to an enzyme that catalyzes the chemical reaction “cytosine+H2O→uracil+NH3” or “5-methyl-cytosine+H2O→thymine+NH3.” As it may be apparent from the reaction formula, such chemical reactions result in a C to U/T nucleobase change. In the context of a gene, such a nucleotide change, or mutation, may in turn lead to an amino acid change in the protein, which may affect the protein's function, e.g., loss-of-function or gain-of-function. In some embodiments, the C to T base editor comprises a Cas14a1 variant provided herein fused to a cytosine deaminase. In some embodiments, the cytosine deaminase domain is fused to the N-terminus of the Cas14a1 variant.

Non-limiting examples of suitable cytosine deaminase domains are provided below, as SEQ ID NOs: 17-50.

Human AID
(SEQ ID NO: 17)
MDSLLMNRRKFLYQFKNVRWAKGRRETYLCYVVKRRDSATSFSLDFGYLR
NKNGCHVELLFLRYISDWDLDPGRCYRVTWFTSWSPCYDCARHVADFLRGNPNLSLRI
FTARLYFCEDRKAEPEGLRRLHRAGVQIAIMTFKDYFYCWNTFVENHERTFKAWEGLH
ENSVRLSRQLRRILLPLYEVDDLRDAFRTLGL
Mouse AID
(SEQ ID NO: 18)
MDSLLMKQKKFLYHFKNVRWAKGRHETYLCYVVKRRDSATSCSLDFGHLR
NKSGCHVELLFLRYISDWDLDPGRCYRVTWFTSWSPCYDCARHVAEFLRWNPNLSLRIF
TARLYFCEDRKAEPEGLRRLHRAGVQIGIMTFKDYFYCWNTFVENRERTFKAWEGLHE
NSVRLTRQLRRILLPLYEVDDLRDAFRMLGF
Dog AID
(SEQ ID NO: 19)
MDSLLMKQRKFLYHFKNVRWAKGRHETYLCYVVKRRDSATSFSLDFGHLR
NKSGCHVELLFLRYISDWDLDPGRCYRVTWFTSWSPCYDCARHVADFLRGYPNLSLRIF
AARLYFCEDRKAEPEGLRRLHRAGVQIAIMTFKDYFYCWNTFVENREKTFKAWEGLHE
NSVRLSRQLRRILLPLYEVDDLRDAFRTLGL
Bovine AID
(SEQ ID NO: 20)
MDSLLKKQRQFLYQFKNVRWAKGRHETYLCYVVKRRDSPTSFSLDFGHLRN
KAGCHVELLFLRYISDWDLDPGRCYRVTWFTSWSPCYDCARHVADFLRGYPNLSLRIFT
ARLYFCDKERKAEPEGLRRLHRAGVQIAIMTFKDYFYCWNTFVENHERTFKAWEGLHE
NSVRLSRQLRRILLPLYEVDDLRDAFRTLGL
Mouse APOBEC-3
(SEQ ID NO: 21)
MGPFCLGCSHRKCYSPIRNLISQETFKFHFKNLGYAKGRKDTFLCYEVTRKD
CDSPVSLHHGVFKNKDNIHAEICFLYWFHDKVLKVLSPREEFKITWYMSWSPCFECAEQ
IVRFLATHHNLSLDIFSSRLYNVQDPETQQNLCRLVQEGAQVAAMDLYEFKKCWKKFV
DNGGRRFRPWKRLLTNFRYQDSKLQEILRPCYIPVPSSSSSTLSNICLTKGLPETRFCVEG
RRMDPLSEEEFYSQFYNQRVKHLCYYHRMKPYLCYQLEQFNGQAPLKGCLLSEKGKQ
HAEILFLDKIRSMELSQVTITCYLTWSPCPNCAWQLAAFKRDRPDLILHIYTSRLYFHWK
RPFQKGLCSLWQSGILVDVMDLPQFTDCWTNFVNPKRPFWPWKGLEIISRRTQRRLRRI
KESWGLQDLVNDFGNLQLGPPMS
Rat APOBEC-3
(SEQ ID NO: 22)
MGPFCLGCSHRKCYSPIRNLISQETFKFHFKNLRYAIDRKDTFLCYEVTRKDC
DSPVSLHHGVFKNKDNIHAEICFLYWFHDKVLKVLSPREEFKITWYMSWSPCFECAEQV
LRFLATHHNLSLDIFSSRLYNIRDPENQQNLCRLVQEGAQVAAMDLYEFKKCWKKFVD
NGGRRFRPWKKLLTNFRYQDSKLQEILRPCYIPVPSSSSSTLSNICLTKGLPETRFCVERR
RVHLLSEEEFYSQFYNQRVKHLCYYHGVKPYLCYQLEQFNGQAPLKGCLLSEKGKQHA
EILFLDKIRSMELSQVIITCYLTWSPCPNCAWQLAAFKRDRPDLILHIYTSRLYFHWKRPF
QKGLCSLWQSGILVDVMDLPQFTDCWTNFVNPKRPFWPWKGLEIISRRTQRRLHRIKES
WGLQDLVNDFGNLQLGPPMS
Rhesus macaque APOBEC-3G
(SEQ ID NO: 23)
MVEPMDPRTFVSNFNNRPILSGLNTVWLCCEVKTKDPSGPPLDAKIFQGKVY
SKAKYHPEMRFLRWFHKWRQLHHDQEYKVTWYVSWSPCTRCANSVATFLAKDPKVT
LTIFVARLYYFWKPDYQQALRILCQKRGGPHATMKIMNYNEFQDCWNKFVDGRGKPF
KPRNNLPKHYTLLQATLGELLRHLMDPGTFTSNFNNKPWVSGQHETYLCYKVERLHND
TWVPLNQHRGFLRNQAPNIHGFPKGRHAELCFLDLIPFWKLDGQQYRVTCFTSWSPCFS
CAQEMAKFISNNEHVSLCIFAARIYDDQGRYQEGLRALHRDGAKIAMMNYSEFEYCWD
TFVDRQGRPFQPWDGLDEHSQALSGRLRAI
Chimpanzee APOBEC-3G
(SEQ ID NO: 24)
MKPHFRNPVERMYQDTFSDNFYNRPILSHRNTVWLCYEVKTKGPSRPPLDA
KIFRGQVYSKLKYHPEMRFFHWFSKWRKLHRDQEYEVTWYISWSPCTKCTRDVATFLA
EDPKVTLTIFVARLYYFWDPDYQEALRSLCQKRDGPRATMKIMNYDEFQHCWSKFVYS
QRELFEPWNNLPKYYILLHIMLGEILRHSMDPPTFTSNFNNELWVRGRHETYLCYEVERL
HNDTWVLLNQRRGFLCNQAPHKHGFLEGRHAELCFLDVIPFWKLDLHQDYRVTCFTSW
SPCFSCAQEMAKFISNNKHVSLCIFAARIYDDQGRCQEGLRTLAKAGAKISIMTYSEFKH
CWDTFVDHQGCPFQPWDGLEEHSQALSGRLRAILQNQGN
Green monkey APOBEC-3G
(SEQ ID NO: 25)
MNPQIRNMVEQMEPDIFVYYFNNRPILSGRNTVWLCYEVKTKDPSGPPLDAN
IFQGKLYPEAKDHPEMKFLHWFRKWRQLHRDQEYEVTWYVSWSPCTRCANSVATFLA
EDPKVTLTIFVARLYYFWKPDYQQALRILCQERGGPHATMKIMNYNEFQHCWNEFVDG
QGKPFKPRKNLPKHYTLLHATLGELLRHVMDPGTFTSNFNNKPWVSGQRETYLCYKVE
RSHNDTWVLLNQHRGFLRNQAPDRHGFPKGRHAELCFLDLIPFWKLDDQQYRVTCFTS
WSPCFSCAQKMAKFISNNKHVSLCIFAARIYDDQGRCQEGLRTLHRDGAKIAVMNYSEF
EYCWDTFVDRQGRPFQPWDGLDEHSQALSGRLRAI
Human APOBEC-3G
(SEQ ID NO: 26)
MKPHFRNTVERMYRDTFSYNFYNRPILSRRNTVWLCYEVKTKGPSRPPLDAK
IFRGQVYSELKYHPEMRFFHWFSKWRKLHRDQEYEVTWYISWSPCTKCTRDMATFLAE
DPKVTLTIFVARLYYFWDPDYQEALRSLCQKRDGPRATMKIMNYDEFQHCWSKFVYSQ
RELFEPWNNLPKYYILLHIMLGEILRHSMDPPTFTFNFNNEPWVRGRHETYLCYEVERM
HNDTWVLLNQRRGFLCNQAPHKHGFLEGRHAELCFLDVIPFWKLDLDQDYRVTCFTSW
SPCFSCAQEMAKFISKNKHVSLCIFTARIYDDQGRCQEGLRTLAEAGAKISIMTYSEFKH
CWDTFVDHQGCPFQPWDGLDEHSQDLSGRLRAILQNQEN
Human APOBEC-3F
(SEQ ID NO: 27)
MKPHFRNTVERMYRDTFSYNFYNRPILSRRNTVWLCYEVKTKGPSRPRLDA
KIFRGQVYSQPEHHAEMCFLSWFCGNQLPAYKCFQITWFVSWTPCPDCVAKLAEFLAE
HPNVTLTISAARLYYYWERDYRRALCRLSQAGARVKIMDDEEFAYCWENFVYSEGQPF
MPWYKFDDNYAFLHRTLKEILRNPMEAMYPHIFYFHFKNLRKAYGRNESWLCFTMEV
VKHHSPVSWKRGVFRNQVDPETHCHAERCFLSWFCDDILSPNTNYEVTWYTSWSPCPE
CAGEVAEFLARHSNVNLTIFTARLYYFWDTDYQEGLRSLSQEGASVEIMGYKDFKYCW
ENFVYNDDEPFKPWKGLKYNFLFLDSKLQEILE
Human APOBEC-3B
(SEQ ID NO: 28)
MNPQIRNPMERMYRDTFYDNFENEPILYGRSYTWLCYEVKIKRGRSNLLWD
TGVFRGQVYFKPQYHAEMCFLSWFCGNQLPAYKCFQITWFVSWTPCPDCVAKLAEFLS
EHPNVTLTISAARLYYYWERDYRRALCRLSQAGARVTIMDYEEFAYCWENFVYNEGQQ
FMPWYKFDENYAFLHRTLKEILRYLMDPDTFTFNFNNDPLVLRRRQTYLCYEVERLDN
GTWVLMDQHMGFLCNEAKNLLCGFYGRHAELRFLDLVPSLQLDPAQIYRVTWFISWSP
CFSWGCAGEVRAFLQENTHVRLRIFAARIYDYDPLYKEALQMLRDAGAQVSIMTYDEF
EYCWDTFVYRQGCPFQPWDGLEEHSQALSGRLRAILQNQGN
Human APOBEC-3C
(SEQ ID NO: 29)
MNPQIRNPMKAMYPGTFYFQFKNLWEANDRNETWLCFTVEGIKRRSVVSW
KTGVFRNQVDSETHCHAERCFLSWFCDDILSPNTKYQVTWYTSWSPCPDCAGEVAEFL
ARHSNVNLTIFTARLYYFQYPCYQEGLRSLSQEGVAVEIMDYEDFKYCWENFVYNDNE
PFKPWKGLKTNFRLLKRRLRESLQ
Human APOBEC-3A
(SEQ ID NO: 30)
MEASPASGPRHLMDPHIFTSNFNNGIGRHKTYLCYEVERLDNGTSVKMDQH
RGFLHNQAKNLLCGFYGRHAELRFLDLVPSLQLDPAQIYRVTWFISWSPCFSWGCAGEV
RAFLQENTHVRLRIFAARIYDYDPLYKEALQMLRDAGAQVSIMTYDEFKHCWDTFVDH
QGCPFQPWDGLDEHSQALSGRLRAILQNQGN
Human APOBEC-3H
(SEQ ID NO: 31)
MALLTAETFRLQFNNKRRLRRPYYPRKALLCYQLTPQNGSTPTRGYFENKKK
CHAEICFINEIKSMGLDETQCYQVTCYLTWSPCSSCAWELVDFIKAHDHLNLGIFASRLY
YHWCKPQQKGLRLLCGSQVPVEVMGFPKFADCWENFVDHEKPLSFNPYKMLEELDKN
SRAIKRRLERIKIPGVRAQGRYMDILCDAEV
Human APOBEC-3D
(SEQ ID NO: 32)
MNPQIRNPMERMYRDTFYDNFENEPILYGRSYTWLCYEVKIKRGRSNLLWD
TGVFRGPVLPKRQSNHRQEVYFRFENHAEMCFLSWFCGNRLPANRRFQITWFVSWNPC
LPCVVKVTKFLAEHPNVTLTISAARLYYYRDRDWRWVLLRLHKAGARVKIMDYEDFA
YCWENFVCNEGQPFMPWYKFDDNYASLHRTLKEILRNPMEAMYPHIFYFHFKNLLKAC
GRNESWLCFTMEVTKHHSAVFRKRGVFRNQVDPETHCHAERCFLSWFCDDILSPNTNY
EVTWYTSWSPCPECAGEVAEFLARHSNVNLTIFTARLCYFWDTDYQEGLCSLSQEGASV
KIMGYKDFVSCWKNFVYSDDEPFKPWKGLQTNFRLLKRRLREILQ
Human APOBEC-1
(SEQ ID NO: 33)
MTSEKGPSTGDPTLRRRIEPWEFDVFYDPRELRKEACLLYEIKWGMSRKIWR
SSGKNTTNHVEVNFIKKFTSERDFHPSMSCSITWFLSWSPCWECSQAIREFLSRHPGVTL
VIYVARLFWHMDQQNRQGLRDLVNSGVTIQIMRASEYYHCWRNFVNYPPGDEAHWPQ
YPPLWMMLYALELHCIILSLPPCLKISRRWQNHLTFFRLHLQNCHYQTIPPHILLATGLIH
PSVAWR
Mouse APOBEC-1
(SEQ ID NO: 34)
MSSETGPVAVDPTLRRRIEPHEFEVFFDPRELRKETCLLYEINWGGRHSVWRH
TSQNTSNHVEVNFLEKFTTERYFRPNTRCSITWFLSWSPCGECSRAITEFLSRHPYVTLFI
YIARLYHHTDQRNRQGLRDLISSGVTIQIMTEQEYCYCWRNFVNYPPSNEAYWPRYPHL
WVKLYVLELYCIILGLPPCLKILRRKQPQLTFFTITLQTCHYQRIPPHLLWATGLK
Rat APOBEC-1
(SEQ ID NO: 35)
MSSETGPVAVDPTLRRRIEPHEFEVFFDPRELRKETCLLYEINWGGRHSIWRH
TSQNTNKHVEVNFIEKFTTERYFCPNTRCSITWFLSWSPCGECSRAITEFLSRYPHVTLFIY
IARLYHHADPRNRQGLRDLISSGVTIQIMTEQESGYCWRNFVNYSPSNEAHWPRYPHLW
VRLYVLELYCIILGLPPCLNILRRKQPQLTFFTIALQSCHYQRLPPHILWATGLK
Petromyzon marinus CDA1 (pmCDA1)
(SEQ ID NO: 36)
MTDAEYVRIHEKLDIYTFKKQFFNNKKSVSHRCYVLFELKRRGERRACFWG
YAVNKPQSGTERGIHAEIFSIRKVEEYLRDNPGQFTINWYSSWSPCADCAEKILEWYNQE
LRGNGHTLKIWACKLYYEKNARNQIGLWNLRDNGVGLNVMVSEHYQCCRKIFIQSSHN
QLNENRWLEKTLKRAEKRRSELSIMIQVKILHTTKSPAV
Evolved pmCDA1 (evoCDA1)
(SEQ ID NO: 37)
MTDAEYVRIHEKLDIYTFKKQFSNNKKSVSHRCYVLFELKRRGERRACFWG
YAVNKPQSGTERGIHAEIFSIRKVEEYLRDNPGQFTINWYSSWSPCADCAEKILEWYNQE
LRGNGHTLKIWVCKLYYEKNARNQIGLWNLRDNGVGLNVMVSEHYQCCRKIFIQSSHN
QLNENRWLEKTLKRAEKRRSELSIMFQVKILHTTKSPAV
Human APOBEC3G D316R_D317R
(SEQ ID NO: 38)
MKPHFRNTVERMYRDTFSYNFYNRPILSRRNTVWLCYEVKTKGPSRPPLDAK
IFRGQVYSELKYHPEMRFFHWFSKWRKLHRDQEYEVTWYISWSPCTKCTRDMATFLAE
DPKVTLTIFVARLYYFWDPDYQEALRSLCQKRDGPRATMKIMNYDEFQHCWSKFVYSQ
RELFEPWNNLPKYYILLHIMLGEILRHSMDPPTFTFNFNNEPWVRGRHETYLCYEVERM
HNDTWVLLNQRRGFLCNQAPHKHGFLEGRHAELCFLDVIPFWKLDLDQDYRVTCFTSW
SPCFSCAQEMAKFISKNKHVSLCIFTARIYRRQGRCQEGLRTLAEAGAKISIMTYSEFKHC
WDTFVDHQGCPFQPWDGLDEHSQDLSGRLRAILQNQEN
Human APOBEC3G chain A
(SEQ ID NO: 39)
MDPPTFTFNFNNEPWVRGRHETYLCYEVERMHNDTWVLLNQRRGFLCNQA
PHKHGFLEGRHAELCFLDVIPFWKLDLDQDYRVTCFTSWSPCFSCAQEMAKFISKNKHV
SLCIFTARIYDDQGRCQEGLRTLAEAGAKISIMTYSEFKHCWDTFVDHQGCPFQPWDGL
DEHSQDLSGRLRAILQ
Human APOBEC3G chain A D120R_D121R
(SEQ ID NO: 40)
MDPPTFTFNFNNEPWVRGRHETYLCYEVERMHNDTWVLLNQRRGFLCNQA
PHKHGFLEGRHAELCFLDVIPFWKLDLDQDYRVTCFTSWSPCFSCAQEMAKFISKNKHV
SLCIFTARIYRRQGRCQEGLRTLAEAGAKISIMTYSEFKHCWDTFVDHQGCPFQPWDGL
DEHSQDLSGRLRAILQ
evo APOBEC1
(SEQ ID NO: 41)
MSSKTGPVAVDPTLRRRIEPHEFEVFFDPRELRKETCLLYEINWGGRHSIWRH
TSQNTNKHVEVNFIEKFTTERYFCPNTRCSITWFLSWSPCGECSRAITEFLSRYPNVTLFIY
IARLYHLANPRNRQGLRDLISSGVTIQIMTEQESGYCWHNFVNYSPSNESHWPRYPHLW
VRLYVLELYCIILGLPPCLNILRRKQSQLTSFTIALQSCHYQRLPPHILWATGLK
YE1
(SEQ ID NO: 42)
MSSETGPVAVDPTLRRRIEPHEFEVFFDPRELRKETCLLYEINWGGRHSIWRH
TSQNTNKHVEVNFIEKFTTERYFCPNTRCSITWFLSYSPCGECSRAITEFLSRYPHVTLFIY
IARLYHHADPENRQGLRDLISSGVTIQIMTEQESGYCWRNFVNYSPSNEAHWPRYPHLW
VRLYVLELYCIILGLPPCLNILRRKQPQLTFFTIALQSCHYQRLPPHILWATGLK
YE2
(SEQ ID NO: 43)
MSSETGPVAVDPTLRRRIEPHEFEVFFDPRELRKETCLLYEINWGGRHSIWRH
TSQNTNKHVEVNFIEKFTTERYFCPNTRCSITWFLSYSPCGECSRAITEFLSRYPHVTLFIY
IARLYHHADPRNRQGLEDLISSGVTIQIMTEQESGYCWRNFVNYSPSNEAHWPRYPHLW
VRLYVLELYCIILGLPPCLNILRRKQPQLTFFTIALQSCHYQRLPPHILWATGLK
YEE
(SEQ ID NO: 44)
MSSETGPVAVDPTLRRRIEPHEFEVFFDPRELRKETCLLYEINWGGRHSIWRH
TSQNTNKHVEVNFIEKFTTERYFCPNTRCSITWFLSYSPCGECSRAITEFLSRYPHVTLFIY
IARLYHHADPENRQGLEDLISSGVTIQIMTEQESGYCWRNFVNYSPSNEAHWPRYPHLW
VRLYVLELYCIILGLPPCLNILRRKQPQLTFFTIALQSCHYQRLPPHILWATGLK
EE
(SEQ ID NO: 45)
MSSETGPVAVDPTLRRRIEPHEFEVFFDPRELRKETCLLYEINWGGRHSIWRH
TSQNTNKHVEVNFIEKFTTERYFCPNTRCSITWFLSWSPCGECSRAITEFLSRYPHVTLFIY
IARLYHHADPENRQGLEDLISSGVTIQIMTEQESGYCWRNFVNYSPSNEAHWPRYPHLW
VRLYVLELYCIILGLPPCLNILRRKQPQLTFFTIALQSCHYQRLPPHILWATGLK
R33A
(SEQ ID NO: 46)
MSSETGPVAVDPTLRRRIEPHEFEVFFDPRELAKETCLLYEINWGGRHSIWRH
TSQNTNKHVEVNFIEKFTTERYFCPNTRCSITWFLSWSPCGECSRAITEFLSRYPHVTLFIY
IARLYHHADPRNRQGLRDLISSGVTIQIMTEQESGYCWRNFVNYSPSNEAHWPRYPHLW
VRLYVLELYCIILGLPPCLNILRRKQPQLTFFTIALQSCHYQRLPPHILWATGLK
R33A + K34A
(SEQ ID NO: 47)
MSSETGPVAVDPTLRRRIEPHEFEVFFDPRELAAETCLLYEINWGGRHSIWRH
TSQNTNKHVEVNFIEKFTTERYFCPNTRCSITWFLSWSPCGECSRAITEFLSRYPHVTLFIY
IARLYHHADPRNRQGLRDLISSGVTIQIMTEQESGYCWRNFVNYSPSNEAHWPRYPHLW
VRLYVLELYCIILGLPPCLNILRRKQPQLTFFTIALQSCHYQRLPPHILWATGLK
AALN
(SEQ ID NO: 48)
MSSETGPVAVDPTLRRRIEPHEFEVFFDPRELAAETCLLYEINWGGRHSIWRH
TSQNTNKHVEVNFIEKFTTERYFCPNTRCSITWFLSWSPCGECSRAITEFLSRYPHVTLFIY
IARLYHLANPRNRQGLRDLISSGVTIQIMTEQESGYCWRNFVNYSPSNEAHWPRYPHLW
VRLYVLELYCIILGLPPCLNILRRKQPQLTFFTIALQSCHYQRLPPHILWATGLK
FERNY
(SEQ ID NO: 49)
MFERNYDPRELRKETYLLYEIKWGKSGKLWRHWCQNNRTQHAEVYFLENIF
NARRENPSTHCSITWYLSWSPCAECSQKIVDFLKEHPNVNLEIYVARLYYHEDERNRQG
LRDLVNSGVTIRIMDLPDYNYCWKTFVSDQGGDEDYWPGHFAPWIKQYSLKL
evo FERNY
(SEQ ID NO: 50)
MFERNYDPRELRKETYLLYEIKWGKSGKLWRHWCQNNRTQHAEVYFLENIF
NARRFNPSTHCSITWYLSWSPCAECSQKIVDFLKEHPNVNLEIYVARLYYPENERNRQG
LRDLVNSGVTIRIMDLPDYNYCWKTFVSDQGGDEDYWPGHFAPWIKQYSLKL

In some embodiments, a base editor fusion protein converts an A to G. In some embodiments, the base editor comprises an adenosine deaminase. An “adenosine deaminase” is an enzyme involved in purine metabolism. It is needed for the breakdown of adenosine from food and for the turnover of nucleic acids in tissues. Its primary function in humans is the development and maintenance of the immune system. An adenosine deaminase catalyzes hydrolytic deamination of adenosine (forming inosine, which base pairs as G) in the context of DNA. There are no known adenosine deaminases that act on DNA. Instead, known adenosine deaminase enzymes only act on RNA (tRNA or mRNA). Evolved deoxyadenosine deaminase enzymes that accept DNA substrates and deaminate dA to deoxyinosine for use in adenosine nucleobase editors have been described, e.g., in PCT Application PCT/US2017/045381, filed Aug. 3, 2017, which published as WO 2018/027078, PCT Application No. PCT/US2019/033848, which published as WO 2019/226953 on May 23, 2019, PCT Application No PCT/US2019/033848, filed May 23, 2019, and PCT Application No. PCT/US2020/028568, filed Apr. 17, 2020; each of which is herein incorporated by reference. Non-limiting examples of evolved adenosine deaminases that accept DNA as substrates are provided below. In some embodiments, an adenosine deaminase comprises any of the following amino acid sequences, or an amino acid sequence at least 70%, at least 75%, at least 80%, at least 85%, at least 90%, at least 95%, at least 99%, or at least 99.9% identical to any of the following amino acid sequences (SEQ ID NOs: 51-118):

ecTadA
(SEQ ID NO: 51)
SEVEFSHEYWMRHALTLAKRAWDEREVPVGAVLVHNNRVIGEGWNRPIGR
HDPTAHAEIMALRQGGLVMQNYRLIDATLYVTLEPCVMCAGAMIHSRIGRVVFGARDA
KTGAAGSLMDVLHHPGMNHRVEITEGILADECAALLSDFFRMRRQEIKAQKKAQSSTD
ecTadA (D108N)
(SEQ ID NO: 52)
SEVEFSHEYWMRHALTLAKRAWDEREVPVGAVLVHNNRVIGEGWNRPIGR
HDPTAHAEIMALRQGGLVMQNYRLIDATLYVTLEPCVMCAGAMIHSRIGRVVFGARNA
KTGAAGSLMDVLHHPGMNHRVEITEGILADECAALLSDFFRMRRQEIKAQKKAQSSTD
ecTadA (D108G)
(SEQ ID NO: 53)
SEVEFSHEYWMRHALTLAKRAWDEREVPVGAVLVHNNRVIGEGWNRPIGR
HDPTAHAEIMALRQGGLVMQNYRLIDATLYVTLEPCVMCAGAMIHSRIGRVVFGARGA
KTGAAGSLMDVLHHPGMNHRVEITEGILADECAALLSDFFRMRRQEIKAQKKAQSSTD
ecTadA (D108V)
(SEQ ID NO: 54)
SEVEFSHEYWMRHALTLAKRAWDEREVPVGAVLVHNNRVIGEGWNRPIGR
HDPTAHAEIMALRQGGLVMQNYRLIDATLYVTLEPCVMCAGAMIHSRIGRVVFGARVA
KTGAAGSLMDVLHHPGMNHRVEITEGILADECAALLSDFFRMRRQEIKAQKKAQSSTD
ecTadA (H8Y, D108N, N127S)
(SEQ ID NO: 55)
SEVEFSYEYWMRHALTLAKRAWDEREVPVGAVLVHNNRVIGEGWNRPIGR
HDPTAHAEIMALRQGGLVMQNYRLIDATLYVTLEPCVMCAGAMIHSRIGRVVFGARNA
KTGAAGSLMDVLHHPGMSHRVEITEGILADECAALLSDFFRMRRQEIKAQKKAQSSTD
ecTadA (H8Y, D108N, N127S, E155D)
(SEQ ID NO: 56)
SEVEFSYEYWMRHALTLAKRAWDEREVPVGAVLVHNNRVIGEGWNRPIGR
HDPTAHAEIMALRQGGLVMQNYRLIDATLYVTLEPCVMCAGAMIHSRIGRVVFGARNA
KTGAAGSLMDVLHHPGMSHRVEITEGILADECAALLSDFFRMRRQDIKAQKKAQSSTD
ecTadA (H8Y, D108N, N127S, E155G)
(SEQ ID NO: 57)
SEVEFSYEYWMRHALTLAKRAWDEREVPVGAVLVHNNRVIGEGWNRPIGR
HDPTAHAEIMALRQGGLVMQNYRLIDATLYVTLEPCVMCAGAMIHSRIGRVVFGARNA
KTGAAGSLMDVLHHPGMSHRVEITEGILADECAALLSDFFRMRRQGIKAQKKAQSSTD
ecTadA (H8Y, D108N, N127S, E155V)
(SEQ ID NO: 58)
SEVEFSYEYWMRHALTLAKRAWDEREVPVGAVLVHNNRVIGEGWNRPIGR
HDPTAHAEIMALRQGGLVMQNYRLIDATLYVTLEPCVMCAGAMIHSRIGRVVFGARNA
KTGAAGSLMDVLHHPGMSHRVEITEGILADECAALLSDFFRMRRQVIKAQKKAQSSTD
ecTadA (A106V, D108N, D147Y, and E155V)
(SEQ ID NO: 59)
SEVEFSHEYWMRHALTLAKRAWDEREVPVGAVLVHNNRVIGEGWNRPIGR
HDPTAHAEIMALRQGGLVMQNYRLIDATLYVTLEPCVMCAGAMIHSRIGRVVFGVRNA
KTGAAGSLMDVLHHPGMNHRVEITEGILADECAALLSYFFRMRRQVIKAQKKAQSSTD
ecTadA (S2A, I49F, A106V, D108N, D147Y, E155V)
(SEQ ID NO: 60)
AEVEFSHEYWMRHALTLAKRAWDEREVPVGAVLVHNNRVIGEGWNRPFGR
HDPTAHAEIMALRQGGLVMQNYRLIDATLYVTLEPCVMCAGAMIHSRIGRVVFGVRNA
KTGAAGSLMDVLHHPGMNHRVEITEGILADECAALLSYFFRMRRQVIKAQKKAQSSTD
ecTadA (H8Y, A106T, D108N, N127S, K160S)
(SEQ ID NO: 61)
SEVEFSYEYWMRHALTLAKRAWDEREVPVGAVLVHNNRVIGEGWNRPIGR
HDPTAHAEIMALRQGGLVMQNYRLIDATLYVTLEPCVMCAGAMIHSRIGRVVFGTRNA
KTGAAGSLMDVLHHPGMSHRVEITEGILADECAALLSDFFRMRRQEIKAQSKAQSSTD
ecTadA (R26G, L84F, A106V, R107H, D108N, H123Y, A142N, A143D, D147Y,
E155V, I156F)
(SEQ ID NO: 62)
SEVEFSHEYWMRHALTLAKRAWDEGEVPVGAVLVHNNRVIGEGWNRPIGR
HDPTAHAEIMALRQGGLVMQNYRLIDATLYVTFEPCVMCAGAMIHSRIGRVVFGVHNA
KTGAAGSLMDVLHYPGMNHRVEITEGILADECNDLLSYFFRMRRQVFKAQKKAQSSTD
ecTadA (E25G, R26G, L84F, A106V, R107H, D108N, H123Y, A142N, A143D,
D147Y, E155V, I156F)
(SEQ ID NO: 63)
SEVEFSHEYWMRHALTLAKRAWDGGEVPVGAVLVHNNRVIGEGWNRPIGR
HDPTAHAEIMALRQGGLVMQNYRLIDATLYVTFEPCVMCAGAMIHSRIGRVVFGVHNA
KTGAAGSLMDVLHYPGMNHRVEITEGILADECNDLLSYFFRMRRQVFKAQKKAQSSTD
ecTadA (E25D, R26G, L84F, A106V, R107K, D108N, H123Y, A142N, A143G,
D147Y, E155V, I156F)
(SEQ ID NO: 64)
SEVEFSHEYWMRHALTLAKRAWDDGEVPVGAVLVHNNRVIGEGWNRPIGR
HDPTAHAEIMALRQGGLVMQNYRLIDATLYVTFEPCVMCAGAMIHSRIGRVVFGVKNA
KTGAAGSLMDVLHYPGMNHRVEITEGILADECNGLLSYFFRMRRQVFKAQKKAQSSTD
ecTadA (R26Q, L84F, A106V, D108N, H123Y, A142N, D147Y, E155V, I156F)
(SEQ ID NO: 65)
SEVEFSHEYWMRHALTLAKRAWDEQEVPVGAVLVHNNRVIGEGWNRPIGR
HDPTAHAEIMALRQGGLVMQNYRLIDATLYVTFEPCVMCAGAMIHSRIGRVVFGVRNA
KTGAAGSLMDVLHYPGMNHRVEITEGILADECNALLSYFFRMRRQVFKAQKKAQSSTD
ecTadA (E25M, R26G, L84F, A106V, R107P, D108N, H123Y, A142N, A143D,
D147Y, E155V, I156F)
(SEQ ID NO: 66)
SEVEFSHEYWMRHALTLAKRAWDMGEVPVGAVLVHNNRVIGEGWNRPIGR
HDPTAHAEIMALRQGGLVMQNYRLIDATLYVTFEPCVMCAGAMIHSRIGRVVFGVPNA
KTGAAGSLMDVLHYPGMNHRVEITEGILADECNDLLSYFFRMRRQVFKAQKKAQSSTD
ecTadA (R26C, L84F, A106V, R107H, D108N, H123Y, A142N, D147Y, E155V,
I156F)
(SEQ ID NO: 67)
SEVEFSHEYWMRHALTLAKRAWDECEVPVGAVLVHNNRVIGEGWNRPIGR
HDPTAHAEIMALRQGGLVMQNYRLIDATLYVTFEPCVMCAGAMIHSRIGRVVFGVHNA
KTGAAGSLMDVLHYPGMNHRVEITEGILADECNALLSYFFRMRRQVFKAQKKAQSSTD
ecTadA (L84F, A106V, D108N, H123Y, A142N, A143L, D147Y, E155V, I156F)
(SEQ ID NO: 68)
SEVEFSHEYWMRHALTLAKRAWDEREVPVGAVLVHNNRVIGEGWNRPIGR
HDPTAHAEIMALRQGGLVMQNYRLIDATLYVTFEPCVMCAGAMIHSRIGRVVFGVRNA
KTGAAGSLMDVLHYPGMNHRVEITEGILADECNLLLSYFFRMRRQVFKAQKKAQSSTD
ecTadA (R26G, L84F, A106V, D108N, H123Y, A142N, D147Y, E155V, I156F)
(SEQ ID NO: 69)
SEVEFSHEYWMRHALTLAKRAWDEGEVPVGAVLVHNNRVIGEGWNRPIGR
HDPTAHAEIMALRQGGLVMQNYRLIDATLYVTFEPCVMCAGAMIHSRIGRVVFGVRNA
KTGAAGSLMDVLHYPGMNHRVEITEGILADECNALLSYFFRMRRQVFKAQKKAQSSTD
ecTadA (R51H, L84F, A106V, D108N, H123Y, D147Y, E155V, 1156F, K157N)
(SEQ ID NO: 70)
SEVEFSHEYWMRHALTLAKRAWDEREVPVGAVLVHNNRVIGEGWNRPIGH
HDPTAHAEIMALRQGGLVMQNYRLIDATLYVTFEPCVMCAGAMIHSRIGRVVFGVRNA
KTGAAGSLMDVLHYPGMNHRVEITEGILADECAALLSYFFRMRRQVFNAQKKAQSSTD
ecTadA (E25A, R26G, L84F, A106V, R107N, D108N, H123Y, A142N, A143E,
D147Y, E155V, I156F)
(SEQ ID NO: 71)
SEVEFSHEYWMRHALTLAKRAWDAGEVPVGAVLVHNNRVIGEGWNRPIGR
HDPTAHAEIMALRQGGLVMQNYRLIDATLYVTFEPCVMCAGAMIHSRIGRVVFGVNNA
KTGAAGSLMDVLHYPGMNHRVEITEGILADECNELLSYFFRMRRQVFKAQKKAQSSTD
ecTadA (N37T, P48T, L84F, A106V, D108N, H123Y, D147Y, E155V, I156F)
(SEQ ID NO: 72)
SEVEFSHEYWMRHALTLAKRAWDEREVPVGAVLVHTNRVIGEGWNRTIGR
HDPTAHAEIMALRQGGLVMQNYRLIDATLYVTFEPCVMCAGAMIHSRIGRVVFGVRNA
KTGAAGSLMDVLHYPGMNHRVEITEGILADECAALLSYFFRMRRQVFKAQKKAQSSTD
ecTadA (N37S, L84F, A106V, D108N, H123Y, D147Y, E155V, 1156F)
(SEQ ID NO: 73)
SEVEFSHEYWMRHALTLAKRAWDEREVPVGAVLVHSNRVIGEGWNRPIGRH
DPTAHAEIMALRQGGLVMQNYRLIDATLYVTFEPCVMCAGAMIHSRIGRVVFGVRNAK
TGAAGSLMDVLHYPGMNHRVEITEGILADECAALLSYFFRMRRQVFKAQKKAQSSTD
ecTadA (H36L, L84F, A106V, D108N, H123Y, D147Y, E155V, 1156F)
(SEQ ID NO: 74)
SEVEFSHEYWMRHALTLAKRAWDEREVPVGAVLVLNNRVIGEGWNRPIGRH
DPTAHAEIMALRQGGLVMQNYRLIDATLYVTFEPCVMCAGAMIHSRIGRVVFGVRNAK
TGAAGSLMDVLHYPGMNHRVEITEGILADECAALLSYFFRMRRQVFKAQKKAQSSTD
ecTadA (H36L, P48L, L84F, A106V, D108N, H123Y, D147Y, E155V, 1156F)
(SEQ ID NO: 75)
SEVEFSHEYWMRHALTLAKRAWDEREVPVGAVLVLNNRVIGEGWNRLIGR
HDPTAHAEIMALRQGGLVMQNYRLIDATLYVTFEPCVMCAGAMIHSRIGRVVFGVRNA
KTGAAGSLMDVLHYPGMNHRVEITEGILADECAALLSYFFRMRRQVFKAQKKAQSSTD
ecTadA (H36L, L84F, A106V, D108N, H123Y, D147Y, E155V, K57N, I156F)
(SEQ ID NO: 76)
SEVEFSHEYWMRHALTLAKRAWDEREVPVGAVLVLNNRVIGEGWNRPIGRH
DPTAHAEIMALRQGGLVMQNYRLIDATLYVTFEPCVMCAGAMIHSRIGRVVFGVRNAK
TGAAGSLMDVLHYPGMNHRVEITEGILADECAALLSYFFRMRRQVFNAQKKAQSSTD
ecTadA (H36L, L84F, A106V, D108N, H123Y, S146C, D147Y, E155V, I156F)
(SEQ ID NO: 77)
SEVEFSHEYWMRHALTLAKRAWDEREVPVGAVLVLNNRVIGEGWNRPIGRH
DPTAHAEIMALRQGGLVMQNYRLIDATLYVTFEPCVMCAGAMIHSRIGRVVFGVRNAK
TGAAGSLMDVLHYPGMNHRVEITEGILADECAALLCYFFRMRRQVFKAQKKAQSSTD
ecTadA (L84F, A106V, D108N, H123Y, S146R, D147Y, E155V, I156F)
(SEQ ID NO: 78)
SEVEFSHEYWMRHALTLAKRAWDEREVPVGAVLVHNNRVIGEGWNRPIGR
HDPTAHAEIMALRQGGLVMQNYRLIDATLYVTFEPCVMCAGAMIHSRIGRVVFGVRNA
KTGAAGSLMDVLHYPGMNHRVEITEGILADECAALLRYFFRMRRQVFKAQKKAQSSTD
ecTadA (N37S, R51H, L84F, A106V, D108N, H123Y, D147Y, E155V, I156F
(SEQ ID NO: 79)
SEVEFSHEYWMRHALTLAKRAWDEREVPVGAVLVHSNRVIGEGWNRPIGHH
DPTAHAEIMALRQGGLVMQNYRLIDATLYVTFEPCVMCAGAMIHSRIGRVVFGVRNAK
TGAAGSLMDVLHYPGMNHRVEITEGILADECAALLSYFFRMRRQVFKAQKKAQSSTD
ecTadA (R51L, L84F, A106V, D108N, H123Y, D147Y, E155V, I156F, K157N
(SEQ ID NO: 80)
SEVEFSHEYWMRHALTLAKRAWDEREVPVGAVLVHNNRVIGEGWNRPIGL
HDPTAHAEIMALRQGGLVMQNYRLIDATLYVTFEPCVMCAGAMIHSRIGRVVFGVRNA
KTGAAGSLMDVLHYPGMNHRVEITEGILADECAALLSYFFRMRRQVFNAQKKAQSSTD
saTadA (D108N)
(SEQ ID NO: 81)
GSHMTNDIYFMTLAIEEAKKAAQLGEVPIGAIITKDDEVIARAHNLRETLQQP
TAHAEHIAIERAAKVLGSWRLEGCTLYVTLEPCVMCAGTIVMSRIPRVVYGADNPKGGC
SGSLMNLLQQSNFNHRAIVDKGVLKEACSTLLTTFFKNLRANKKSTN
saTadA (D107A_D108N)
(SEQ ID NO: 82)
GSHMTNDIYFMTLAIEEAKKAAQLGEVPIGAIITKDDEVIARAHNLRETLQQP
TAHAEHIAIERAAKVLGSWRLEGCTLYVTLEPCVMCAGTIVMSRIPRVVYGAANPKGGC
SGSLMNLLQQSNFNHRAIVDKGVLKEACSTLLTTFFKNLRANKKSTN
saTadA (G26P_D107A_D108N)
(SEQ ID NO: 83)
GSHMTNDIYFMTLAIEEAKKAAQLPEVPIGAIITKDDEVIARAHNLRETLQQPT
AHAEHIAIERAAKVLGSWRLEGCTLYVTLEPCVMCAGTIVMSRIPRVVYGAANPKGGCS
GSLMNLLQQSNFNHRAIVDKGVLKEACSTLLTTFFKNLRANKKSTN
saTadA (G26P_D107A_D108N_S142A)
(SEQ ID NO: 84)
GSHMTNDIYFMTLAIEEAKKAAQLPEVPIGAIITKDDEVIARAHNLRETLQQPT
AHAEHIAIERAAKVLGSWRLEGCTLYVTLEPCVMCAGTIVMSRIPRVVYGAANPKGGCS
GSLMNLLQQSNFNHRAIVDKGVLKEACATLLTTFFKNLRANKKSTN
saTadA (D107A_D108N_S142A)
(SEQ ID NO: 85)
GSHMTNDIYFMTLAIEEAKKAAQLGEVPIGAIITKDDEVIARAHNLRETLQQP
TAHAEHIAIERAAKVLGSWRLEGCTLYVTLEPCVMCAGTIVMSRIPRVVYGAANPKGGC
SGSLMNLLQQSNFNHRAIVDKGVLKEACATLLTTFFKNLRANKKSTN
ecTadA (P48S)
(SEQ ID NO: 86)
SEVEFSHEYWMRHALTLAKRAWDEREVPVGAVLVHNNRVIGEGWNRSIGR
HDPTAHAEIMALRQGGLVMQNYRLIDATLYVTLEPCVMCAGAMIHSRIGRVVFGARDA
KTGAAGSLMDVLHHPGMNHRVEITEGILADECAALLSDFFRMRRQEIKAQKKAQSSTD
ecTadA (P48T)
(SEQ ID NO: 87)
SEVEFSHEYWMRHALTLAKRAWDEREVPVGAVLVHNNRVIGEGWNRTIGR
HDPTAHAEIMALRQGGLVMQNYRLIDATLYVTLEPCVMCAGAMIHSRIGRVVFGARDA
KTGAAGSLMDVLHHPGMNHRVEITEGILADECAALLSDFFRMRRQEIKAQKKAQSSTD
ecTadA (P48A)
(SEQ ID NO: 88)
SEVEFSHEYWMRHALTLAKRAWDEREVPVGAVLVHNNRVIGEGWNRAIGR
HDPTAHAEIMALRQGGLVMQNYRLIDATLYVTLEPCVMCAGAMIHSRIGRVVFGARDA
KTGAAGSLMDVLHHPGMNHRVEITEGILADECAALLSDFFRMRRQEIKAQKKAQSSTD
ecTadA (A142N)
(SEQ ID NO: 89)
SEVEFSHEYWMRHALTLAKRAWDEREVPVGAVLVHNNRVIGEGWNRPIGR
HDPTAHAEIMALRQGGLVMQNYRLIDATLYVTLEPCVMCAGAMIHSRIGRVVFGARDA
KTGAAGSLMDVLHHPGMNHRVEITEGILADECNALLSDFFRMRRQEIKAQKKAQSSTD
ecTadA (W23R)
(SEQ ID NO: 90)
SEVEFSHEYWMRHALTLAKRARDEREVPVGAVLVHNNRVIGEGWNRPIGRH
DPTAHAEIMALRQGGLVMQNYRLIDATLYVTLEPCVMCAGAMIHSRIGRVVFGARDAK
TGAAGSLMDVLHHPGMNHRVEITEGILADECAALLSDFFRMRRQEIKAQKKAQSSTD
ecTadA (W23L)
(SEQ ID NO: 91)
SEVEFSHEYWMRHALTLAKRALDEREVPVGAVLVHNNRVIGEGWNRPIGRH
DPTAHAEIMALRQGGLVMQNYRLIDATLYVTLEPCVMCAGAMIHSRIGRVVFGARDAK
TGAAGSLMDVLHHPGMNHRVEITEGILADECAALLSDFFRMRRQEIKAQKKAQSSTD
ecTadA (R152P)
(SEQ ID NO: 92)
SEVEFSHEYWMRHALTLAKRAWDEREVPVGAVLVHNNRVIGEGWNRPIGR
HDPTAHAEIMALRQGGLVMQNYRLIDATLYVTLEPCVMCAGAMIHSRIGRVVFGARDA
KTGAAGSLMDVLHHPGMNHRVEITEGILADECAALLSDFFRMPRQEIKAQKKAQSSTD
ecTadA (R152H)
(SEQ ID NO: 93)
SEVEFSHEYWMRHALTLAKRAWDEREVPVGAVLVHNNRVIGEGWNRPIGR
HDPTAHAEIMALRQGGLVMQNYRLIDATLYVTLEPCVMCAGAMIHSRIGRVVFGARDA
KTGAAGSLMDVLHHPGMNHRVEITEGILADECAALLSDFFRMHRQEIKAQKKAQSSTD
ecTadA (L84F, A106V, D108N, H123Y, D147Y, E155V, 1156F)
(SEQ ID NO: 94)
SEVEFSHEYWMRHALTLAKRAWDEREVPVGAVLVHNNRVIGEGWNRPIGR
HDPTAHAEIMALRQGGLVMQNYRLIDATLYVTFEPCVMCAGAMIHSRIGRVVFGVRNA
KTGAAGSLMDVLHYPGMNHRVEITEGILADECAALLSYFFRMRRQVFKAQKKAQSSTD
ecTadA (H36L, R51L, L84F, A106V, D108N, H123Y, S146C, D147Y, E155V,
I156F, K157N)
(SEQ ID NO: 95)
SEVEFSHEYWMRHALTLAKRAWDEREVPVGAVLVLNNRVIGEGWNRPIGLH
DPTAHAEIMALRQGGLVMQNYRLIDATLYVTFEPCVMCAGAMIHSRIGRVVFGVRNAK
TGAAGSLMDVLHYPGMNHRVEITEGILADECAALLCYFFRMRRQVFNAQKKAQSSTD
ecTadA (H36L, P48S, R51L, L84F, A106V, D108N, H123Y, S146C, D147Y,
E155V, I156F, K157N)
(SEQ ID NO: 96)
SEVEFSHEYWMRHALTLAKRAWDEREVPVGAVLVLNNRVIGEGWNRSIGLH
DPTAHAEIMALRQGGLVMQNYRLIDATLYVTFEPCVMCAGAMIHSRIGRVVFGVRNAK
TGAAGSLMDVLHYPGMNHRVEITEGILADECAALLCYFFRMRRQVFNAQKKAQSSTD
ecTadA (H36L, P48A, R51L, L84F, A106V, D108N, H123Y, S146C, D147Y,
E155V, I156F, K157N)
(SEQ ID NO: 97)
SEVEFSHEYWMRHALTLAKRAWDEREVPVGAVLVLNNRVIGEGWNRAIGL
HDPTAHAEIMALRQGGLVMQNYRLIDATLYVTFEPCVMCAGAMIHSRIGRVVFGVRNA
KTGAAGSLMDVLHYPGMNHRVEITEGILADECAALLCYFFRMRRQVFNAQKKAQSSTD
ecTadA (W23L, H36L, P48A, R51L, L84F, A106V, D108N, H123Y, S146C,
D147Y, R152P, E155V, I156F, K157N)
(SEQ ID NO: 98)
SEVEFSHEYWMRHALTLAKRALDEREVPVGAVLVLNNRVIGEGWNRAIGLH
DPTAHAEIMALRQGGLVMQNYRLIDATLYVTFEPCVMCAGAMIHSRIGRVVFGVRNAK
TGAAGSLMDVLHYPGMNHRVEITEGILADECAALLCYFFRMPRQVFNAQKKAQSSTD
ecTadA (W23R, H36L, P48A, R51L, L84F, A106V, D108N, H123Y, S146C,
D147Y, R152P, E155V, I156F, K157N)
(SEQ ID NO: 99)
SEVEFSHEYWMRHALTLAKRARDEREVPVGAVLVLNNRVIGEGWNRAIGLH
DPTAHAEIMALRQGGLVMQNYRLIDATLYVTFEPCVMCAGAMIHSRIGRVVFGVRNAK
TGAAGSLMDVLHYPGMNHRVEITEGILADECAALLCYFFRMPRQVFNAQKKAQSSTD
Staphylococcus aureus TadA:
(SEQ ID NO: 100)
MGSHMTNDIYFMTLAIEEAKKAAQLGEVPIGAIITKDDEVIARAHNLRETLQQ
PTAHAEHIAIERAAKVLGSWRLEGCTLYVTLEPCVMCAGTIVMSRIPRVVYGADDPKGG
CSGSLMNLLQQSNFNHRAIVDKGVLKEACSTLLTTFFKNLRANKKSTN
Bacillus subtilis TadA:
(SEQ ID NO: 101)
MTQDELYMKEAIKEAKKAEEKGEVPIGAVLVINGEIIARAHNLRETEQRSIAH
AEMLVIDEACKALGTWRLEGATLYVTLEPCPMCAGAVVLSRVEKVVFGAFDPKGGCS
GTLMNLLQEERFNHQAEVVSGVLEEECGGMLSAFFRELRKKKKAARKNLSE
Salmonella typhimurium (S. typhimurium) TadA:
(SEQ ID NO: 102)
MPPAFITGVTSLSDVELDHEYWMRHALTLAKRAWDEREVPVGAVLVHNHR
VIGEGWNRPIGRHDPTAHAEIMALRQGGLVLQNYRLLDTTLYVTLEPCVMCAGAMVHS
RIGRVVFGARDAKTGAAGSLIDVLHHPGMNHRVEIIEGVLRDECATLLSDFFRMRRQEI
KALKKADRAEGAGPAV
Shewanella putrefaciens (S. putrefaciens) TadA:
(SEQ ID NO: 103)
MDEYWMQVAMQMAEKAEAAGEVPVGAVLVKDGQQIATGYNLSISQHDPT
AHAEILCLRSAGKKLENYRLLDATLYITLEPCAMCAGAMVHSRIARVVYGARDEKTGA
AGTVVNLLQHPAFNHQVEVTSGVLAEACSAQLSRFFKRRRDEKKALKLAQRAQQGIE
Haemophilus influenzae F3031 (H. influenzae) TadA:
(SEQ ID NO: 104)
MDAAKVRSEFDEKMMRYALELADKAEALGEIPVGAVLVDDARNIIGEGWNL
SIVQSDPTAHAEIIALRNGAKNIQNYRLLNSTLYVTLEPCTMCAGAILHSRIKRLVFGASD
YKTGAIGSRFHFFDDYKMNHTLEITSGVLAEECSQKLSTFFQKRREEKKIEKALLKSLSD
K
Caulobacter crescentus (C. crescentus) TadA:
(SEQ ID NO: 105)
MRTDESEDQDHRMMRLALDAARAAAEAGETPVGAVILDPSTGEVIATAGNG
PIAAHDPTAHAEIAAMRAAAAKLGNYRLTDLTLVVTLEPCAMCAGAISHARIGRVVFGA
DDPKGGAVVHGPKFFAQPTCHWRPEVTGGVLADESADLLRGFFRARRKAKI
Geobacter sulfurreducens (G. sulfurreducens) TadA:
(SEQ ID NO: 106)
MSSLKKTPIRDDAYWMGKAIREAAKAAARDEVPIGAVIVRDGAVIGRGHNL
REGSNDPSAHAEMIAIRQAARRSANWRLTGATLYVTLEPCLMCMGAIILARLERVVFGC
YDPKGGAAGSLYDLSADPRLNHQVRLSPGVCQEECGTMLSDFFRDLRRRKKAKATPAL
FIDERKVPPEP
Streptococcus pyogenes (S. pyogenes) TadA
(SEQ ID NO: 107
MPYSLEEQTYFMQEALKEAEKSLQKAEIPIGCVIVKDGEIIGRGHNAREESNQ
AIMHAEIMAINEANAHEGNWRLLDTTLFVTIEPCVMCSGAIGLARIPHVIYGASNQKFGG
ADSLYQILTDERLNHRVQVERGLLAADCANIMQTFFRQGRERKKIAKHLIKEQSDPFD
TadA 7.10:
(SEQ ID NO: 108)
SEVEFSHEYWMRHALTLAKRARDEREVPVGAVLVLNNRVIGEGWNRAIGLH
DPTAHAEIMALRQGGLVMQNYRLIDATLYVTFEPCVMCAGAMIHSRIGRVVFGVRNAK
TGAAGSLMDVLHYPGMNHRVEITEGILADECAALLCYFFRMPRQVFNAQKKAQSSTD
TadA 7.10 (V106W) (E. coli)
(SEQ ID NO: 109)
SEVEFSHEYWMRHALTLAKRARDEREVPVGAVLVLNNRVIGEGWNRAIGLH
DPTAHAEIMALRQGGLVMQNYRLIDATLYVTFEPCVMCAGAMIHSRIGRVVFGWRNA
KTGAAGSLMDVLHYPGMNHRVEITEGILADECAALLCYFFRMPRQVFNAQKKAQSSTD
TadA-8e (E. coli)
(SEQ ID NO: 110)
SEVEFSHEYWMRHALTLAKRARDEREVPVGAVLVLNNRVIGEGWNRAIGLH
DPTAHAEIMALRQGGLVMQNYRLIDATLYVTFEPCVMCAGAMIHSRIGRVVFGVRNSK
RGAAGSLMNVLNYPGMNHRVEITEGILADECAALLCDFYRMPRQVFNAQKKAQSSIN
TadA-8e(V106W) (E. coli)
(SEQ ID NO: 111)
SEVEFSHEYWMRHALTLAKRARDEREVPVGAVLVLNNRVIGEGWNRAIGLH
DPTAHAEIMALRQGGLVMQNYRLIDATLYVTFEPCVMCAGAMIHSRIGRVVFGWRNSK
RGAAGSLMNVLNYPGMNHRVEITEGILADECAALLCDFYRMPRQVFNAQKKAQSSIN
Aquifex aeolicus (A. aeolicus) TadA
(SEQ ID NO: 112)
MGKEYFLKVALREAKRAFEKGEVPVGAIIVKEGEIISKAHNSVEELKDPTAHA
EMLAIKEACRRLNTKYLEGCELYVTLEPCIMCSYALVLSRIEKVIFSALDKKHGGVVSVF
NILDEPTLNHRVKWEYYPLEEASELLSEFFKKLRNNII
Tad1
(SEQ ID NO: 113)
SEVEFSHEYWMRHALTLAKRARDEGEVPVGAVLVLNNRVIGEGWNRAIGLY
DPTAHAEIMALRQGGLVMQNYRLIDATLYVTFEPCVMCAGAMIHSRIGRVVFGVRNSK
RGAAGSLMNVLNYPGMDHRVEITEGILADECAALLCDFYRMPRQVFNAQKKAQSSIN
Tad2
(SEQ ID NO: 114)
SEVEFSHEYWMRHALTLAKRARDEGEVPVGAVLVLNNRVIGEGWNRAIGLH
DPTAHAEIMALRQGGLVMQNYRLIDATLYVTFEPCVMCAGAMIHSRIGRVVFGVRNSK
RGAAGSLMNVLNYPGMDHRVEITEGILADECAALLCDFYRMPRQVFNAQKKAQSSIN
Tad3
(SEQ ID NO: 115)
SEVEFSHEYWMRHALTLAKRARDEREVPVGAVLVLNNRVIGEGWNRAIGLH
DPTAHAEIMALRQGGLVMQNYGLIDATLYVTFEPCVMCAGAIIHSRIGRVVFGVRNSKR
GAAGSLMNVLNYPGMNHRVEITEGILADECAALLCDFYRMPRQVFNAQKKAQSSIN
Tad4
(SEQ ID NO: 116)
SEVEFSHEYWMRHALTLAKRARDEREVPVGAVLVLNNRVIGEGWNRAIGLH
DPTAHAEIMALRQGGLVMQNYRLIDATLYVTFEPCVMCAGAMIHSRIGRVVFGVRNSK
RGAAGSLMNVLNYPGMDHRVEITEGILADECAALLCDFYRMPRQVFNAQKKAQSSIN
Tad6
(SEQ ID NO: 117)
SEVEFSHEYWMRHALTLAKRARDEGEVPVGAVLVLNNRVIGEGWNRAIGLY
DPTAHAEIMALRQGGLVMQNYGLIDATLYVTFEPCVMCAGAMIHSRIGRVVFGVRNSK
RGAAGSLMNVLNYPGMDHRVEITEGILADECAALLCDFYRMPRQVFNAQKKAQSSIN
Tad6-SR
(SEQ ID NO: 118)
SEVEFSHEYWMRHALTLAKRARDEGEVPVGAVLVLNNRVIGEGWNRAIGLY
DPTAHAEIMALRQGGLVMQNYGLIDATLYSTFEPCVMCAGAMIHSRIGRVVFGVRNSK
RGAAGSLMNVLNYPGMDHRVEITEGILADECAALLCDFYRMPRRVFNAQKKAQSSIN

In some aspects, the fusion proteins of the present disclosure comprise cytidine base editors (CBEs) comprising a napDNAbp domain (e.g., any of the Cas14a1 variants provided herein) and a cytosine deaminase domain that enzymatically deaminates a cytosine nucleobase of a C:G nucleobase pair to a uracil. The uracil may be subsequently converted to a thymine (T) by the cell's DNA repair and replication machinery. The mismatched guanine (G) on the opposite strand may subsequently be converted to an adenine (A) by the cell's DNA repair and replication machinery. In this manner, a target C:G nucleobase pair is ultimately converted to a T:A nucleobase pair. Other cytosine deaminase domains besides those provided herein are known in the art, and a person of ordinary skill in the art would recognize which cytosine deaminase domains could be used in the fusion proteins of the present disclosure.

The CBE fusion proteins described herein may further comprise one or more nuclear localization signals (NLSs) and/or one or more uracil glycosylase inhibitor (UGI) domains. Thus, the base editor fusion proteins may comprise the structure: NH₂-[first nuclear localization sequence]-[cytosine deaminase domain]-[napDNAbp domain]-[first UGI domain]-[second UGI domain]-[second nuclear localization sequence]-COOH, wherein each instance of “]-[” indicates the presence of an optional linker sequence. The CBE fusion proteins of the present disclosure may comprise modified (or evolved) cytosine deaminase domains, such as deaminase domains that recognize an expanded PAM sequence, have improved efficiency of deaminating 5′-GC targets, and/or make edits in a narrower target window.

In some aspects, the fusion proteins of the disclosure comprise an adenine base editor. Some aspects of the disclosure provide fusion proteins that comprise a nucleic acid programmable DNA binding protein (napDNAbp), such as any of the Cas14a1 variants provided herein, and at least two adenosine deaminase domains. Without wishing to be bound by any particular theory, dimerization of adenosine deaminases (e.g., in cis or in trans) may improve the ability (e.g., efficiency) of the fusion protein to modify a nucleic acid base (for example, to deaminate adenine). In some embodiments, any of the fusion proteins may comprise 2, 3, 4, or 5 adenosine deaminase domains. In some embodiments, any of the fusion proteins provided herein comprises two adenosine deaminases. In some embodiments, any of the fusion proteins provided herein contain only two adenosine deaminases. In some embodiments, the adenosine deaminases are the same. In some embodiments, the adenosine deaminases are any of the adenosine deaminases provided herein. In some embodiments, the adenosine deaminases are different. Other adenosine deaminase domains besides those provided herein are known in the art, and a person of ordinary skill in the art would recognize which adenosine deaminase domains could be used in the fusion proteins of the present disclosure.

In some embodiments, the general architecture of exemplary fusion proteins with a first adenosine deaminase, a second adenosine deaminase, and a napDNAbp (e.g., any of the Cas14a1 variants provided herein) comprises any one of the following structures, where NLS is a nuclear localization sequence (e.g., any NLS provided herein), NH₂ is the N-terminus of the fusion protein, and COOH is the C-terminus of the fusion protein: NH₂-[first adenosine deaminase]-[second adenosine deaminase]-[napDNAbp]-COOH; NH₂-[first adenosine deaminase]-[napDNAbp]-[second adenosine deaminase]-COOH; NH₂-[napDNAbp]-[first adenosine deaminase]-[second adenosine deaminase]-COOH; NH₂-[second adenosine deaminase]-[first adenosine deaminase]-[napDNAbp]-COOH; NH₂-[second adenosine deaminase]-[napDNAbp]-[first adenosine deaminase]-COOH; NH₂-[napDNAbp]-[second adenosine deaminase]-[first adenosine deaminase]-COOH.

In some embodiments, the fusion proteins provided herein do not comprise a linker.

In some embodiments, a linker is present between one or more of the domains or proteins (e.g., first adenosine deaminase, second adenosine deaminase, and/or napDNAbp). In some embodiments, the “]-[” used in the general architecture above indicates the presence of an optional linker. Exemplary fusion proteins comprising a first adenosine deaminase, a second adenosine deaminase, a napDNAbp, and an NLS are provided: NH₂-[NLS]-[first adenosine deaminase]-[second adenosine deaminase]-[napDNAbp]-COOH; NH₂-[first adenosine deaminase]-[NLS]-[second adenosine deaminase]-[napDNAbp]-COOH; NH₂-[first adenosine deaminase]-[second adenosine deaminase]-[NLS]-[napDNAbp]-COOH; NH₂-[first adenosine deaminase]-[second adenosine deaminase]-[napDNAbp]-[NLS]-COOH; NH₂-[NLS]-[first adenosine deaminase]-[napDNAbp]-[second adenosine deaminase]-COOH; NH₂-[first adenosine deaminase]-[NLS]-[napDNAbp]-[second adenosine deaminase]-COOH; NH₂-[first adenosine deaminase]-[napDNAbp]-[NLS]-[second adenosine deaminase]-COOH; NH₂-[first adenosine deaminase]-[napDNAbp]-[second adenosine deaminase]-[NLS]-COOH; NH₂-[NLS]-[napDNAbp]-[first adenosine deaminase]-[second adenosine deaminase]-COOH; NH₂-[napDNAbp]-[NLS]-[first adenosine deaminase]-[second adenosine deaminase]-COOH; NH₂-[napDNAbp]-[first adenosine deaminase]-[NLS]-[second adenosine deaminase]-COOH; NH₂-[napDNAbp]-[first adenosine deaminase]-[second adenosine deaminase]-[NLS]-COOH; NH₂-[NLS]-[second adenosine deaminase]-[first adenosine deaminase]-[napDNAbp]-COOH; NH₂-[second adenosine deaminase]-[NLS]-[first adenosine deaminase]-[napDNAbp]-COOH; NH₂-[second adenosine deaminase]-[first adenosine deaminase]-[NLS]-[napDNAbp]-COOH; NH₂-[second adenosine deaminase]-[first adenosine deaminase]-[napDNAbp]-[NLS]-COOH; NH₂-[NLS]-[second adenosine deaminase]-[napDNAbp]-[first adenosine deaminase]-COOH; NH₂-[second adenosine deaminase]-[NLS]-[napDNAbp]-[first adenosine deaminase]-COOH; NH₂-[second adenosine deaminase]-[napDNAbp]-[NLS]-[first adenosine deaminase]-COOH; NH₂-[second adenosine deaminase]-[napDNAbp]-[first adenosine deaminase]-[NLS]-COOH; NH₂-[NLS]-[napDNAbp]-[second adenosine deaminase]-[first adenosine deaminase]-COOH; NH₂-[napDNAbp]-[NLS]-[second adenosine deaminase]-[first adenosine deaminase]-COOH; NH₂-[napDNAbp]-[second adenosine deaminase]-[NLS]-[first adenosine deaminase]-COOH; NH₂-[napDNAbp]-[second adenosine deaminase]-[first adenosine deaminase]-[NLS]-COOH.

### A-TO-C Transversion Base-Editors

In various embodiments, the present disclosure provides A-to-C(or T-to-G) transversion base editor fusion proteins comprising (i) a nucleic acid programmable DNA binding protein (napDNAbp), and (ii) a nucleobase modification domain capable of facilitating the conversion of a A:T nucleobase pair to a C:G nucleobase pair in a target nucleotide sequence, e.g., a genome, such as those described in U.S. Patent Application U.S. Ser. No. 62/814,766 filed Mar. 6, 2019 and International Patent Application No. PCT/US2020/021362 filed on Mar. 6, 2020, both of which are herein incorporated by reference in their entirety.

In various embodiments, the nucleobase modification domain is an adenine oxidase, which enzymatically converts an adenine nucleobase of an A:T nucleobase pair to an 8-oxoadenine, which is subsequently converted by the cell's DNA repair and replication machinery to a cytosine, ultimately converting the A:T nucleobase pair to a C:G nucleobase pair.

The various domains of the transversion fusion proteins described herein (e.g., the Cas9 domain or the nucleobase modification domains) may be obtained as a result of mutagenizing a reference or starting-point base editor (or a component or domain thereof) by a directed evolution process, e.g., a continuous evolution method (e.g., PACE) or a non-continuous evolution method (e.g., PANCE or other discrete plate-based selections). In various embodiments, the disclosure provides a base editor that has one or more amino acid variations introduced into its amino acid sequence relative to the amino acid sequence of the reference or starting-point base editor. The base editor may include variants in one or more components or domains of the base editor (e.g., variants introduced into a Cas9 domain, an adenine oxidase domain, an inhibitor of base excision repair (iBER) domain, or a variant introduced into combinations of these domains). For example, the nucleobase modification domain may be evolved from a reference protein that is an RNA modifying enzyme (e.g., an N1-methyladenosine modification enzyme or a 5-methylcytosine modification enzyme) and evolved using PACE, PANCE, or other plate-based evolution methods to obtain a DNA modifying version of the nucleobase modification domain, which can then be used in the fusion proteins described herein.

### Adenine Oxidases

In various embodiments, the ACBE and TGBE transversion base editors provided herein comprise an adenine oxidase nucleobase modification domain. An adenine oxidase is an enzyme that has catalytic activity in oxidizing an adenosine nucleobase substrate. Oxidation reactions catalyzed by the exemplary enzymes of the present disclosure may comprise transfers of oxo (═O) substituents to the adenosine nucleobase, which creates an aldehyde, 8-oxoadenine. Exemplary oxidases of this disclosure catalyze oxidation reactions at the 8 position of adenosine. The 8 position of adenine is the most readily oxidized position on the nucleobase. See Saladino, R. et al., A new and efficient synthesis of 8-hydroxypurine derivatives by dimethyldioxirane oxidation, Tet. Lett. (1995) 36: 2665-2668; Chang, W.-C. et al., Mechanistic Investigation of a Non-Heme Iron Enzyme Catalyzed Epoxidation in (−)-4′-Methoxycyclopenin Biosynthesis, J. Am. Chem. Soc. (2016) 138(33): 10390-10393, the entire contents of each of which is herein incorporated by reference.

The adenine oxidases of the present disclosure may be modified from wild-type reference proteins, which include 5-methylcytosine, Ni-methyladenosine and xanthine modification enzymes. Other modification enzymes that may serve as reference proteins are N⁴-acetylcytosine- and 2-thiocytosine-installing RNA-modification enzymes. See Ito, S. et al. Human NAT10 Is an ATP-dependent RNA Acetyltransferase Responsible for N4-Acetylcytidine Formation in 18 S Ribosomal RNA (rRNA). J. Biol. Chem. 2014, 289, 35724-35730; and Čavužić, V.; Liu, Y., Biosynthesis of Sulfur-Containing tRNA Modifications: A Comparison of Bacterial, Archaeal, and Eukaryotic Pathways. Biomolecules 2017, 7, 27, each of which is herein incorporated by reference. Wild-type reference proteins may be those from E. coli, S. cyanogenus, yeast, mouse, human, or another organism, including other bacteria. See also Falnes, P. Ø.; Rognes, T. DNA repair by bacterial AlkB proteins, Res. Microbiol. (2003) 154(8): 531-538; Ito, S. et al., Tet proteins can convert 5-methylcytosine to 5-formylcytosine and 5-carboxylcytosine, Science (2011) 333(6047): 1300-1303; Fortini, P. et al., 8-Oxoguanine DNA damage: at the crossroad of alternative repair pathways, Mutat. Res. (2003) 531(1-2): 127-39; Leonard, G. A. et al., Conformation of guanine-8-oxoadenine base pairs in the crystal structure of d(CGCGAATT(08A)GCG), Biochem. (1992) 31(36): 8415-8420; Ohe, T. & Watanabe, Y. Purification and Properties of Xanthine Dehydrogenase from Streptomyces cyanogenus, J. Biochem. 86:45-53, (1979), the entire contents of each of which is herein incorporated by reference.

Modified adenine oxidases include variants with at least 80%, at least 85%, at least 90%, at least 95% or at least 99% sequence identity to a wild-type adenine oxidase. In other embodiments, modified adenine oxidases may be obtained by altering or evolving a reference protein using a continuous evolution process (e.g., PACE) or non-continuous evolution process (e.g., PANCE or discrete plate-based selections) described herein so that the oxidase is effective on a nucleic acid target. 8-oxopurines, common products of oxidative DNA damage, tend to rotate around the glycosidic bond to adopt the syn conformation, presenting the Hoogsteen edge for base pairing. The Hoogsteen edge of 8-oxoA and the Watson-Crick edge of G form a base pair featuring two three-center hydrogen bonding systems. The 8-oxoA:G pair makes a minimal perturbation to the DNA double helix. Consequently, polymerases misread 8-oxoA and pair it with G, eventually resulting in an A:T to C:G transversion mutation. See Kamiya, H. et al., 8-Hydroxyadenine (7,8-dihydro-8-oxoadenine) induces misincorporation in in vitro DNA synthesis and mutations in NIH 3T3 cells, Nucleic Acids Res. (1995) 23(15): 2893-2895; Tan, X., Grollman, A. P., & Shibutani, S., Comparison of the mutagenic properties of 8-oxo-7,8-dihydro-2′-deoxyadenosine and 8-oxo-7,8-dihydro-2′-deoxyguanosine DNA lesions in mammalian cells, Carcinogenesis (1999) 20(12): 2287-2292; Leonard, G. A. et al., Conformation of guanine-8-oxoadenine base pairs in the crystal structure of d(CGCGAATT(08A)GCG), Biochem. (1992) 31(36): 8415-8420, the entire contents of each of which is herein incorporated by reference.

Exemplary adenine oxidases include, but are not limited to, α-ketoglutarate-dependent iron oxidases, molybdopterin-dependent oxidases, heme iron oxidases, and flavin monooxygenases. See Rashidi, M. R. & Soltani, S., An overview of aldehyde oxidase: an enzyme of emerging importance in novel drug discovery, Expert Opin. Drug Discov. (2017) 12(3): 305-316; Coon, M. J., Cytochrome P450: nature's most versatile biological catalyst, Annu. Rev. Pharmacol. Taxicol. (2005) 45: 1-25; Eswaramoorthy, S. et al., Mechanism of action of a flavin-containing monooxygenase, Proc. Natl. Acad. Sci. (2006) 103(26): 9832-9837, the entire contents of each of which is herein incorporated by reference.

Exemplary α-ketoglutarate-dependent iron oxidases include AlkbH (ABH) family oxidases, which include human AlkBH3, is to clear Ni-methylation from adenine in DNA and RNA. These non-heme enzymes perform methyl group C—H hydroxylation on DNA and RNA via an active Fe(IV)-oxo intermediate formed through an iron cofactor. The resulting hemiaminal breaks down to release formaldehyde and the demethylated adenine base. ABH3 is selective for ssDNA over dsDNA, a characteristic of exocyclic amine-hydrolyzing enzymes that likely contributes to the selective modification of bases in the targeted ssDNA loop of the ternary Cas9-sgRNA-DNA complex. The TET oxidases are structurally related α-ketoglutarate-dependent iron oxidases and perform C—H hydroxylation on 5-methylcytosine as the first step in removing this important epigenetic marker. Oxidized forms of 5-methylcytosine are recognized by DNA glycosylases and hydrolytically removed, to be replaced eventually by unmethylated cytosine. Without being bound by a particular theory, in the absence of a labile C—H bond substrate, the Fe(IV)-oxo species of the cofactor-enzyme may be induced to transfer the oxo group from the non-heme Fe(IV) center to the 8 position of adenine. This potential mechanism involves the formation of a 7,8-oxaziridine intermediate, which rearranges spontaneously to the desired 8-oxoadenine.

Exemplary molybdopterin-dependent oxidases that selectively oxidize adenine at the 8 position include xanthine dehydrogenases and aldehyde oxidases. In eukaryotes, these enzymes utilize a monophosphate pyranopterin cofactor, which complexes with a molybdenum to form molybdenum cofactor (Moco). These oxidases may effect alkene/arene epoxidation reactions in natural product biosynthesis pathways via similar oxo group transfer mechanisms as those of the non-heme ABH and TET iron oxidases.

Exemplary heme iron oxidases that selectively oxidize adenine at the 8 position include cytochrome P450 enzymes.

### G-to-T Transversion Base-Editors

In various embodiments, the present disclosure provides G-to-T (or C-to-A) transversion base editor fusion proteins, such as those described in U.S. Provisional Patent Application, U.S. Ser. No. 62/768,062, filed Nov. 15, 2018, International Patent Application No. PCT/US2019/061685, filed Nov. 15, 2019, and U.S. patent application U.S. Ser. No. 17/294,287, filed May 14, 2021, all of which are hereby incorporated by reference in their entirety.

In some embodiments, the fusion proteins comprise (i) a nucleic acid programmable DNA binding protein (napDNAbp), and (ii) a nucleobase modification moiety that is capable of facilitating the conversion of a G to a T in a target nucleotide sequence, e.g., a genome (or equivalently, which is capable of facilitating the conversion of a G:C nucleobase pair to a T:A nucleobase pair). In various embodiments, the nucleobase modification moiety can be a guanine oxidase, which enzymatically converts a guanine nucleobase of a G:C nucleobase pair to 8-oxo-guanine, which then is subsequently processed by the cell's DNA repair and replication machinery to a thymine, thereby converting the G:C nucleobase pair to a T:A nucleobase pair. In other embodiments, the nucleobase modification moiety can be a guanine methyltransferase, which enzymatically converts a guanine nucleobase of a G:C nucleobase pair to 8-methyl-guanine, which then is subsequently processed by the cell's DNA repair and replication machinery to a thymine, thereby converting the G:C nucleobase pair to a T:A nucleobase pair. In still other embodiments, the nucleobase modification moiety can be a guanine methyltransferase, which enzymatically converts a guanine nucleobase of a G:C nucleobase pair to a Ni-methyl-guanine or to an N2,N2-dimethyl-guanine, which then is subsequently processed by the cell's DNA repair and replication machinery to a thymine, thereby converting the G:C nucleobase pair to a T:A nucleobase pair.

The various domains of the transversion fusion proteins described herein (e.g., the Cas9 domain or the nucleobase modification domains) can be obtained as a result of mutagenizing a reference or starting-point base editor (or a component or domain thereof) by a directed evolution process, e.g., a continuous evolution method (e.g., PACE) or PANCE. In various embodiments, the disclosure provides an evolved base editor that has one or more amino acid variations introduced into its amino acid sequence relative to the amino acid sequence of the reference or starting-point base editor. The evolved base editor may include variants in one or more components or domains of the base editor (e.g., variants introduced into a Cas9 domain, a guanine oxidase domain, or 8-oxoguanine glycosylase (OGG) inhibitor domain, or variants introduced into combinations of these domains). For example, the nucleobase modification domain can be evolved from a reference protein that is an RNA modifying enzyme and evolved using PACE of PANCE to obtain a DNA modifying version of the nucleobase modification domain, which can then be used in the fusion proteins described herein.

In one embodiment, the guanine oxidase is a wild-type guanine oxidase, or a variant thereof, that oxidizes a guanine in DNA. In certain embodiments, the guanine oxidase is a xanthine dehydrogenase, or a variant thereof. In certain embodiments, the xanthine dehydrogenase is a Streptomyces cyanogenus xanthine dehydrogenase (ScXDH) or variant thereof. In other embodiments, the xanthine dehydrogenase or variant thereof is derived from C. capitata, N. crassa, M. hansupus, E. cloacae, S. snoursei, S. albulus, S. himastatinicus, or S. lividans.

In various embodiments, the fusion protein further comprises an 8-oxoguanine glycosylase (OGG) inhibitor. In certain embodiments, the OGG inhibitor binds to 8-oxoguanine (8-oxo-G) and may comprise a catalytically inactive OGG enzyme. In various embodiments, the base editor fusion proteins described herein can comprise any of the following structures: NH₂-[napDNAbp]-[guanine oxidase]-COOH; NH₂-[guanine oxidase]-[napDNAbp]-COOH; NH₂-[OGG inhibitor]-[napDNAbp]-[guanine oxidase]-COOH; NH₂-[napDNAbp]-[OGG inhibitor]-[guanine oxidase]-COOH; NH₂-[napDNAbp]-[guanine oxidase]-[OGG inhibitor]-COOH; NH₂-[OGG inhibitor]-[guanine oxidase]-[napDNAbp]-COOH; NH₂-[guanine oxidase]-[OGG inhibitor]-[napDNAbp]-COOH; or NH₂-[guanine oxidase]-[napDNAbp]-[OGG inhibitor]-COOH; wherein each instance of “-” comprises an optional linker.

In another embodiment, the base editor fusion protein comprises (i) a nucleic acid programmable DNA binding protein (napDNAbp), and (ii) a guanine methyltransferase. In various embodiments of the base editor fusion proteins, the guanine methyltransferase is a wild-type guanine methyltransferase. In certain embodiments, the guanine methyltransferase is a wild-type RlmA, or a variant thereof, that methylates a guanine in DNA. In certain embodiments, the RlmA is a Escherichia coli RlmA, or a variant thereof.

In one embodiment, the guanine methyltransferase is a dimethyl transferase that methylates a guanine to N2,N2-dimethylguanine. In various embodiments, the dimethyl transferase is a Trm1, or a variant thereof, that methylates a guanine in DNA. In other embodiments, the dimethyl transferase is a Aquifex aeolicus Trm1 or variant thereof. In certain embodiments, the dimethyl transferase is a human Trm1 or variant thereof. In certain embodiments, the dimethyl transferase is a Saccharomyces cerevisiae Trm1 or variant thereof.

In one embodiment, the guanine methyltransferase methylates a guanine to Ni-methyl-guanine. In various embodiments, the methyltransferase is a RlmA, a TrmT10A, a Termed, or variants thereof, that methylates a guanine in DNA. In various embodiments, the methyltransferase is an Escherichia coli RlmA, human TrmT10A, Escherichia coli Termed, M. Jannaschii Trm5b or P. Abyssi Trm5b. In certain embodiments, the methyltransferase is an Escherichia coli Termed having one or more of the following mutations: M149V, G189V, and E194K.

In various embodiments, the base editor fusion proteins described herein can comprise any of the following structures: NH₂-[napDNAbp]-[guanine methyltransferase]-COOH; NH₂-[guanine methyltransferase]-[napDNAbp]-COOH; NH₂-[ALRE inhibitor]-[napDNAbp]-[guanine oxidase]-COOH; NH₂-[napDNAbp]-[ALRE inhibitor]-[guanine oxidase]-COOH; NH₂-[napDNAbp]-[guanine oxidase]-[ALRE inhibitor]-COOH; NH₂-[ALRE inhibitor]-[guanine oxidase]-[napDNAbp]-COOH; NH₂-[guanine oxidase]-[ALRE inhibitor]-[napDNAbp]-COOH; or NH₂-[guanine oxidase]-[napDNAbp]-[ALRE inhibitor]-COOH; wherein each instance of “-” comprises an optional linker.

In still another embodiment, the guanine methyltransferase methylates a guanine to 8-methyl-guanine. 8-methyl-guanine induces steric rotation of the damaged G, forcing base pairing with the Hoogsteen face of 8-methyl-guanine. As a result and through the cell's replication and repair processes, 8-methyl-guanine pairs with A and results in a G-to-T transversion. In certain embodiments, the guanine methyltransferase is a wild-type Cfr, or a variant thereof, that methylates a guanine in DNA. In certain embodiments, the Cfr is a Staphylococcus scirui Cfr, or a variant thereof.

In some embodiments, any of the base editor proteins provided herein may further comprise one or more additional nucleobase modification moieties, such as, for example, an inhibitor of 8-oxoguanine glycosylase (OGG) domain. Without wishing to be bound by any particular theory, the OGG inhibitor domain may inhibit or prevent base excision repair of a oxidized guanine residue, which may improve the activity or efficiency of the base editor. Additional base editor functionalities are further described herein.

### Guanine Oxidases

In various embodiments, the transversion base editors provided herein comprise one or more nucleobase modification domains (e.g., guanine oxidase). Optionally, these domains may be obtained by evolving a reference version (e.g., an RNA modification enzyme) evolved using a continuous evolution process (e.g., PACE) described herein so that the nucleobase modification domain is effective on a DNA target.

In various embodiments, the nucleobase modification moiety may be any protein, enzyme, or polypeptide (or functional fragment thereof) which is capable of modifying a nucleobase. Nucleobase modification moieties can be naturally occurring or recombinant. Exemplary nucleobase modification moieties include, but are not limited to, a guanine oxidase. In some embodiments the modification moiety is a guanine oxidase (e.g., ScXDH), or an evolved variant thereof.

### Guanine Methyltransferases

In various embodiments, the transversion base editors provided herein comprise one or more nucleobase modification moieties (e.g., guanine methyltransferase). Optionally, these moieties may be evolved using a continuous evolution process (e.g., PACE or PANCE) described herein.

In various embodiments, the nucleobase modification moiety may be any protein, enzyme, or polypeptide (or functional fragment thereof) which is capable of modifying a nucleobase. Nucleobase modification moieties can be naturally occurring, or can be engineered or modified. A nucleobase modification moiety can have one or more types of enzymatic activities, including, but not limited to, endonuclease activity, polymerase activity, ligase activity, replication activity, or proofreading activity. Nucleobase modification moieties can also include DNA or RNA-modifying enzymes and/or mutagenic enzymes, such as, DNA methylases and alkylating enzymes (i.e., guanine methyltransferases), which covalently modify nucleobases leading in some cases to mutagenic corrections by way of normal cellular DNA repair and replication processes. Exemplary nucleobase modification moieties include, but are not limited to, a guanine methyltransferase, a nuclease, a nickase, a recombinase, a methylase, an acetylase, an acetyltransferase, a transcriptional activator, or a transcriptional repressor domain. In some embodiments the nucleobase modification moiety is a guanine methyltransferase (e.g., RlmA (E. coli)), or an evolved variant thereof.

### T-to-G Transversion Base-Editors

In various embodiments, the nucleotide modification domain is a transglycosylase that enzymatically exchanges a thymine nucleobase of a T:A nucleobase pair with a guanine, such as those disclosed in U.S. Provisional Patent Application, U.S. Ser. No. 62/887,307, filed Aug. 15, 2019 and International Patent Application No. PCT/US2020/046320, filed Aug. 14, 2020, both of which are herein incorporated by reference in their entirety. In other embodiments, the transglycosylase enzymatically exchanges a thymine nucleobase of a T:A nucleobase pair with a 7-deazaguanine derivative, which is subsequently converted by the cell's DNA repair and replication machinery to a guanine. In both of these embodiments, the T:A nucleobase pair is ultimately converted to a G:C nucleobase pair.

The various domains of the transversion fusion proteins described herein (e.g., the Cas9 domain or the nucleotide modification domains) may be obtained as a result of mutagenizing a reference base editor (or a component or domain thereof) by a directed evolution process, e.g., a continuous evolution method (e.g., PACE) or a non-continuous evolution method (e.g., PANCE or other discrete plate-based selections). In various embodiments, the disclosure provides a base editor that has one or more amino acid variations introduced into its amino acid sequence relative to the amino acid sequence of the reference base editor. The base editor may include variants in one or more components or domains of the base editor (e.g., variants introduced into a Cas9 domain, variants introduced into a transglycosylase domain, or a variant introduced into both of these domains).

The nucleotide modification domain may be engineered in any way known to those of skill in the art. For example, the nucleotide modification domain may be evolved from a reference protein that is an RNA modifying enzyme (e.g., a tRNA guanine transglycosylase) and evolved using PACE, PANCE, or other plate-based evolution methods to obtain a DNA modifying version of the nucleotide modification domain, which can then be used in the fusion proteins described herein. For example, the disclosed transglycosylase variants may be at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the reference enzyme. In some embodiments, the transglycosylase variant may have 1, 2, 3, 4, 5, 6,7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a reference transglycosylase. In other embodiments, the transglycosylase variant comprises multiple amino acid stretches having about 99.9% identity, followed by one or more stretches having at least about 90% or at least about 95% identity, followed by stretches of having about 99.9% identity, to the corresponding amino acid sequence of the reference transglycosylase.

### Transglycosylases

In various embodiments, the TGBE (and ACBE) base editors provided herein comprise a transglycosylase nucleotide modification domain. Any transglycosylase that is adapted to accept guanine nucleotide substrates are useful in the base editors and methods of editing disclosed herein. The tranglycosylase may comprise a naturally-occurring or engineered transglycosylase, e.g. an engineered guanine transglycosylase. A guanine transglycosylase is an enzyme that catalyzes the substitution of a queuine (abbreviated Q) (or precursor of queuine) nucleobase analog for a guanine nucleobase in a polynucleotide substrate. This reaction forms a queuosine (or prequeuosine) nucleoside.

An exemplary bacterial transglycosylase, tRNA guanine transglycosylase (TGT) catalyzes the exchange of prequeuinei for guanine 34 in the UGU sequence of the anticodon loop of a tRNA. See Nonekowski, Kung & Gracia, The Escherichia coli tRNA-Guanine Transglycosylase Can Recognize and Modify DNA, J. Biol. Chem., 277(9):7178-82 (2002), incorporated herein by reference. Guanine 34 occupies the first anticodon position of the tRNA, which pairs with the third, “wobble” position in a complementary codon. The mechanism of the base exchange reaction catalyzed by E. coli TGT involves a covalent TGT-RNA complex that is thermodynamically and kinetically stable, wherein the Asp₂₆₄ residue of the enzyme is bound to the 1′ position of the ribose ring. See Garcia, Chervin & Kittendorf, Identification of the Rate-Determining Step of tRNA-Guanine Transglycosylase from Escherichia coli, Biochemistry 2009, 48, 11243-11251, incorporated herein by reference. In the next step, a 7-amino-methyl-7-deazaguanine (abbreviated preQ1) replaces the aspartate active site residue, releasing the TGT. Finally, PreQ₁ is converted to Q. When preQi is absent, TGT is also capable of using 7-cyano-7-deazaguanine (preQ₀) as the second nucleobase substrate for this reaction. PreQ₀ is a common precursor of queuosine (Q) and archaeosine (G+).

The prokaryotic TGT is capable of recognizing and exchanging a deoxyguanine nucleobase within a dU-G-dU trinucleotide sequence in a DNA hairpin substrate (dU=2′deoxyuridine). See Nonekowski, Kung & Gracia, J. Biol. Chem. (2002). This establishes that TGT recognition is not critically dependent on a ribose backbone. Further, it is demonstrated in the Examples provided herein that wild-type TGT is capable of editing target guanines in non-UGU sequences in DNA hairpins.

In eukaryotes, the preQi intermediate may be converted to a glycosylated queuosine product (glycosyl-Q).

A separate transglycosylase, the prokaryotic DpdA protein, is expressed from “gene A” located in a ˜20 kb “dpd” gene cluster that also contains preQ₀ synthesis and DNA metabolism genes. See Thiaville, et al., Novel genomic island modifies DNA with 7-deazaguanine derivatives, PNAS, 113(11):E1452-9 (2016). This gene cluster is found in genomic islands. The DpdA enzyme catalyzes the exchange of preQ₀ (or 7-amido-7-deazaguanine (ADG)) for guanine in bacterial and bacteriophage genomic DNA. The core of DpdA shows significant similarity to the TGT enzyme, as the key aspartate residues that catalyze the base exchange (Asp102 and Asp280 of Zymomonas mobilis TGT and Asp95 and Asp249 of Pyrococcus horikoshii TGT), as well as the zinc binding site (CXCXXCX₂₂H motif), are conserved in DpdAs.

Prokaryotic DpdA is capable of recognizing and exchanging a deoxyguanine nucleobase in a DNA substrate with preQ₀. The product of this base exchange reaction, dPreQ₀ nucleoside (i.e., 7-deazaguanine derivative nucleoside), were recently discovered in bacterial DNA. The product of a similar base exchange reaction, deoxyarchaeosine (dG⁺), was recently discovered in phage DNA. See id. More recently, it was confirmed that three genes of the S. Montevideo dpd gene cluster—dpd genes A, B, and C, which may encode a DpdAB complex and DpdC enzyme—are required for the formation of preQ₀ and ADG in DNA. See Yuan et al., Identification of the minimal bacterial 2′-deoxy-7-amido-7-deazaguanine synthesis machinery, Mol. Microbiol., 110(3):469-483 (2018).

The transglycosylases useful in the present disclosure may be modified from wild-type reference proteins, which include TGT and DpdA, to recognize and excise a target thymine base in DNA as a first nucleobase substrate. In the disclosed TGBEs, the target thymine is replaced with a guanine. It is believed that wild-type and evolved variant transglycosylases are capable of inserting guanine into DNA (i.e., as a second nucleobase substrate) because this step represents the chemical reverse of the first recognition step of the native guanine base excision reaction. Thus, evolved TGT and DpdA variants that recognize and excise a thymine base in DNA are provided in the present disclosure. Wild-type reference transglycosylases may be those from E. coli, S. Montevideo, bacteriophage (such as E. coli phage 9g), yeast, mouse, human, or another organism, including other bacteria and bacteriophages.

Modified transglycosylases include variants with at least 80%, at least 85%, at least 90%, at least 95% or at least 99% sequence identity to a wild-type transglycosylase. In other embodiments, modified transglycosylases may be obtained by altering or evolving a reference protein using a continuous evolution process (e.g., PACE) or non-continuous evolution process (e.g., PANCE or discrete plate-based selections) described herein so that the transglycosylase is effective on a thymine base of a nucleic acid target (e.g., a DNA target).

Based on the mechanisms elucidated immediately above with respect to wild-type TGT and DpdA base exchange involving a guanine first nucleobase substrate, the following mechanism is proposed for disclosed TGT and DpdA variants that recognize a thymine first nucleobase substrate (without wishing to be bound by any particular theory). First, the TGT (or DpdA) variant excises the thymine from 1′ position of the deoxyribose sugar and covalently bonds to the sugar, thus forming a covalent intermediate (for instance, TGT-DNA in cases where the transglycosylase is a TGT). This intermediate may be formed at an active site aspartate residue of the TGT (or DpdA) variant. Subsequently, a free guanine excises the active site residue in a nucleophilic attack, reforming a glycosidic bond.

In some embodiments (e.g., in prokaryotes), the disclosed TGT and DpdA variants uses free deazaguanine derivatives, such as PreQ₀ or PreQ₁, to excise the thymine and form a 2′-deoxy-7-cyano-7-deazaguanosine (dPreQ₀) or 2′-deoxy-7-amino-methyl-7-deazaguanosine (dPreQ₁) product. During a subsequent round of replication, the cell's mismatch repair machinery converts the dPreQ₀ or dPreQ₁ to a guanosine, thereby completing the T-to-G change. Deazaguanines and their derivatives are not normally found in eukaryotic cells. Because guanine is much more abundant in the eukaryotic nucleus than any deazaguanine derivative, this reaction is expected to proceed through a guanine nucleobase substrate in eukaryotes, and not through a deazaguanine derivative. As such, in mammalian cells, this reaction is expected to proceed through a guanine nucleobase substrate.

In certain embodiments, the transglycosylase is a bacterial TGT, or a variant thereof. Exemplary transglycosylases include, but are not limited to, E. coli TGT, Pyrococcus horikoshii TGT, Zymomonas mobilis TGT, E. coli DpdA,Salmonella enterica serovar Montevideo DpdA, Streptomyces sp. FXJ7.023 DpdA, Nocardioidaceae bacterium Broad-1 DpdA, Desulfurobacterium thermolithotrophum DpdA, Cyanothece sp. CCY0110 DpdA, E. coli phage 9g DpdA, Streptococcus pneumoniae phage Dp-1 DpdA, Mycobacterium smegmatis phage Suffolk DpdA, Mycobacterium avium phage Hedgerow DpdA, Paenibacillus glucanolyticus phage PG1 DpdA, Sulfolobus islandicus phage SIRV1 DpdA, or Bacillus cereus phage BCD7 DpdA, or a variant thereof.

### A-to-T Transversion Base Editors

In various embodiments, the present disclosure provides T-to-A (or A-to-T) transversion base editor fusion protein, such as those described in U.S. Provisional Patent Application U.S. Ser. No. 62/814,793 filed on Mar. 6, 2019, International Patent Application No. PCT/US2020/021398 filed on Mar. 6, 2020, and U.S. patent application U.S. Ser. No. 17/436,048 filed on Sep. 2, 2021, all of which are hereby incorporated by reference in their entirety.

In some embodiments, the fusion proteins compries (i) a nucleic acid programmable DNA binding protein (napDNAbp), and (ii) a nucleobase modification domain capable of facilitating the conversion of a A:T nucleobase pair to a T:A nucleobase pair in a target nucleotide sequence, e.g., a genome.

In various embodiments, the nucleobase modification domain may be an adenosine methyltransferase, which enzymatically converts an adenosine nucleoside of an A:T nucleobase pair to N1-methyladenosine, which then is subsequently processed by the cell's DNA repair and replication machinery to a thymine, thereby converting the A:T nucleobase pair to a T:A nucleobase pair.

The various domains of the transversion fusion proteins described herein (e.g., the Cas9 domain or the nucleobase modification domains) may be obtained as a result of mutagenizing a reference or starting-point base editor (or a component or domain thereof) by an evolution or modification strategy. Such strategies include a directed evolution process, e.g., a continuous evolution method (e.g., PACE) or a non-continuous evolution method (e.g., PANCE or other discrete plate-based selections). In various embodiments, the disclosure provides a base editor that has one or more amino acid variations introduced into its amino acid sequence relative to the amino acid sequence of the reference or starting-point base editor. The base editor may include variants in one or more components or domains of the base editor (e.g., variants introduced into a Cas9 domain, an adenosine methyltransferase domain, an inhibitor of DNA alkylation repair (iDAR) domain, or variants introduced into combinations of these domains). For example, the nucleobase modification domain may be evolved from a reference protein that is an RNA modifying enzyme (e.g., a mRNA or tRNA methyltransferase) and evolved using PACE, PANCE, or other plate-based evolution methods to obtain a DNA modifying version of the nucleobase modification domain, which can then be used in the fusion proteins described herein.

### Adenosine Methyltransferases

In various embodiments, the transversion base editors provided herein comprise an adenosine methyltransferase. The adenosine methyltransferase may be modified from its wild type form. Modified methyltransferases may be obtained by, e.g., evolving a reference version (e.g., an RNA modification enzyme, such as an mRNA and/or tRNA methyltransferase) using a continuous evolution process (e.g., PACE) or non-continuous evolution process (e.g., PANCE or plate-based selections) described herein so that the methyltransferase domain is effective on a nucleic acid target. See Zhang C. & Jia, G., Reversible RNA Modification N1-methyladenosine (m¹A) in mRNA and tRNA, Genomics Proteomics Bioinformatics 16:155-161 (2018), the contents of which is herein incorporated by reference in its entirety.

In some embodiments, the modification domain is a TRM61 monomer (e.g., human or S. cerevisiae), or a TRM6/61A dimer (e.g., human or S. cerevisiae), or evolved a variant thereof.

The desired adenosine methylation reaction produces an N1-methyladenosine (mlA). The presence of an adenine base on the unmutated strand induces the steric rotation of the N1-methyladenosine product to the Hoogsteen orientation in order to base pair with an adenine base on the non-edited strand. See Chawla M. et al., An atlas of RNA base pairs involving modified nucleobases with optimal geometries and accurate energies, Nucleic Acid Res. (2015), the disclosure of which is herein incorporated by reference in its entirety.

### A-to-T Transversion Base-Editors

In various embodiments, the present disclosure provides A-to-T (or T-to-A) transversion base editor fusion proteins, such as those described in U.S. Provisional Patent Application U.S. Ser. No. 62/814,800, filed Mar. 6, 2019, and International Patent Application No. PCT/US2020/021405, filed Mar. 6, 2020, both of which are herein incorporated by reference in their entirety.

In some embodiments, the fusion protein comprises (i) a nucleic acid programmable DNA binding protein (napDNAbp), and (ii) a nucleobase modification domain capable of facilitating the conversion of a A:T nucleobase pair to a T:A nucleobase pair in a target nucleotide sequence, e.g., a genome.

In various embodiments, the nucleobase modification domain may comprise a deaminase and a glycosylase, which enzymatically removes the inosine product of a catalyzed deamination of an adenine nucleobase in a A:T nucleobase pair, creating an apurinic site that may be replaced by the cell's DNA repair and replication machinery to a T:A nucleobase pair.

In various embodiments, the nucleobase modification domain is a thymine alkyltransferase, which enzymatically converts a thymine nucleobase of a T:A nucleobase pair to an alkylated thymine, which then is subsequently processed by the cell's DNA repair and replication machinery to an adenine, ultimately converting the T:A nucleobase pair to an A:T nucleobase pair.

The various domains of the transversion fusion proteins described herein (e.g., the Cas9 domain or the nucleobase modification domains) may be obtained as a result of mutagenizing a reference or starting-point base editor (or a component or domain thereof) by an evolution or modification strategy. Such strategies include a directed evolution process, e.g., a continuous evolution method (e.g., PACE) or a non-continuous evolution method (e.g., PANCE or other discrete plate-based selections). In various embodiments, the disclosure provides a base editor that has one or more amino acid variations introduced into its amino acid sequence relative to the amino acid sequence of the reference or starting-point base editor. The base editor may include variants in one or more components or domains of the base editor (e.g., variants introduced into a Cas9 domain, a deaminase domain, a glycosylase domain, a thymine alkyltransferase domain, an inhibitor of DNA alkylation repair (iDAR) domain, or variants introduced into combinations of these domains). For example, the nucleobase modification domain may be evolved from a reference protein that is a DNA modifying enzyme (e.g., a glycosylase that has as its substrate alkylated DNA) and evolved using PACE, PANCE, or other plate-based evolution methods to obtain a DNA modifying version of the nucleobase modification domain, which can then be used in the fusion proteins described herein. Alternatively, the nucleobase modification domain may be evolved from a reference protein that is an RNA modifying enzyme (e.g., uridine rRNA methyltransferases) and evolved using PACE, PANCE, or other plate-based evolution methods to obtain a DNA modifying version of the nucleobase modification domain, which can then be used in the fusion proteins described herein.

### Glycosylases

In various embodiments, the transversion base editors provided herein comprise a glycosylase. The glycosylase may be modified from its wild type form. Modified glycosylases may be obtained by, e.g., evolving a reference version (e.g., an alkylated DNA glycosylase enzyme) using a continuous evolution process (e.g., PACE) or non-continuous evolution process (e.g., PANCE or plate-based selections) described herein so that the glycosylase is effective on a nucleic acid target.

Exemplary glycosylases include, but are not limited to, a DNA glycosylase. In some embodiments, the glycosylase is an inosine excision enzyme (e.g., MPG), or an evolved variant thereof. In some embodiments, the glycosylase comprises an inosine excision enzyme and a TadA adenosine deaminase homodimer, or a variant thereof.

### Thymine Alkyltransferases

In various embodiments, the transversion base editors provided herein comprise a thymine alkyltransferase. The thymine alkyltransferase may be modified from its wild type form. Modified thymine alkyltransferases may be obtained by, e.g., evolving a reference version (e.g., an RNA modification enzyme such as a ribosomal RNA alkyltransferase) using a continuous evolution process (e.g., PACE) or non-continuous evolution process (e.g., PANCE or discrete plate-based selections) described herein so that the alkyltransferase is effective on a nucleic acid target. See Sharma et al., Identification of novel methyltransferases, Bmt5 and Bmt6, responsible for the m³U methylations of 25S rRNA in Saccharomyces cerevisiae, Nucleic Acid Res. (2014) 42(5): 3246-3260 and Meyer et al., Ribosome biogenesis factor Tsr3 is the aminocarboxypropyl transferase responsible for 18S rRNA hypermodification in yeast and humans, Nucleic Acid Res. (2016) 44(9): 4304-4316, the entire contents of each of which is herein incorporated by reference.

In some embodiments, the nucleobase modification domain is a thymine alkyltransferase (e.g., RsmE (E. coli)), or an evolved variant thereof.

The desired thymine alkylation reaction, i.e., the reaction that produces an N3-methyl-thymine, N3-carboxymethyl thymine, or N3-3-amino-3-carboxypropyl thymine product, may be selected based on the relevant enzyme and S-adenosyl-methionine (SAM) cofactor used in the reaction. To yield an N3-methyl-thymine product, an unmodified SAM is used with an Escherichia coli RsmE, a Saccharomyces cerevisiae Bmt5 or a Saccharomyces cerevisiae Bmt6, or a variant thereof. To yield an N3-3-amino-3-carboxypropyl thymine product, an unmodified SAM is used with a Tsr3 aminocaroboxypropyl transferase, or variant thereof. To yield an N3-carboxymethyl thymine, a SAM cofactor modified to include a carboxymethyl domain on the S⁺ center may be used. A variant of an Escherichia coli RsmE, a Saccharomyces cerevisiae Bmt5 or a Saccharomyces cerevisiae Bmt6 that has been evolved using a continuous evolution process (e.g., PACE) to accept a carboxylated SAM cofactor may be used.

### Additional Base Editor Elements

### Linkers

In certain embodiments, linkers may be used to link any of the peptides or peptide domains or domains of the base editor (e.g., domain A covalently linked to domain B which is covalently linked to domain C).

As defined above, the term “linker,” as used herein, refers to a chemical group or a molecule linking two molecules or domains, e.g., a binding domain and a cleavage domain of a nuclease. In some embodiments, a linker joins a gRNA binding domain of a napDNAbp and the catalytic domain of a recombinase. In some embodiments, a linker joins a dCas9 and base editor domain (e.g., an adenine deaminase). Typically, the linker is positioned between, or flanked by, two groups, molecules, or other domains and connected to each one via a covalent bond, thus connecting the two. In some embodiments, the linker is an amino acid or a plurality of amino acids (e.g., a peptide or protein). In some embodiments, the linker is an organic molecule, group, polymer, or chemical domain. Chemical domains include, but are not limited to, disulfide, hydrazone, thiol and azo domains. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. In some embodiments, the linker is a molecule in length. Longer or shorter linkers are also contemplated.

The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic domain (e.g., cyclopentane, cyclohexane). In other embodiments, the linker comprises a polyethylene glycol domain (PEG). In other embodiments, the linker comprises amino acids. In certain embodiments, the linker comprises a peptide. In certain embodiments, the linker comprises an aryl or heteroaryl domain. In certain embodiments, the linker is based on a phenyl ring. The linker may include functionalized domains to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some other embodiments, the linker comprises the amino acid sequence (GGGGS)ₙ (SEQ ID NO: 119), (G)ₙ(SEQ ID NO: 120), (EAAAK)ₙ (SEQ ID NO: 121), (GGS)ₙ (SEQ ID NO: 122), (SGGS)ₙ(SEQ ID NO: 123), (XP)ₙ (SEQ ID NO: 124), or any combination thereof, wherein n is independently an integer between 1 and 30, and wherein X is any amino acid. In some embodiments, the linker comprises the amino acid sequence (GGS)ₙ(SEQ ID NO: 125), wherein n is 1, 3, or 7. In some embodiments, the linker comprises the amino acid sequence SGSETPGTSESATPES (SEQ ID NO: 126). In some embodiments, the linker comprises the amino acid sequence SGGSSGGSSGSETPGTSESATPESSGGSSGGS (SEQ ID NO: 127). In some embodiments, the linker comprises the amino acid sequence SGGSGGSGGS (SEQ ID NO: 128). In some embodiments, the linker comprises the amino acid sequence SGGS (SEQ ID NO: 129).

In some embodiments, the fusion protein comprises the structure [domain B]-[optional linker sequence]-[domain A]-[optional linker sequence], or [domain A]-[optional linker sequence]-[domain B].

In some embodiments, the fusion protein comprises the structure [domain B]-[optional linker sequence]-[domain A]-[optional linker sequence]-[domain C]; [domain B]-[optional linker sequence]-[domain C]-[optional linker sequence]-[domain A]; [domain C]-[optional linker sequence]-[domain B]-[optional linker sequence]-[domain A]; [domain C]-[optional linker sequence]-[domain A]-[optional linker sequence]-[domain B]; [domain A]-[optional linker sequence]-[domain C]-[optional linker sequence]-[domain B]; or [domain A]-[optional linker sequence]-[domain B]-[optional linker sequence]-[domain C].

In some embodiments, the fusion protein comprises one or more nuclear localization sequences, and comprises the structure [domain B]-[optional linker sequence]-[domain A]-[optional linker sequence]-[domain C]-[optional linker sequence]-[NLS]; [NLS]-[optional linker sequence]-[domain B]-[optional linker sequence]-[domain A]-[optional linker sequence]-[domain C]; [domain B]-[optional linker sequence]-[iBER]-[optional linker sequence]-[domain A]-[optional linker sequence]-[NLS]; [NLS]-[optional linker sequence]-[domain B]-[optional linker sequence]-[domain C]-[optional linker sequence]-[domain A]; [NLS]-[optional linker sequence]-[domain C]-[optional linker sequence]-[domain B]-[optional linker sequence]-[domain A]; [domain C]-[optional linker sequence]-[domain B]-optional linker sequence]-[domain A ckase]-[optional linker sequence]-[NLS]; [domain C]-[optional linker sequence]-[domain A]-[optional linker sequence]-[domain B]-[optional linker sequence]-[NLS]; [NLS]-[optional linker sequence]-[domain C]-[optional linker sequence]-[domain A]-[optional linker sequence]-[domain B]; [NLS]-[optional linker sequence]-[domain A]-[optional linker sequence]-[domain C]-[optional linker sequence]-[domain B]; [domain A]-[optional linker sequence]-[domain C]-[optional linker sequence]-[domain B]-[optional linker sequence]-[NLS]; [NLS]-[optional linker sequence]-[domain A]-[optional linker sequence]-[domain B]-[optional linker sequence]-[domain C]; or [domain A]-[optional linker sequence]-[domain B]-[optional linker sequence]-[domain C]-[optional linker sequence]-[NLS].

### Nuclear Localization Signal

In various embodiments, the base editors disclosed herein further comprise one or more additional base editor elements, e.g., a nuclear localization signal(s), an inhibitor of base excision repair, and/or a heterologous protein domain.

In various embodiments, the base editors disclosed herein further comprise one or more, preferably, at least two nuclear localization signals. In certain embodiments, the base editors comprise at least two NLSs. In embodiments with at least two NLSs, the NLSs can be the same NLSs, or they can be different NLSs. In addition, the NLSs may be expressed as part of a fusion protein with the remaining portions of the base editors. The location of the NLS fusion can be at the N-terminus, the C-terminus, or within a sequence of a base editor (e.g., inserted between the encoded napDNAbp domain (e.g., Cas9) and a DNA nucleobase modification domain (e.g., an adenine deaminase)).

The NLSs may be any known NLS sequence in the art. The NLSs may also be any future-discovered NLSs for nuclear localization. The NLSs also may be any naturally-occurring NLS, or any non-naturally occurring NLS (e.g., an NLS with one or more desired mutations).

A nuclear localization signal or sequence (NLS) is an amino acid sequence that tags, designates, or otherwise marks a protein for import into the cell nucleus by nuclear transport. Typically, this signal consists of one or more short sequences of positively charged lysines or arginines exposed on the protein surface. Different nuclear localized proteins may share the same NLS. An NLS has the opposite function of a nuclear export signal (NES), which targets proteins out of the nucleus. A nuclear localization signal can also target the exterior surface of a cell. Thus, a single nuclear localization signal can direct the entity with which it is associated to the exterior of a cell and to the nucleus of a cell. Such sequences can be of any size and composition, for example, more than 25, 25, 15, 12, 10, 8, 7, 6, 5, or 4 amino acids, but will preferably comprise at least a four to eight amino acid sequence known to function as a nuclear localization signal (NLS).

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., International PCT application PCT/EP2000/011690, filed Nov. 23, 2000, published as WO/2001/038547 on May 31, 2001, the contents of which are incorporated herein by reference. In some embodiments, an NLS comprises the amino acid sequence PKKKRKV (SEQ ID NO: 130), MDSLLMNRRKFLYQFKNVRWAKGRRETYLC (SEQ ID NO: 131), KRTADGSEFESPKKKRKV (SEQ ID NO: 132), or KRTADGSEFEPKKKRKV (SEQ ID NO: 133). In other embodiments, NLS comprises the amino acid sequences NLSKRPAAIKKAGQAKKKK (SEQ ID NO: 134), PAAKRVKLD (SEQ ID NO: 135), RQRRNELKRSF (SEQ ID NO: 136), NQSSNFGPMKGGNFGGRSSGPYGGGGQYFAKPRNQGGY (SEQ ID NO: 137).

In one aspect of the invention, a base editor may be modified with one or more nuclear localization signals (NLS), preferably at least two NLSs. In certain embodiments, the base editors are modified with two or more NLSs. The invention contemplates the use of any nuclear localization signal known in the art at the time of the invention, or any nuclear localization signal that is identified or otherwise made available in the state of the art after the time of the instant filing. A representative nuclear localization signal is a peptide sequence that directs the protein to the nucleus of the cell in which the sequence is expressed. A nuclear localization signal is predominantly basic, can be positioned almost anywhere in a protein's amino acid sequence, generally comprises a short sequence of four amino acids (Autieri & Agrawal, (1998) J. Biol. Chem. 273: 14731-37, incorporated herein by reference) to eight amino acids, and is typically rich in lysine and arginine residues (Magin et al., (2000) Virology 274: 11-16, incorporated herein by reference). Nuclear localization signals often comprise proline residues. A variety of nuclear localization signals have been identified and have been used to effect transport of biological molecules from the cytoplasm to the nucleus of a cell. See, e.g., Tinland et al., (1992) Proc. Natl. Acad. Sci. U.S.A. 89:7442-46; Moede et al., (1999) FEBS Lett. 461:229-34, which is incorporated by reference. Translocation is currently thought to involve nuclear pore proteins.

Most NLSs can be classified in three general groups: (i) a monopartite NLS exemplified by the SV40 large T antigen NLS (PKKKRKV (SEQ ID NO: 138)); (ii) a bipartite motif consisting of two basic domains separated by a variable number of spacer amino acids and exemplified by the Xenopus nucleoplasmin NLS (KRXXXXXXXXXXKKKL (SEQ ID NO: 139), where X is any amino acid); and (iii) noncanonical sequences such as M9 of the hnRNP Al protein, the influenza virus nucleoprotein NLS, and the yeast Gal4 protein NLS (Dingwall and Laskey, 1991).

Nuclear localization signals appear at various points in the amino acid sequences of proteins. NLS's have been identified at the N-terminus, the C-terminus, and in the central region of proteins. Thus, the specification provides base editors that may be modified with one or more NLSs at the C-terminus, the N-terminus, as well as at in internal region of the base editor. The residues of a longer sequence that do not function as component NLS residues should be selected so as not to interfere, for example tonically or sterically, with the nuclear localization signal itself. Therefore, although there are no strict limits on the composition of an NLS-comprising sequence, in practice, such a sequence can be functionally limited in length and composition.

The present disclosure contemplates any suitable means by which to modify a base editor to include one or more NLSs. In one aspect, the base editors can be engineered to express a base editor protein that is translationally fused at its N-terminus or its C-terminus (or both) to one or more NLSs, i.e., to form a base editor-NLS fusion construct. In other embodiments, the base editor-encoding nucleotide sequence can be genetically modified to incorporate a reading frame that encodes one or more NLSs in an internal region of the encoded base editor. In addition, the NLSs may include various amino acid linkers or spacer regions encoded between the base editor and the N-terminally, C-terminally, or internally-attached NLS amino acid sequence, e.g, and in the central region of proteins. Thus, the present disclosure also provides for nucleotide constructs, vectors, and host cells for expressing fusion proteins that comprise a base editor and one or more NLSs.

The base editors described herein may also comprise nuclear localization signals which are linked to a base editor through one or more linkers, e.g., and polymeric, amino acid, nucleic acid, polysaccharide, chemical, or nucleic acid linker element. The linkers within the contemplated scope of the disclosure are not intended to have any limitations and can be any suitable type of molecule (e.g., polymer, amino acid, polysaccharide, nucleic acid, lipid, or any synthetic chemical linker domain) and be joined to the base editor by any suitable strategy that effectuates forming a bond (e.g., covalent linkage, hydrogen bonding) between the base editor and the one or more NLSs.

The base editors described herein also may include one or more additional elements. In certain embodiments, an additional element may comprise an effector of base repair.

In certain embodiments, the base editors described herein may comprise an inhibitor of base excision repair. The term “inhibitor of base excision repair” or “iBER” refers to a protein that is capable of inhibiting the activity of a nucleic acid repair enzyme, for example a base excision repair enzyme. Mammalian cells clear 8-oxoadenine lesions that arise naturally from oxidative DNA damage by action of thymine-DNA glycosylase (TDG), which hydrolytically cleaves the glycosidic bond of the damaged base, leaving behind an abasic site. Abasic sites are excised by AP lyase during the base excision repair process, introducing a break in the modified DNA strand. If this occurs before mismatch repair machinery locates the nick left by an nCas9 domain, as in the fusion proteins disclosed herein, in the non-edited strand, a double strand break is generated, which could lead to undesired indels during repair. Competitive base excision repair may interfere with 8-oxoadenine-mediated base editing. Accordingly, in exemplary embodiments, an iBER is fused to the fusion proteins disclosed herein, to compete for binding of the 8-oxoadenine lesion with active, endogenous excision repair enzymes, preventing or slowing base excision repair.

In some embodiments, the iBER is an inhibitor of 8-oxoadenine base excision repair. Exemplary iBERs include OGG inhibitors, MUG inhibitors, and TDG inhibitors. Exemplary iBERs include inhibitors of hOGGI, hTDG, ecMUG, APEl, Endo III, Endo IV, Endo V, Endo VIII, Fpg, hNEIL1, T7 EndoI, T4PDG, UDG, hSMUG1, and hAAG. In some embodiments, the iBER may be a catalytically inactive OGG, a catalytically inactive TDG, a catalytically inactive MUG, or small molecule or peptide inhibitor of OGG, TDG, or MUG, or a variant thereof.

In particular embodiments, the iBER is a catalytically inactive TDG. Exemplary catalytically inactive TDGs include mutagenized variants of wild-type TDG (SEQ ID NO: 140) that bind DNA nucleobases, including 8-oxoadenine, but lack DNA glycosylase activity.

TDG (human) (wild-type)
(SEQ ID NO: 140)
MEAENAGSYSLQQAQAFYTFPFQQLMAEAPNMAVVNEQQMPEEVPAPAP
AQEPVQEAPKGRKRKPRTTEPKQPVEPKKPVESKKSGKSAKSKEKQEKI
TDTFKVKRKVDRFNGVSEAELLTKTLPDILTFNLDIVIIGINPGLMAAY
KGHHYPGPGNHFWKCLFMSGLSEVQLNHMDDHTLPGKYGIGFTNMVERT
TPGSKDLSSKEFREGGRILVQKLQKYQPRIAVFNGKCIYEIFSKEVFGV
KVKNLEFGLQPHKIPDTETLCYVMPSSSARCAQFPRAQDKVHYYIKLKD
LRDQLKGIERNMDVQEVQYTFDLQLAQEDAKKMAVKEEKYDPGYEAAYG
GAYGENPCSSEPCGFSSNGLIESVELRGESAFSGIPNGQWMTQSFTDQI
PSFSNHCGTQEQEEESHA

Exemplary catalytically inactive MUGs include mutagenized variants of wild-type MUG (SEQ ID NO: 141) that bind DNA nucleobases, including 8-oxoadenine, but lack DNA glycosylase activity.

E. coli MUG (wild-type)
(SEQ ID NO: 141)
MVEDILAPGLRVVFCGINPGLSSAGTGFPFAHPANRFWKVIYQAGFTDR
QLKPQEAQHLLDYRCGVTKLVDRPTVQANEVSKQELHAGGRKLIEKIED
YQPQALAILGKQAYEQGFSQRGAQWGKQTLTIGSTQIWVLPNPSGLSRV
SLEKLVEAYRELDQALVVRGR

Some exemplary suitable inhibitors of base excision repair, that may be fused to Cas9 domains according to embodiments of this disclosure are provided below. An exemplary catalytically inactive hTDG is an N140A mutant of SEQ ID NO: 140, shown below as SEQ ID NO: 142. Analogously, an exemplary catalytically inactive ecMUG is an N18A mutant of SEQ ID NO: 141, shown below as SEQ ID NO: 143.

Catalytically inactive TDG (human)
(SEQ ID NO: 142)
MEAENAGSYSLQQAQAFYTFPFQQLMAEAPNMAVVNEQQMPEEVPAPAP
AQEPVQEAPKGRKRKPRTTEPKQPVEPKKPVESKKSGKSAKSKEKQEKI
TDTFKVKRKVDRFNGVSEAELLTKTLPDILTFNLDIVIIGIAPGLMAAY
KGHHYPGPGNHFWKCLFMSGLSEVQLNHMDDHTLPGKYGIGFTNMVERT
TPGSKDLSSKEFREGGRILVQKLQKYQPRIAVFNGKCIYEIFSKEVFGV
KVKNLEFGLQPHKIPDTETLCYVMPSSSARCAQFPRAQDKVHYYIKLKD
LRDQLKGIERNMDVQEVQYTFDLQLAQEDAKKMAVKEEKYDPGYEAAYG
GAYGENPCSSEPCGFSSNGLIESVELRGESAFSGIPNGQWMTQSFTDQI
PSFSNHCGTQEQEEESHA
Catalytically inactive E. coli MUG
(SEQ ID NO: 143)
MVEDILAPGLRVVFCGIAPGLSSAGTGFPFAHPANRFWKVIYQAGFTDR
QLKPQEAQHLLDYRCGVTKLVDRPTVQANEVSKQELHAGGRKLIEKIED
YQPQALAILGKQAYEQGFSQRGAQWGKQTLTIGSTQIWVLPNPSGLSRV
SLEKLVEAYRELDQALVVRG

Other exemplary iBERs comprise variants with at least 80%, at least 85%, at least 90%, at least 95%, or at least 99% sequence identity to wild-type hTDG and ecMUG, above. Other exemplary iBERs comprise variants with at least 80%, at least 85%, at least 90%, at least 95%, or at least 99% sequence identity to wild-type hOGGI, UDG, hSMUG1, and hAAG.

In some embodiments, the base editor described herein may comprise one or more protein domains (e.g., about or more than about 1, 2, 3, 4, 5, 6, 7, 8, 9, 10, or more domains in addition to the base editor components). A base editor may comprise any additional protein sequence, and optionally a linker sequence between any two domains. Examples of protein domains that may be fused to a base editor or component thereof (e.g., the napDNAbp domain, the nucleobase modification domain, or the NLS domain) include, without limitation, epitope tags and reporter gene sequences. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A base editor may be fused to a gene sequence encoding a protein or a fragment of a protein that bind DNA molecules or bind other cellular molecules, including, but not limited to, maltose binding protein (MBP), S-tag, Lex A DNA binding domain (DBD) fusions, GAL4 DNA binding domain fusions, and herpes simplex virus (HSV) BP16 protein fusions. Additional domains that may form part of a base editor are described in US Patent Publication No. 2011/0059502, published Mar. 10, 2011, and incorporated herein by reference in its entirety.

In an aspect of the invention, a reporter gene which includes, but is not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP), may be introduced into a cell to encode a gene product which serves as a marker by which to measure the alteration or modification of expression of the gene product. In a further embodiment of the invention, the DNA molecule encoding the gene product may be introduced into the cell via a vector. In certain embodiments of the invention the gene product is luciferase. In a further embodiment of the invention the expression of the gene product is decreased.

Other exemplary features that may be present are tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, bgh-PolyA tags, polyhistidine tags, and also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein comprises one or more His tags.

### Guide Sequence (e.g., a Guide RNA)

In various embodiments, the transversion base editors may be complexed, bound, or otherwise associated with (e.g., via any type of covalent or non-covalent bond) one or more guide sequences, i.e., the sequence which becomes associated or bound to the base editor and directs its localization to a specific target sequence having complementarity to the guide sequence or a portion thereof. The particular design embodiments of a guide sequence will depend upon the nucleotide sequence of a genomic target site of interest (i.e., the desired site to be edited) and the type of napDNAbp (e.g., type of Cas protein) present in the base editor, among other factors, such as PAM sequence locations, percent G/C content in the target sequence, the degree of microhomology regions, secondary structures, etc.

In general, a guide sequence is any polynucleotide sequence having sufficient complementarity with a target polynucleotide sequence to hybridize with the target sequence and direct sequence-specific binding of a napDNAbp (e.g., a Cas9, Cas9 homolog, or Cas9 variant) to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length.

In some embodiments, a guide sequence is less than about 75, 50, 45, 40, 35, 30, 25, 20, 15, 12, or fewer nucleotides in length. The ability of a guide sequence to direct sequence-specific binding of a base editor to a target sequence may be assessed by any suitable assay. For example, the components of a base editor, including the guide sequence to be tested, may be provided to a host cell having the corresponding target sequence, such as by transfection with vectors encoding the components of a base editor disclosed herein, followed by an assessment of preferential cleavage within the target sequence, such as by Surveyor assay as described herein. Similarly, cleavage of a target polynucleotide sequence may be evaluated in a test tube by providing the target sequence, components of a base editor, including the guide sequence to be tested and a control guide sequence different from the test guide sequence, and comparing binding or rate of cleavage at the target sequence between the test and control guide sequence reactions. Other assays are possible, and will occur to those skilled in the art.

A guide sequence may be selected to target any target sequence. In some embodiments, the target sequence is a sequence within a genome of a cell. Exemplary target sequences include those that are unique in the target genome. For example, for the S. pyogenes Cas9, a unique target sequence in a genome may include a Cas9 target site of the form MMMMMMMMNNNNNNNNNNNNXGG (SEQ ID NO: 144) where NNNNNNNNNNNNXGG (N is A, G, T, or C; and X can be anything) (SEQ ID NO: 145) has a single occurrence in the genome. A unique target sequence in a genome may include an S. pyogenes Cas9 target site of the form MMMMMMMMMNNNNNNNNNNNXGG (SEQ ID NO: 146) where NNNNNNNNNNNXGG (N is A, G, T, or C; and X can be anything) (SEQ ID NO: 147) has a single occurrence in the genome. For the S. thermophilus CRISPR1Cas9, a unique target sequence in a genome may include a Cas9 target site of the form MMMMMMMMNNNNNNNNNNNNXXAGAAW (SEQ ID NO: 148) where NNNNNNNNNNNNXXAGAAW (N is A, G, T, or C; X can be anything; and W is A or T) (SEQ ID NO: 149) has a single occurrence in the genome. A unique target sequence in a genome may include an S. thermophilus CRISPR 1 Cas9 target site of the form MMMMMMMMMNNNNNNNNNNNXXAGAAW (SEQ ID NO: 150) where NNNNNNNNNNNXXAGAAW (N is A, G, T, or C; X can be anything; and W is A or T) (SEQ ID NO: 151) has a single occurrence in the genome. For the S. pyogenes Cas9, a unique target sequence in a genome may include a Cas9 target site of the form MMMMMMMMNNNNNNNNNNNNXGGXG (SEQ ID NO: 152) where NNNNNNNNNNNNXGGXG (N is A, G, T, or C; and X can be anything) (SEQ ID NO: 153) has a single occurrence in the genome. A unique target sequence in a genome may include an S. pyogenes Cas9 target site of the form MMMMMMMMMNNNNNNNNNNNXGGXG (SEQ ID NO: 154) where NNNNNNNNNNNXGGXG (N is A, G, T, or C; and X can be anything) (SEQ ID NO: 155) has a single occurrence in the genome. In each of these sequences “M” may be A, G, T, or C, and need not be considered in identifying a sequence as unique.

In some embodiments, a guide sequence is selected to reduce the degree of secondary structure within the guide sequence. Secondary structure may be determined by any suitable polynucleotide folding algorithm. Some programs are based on calculating the minimal Gibbs free energy. An example of one such algorithm is mFold, as described by Zuker & Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see, e.g., A. R. Gruber et al., 2008, Cell 106(1): 23-24; and PA Carr & GM Church, 2009, Nature Biotechnology 27(12): 1151-62). Additional algorithms may be found in Chuai, G. et al., DeepCRISPR: optimized CRISPR guide RNA design by deep learning, Genome Biol. 19:80 (2018), and U.S. application Ser. No. 61/836,080 and U.S. Pat. No. 8,871,445, issued Oct. 28, 2014, the entireties of each of which are incorporated herein by reference.

In general, a tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a complex at a target sequence, wherein the complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. Preferred loop forming sequences for use in hairpin structures are four nucleotides in length, and most preferably have the sequence GAAA. However, longer or shorter loop sequences may be used, as may alternative sequences. The sequences preferably include a nucleotide triplet (for example, AAA), and an additional nucleotide (for example C or G). Examples of loop forming sequences include CAAA and AAAG. In an embodiment of the invention, the transcript or transcribed polynucleotide sequence has at least two or more hairpins. In certain embodiments, the transcript has two, three, four or five hairpins. In a further embodiment of the invention, the transcript has at most five hairpins. In some embodiments, the single transcript further includes a transcription termination sequence; preferably this is a polyT sequence, for example six T nucleotides. Further non-limiting examples of single polynucleotides comprising a guide sequence, a tracr mate sequence, and a tracr sequence are as follows (listed 5′ to 3′), where “N” represents a base of a guide sequence, the first block of lower case letters represent the tracr mate sequence, and the second block of lower case letters represent the tracr sequence, and the final poly-T sequence represents the transcription terminator:

(1) NNNNNNNNgtttttgtactctcaagatttaGAAAtaaatcttgcagaagctacaaagataaggctt catgccgaaatcaacaccctgtcattttatggcagggtgttttcgttatttaaTTTTTT (SEQ ID NO: 156); (2) NNNNNNNNNNNNNNNNNNgtttttgtactctcaGAAAtgcagaagctacaaagataaggcttcatgccgaaatca acaccctgtcattttatggcagggtgttttcgttatttaaTTTTTT (SEQ ID NO: 157); (3) NNNNNNNNNNNNNNNNNNNNgtttttgtactctcaGAAAtgcagaagctacaaagataaggcttcatgccgaaatca acaccctgtcattttatggcagggtgtTTTTT (SEQ ID NO: 158); (4) NNNNNNNNNNNNNNNNNNNNgttttagagctaGAAAtagcaagttaaaataaggctagtccgttatcaacttgaaaa agtggcaccgagtcggtgcTTTTTT (SEQ ID NO: 159); (5) NNNNNNNNNNNNNNNNNNNNgttttagagctaGAAATAGcaagttaaaataaggctagtccgttatcaacttgaa aaagtgTTTTTTT (SEQ ID NO: 160); and (6) NNNNNNNNNNNNNNNNNNNNgttttagagctagAAATAGcaagttaaaataaggctagtccgttatcaTTTTT TTT (SEQ ID NO: 161). In some embodiments, sequences (1) to (3) are used in combination with Cas9 from S. thermophilus CRISPR1. In some embodiments, sequences (4) to (6) are used in combination with Cas9 from S. pyogenes. In some embodiments, the tracr sequence is a separate transcript from a transcript comprising the tracr mate sequence.

It will be apparent to those of skill in the art that in order to target any of the fusion proteins comprising a Cas9 domain and an DNA nucleobase modification domain, as disclosed herein, to a target site, e.g., a site comprising a point mutation to be edited, it is typically necessary to co-express the fusion protein together with a guide RNA, e.g., an sgRNA. As explained in more detail elsewhere herein, a guide RNA typically comprises a tracrRNA framework allowing for Cas9 binding, and a guide sequence, which confers sequence specificity to the Cas9:nucleic acid editing enzyme/domain fusion protein.

In some embodiments, the guide RNA comprises a structure 5′-[guide sequence]-guuuuagagcuagaaauagcaaguuaaaauaaggcuaguccguuaucaacuugaaaaaguggcaccgagucggugcuuuuu-3′ (SEQ ID NO: 162), wherein the guide sequence comprises a sequence that is complementary to the target sequence. See U.S. Publication No. 2015/0166981, published Jun. 18, 2015, the disclosure of which is incorporated by reference herein in its entirety. The guide sequence is typically 20 nucleotides long. The sequences of suitable guide RNAs for targeting Cas9:nucleic acid editing enzyme/domain fusion proteins to specific genomic target sites will be apparent to those of skill in the art based on the instant disclosure. Such suitable guide RNA sequences typically comprise guide sequences that are complementary to a nucleic sequence within 50 nucleotides upstream or downstream of the target nucleotide to be edited. Some exemplary guide RNA sequences suitable for targeting any of the provided fusion proteins to specific target sequences are provided herein. Additional guide sequences are well known in the art and may be used with the base editors described herein. Additional exemplary guide sequences are disclosed in, for example, Jinek M., et al., Science 337:816-821(2012); Mali P, Esvelt K M & Church G M (2013) Cas9 as a versatile tool for engineering biology, Nature Methods, 10, 957-963; Li J F et al., (2013) Multiplex and homologous recombination-mediated genome editing in Arabidopsis and Nicotiana benthamiana using guide RNA and Cas9, Nature Biotechnology, 31, 688-691; Hwang, W. Y. et al., Efficient genome editing in zebrafish using a CRISPR-Cas system, Nature Biotechnology 31, 227-229 (2013); Cong L et al., (2013) Multiplex genome engineering using CRIPSR/Cas systems, Science, 339, 819-823; Cho S W et al., (2013) Targeted genome engineering in human cells with the Cas9 RNA-guided endonuclease, Nature Biotechnology, 31, 230-232; Jinek, M. et al., RNA-programmed genome editing in human cells, eLife 2, e00471 (2013); Dicarlo, J. E. et al., Genome engineering in Saccharomyces cerevisiae using CRISPR-Cas systems. Nucleic Acid Res. (2013); Briner A E et al., (2014) Guide RNA functional modules direct Cas9 activity and orthogonality, Mol Cell, 56, 333-339, the entire contents of each of which are herein incorporated by reference.

### Increasing Expression

The invention relates in various aspects to methods of making the disclosed base editors by various modes of manipulation that include, but are not limited to, codon optimization of one or more domains of the base editors (e.g., of an adenine deaminase) to achieve greater expression levels in a cell. The base editors contemplated herein can include modifications that result in increased expression through codon optimization and ancestral reconstruction analysis.

In some embodiments, the base editors (or a component thereof) is codon optimized for expression in particular cells, such as eukaryotic cells. The eukaryotic cells may be those of or derived from a particular organism, such as a mammal, including, but not limited to, human, mouse, rat, rabbit, dog, or non-human primate. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database,” and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a CRISPR enzyme correspond to the most frequently used codon for a particular amino acid. In some embodiments, nucleic acid constructs are codon-optimized for expression in HEK293T cells. In some embodiments, nucleic acid constructs are codon-optimized for expression in human cells.

In other embodiments, the base editors of the invention have improved expression (as compared to non-modified or state of the art counterpart editors) as a result of ancestral sequence reconstruction analysis. Ancestral sequence reconstruction (ASR) is the process of analyzing modern sequences within an evolutionary/phylogenetic context to infer the ancestral sequences at particular nodes of a tree. These ancient sequences are most often then synthesized, recombinantly expressed in laboratory microorganisms or cell lines, and then characterized to reveal the ancient properties of the extinct biomolecules. This process has produced tremendous insights into the mechanisms of molecular adaptation and functional divergence. Despite such insights, a major criticism of ASR is the general inability to benchmark accuracy of the implemented algorithms. It is difficult to benchmark ASR for many reasons. Notably, genetic material is not preserved in fossils on a long enough time scale to satisfy most ASR studies (many millions to billions of years ago), and it is not yet physically possible to travel back in time to collect samples. Reference can be made to Cai et al., “Reconstruction of ancestral protein sequences and its applications,” BMC Evolutionary Biology 2004, 4:33; and Zakas et al., “Enhancing the pharmaceutical properties of protein drugs by ancestral sequence reconstruction,” Nature Biotechnology, 35-37 (2017), each of which are incorporated herein by reference.

There are many software packages available which can perform ancestral state reconstruction. Generally, these software packages have been developed and maintained through the efforts of scientists in related fields and released under free software licenses. The following list is not meant to be a comprehensive itemization of all available packages, but provides a representative sample of the extensive variety of packages that implement methods of ancestral reconstruction with different strengths and features: PAML (Phylogenetic Analysis by Maximum Likelihood, available at //abacus.gene.ucl.ac.uk/software/paml.html), BEAST (Bayesian evolutionary analysis by sampling trees, available at //www.beast2.org/wiki/index.php/Main_Page), and Diversitree (FitzJohn RG, 2012. Diversitree: comparative phylogenetic analyses of diversification in R. Methods in Ecology and Evolution), and HyPHy (Hypothesis testing using phylogenies, available at //hyphy.org/w/index.php/Main_Page).

The above description is meant to be non-limiting with regard to making base editors having increased expression, and thereby increase editing efficiencies.

### Increasing Base Editor Efficiencies

Some embodiments of the disclosure are based on the recognition that any of the base editors provided herein are capable of modifying a specific nucleobase without generating a significant proportion of indels. An “indel”, as used herein, refers to the insertion or deletion of a nucleobase within a nucleic acid. Such insertions or deletions can lead to frame shift mutations within a coding region of a gene. In some embodiments, it is desirable to generate base editors that efficiently modify (e.g., oxidize) a specific nucleotide within a nucleic acid, without generating a large number of insertions or deletions (i.e., indels) in the nucleic acid. In certain embodiments, any of the base editors provided herein are capable of generating a greater proportion of intended modifications (e.g., point mutations) versus indels. In some embodiments, the base editors provided herein are capable of generating a ratio of intended point mutations to indels that is greater than 1:1. In some embodiments, the base editors provided herein are capable of generating a ratio of intended point mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more. The number of intended mutations and indels may be determined using any suitable method, for example the methods used in the below Examples. In some embodiments, to calculate indel frequencies, sequencing reads are scanned for exact matches to two 10-bp sequences that flank both sides of a window in which indels might occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively.

In some embodiments, the base editors provided herein are capable of limiting formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor. In some embodiments, any of the base editors provided herein are capable of limiting the formation of indels at a region of a nucleic acid to less than 1%, less than 1.5%, less than 2%, less than 2.5%, less than 3%, less than 3.5%, less than 4%, less than 4.5%, less than 5%, less than 6%, less than 7%, less than 8%, less than 9%, less than 10%, less than 12%, less than 15%, or less than 20%. The number of indels formed at a nucleic acid region may depend on the amount of time a nucleic acid (e.g., a nucleic acid within the genome of a cell) is exposed to a base editor. In some embodiments, a number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a nucleic acid (e.g., a nucleic acid within the genome of a cell) to a base editor.

Some embodiments of the disclosure are based on the recognition that any of the base editors provided herein are capable of efficiently generating an intended mutation, such as a point mutation, in a nucleic acid (e.g., a nucleic acid within a genome of a subject) without generating a significant number of unintended mutations, such as unintended point mutations. In some embodiments, an intended mutation is a mutation that is generated by a specific base editor bound to a gRNA, specifically designed to generate the intended mutation. In some embodiments, the intended mutation is a mutation associated with a disease, disorder, or condition. In some embodiments, the intended mutation is an adenine (A) to cytosine (C) point mutation associated with a disease, disorder, or condition. In some embodiments, the intended mutation is a thymine (T) to guanine (G) point mutation associated with a disease, disorder, or condition. In some embodiments, the intended mutation is an adenine (A) to cytosine (C) point mutation within the coding region of a gene. In some embodiments, the intended mutation is a thymine (T) to guanine (G) point mutation within the coding region of a gene. In some embodiments, the intended mutation is a point mutation that generates a stop codon, for example, a premature stop codon within the coding region of a gene. In some embodiments, the intended mutation is a mutation that eliminates a stop codon. In some embodiments, the intended mutation is a mutation that alters the splicing of a gene. In some embodiments, the intended mutation is a mutation that alters the regulatory sequence of a gene (e.g., a gene promotor or gene repressor). In some embodiments, any of the base editors provided herein are capable of generating a ratio of intended mutations to unintended mutations (e.g., intended point mutations:unintended point mutations) that is greater than 1:1. In some embodiments, any of the base editors provided herein are capable of generating a ratio of intended mutations to unintended mutations (e.g., intended point mutations:unintended point mutations) that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 150:1, at least 200:1, at least 250:1, at least 500:1, or at least 1000:1, or more.

Some embodiments of the disclosure are based on the recognition that the formation of indels in a region of a nucleic acid may be limited by nicking the non-edited strand opposite to the strand in which edits are introduced. This nick serves to direct mismatch repair machinery to the non-edited strand, ensuring that the chemically modified nucleobase is not interpreted as a lesion by the machinery. This nick may be created by the use of an nCas9. The methods provided in this disclosure comprise cutting (or nicking) the non-edited strand of the double-stranded DNA, for example, wherein the one strand comprises the T of the target A:T nucleobase pair. It should be appreciated that the characteristics of the base editors described in the “Editing DNA or RNA” section, herein, may be applied to any of the fusion proteins, or methods of using the fusion proteins provided herein.

### Vectors and Reagents

Several embodiments of the making and using of the base editors of the invention relate to vector systems comprising one or more vectors, or vectors as such. Vectors may be designed to clone and/or express the base editors as disclosed herein. Vectors may also be designed to clone and/or express one or more gRNAs having complementarity to the target sequence, as disclosed herein. Vectors may also be designed to transfect the base editors and gRNAs of the disclosure into one or more cells, e.g., a target diseased eukaryotic cell for treatment with the base editor systems and methods disclosed herein.

Vectors can be designed for expression of base editor transcripts (e.g., nucleic acid transcripts, proteins, or enzymes) in prokaryotic or eukaryotic cells. For example, base editor transcripts can be expressed in bacterial cells such as Escherichia coli, insect cells (using baculovirus expression vectors), yeast cells, or mammalian cells. Suitable host cells are discussed further in Goeddel, Gene Expression Technology: Methods in Enzymology 185, Academic Press. San Diego, Calif. (1990). Alternatively, expression vectors encoding one or more base editors described herein can be transcribed and translated in vitro, for example using T7 promoter regulatory sequences and T7 polymerase.

Vectors may be introduced and propagated in prokaryotic cells. In some embodiments, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). In some embodiments, a prokaryote is used to amplify copies of a vector and express one or more nucleic acids, such as to provide a source of one or more proteins for delivery to a host cell or host organism. Expression of proteins in prokaryotes is most often carried out in Escherichia coli with vectors containing constitutive or inducible promoters directing the expression of either fusion or non-fusion proteins.

Fusion expression vectors also may be used to express the base editors of the disclosure. Such vectors generally add a number of amino acids to a protein encoded therein, such as to the amino terminus of the recombinant protein. Such fusion vectors may serve one or more purposes, such as: (i) to increase expression of a recombinant protein; (ii) to increase the solubility of a recombinant protein; and (iii) to aid in the purification of a recombinant protein by acting as a ligand in affinity purification. Often, in fusion expression vectors, a proteolytic cleavage site is introduced at the junction of the fusion domain and the recombinant protein to enable separation of the recombinant protein from the fusion domain subsequent to purification of the fusion protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., Gene Expression Technology: Methods in Enzymology 185, Academic Press, San Diego, Calif. (1990) 60-89).

In some embodiments, a vector is a yeast expression vector for expressing the base editors described herein. Examples of vectors for expression in yeast Saccharomyces cerivisae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

In some embodiments, a vector drives protein expression in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In some embodiments, a vector is capable of driving expression of one or more sequences in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., Molecular Cloning: A Laboratory Manual. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter, U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

### Pharmaceutical Compositions

Other embodiments of the present disclosure relate to pharmaceutical compositions comprising any of the fusion proteins or the fusion protein-gRNA complexes described herein.

The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g., for specific delivery, increasing half-life, or other therapeutic compounds).

In some embodiments, any of the fusion proteins, gRNAs, and/or complexes described herein are provided as part of a pharmaceutical composition. In some embodiments, the pharmaceutical composition comprises any of the fusion proteins provided herein. In some embodiments, the pharmaceutical composition comprises any of the complexes provided herein. In some embodiments pharmaceutical composition comprises a gRNA, a napDNAbp-dCas9 fusion protein, and a pharmaceutically acceptable excipient. In some embodiments pharmaceutical composition comprises a gRNA, a napDNAbp-nCas9 fusion protein, and a pharmaceutically acceptable excipient. Pharmaceutical compositions may optionally comprise one or more additional therapeutically active substances.

In some embodiments, compositions provided herein are administered to a subject, for example, to a human subject, in order to effect a targeted genomic modification within the subject. In some embodiments, cells are obtained from the subject and contacted with any of the pharmaceutical compositions provided herein. In some embodiments, cells removed from a subject and contacted ex vivo with a pharmaceutical composition are re-introduced into the subject, optionally after the desired genomic modification has been effected or detected in the cells. Methods of delivering pharmaceutical compositions comprising nucleases are known, and are described, for example, in U.S. Pat. Nos. 6,453,242; 6,503,717; 6,534,261; 6,599,692; 6,607,882; 6,689,558; 6,824,978; 6,933,113; 6,979,539; 7,013,219; and 7,163,824, the disclosures of all of which are incorporated by reference herein in their entireties. Although the descriptions of pharmaceutical compositions provided herein are principally directed to pharmaceutical compositions which are suitable for administration to humans, it will be understood by the skilled artisan that such compositions are generally suitable for administration to animals or organisms of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, domesticated animals, pets, and commercially relevant mammals such as cattle, pigs, horses, sheep, cats, dogs, mice, and/or rats; and/or birds, including commercially relevant birds such as chickens, ducks, geese, and/or turkeys.

Formulations of the pharmaceutical compositions described herein may be prepared by any method known or hereafter developed in the art of pharmacology. In general, such preparatory methods include the step of bringing the active ingredient(s) into association with an excipient and/or one or more other accessory ingredients, and then, if necessary and/or desirable, shaping and/or packaging the product into a desired single- or multi-dose unit.

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro (Lippincott, Williams & Wilkins, Baltimore, MD, 2006; incorporated in its entirety herein by reference) discloses various excipients used in formulating pharmaceutical compositions and known techniques for the preparation thereof. See also PCT application PCT/US2010/055131 (Publication No. WO/2011053982), filed Nov. 2, 2010, which is incorporated herein by reference, for additional suitable methods, reagents, excipients and solvents for producing pharmaceutical compositions comprising a nuclease. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

As used here, the term “pharmaceutically acceptable carrier” means a pharmaceutically acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants may also be present in the formulation. The terms such as “excipient”, “carrier”, “pharmaceutically acceptable carrier” or the like are used interchangeably herein.

In some embodiments, the pharmaceutical composition is formulated for delivery to a subject, e.g., for gene editing. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site. In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the invention in lyophilized form and (b) a second container containing a pharmaceutically acceptable diluent (e.g., sterile water) for injection. The pharmaceutically acceptable diluent can be used for reconstitution or dilution of the lyophilized compound of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierceable by a hypodermic injection needle. The active agent in the composition is a compound of the invention. In some embodiments, the label on or associated with the container indicates that the composition is used for treating the disease of choice. The article of manufacture may further comprise a second container comprising a pharmaceutically acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

### Kits and Cells

This disclosure provides kits comprising any one of the compositions, complexes, gRNAs, polynucleotides, vectors, and/or cells disclosed herein. Some embodiments of this disclosure provide kits comprising a nucleic acid construct comprising a nucleotide sequence encoding an enzyme domain-napDNAbp fusion protein capable inserting a single transition and/or transversion mutation into a DNA sequence encoding an endogenous tRNA. In some embodiments, the nucleotide sequence encodes any of the enzyme domains provided herein. In some embodiments, the nucleotide sequence comprises a heterologous promoter that drives expression of the fusion protein. The nucleotide sequence may further comprise a heterologous promoter that drives expression of the gRNA, or a heterologous promoter that drives expression of the fusion protein and the gRNA.

In some embodiments, the kit further comprises an expression construct encoding a guide nucleic acid backbone, e.g., a guide RNA backbone, wherein the construct comprises a cloning site positioned to allow the cloning of a nucleic acid sequence identical or complementary to a target sequence into the guide nucleic acid, e.g., guide RNA backbone. In some embodiments, the kit further comprises an expression construct comprising a nucleotide sequence encoding an iBER.

The disclosure further provides kits comprising a fusion protein as provided herein, a gRNA having complementarity to a target sequence, and one or more of the following: cofactor proteins, buffers, media, and target cells (e.g. human cells). Kits may comprise combinations of several or all of the aforementioned components.

Some embodiments of this disclosure provide cells comprising any of the polynucleotides, complexes, gRNAs, and/or vectors disclosed herein. In some embodiments, the cells comprise a nucleotide that encodes any of the fusion proteins provided herein. In some embodiments, the cells comprise any of the nucleotides or vectors provided herein.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, ClR, Rat6, CV1, RPTE, A10, T24, J82, A375, ARH-77, Calul, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A 172, A20, A253, A431, A-549, ALC, B16, B35, BCP-1 cells, BEAS-2B, bEnd.3, BHK-21, BR 293. BxPC3. C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalclc7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK 11, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

eVLPs

Aspects of the present disclosure further relate to eVLPs, for example, to deliver the base editors to a subject in need thereof. In various embodiments, the eVLPs (e.g., BE-VLPs) consist of a supra-molecular assembly comprising (a) an envelope comprising (i) a lipid membrane (e.g., single-layer or bi-layer membrane) and a (ii) viral envelope glycoprotein and (b) a multi-protein core region enclosed by the envelope and comprising (i) a Gag protein, (ii) a Gag-Pro-Pol protein (with the “Pro” component bi (, and (iii) a Gag-cargo fusion protein comprising a Gag protein fused to a cargo protein (e.g., a napDNAbp or BE) via a cleavable linker (e.g., a protease-cleavable linker). In various embodiments, the cargo protein is a napDNAbp (e.g., Cas9). In other embodiments, the cargo protein is a base editor. In various other embodiments, the multi-protein core region of the VLPs further comprises one or more guide RNA molecules which are complexed with the napDNAbp or the base editor to form a ribonucleoprotein (RNP). In various embodiments, the VLPs are prepared in a producer cell that is transiently transformed with plasmid DNA that encodes the various protein and nucleic acid (sgRNA) components of the VLPs. Without being bound by theory, the components self-assemble at the cell membrane and bud out in accordance with the naturally occurring mechanism of budding (e.g., retroviral budding or the budding mechanism of other envelope viruses) in order to release from the cell fully-matured VLPs. Once formed, the Gag-Pol-Pro cleaves the protease-sensitive linker of the Gag-cargo (i.e., [Gag]-[cleavable linker]-[cargo], wherein the cargo can be BE-RNP or a napDNAbp RNP) thereby releasing the BE RNP and/or napDNAbp RNA, as the case may be, within the VLP. Once the VLP is administered to a recipient cell and taken up by said recipient cell, the contents of the VLP are released, e.g., released BE RNP and/or napDNAbp RNP. Once in the cell, the RNPs may translocate to the nuclease of the cell (in particular, where NLSs are included on the RNPs), where DNA editing may occur at target sites specified by the guide RNA. Various embodiments comprise one or more improvements.

In one improvement, the protease-cleavable linker is optimized to improve cleavage efficiency after VLP maturation, as demonstrated herein for v.2 VLPs (or “second generation” VLPs).

In another improvement, the Gag-cargo fusion (e.g., Gag-BE) further comprises one or more nuclear export signals at one or more locations along the length of the fusion polypeptide protein which may be joined by a cleavable linker such that during VLP assembly in the producer cell, the Gag-cargo fusions (due to presence of competing NLS signals) do not accumulate in the nucleus of the producer cells but instead are available in the cytoplasm to undergo the VLP assembly process at the cell membrane. Once inside the matured VLPs following release from the producer cell, the NES may be cleaved by Gag-Pro-Pol thereby separating the cargo (e.g., napDNAbp or a BE) from the NES. Upon delivery to a recipient cell, therefore, the cargo (e.g., napDNAbp or BE, typically flanked with one or more NLS elements) will not comprise an NES element, which may otherwise prohibit the transport of the cargo into the nuclease and hinder gene editing activity. This is exemplified as v.3 VLPs described herein (or “third generation” VLPs).

In another improvement, as demonstrated by v.4 VLPs (or “fourth generation” VLPs) described herein, the inventors found an optimized stoichiometry ratio of Gag-cargo fusion to Gag-Pro-Pol fusion protein which balances the amount of Gag-cargo available to be packaged into VLPs with the amount of retrovirus protease (the “Pro” in the Gag-Pro-Pol fusion) required for VLP maturation. In one embodiment, the optimized ratio of Gag-cargo fusion to Gag-Pro-Pol fusion protein is achieved by the appropriate ratio of plasmids encoding each component which are transiently delivered to the producer cells. In one embodiment, to modulate the stoichiometry of the Gag-cargo fusion to Gag-Pro-Pol fusion, the ratio of the plasmid encoding Gag-cargo (e.g., Gag-3xNES-ABE8e) to wild-type MMLV gag-pro-pol plasmids transfected for VLP production was varied. It was found that increasing the amount of gag-cargo plasmid beyond the original proportion used for producing v3.4 BE-eVLPs (38% Gag-cargo plasmid and 62% gag-pro-pol plasmid) did not improve editing efficiencies. Decreasing the proportion of gag-cargo plasmid from 38% to 25% modestly improved editing efficiencies. However, further decreasing the proportion of gag-cargo plasmid below 25% reduced editing efficiencies. These results are consistent with a model in which an optimal gag-cargo:gag-pro-pol stoichiometry balances the amount of gag-cargo available to be packaged into VLPs with the amount of MMLV protease (the “pro” in gag-pro-pol) required for VLP maturation. In one embodiment, the results of this final round of optimization revealed a fourth-generation (v4) BE-eVLP formulation, which combines the optimal gag-BE:gag-pro-pol stoichiometry (25% gag-BE) with the v3.4 BE-eVLP architecture.

Accordingly, in one aspect, the present disclosure provides a eVLP comprising an (a) envelope and (b) a multi-protein core, wherein the envelope comprises a lipid membrane (e.g., a lipid mono or bi-layer membrane) and a viral envelope glycoprotein and wherein the multi-protein core comprises a Gag (e.g., a retroviral Gag), a group-specific antigen (gag) protease (pro) polyprotein (i.e., “Gag-Pro-Pol”) and a fusion protein comprising a Gag-cargo (e.g., Gag-napDNAbp or Gag-BE). In various embodiments, the Gag-cargo may comprise a ribonucleoprotein cargo, e.g., a napDNAbp or a BE complexed with a guide RNA. In still further embodiments, the Gag-cargo (e.g., Gag fused to a napDNAbp or a BE) may comprise one or more NLS sequences and/or one or more NES sequences to regulate the cellular location of the cargo in a cell. An NLS sequence will facilitate the transport of the cargo into the cell's nuclease to facilitate editing. A NES will do the opposite, i.e., transport the cargo out from the nucleus, and/or prevent the transport of the cargo into the nucleus. In certain embodiments, the NES may be coupled to the fusion protein by a cleavable linker (e.g., a protease linker) such that during assembly in a producer cell, the NES signals operates to keep the cargo in the cytoplasm and available for the packaging process. However, once matured VLPs are budded out or released from a producer cell in a mature form, the cleavable linker joining the NES may be cleaved, thereby removing the association of NES with the cargo. Thus, without an NES, the cargo will translocate to the nuclease with its NLS sequences, thereby facilitating editing. Various napDNAbps may be used in the systems of the present disclosure. In some embodiments, the napDNAbp is a Cas9 protein (e.g., a Cas9 nickase, dead Cas9 (dCas9), or another Cas9 variant as described herein). In some embodiments, the Cas9 protein is bound to a guide RNA (gRNA). The fusion protein may further comprise other protein domains, such as effector domains. In some embodiments, the fusion protein further comprises a deaminase domain (e.g., an adenosine deaminase domain or a cytosine deaminase domain). In certain embodiments, the fusion protein comprises a base editor, such as ABE8e, or any of the other base editors described herein or known in the art.

In some embodiments, the fusion protein comprises more than one NES (e.g., two NES, three NES, four NES, five NES, six NES, seven NES, eight NES, nine NES, or ten or more NES). In certain embodiments, the fusion protein further comprises a nuclear localization sequence (NLS), or more than one NLS (e.g., two NLS, three NLS, four NLS, five NLS, six NLS, seven NLS, eight NLS, nine NLS, or ten or more NLS). In certain embodiments, the fusion protein may comprising at least one NES and one NLS.

The Gag-cargo fusion proteins described herein comprise one or more cleavable linkers. In one embodiment, the Gag-cargo fusion proteins comprise a cleavable linker joining the Gag to the cargo, such that once the Gag-cargo fusion has been packaged in mature VLPs (which will also contain the Gag-Pro-Pol, the protease activity can cleave the Gag-cargo cleavable linker, thereby releasing the cargo. In some embodiments, a cleavable linker may also be provided in such a location such that when the cleavable linker is cleaved (e.g., by the Gag-Pro-Pol protein), the NES is separated away from the cargo protein. Such an arrangement of the fusion protein allows the fusion protein to be exported from the nucleus of a producing cell during BE-VLP production, and the NES can later be cleaved from the fusion protein after delivery to a target cell, releasing the BE and allowing it to enter the nucleus of the target cell. In some embodiments, the cleavable linker comprises a protease cleavage site (e.g., a Moloney murine leukemia virus (MMLV) protease cleavage site or a Friend murine leukemia virus (FMLV) protease cleavage site). Various protease cleavage sites can be used in the fusion proteins of the present disclosure. In certain embodiments, the protease cleavage site comprises the amino acid sequence TSTLLMENSS (SEQ ID NO: 163), PRSSLYPALTP (SEQ ID NO: 164), VQALVLTQ (SEQ ID NO: 165), PLQVLTLNIERR (SEQ ID NO: 166), or an amino acid sequence at least 90% identical to any one of SEQ ID NOs: 163-166. In some embodiments, the cleavable linker of the fusion protein is cleaved by the protease of the gag-pro polyprotein. In certain embodiments, the cleavable linker of the fusion protein is not cleaved by the protease of the gag-pro polyprotein until the BE-VLP has been assembled and delivered into a target cell. In some embodiments, the gag-pro polyprotein of the BE-VLPs described herein comprises an MMLV gag-pro polyprotein or an FMLV gag-pro polyprotein. In some embodiments, the gag nucleocapsid protein of the fusion protein in the BE-VLPs described herein comprises an MMLV gag nucleocapsid protein or an FMLV gag nucleocapsid protein.

In certain embodiments, the fusion protein comprises the following non-limiting structures:
[gag nucleocapsid protein]-[1X-3X NES]-[cleavable linker]-[NLS]-[deaminase domain]-[napDNAbp]-[NLS], wherein]-[comprises an optional linker (e.g., an amino acid linker, or any of the linkers provided herein);
[1X-3X NES]-[gag nucleocapsid protein]-[cleavable linker]-[NLS]-[deaminase domain]-[napDNAbp]-[NLS], wherein]-[comprises an optional linker (e.g., an amino acid linker, or any of the linkers provided herein); or
[gag nucleocapsid protein]-[1X-3X NES]-[cleavable linker]-[NLS]-[deaminase domain]-[napDNAbp]-[NLS]-[cleavable linker]-[1X-3X NES], wherein]-[comprises an optional linker (e.g., an amino acid linker, or any of the linkers provided herein).

The eVLPs (e.g., the BE-VLPs) provided by the present disclosure comprise an outer encapsulation layer (or envelope layer) comprising a viral envelope glycoprotein. Any viral envelope glycoprotein described herein, or known in the art, may be used in the BE-VLPs of the present disclosure. In some embodiments, the viral envelope glycoprotein is an adenoviral envelope glycoprotein, an adeno-associated viral envelope glycoprotein, a retroviral envelope glycoprotein, or a lentiviral envelope glycoprotein. In certain embodiments, the viral envelope glycoprotein is a retroviral envelope glycoprotein. In some embodiments, the viral envelope glycoprotein is a vesicular stomatitis virus G protein (VSV-G), a baboon retroviral envelope glycoprotein (BaEVRless), a FuG-B2 envelope glycoprotein, an HIV-1 envelope glycoprotein, or an ecotropic murine leukemia virus (MLV) envelope glycoprotein. In some embodiments, the viral envelope glycoprotein targets the system to a particular cell type (e.g., immune cells, neural cells, retinal pigment epithelium cells, etc.). For example, using different envelope glycoproteins in the eVLPs described herein may alter their cellular tropism, allowing the BE-VLPs to be targeted to specific cell types. In some embodiments, the viral envelope glycoprotein is a VSV-G protein, and the VSV-G protein targets the system to retinal pigment epithelium (RPE) cells. In some embodiments, the viral envelope glycoprotein is an HIV-1 envelope glycoprotein, and the HIV-1 envelope glycoprotein targets the system to CD4+ cells. In some embodiments, the viral envelope glycoprotein is a FuG-B2 envelope glycoprotein, and the FuG-B2 envelope glycoprotein targets the system to neurons.

It will be appreciated that general methods are known in the art for producing viral vector particles, which generally contain coding nucleic acids of interest, may also be used for producing the virus-derived particles according to the present invention, which do not contain coding nucleic acids of interest but instead are designed to deliver a protein cargo (e.g., a BE RNP).

Conventional viral vector particles encompass retroviral, lentiviral, adenoviral and adeno-associated viral vector particles that are well known in the art. For a review of various viral vector particles that may be used, the one skilled in the art may notably refer to Kushnir et al. (2012, Vaccine, Vol. 31: 58-83), Zeltons (2013, Mol Biotechnol, Vol. 53: 92-107), Ludwig et al. (2007, Curr Opin Biotechnol, Vol. 18(no 6): 537-55) and Naskalaska et al. (2015, Polish Journal of Microbology, Vol. 64 (no 1): 3-13). Further, references to various methods using virus-derived particles for delivering proteins to cells are found by the one skilled in the art in the article of Maetzig et al. (2012, Current Gene Therapy, Vol. 12: 389-409) as well as the article of Kaczmarczyk et al. (2011, Proc Natl Acad Sci USA, Vol. 108 (no 41): 16998-17003).

Generally, a virus-like particle that is used according to the present disclosure, which virus-like particle may also be termed “virus-derived particle,” is formed by one or more virus-derived structural protein(s) and/or one more virus-derived envelope protein.

A virus-like particle that is used according to the present invention is replication incompetent in a host cell wherein it has entered.

In preferred embodiments, a virus-like particle is formed by one or more retrovirus-derived structural protein(s) and optionally one or more virus-derived envelope protein(s).

In preferred embodiments, the virus-derived structural protein is a retroviral Gag protein or a peptide fragment thereof. As it is known in the art, Gag and Gag/pol precursors are expressed from full length genomic RNA as polyproteins, which require proteolytic cleavage, mediated by the retroviral protease (PR), to acquire a functional conformation. Further, Gag, which is structurally conserved among the retroviruses, is composed of at least three protein units: matrix protein (MA), capsid protein (CA) and nucleocapsid protein (NC), whereas Pol consists of the retroviral protease, (PR), the retrotranscriptase (RT) and the integrase (IN).

In some embodiments, a virus-derived particle comprises a retroviral Gag protein but does not comprise a Pol protein.

As it is known in the art, the host range of retroviral vector, including lentiviral vectors, may be expanded or altered by a process known as pseudotyping. Pseudotyped lentiviral vectors consist of viral vector particles bearing glycoproteins derived from other enveloped viruses. Such pseudotyped viral vector particles possess the tropism of the virus from which the glycoprotein is derived.

In some embodiments, a virus-like particle is a pseudotyped virus-like particle comprising one or more viral structural protein(s) or viral envelope protein(s) imparting a tropism to the said virus-like particle for certain eukaryotic cells. A pseudotyped virus-like particle as described herein may comprise, as the viral protein used for pseudotyping, a viral envelope protein selected in a group comprising VSV-G protein, Measles virus HA protein, Measles virus F protein, Influenza virus HA protein, Moloney virus MLV-A protein, Moloney virus MLV-E protein, Baboon Endogenous retrovirus (BAEV) envelope protein, Ebola virus glycoprotein and foamy virus envelope protein, or a combination of two or more of these viral envelope proteins.

A well-known illustration of pseudotyping viral vector particles consists of the pseudotyping of viral vector particles with the vesicular stomatitis virus glycoprotein (VSV-G). For the pseudotyping of viral vector particles, the one skilled in the art may notably refer to Yee et al. (1994, Proc Natl Acad Sci, USA, Vol. 91: 9564-9568) Cronin et al. (2005, Curr Gene Ther, Vol. 5(no 4): 387-398), which are incorporated herein by reference.

For producing virus-like particles, and more precisely VSV-G pseudotypes virus-like particles, for delivering protein(s) of interest into target cells, the one skilled in the art may refer to Mangeot et al. (2011, Molecular Therapy, Vol. 19 (no 9): 1656-1666).

In some embodiments, a virus-like particle further comprises a viral envelope protein, wherein either (i) the said viral envelope protein originates from the same virus as the viral structural protein, e.g., originates from the same virus as the viral Gag protein, or (ii) the said viral envelope protein originates from a virus distinct from the virus from which originates the viral structural protein, e.g. originates from a virus distinct from the virus from which originates the viral Gag protein.

As it is readily understood by the one skilled in the art, a virus-like particle that is used according to the disclosure may be selected in a group comprising Moloney murine leukemia virus-derived vector particles, Bovine immunodeficiency virus-derived particles, Simian immunodeficiency virus-derived vector particles, Feline immunodeficiency virus-derived vector particles, Human immunodeficiency virus-derived vector particles, Equine infection anemia virus-derived vector particles, Caprine arthritis encephalitis virus-derived vector particle, Baboon endogenous virus-derived vector particles, Rabies virus-derived vector particles, Influenza virus-derived vector particles, Norovirus-derived vector particles, Respiratory syncytial virus-derived vector particles, Hepatitis A virus-derived vector particles, Hepatitis B virus-derived vector particles, Hepatitis E virus-derived vector particles, Newcastle disease virus-derived vector particles, Norwalk virus-derived vector particles, Parvovirus-derived vector particles, Papillomavirus-derived vector particles, Yeast retrotransposon-derived vector particles, Measles virus-derived vector particles, and bacteriophage-derived vector particles.

In particular, a virus-like particle that is used according to the invention is a retrovirus-derived particle. Such retrovirus may be selected among Moloney murine leukemia virus, Bovine immunodeficiency virus, Simian immunodeficiency virus, Feline immunodeficiency virus, Human immunodeficiency virus, Equine infection anemia virus, and Caprine arthritis encephalitis virus.

In another embodiment, a virus-like particle that is used according to the disclosure is a lentivirus-derived particle. Lentiviruses belong to the retroviruses family and have the unique ability of being able to infect non-dividing cells.

Such lentivirus may be selected among Bovine immunodeficiency virus, Simian immunodeficiency virus, Feline immunodeficiency virus, Human immunodeficiency virus, Equine infection anemia virus, and Caprine arthritis encephalitis virus.

For preparing Moloney murine leukemia virus-derived vector particles, one skilled in the art may refer to the methods disclosed by Sharma et al. (1997, Proc Natl Acad Sci USA, Vol. 94: 10803+-10808), Guibingua et al. (2002, Molecular Therapy, Vol. 5(no 5): 538-546), which are incorporated herein by reference. Moloney murine leukemia virus-derived (MLV-derived) vector particles may be selected in a group comprising MLV-A-derived vector particles and MLV-E-derived vector particles.

For preparing Bovine Immunodeficiency virus-derived vector particles, the one skilled in the art may refer to the methods disclosed by Rasmussen et al. (1990, Virology, Vol. 178(no 2): 435-451), which is incorporated herein by reference.

For preparing Simian immunodeficiency virus-derived vector particles, including VSV-G pseudotyped SIV virus-derived particles, the one skilled in the art may notably refer to the methods disclosed by Mangeot et al. (2000, Journal of Virology, Vol. 71(no 18): 8307-8315), Negre et al. (2000, Gene Therapy, Vol. 7: 1613-1623) Mangeot et al. (2004, Nucleic Acids Research, Vol. 32 (no 12), e102), which are incorporated herein by reference.

For preparing Feline Immunodeficiency virus-derived vector particles, the one skilled in the art may notably refer to the methods disclosed by Saenz et al. (2012, Cold Spring Harb Protoc, (1): 71-76; 2012, Cold Spring Harb Protoc, (1): 124-125; 2012, Cold Spring Harb Protoc, (1): 118-123), which are incorporated herein by reference.

For preparing Human immunodeficiency virus-derived vector particles, the one skilled in the art may notably refer to the methods disclosed by Jalaguier et al. (2011, PlosOne, Vol. 6(no 11), e28314), Cervera et al. (J Biotechnol, Vol. 166(no 4): 152-165), Tang et al. (2012, Journal of Virology, Vol. 86(no 14): 7662-7676), which are incorporated herein by reference.

For preparing Equine infection anemia virus-derived vector particles, the one skilled in the art may notably refer to the methods disclosed by Olsen (1998, Gene Ther, Vol. 5(no 11): 1481-1487), which are incorporated herein by reference.

For preparing Caprine arthritis encephalitis virus-derived vector particles, the one skilled in the art may notably refer to the methods disclosed by Mselli-Lakhal et al. (2006, J Virol Methods, Vol. 136(no 1-2): 177-184), which are incorporated herein by reference.

For preparing Baboon endogenous virus-derived vector particles, the one skilled in the art may notably refer to the methods disclosed by Girard-Gagnepain et al. (2014, Blood, Vol. 124(no 8): 1221-1231), which is incorporated herein by reference.

For preparing Rabies virus-derived vector particles, the one skilled in the art may notably refer to the methods disclosed by Kang et al. (2015, Viruses, Vol. 7: 1134-1152, doi:10.3390/v7031134), Fontana et al. (2014, Vaccine, Vol. 32(no 24): 2799-27804) or to the PCT application published under no WO 2012/0618, which is incorporated herein by reference.

For preparing Influenza virus-derived vector particles, the one skilled in the art may notably refer to the methods disclosed by Quan et al. (2012, Virology, Vol. 430: 127-135) and to Latham et al. (2001, Journal of Virology, Vol. 75(no 13): 6154-6155), which is incorporated herein by reference.

For preparing Norovirus-derived vector particles, the one skilled in the art may notably refer to the methods disclosed by Tomd-Amat et al., (2014, Microbial Cell Factories, Vol. 13: 134-142), which is incorporated herein by reference.

For preparing Respiratory syncytial virus-derived vector particles, the one skilled in the art may notably refer to the methods disclosed by Walpita et al. (2015, PlosOne, DOI: 10.1371/journal.pone.0130755), which is incorporated herein by reference.

For preparing Hepatitis B virus-derived vector particles, the one skilled in the art may notably refer to the methods disclosed by Hong et al. (2013, Viruses, Vol. 87(no 12): 6615-6624), which is incorporated herein by reference.

For preparing Hepatitis E virus-derived vector particles, the one skilled in the art may notably refer to the methods disclosed by Li et al. (1997, Journal of Virology, Vol. 71(no 10): 7207-7213), which is incorporated herein by reference.

For preparing Newcastle disease virus-derived vector particles, the one skilled in the art may notably refer to the methods disclosed by Murawski et al. (2010, Journal of Virology, Vol. 84(no 2): 1110-1123), which is incorporated herein by reference.

For preparing Norwalk virus-derived vector particles, the one skilled in the art may notably refer to the methods disclosed by Herbst-Kralovetz et al. (2010, Expert Rev Vaccines, Vol. 9(no 3): 299-307), which is incorporated herein by reference.

For preparing Parvovirus-derived vector particles, the one skilled in the art may notably refer to the methods disclosed by Ogasawara et al. (2006, In Vivo, Vol. 20: 319-324), which is incorporated herein by reference.

For preparing Papillomavirus-derived vector particles, the one skilled in the art may notably refer to the methods disclosed by Wang et al. (2013, Expert Rev Vaccines, Vol. 12(no 2): doi:10.1586/erv.12.151), which is incorporated herein by reference.

A virus-like particle that is used herein comprises a Gag protein, and most preferably a Gag protein originating from a virus selected in a group comprising Rous Sarcoma Virus (RSV) Feline Immunodeficiency Virus (FIV), Simian Immunodeficiency Virus (SIV), Moloney Leukemia Virus (MLV) and Human Immunodeficiency Viruses (HIV-1 and HIV-2) especially Human Immunodeficiency Virus of type 1 (HIV-1).

In some embodiments, a virus-like particle may also comprise one or more viral envelope protein(s). The presence of one or more viral envelope protein(s) may impart to the said virus-derived particle a more specific tropism for the cells which are targeted, as it is known in the art. The one or more viral envelope protein(s) may be selected in a group comprising envelope proteins from retroviruses, envelope proteins from non-retroviral viruses, and chimeras of these viral envelope proteins with other peptides or proteins. An example of a non-lentiviral envelope glycoprotein of interest is the lymphocytic choriomeningitis virus (LCMV) strain WE54 envelope glycoprotein. These envelope glycoproteins increase the range of cells that can be transduced with retroviral derived vectors.

### EXAMPLES

### Example 1. Base Editing Conversion of Endogenous tRNAs to Suppressor tRNAs in HEK293T cells

To demonstrate the validity of BERT, a base editing guide RNAs were designed targeting two endogenous tRNAs, Gln-TTG-4-1 and Gln-CTG-6-1, to effectuate mutations in their anticodons to TTA and CTA, respectively. These gRNAs were delivered alongside an optimized base editor enzyme²⁹ to HEK293T cells. Subsequent sequencing showed that approximately 20% of the reads exhibited the desired edit with less than 1% indels (See FIG. 1).

### Example 2. Demonstration of BERT Using an eGFP Reporter Assay

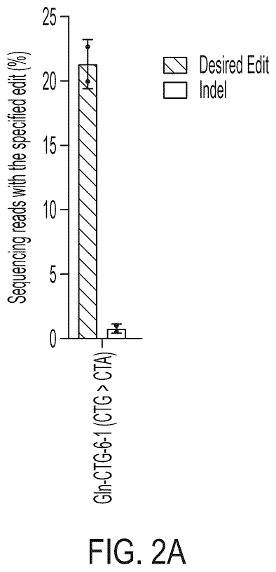

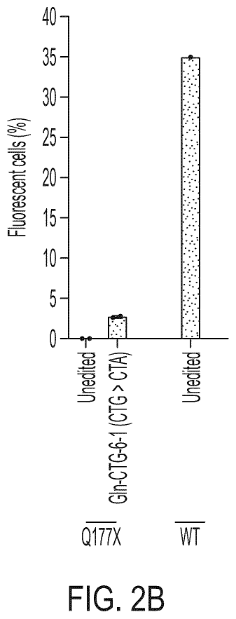

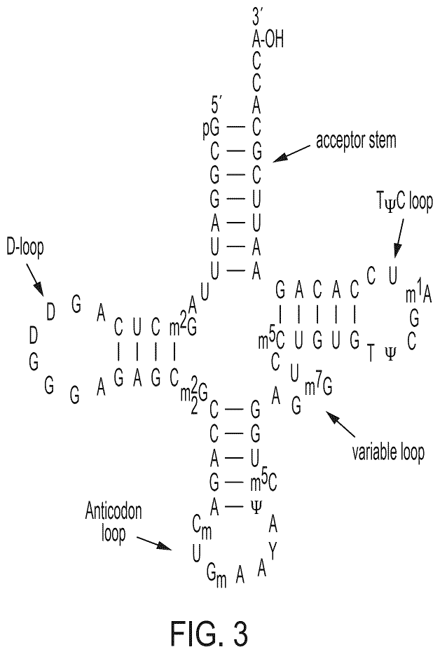

A base editing guide RNA compatible with NG-Cas9 was designed to target the endogenous Gln-CTG-6-1 tRNA, converting the anticodon to CTA. This guide RNA was co-delivered with the NG-Cas9 TadCBEd to HEK293T cells. Forty-eight hours after the editing components were delivered, a reporter plasmid encoding an eGFP cassette with a PTC was transfected into the edited cells and unedited control cells (see FIG. 2). The frequency of cells exhibiting readthrough was quantified using fluorescence-activated cell sorting (FACS, FIG. 2B) and editing efficiency was quantified using amplicon sequencing (FIG. 2A). In Gln-CTG-6-1 edited cells fluorescent signal was 7.7% of wild type eGFP control cell populations, respectively (FIG. 2B). Together, these data support BERT as a viable strategy to elicit PTC readthrough.

### REFERENCES

Mort, M., Ivanov, D., Cooper, D. N. & Chuzhanova, N. A. A meta-analysis of nonsense mutations causing human genetic disease. Hum Mutat 29, 1037-1047 (2008).
Karijolich, J. & Yu, Y. T. Therapeutic suppression of premature termination codons: mechanisms and clinical considerations (review). Int J Mol Med 34, 355-362 (2014).
Banskota, S. et al. Engineered virus-like particles for efficient in vivo delivery of therapeutic proteins. Cell 185, 250-265 e216 (2022).
Krishnamurthy, S. et al. Functional correction of CFTR mutations in human airway epithelial cells using adenine base editors. Nucleic Acids Res 49, 10558-10572 (2021).
Osborn, M. J. et al. Base Editor Correction of COL7A1 in Recessive Dystrophic Epidermolysis Bullosa Patient-Derived Fibroblasts and iPSCs. J Invest Dermatol 140, 338-347 e335 (2020).
Porter, J. J., Heil, C. S. & Lueck, J. D. Therapeutic promise of engineered nonsense suppressor tRNAs. Wiley Interdiscip Rev RNA 12, e1641 (2021).
Liu, C. C. & Schultz, P. G. Adding new chemistries to the genetic code. Annu Rev Biochem 79, 413-444 (2010).
Wang, J. et al. AAV-delivered suppressor tRNA overcomes a nonsense mutation in mice. Nature 604, 343-348 (2022).
Lueck, J. D. et al. Engineered transfer RNAs for suppression of premature termination codons. Nat Commun 10, 822 (2019).
Buvoli, M., Buvoli, A. & Leinwand, L. A. Suppression of nonsense mutations in cell culture and mice by multimerized suppressor tRNA genes. Mol Cell Biol 20, 3116-3124 (2000).
Torres, A. G., Reina, O., Stephan-Otto Attolini, C. & Ribas de Pouplana, L. Differential expression of human tRNA genes drives the abundance of tRNA-derived fragments. Proc Natl Acad Sci USA 116, 8451-8456 (2019).
Iben, J. R. & Maraia, R. J. tRNA gene copy number variation in humans. Gene 536, 376-384 (2014).
Berg, M. D. & Brandl, C. J. Transfer RNAs: diversity in form and function. RNA Biol 18, 316-339 (2021).
Himeno, H., Yoshida, S., Soma, A. & Nishikawa, K. Only one nucleotide insertion to the long variable arm confers an efficient serine acceptor activity upon Saccharomyces cerevisiae tRNA(Leu) in vitro. J Mol Biol 268, 704-711 (1997).
Anzalone, A. V. et al. Search-and-replace genome editing without double-strand breaks or donor DNA. Nature 576, 149-157 (2019).
Anzalone, A. V. et al. Programmable deletion, replacement, integration and inversion of large DNA sequences with twin prime editing. Nat Biotechnol 40, 731-740 (2022).
Chan, P. P. & Lowe, T. M. GtRNAdb 2.0: an expanded database of transfer RNA genes identified in complete and draft genomes. Nucleic Acids Res 44, D184-189 (2016).
Nelson, J. W. et al. Engineered pegRNAs improve prime editing efficiency. Nat Biotechnol (2021).
Doman, J. L., Sousa, A. A., Randolph, P. B., Chen, P. J. & Liu, D. R. Designing and executing prime editing experiments in mammalian cells. Nat Protoc 17, 2431-2468 (2022).
Duvoisin, R. et al. Human U6 promoter drives stronger shRNA activity than its schistosome orthologue in Schistosoma mansoni and human fibrosarcoma cells. Transgenic Res 21, 511-521 (2012).
Yarnall, M. T. N. et al. Drag-and-drop genome insertion of large sequences without double-strand DNA cleavage using CRISPR-directed integrases. Nat Biotechnol (2022).
Durrant, M. G. et al. Systematic discovery of recombinases for efficient integration of large DNA sequences into the human genome. Nat Biotechnol (2022).
Klompe, S. E., Vo, P. L. H., Halpin-Healy, T. S. & Sternberg, S. H. Transposon-encoded CRISPR-Cas systems direct RNA-guided DNA integration. Nature 571, 219-225 (2019).
Strecker, J. et al. RNA-guided DNA insertion with CRISPR-associated transposases. Science 365, 48-53 (2019).
Tou, C. J. & Kleinstiver, B. P. Recent Advances in Double-Strand Break-Free Kilobase-Scale Genome Editing Technologies. Biochemistry (2022).
Chen, P. J. & Liu, D. R. Prime editing for precise and highly versatile genome manipulation. Nat Rev Genet (2022).
Clarke, L. A. et al. The effect of premature termination codon mutations on CFTR mRNA abundance in human nasal epithelium and intestinal organoids: a basis for read-through therapies in cystic fibrosis. Hum Mutat 40, 326-334 (2019).
Buckley, R. H. The multiple causes of human SCID. J Clin Invest 114, 1409-1411 (2004).
Chen, P. J. et al. Enhanced prime editing systems by manipulating cellular determinants of editing outcomes. Cell 184, 5635-5652.e5629 (2021).
Anzalone, A. V., Koblan, L. W. & Liu, D. R. Genome editing with CRISPR-Cas nucleases, base editors, transposases and prime editors. Nat Biotechnol 38, 824-844 (2020).
Gaudelli, N. M. et al. Programmable base editing of A*T to G*C in genomic DNA without DNA cleavage. Nature 551, 464-471 (2017).
Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
Newby, G. A. & Liu, D. R. In vivo somatic cell base editing and prime editing. Mol Ther 29, 3107-3124 (2021).
Rees, H. A. & Liu, D. R. Base editing: precision chemistry on the genome and transcriptome of living cells. Nat Rev Genet 19, 770-788 (2018).
Koblan, L. W. et al. Efficient C*G-to-G*C base editors developed using CRISPRi screens, target-library analysis, and machine learning. Nat Biotechnol 39, 1414-1425 (2021).
Nishimasu, H. et al. Engineered CRISPR-Cas9 nuclease with expanded targeting space. Science 361, 1259-1262 (2018).
Neugebauer, M. E. et al. Evolution of an adenine base editor into a small, efficient cytosine base editor with low off-target activity. Nat Biotechnol (2022).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim may be modified to include one or more limitations found in any other claim that is dependent on the same base claim. Where elements are presented as lists, e.g., in Markush group format, each subgroup of the elements is also disclosed, and any element(s) may be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention may be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A method for editing a DNA sequence encoding an endogenous tRNA at a target site, the method comprising contacting the DNA sequence at the target site with a base editor and guide RNA, wherein the base editor installs a mutation at the target site, relative to the unedited DNA sequence, thus converting the encoded tRNA into an encoded suppressor tRNA.

2. A method for editing a DNA sequence encoding an endogenous tRNA at a target site, the method comprising contacting the DNA sequence at the target site with a base editor and guide RNA, wherein the base editor installs a mutation at the target site, relative to the unedited DNA sequence, thus converting the encoded tRNA into an encoded suppressor tRNA, wherein the DNA sequence is any sequence listed in Table 1.

3. The method of claims 1 or 2, wherein the DNA sequence encoding the tRNA molecule is a redundant and dispensable DNA sequence.

4. The method of any one of claims 1-3, wherein the target site in the DNA sequence encodes one or more domains of the tRNA.

5. The method of any one of claim 4, wherein the domain is a D-arm domain of the tRNA molecule.

6. The method of claims 4 or 5, wherein the domain is a variable arm domain of the tRNA molecule.

7. The method of any one of claims 4-6, wherein domain is a T-arm domain of the tRNA molecule.

8. The method of any one of claim 4-7, wherein the domain is an anticodon sequence of the tRNA molecule.

9. The method of claim 8, wherein the tRNA anticodon comprises the sequence 3′-X1-X2-X3-5′.

10. The method of claim 9, wherein the mutation is a single transition mutation (e.g., base substitution) in the DNA sequence encoding the tRNA anticodon, wherein the single transition mutation converts the encoded tRNA anticodon sequence into an encoded nonsense suppressor anticodon sequence.

11. The method of claim 10, wherein the single transition mutation is selected from the groups consisting of a C>T mutation, T>C mutation, A>G mutation, and G>A mutation.

12. The method of any one of claims 8-11, wherein the mutation is a single transversion mutation (e.g., base substitution) in the DNA sequence encoding the tRNA anticodon, wherein the single transversion mutation converts the encoded endogenous tRNA anticodon sequence into an encoded nonsense suppressor anticodon sequence.

13. The method of claim 12, wherein the single transversion mutation is selected from the group consisting of an A>C mutation, T>G mutation, G>T mutation, C>A mutation, C>G mutation, G>C mutation, A>T mutation, and T>A mutation.

14. The method of any one of claims 9-13, wherein the mutation occurs at X1 and is selected from the group consisting of G>A, C>A, and U>A, relative to the unedited DNA sequence.

15. The method of claim 14, wherein X2 is C and X3 is U.

16. The method of claim 14, wherein X2 is U and X3 is C.

17. The method of claim 14, wherein X2 is U and X3 is U.

18. The method of any one of claims 9-17, wherein the mutation occurs at X2 and is selected from the group consisting of A>C, G>C, and U>C, relative to the unedited DNA sequence.

19. The method of claim 18, wherein X1 is A and X3 is U.

20. The method of any one of claims 9-19, wherein the mutation occurs at X2 and is selected from the group consisting of A>U, G>U, or C>U, relative to the unedited DNA sequence.

21. The method of claim 20, wherein X1 is A, and X3 is C.

22. The method of claim 20, wherein X1 is A and X3 is U.

23. The method of any one of claims 9-22, wherein the mutation occurs at X3 and is selected from the group consisting of A>U, G>U, and C>U.

24. The method of claim 23, wherein X1 is A and X2 is C.

25. The method of claim 23, wherein X1 is A and X2 is U.

26. The method of any one of claims 9-25, wherein the mutation occurs at X3 and is selected from the group consisting of U>C, A>C, and G>C.

27. The method of claim 26, wherein X1 is A and X2 is U.

28. The method of any one of claims 10-27, wherein the nonsense suppressor anticodon is 5′-UUA-3′.

29. The method of any one of claims 10-28, wherein the nonsense suppressor anticodon is 5′-UCA-3′.

30. The method of any one of claims 10-29, wherein the nonsense suppressor anticodon is 5′-CUA-3′.

31. The method of any one of claims 10-30, wherein the nonsense suppressor anticodon is configured to bind to a premature termination codon sequence.

32. The method of claim 31, wherein the premature termination codon sequence is 5′-UAA-3′.

33. The method of claims 31 or 32, wherein the premature termination codon sequence is 5′-UGA-3′.

34. The method of any one of claims 31-33, wherein the premature termination codon sequence is 5′-UAG-3′.

35. The method of any one of claims 4-34, wherein the domain is an acceptor stem domain of the tRNA molecule.

36. The method of claim 35, wherein the acceptor stem domain comprises a mutation that changes the identity of an amino acid charged to the tRNA.

37. The method of claim 36, wherein the mutation is a C70U mutation.

38. The method of claims 36 or 37, wherein the mutation charges the tRNA with an alanine.

39. The method of any one of claims 1-38, wherein the gRNA comprises a spacer sequence with at least 90%, at least 95%, at least 98%, at least 99%, at least 99.5%, or at least 99.8% sequence identity to any sequence listed in Table 2.

40. A method for installing one or more edits in a DNA sequence encoding an endogenous tRNA at one or more target sites, the method comprising contacting the DNA sequence at the one or more target sites with one or more base editors and one or more guide RNAs, wherein the one or more base editors install a base substitution at the one or more target sites, relative to the unedited DNA sequence.

41. The method of claim 40, wherein the base substitution is a single transition substitution in the DNA sequence encoding an anticodon sequence of the endogenous tRNA.

42. The method of claim 41, wherein the single transition mutation is selected from the groups consisting of a C>T mutation, T>C mutation, A>G mutation, and G>A mutation.

43. The method of any one of claims 40-42, wherein the base substitution is a single transversion substitution in the DNA sequence encoding the anticodon sequence of the endogenous tRNA.

44. The method of claim 43, wherein the single transversion mutation is selected from the group consisting of an A>C mutation, T>G mutation, G>T mutation, C>A mutation, C>G mutation, G>C mutation, A>T mutation, and T>A mutation.

45. The method any one of claims 40-44, wherein the one or more base editors install the one or more edits a the one or more target sites sequentially.

46. The method of any one of claims 40-45, wherein the one or more base editors install the one or more edits at the one or more target sites simultaneously.

47. An edited tRNA, wherein the edited tRNA comprises a nonsense suppressor anticodon sequence.

48. The edited tRNA of claim 47, wherein the edited tRNA is charged with an amino acid selected from the group consisting of alanine, arginine, asparagine, aspartic acid, cysteine, glutamine, glutamic acid, glycine, histidine, isoleucine, leucine, methionine, phenylalanine, proline, serine, threonine, tryptophan, tyrosine, valine, pyrrolysine, and selenocysteine.

49. The edited tRNA of claims 47 or 48, wherein the edited tRNA is charged with a non-natural amino acid.

50. The edited tRNA of any one of claims 47-49, wherein the nonsense suppressor anticodon is selected from the group consisting of 5′-UUA-3′, 5′-UCA-3′, and 5′-CUA-3′.

51. A composition comprising a base editor and a guide RNA (gRNA), wherein the gRNA is configured to bind to a DNA sequence encoding an endogenous tRNA.

52. The composition of claim 51, wherein spacer sequence comprises at least 90%, at least 95%, at least 98%, at least 99%, at least 99.5%, or at least 99.8% sequence identity to any sequence listed in Table 2.

53. A gRNA comprising a spacer sequence that binds to a complementary strand of a target DNA and a gRNA core that mediates binding of a base editor to the DNA, wherein the gRNA is configured to bind to a DNA sequence encoding an endogenous tRNA.

54. The gRNA of claim 53, wherein spacer sequence comprises at least 90%, at least 95%, at least 98%, at least 99%, at least 99.5%, or at least 99.8% sequence identity to any sequence listed in Table 2.

55. A complex comprising a base editor and a gRNA, wherein the gRNA comprises a spacer sequence, wherein the spacer sequence is configured to bind to a DNA sequence encoding an endogenous tRNA.

56. The complex of claim 55, wherein spacer sequence comprises at least 90%, at least 95%, at least 98%, at least 99%, at least 99.5%, or at least 99.8% sequence identity to any sequence listed in Table 2.

57. A polynucleotide comprising a first nucleic acid sequence encoding a guide RNA (gRNA), wherein the gRNA is configured to bind to a DNA sequence encoding an endogenous tRNA.

58. The polynucleotide of claim 57, wherein the gRNA comprises a spacer sequence with at least 90%, at least 95%, at least 98%, at least 99%, at least 99.5%, or at least 99.8% sequence identity to any sequence listed in Table 2.

59. A cell comprising a polynucleotide of claims 57 or 58, a complex of claims 55 or 56, a gRNA of claims 53 or 54, or any combination thereof.

60. The cell of claim 59, wherein the cell is an animal cell.

61. The cell of claim 60, wherein the animal cell is a mammalian cell, a non-human primate cell, or a human cell.

62. The cell of claim 59, wherein the cell is a plant cell.

63. A pharmaceutical composition comprising a gRNA of claims 53 or 54, a complex of claims 55 or 56, a polynucleotide of claims 57 or 58, a cell of any one of claims 56-59, or any combination thereof, and a pharmaceutical excipient.

64. A kit comprising a gRNA of claims 53 or 54, a complex of claim 53, a complex of claims 55 or 56, a polynucleotide of claims 57 or 58, a cell of any one of claims 56-59, or a composition of claim 63, and instructions for editing one or more DNA sequences encoding one or more domains of a tRNA by base editing.

65. A method for producing a suppressor tRNA molecules from an endogenous tRNA molecule using base editing in a subject in need thereof, the method comprising administering to the subject: (i) a base editor and (ii) a guide RNA, wherein the base editor and the gRNA install a mutation at a target site in a DNA sequence encoding the tRNA molecule, wherein installation of the mutation converts the endogenous tRNA molecule into the suppressor tRNA molecule.

66. A method for changing the amino acid that is charged onto a tRNA in a subject in need thereof, the method comprising administering to the subject: (i) a base editor and (ii) a guide RNA (gRNA), wherein the base editor and gRNA form a base editing complex, wherein the base editing complex binds to a DNA sequence encoding an acceptor stem domain of the tRNA, wherein the base editing complex installs a mutation in the DNA sequence encoding the acceptor stem domain, and wherein the mutation results in the replacement of a cognate amino acid with a non-cognate amino acid.

67. The method of claim 66, wherein the target site of the DNA sequence encodes a D-arm domain of the tRNA molecule.

68. The method of claims 66 or 67, wherein the target site of the DNA sequence encodes a variable arm domain of the tRNA molecule.

69. The method of any one of claims 66-68, wherein the target site of the DNA sequence encodes a T-arm domain of the tRNA molecule.

70. The method of any one of claims 66-69, wherein the target site in the DNA sequence encodes an acceptor stem domain of the tRNA molecule.

71. The method of any one of claims 66-70, wherein the mutation comprises a transition mutation.

72. The method of claim 71, wherein the transition mutation is a C70U mutation in the acceptor stem domain of the tRNA molecule.

73. The method of claim 72, wherein the C70U mutation results in replacing the cognate amino acid with the non-cognate amino acid alanine.

74. A method for treating a disease caused by premature termination codons in a subject in need thereof, the method comprising administering to the subject (i) a base editor and (ii) a guide RNA, wherein the base editor and guide RNA form a base editor complex, wherein the base editor complex mutates a target DNA sequence encoding one or more domains of a tRNA to produce a suppressor tRNA, wherein the suppressor tRNA comprises an anticodon sequence complementary to an ochre stop codon, an opal stop codon, or an amber stop codon.

75. The method of claim 74, wherein the one or more domains comprises an anticodon sequence.

76. The method of claim 75, wherein the tRNA anticodon sequence has the general formula: 3′-X1-X2-X3-5′ and wherein X1, X2, and X3 are selected from the group consisting of A, C, G, and U.

77. The method of claim 76, wherein the mutation occurs at X1 and is selected from the group consisting of G>A, C>A, or U>A, relative to the unedited tRNA.

78. The method of claim 77, wherein X2 is C and X3 is U.

79. The method of claims 77 or 78, wherein X2 is U and X3 is C.

80. The method of any one of claims 77-79, wherein X2 is U and X3 is U.

81. The method of any one of claims 76-80, wherein the mutation occurs at X2 and is selected from the group consisting of A>C, G>C, and U>C, relative to the unedited tRNA.

82. The method of claim 81, wherein X1 is A and X3 is U.

83. The method of any one of claims 76-82, wherein the mutation occurs at X2 and is selected from the group consisting of A>U, G>U, or C>U, relative to the unedited tRNA.

84. The method of claim 83, wherein X1 is A, and X3 is C.

85. The method of claim 83 or 84, wherein X1 is A and X3 is U.

86. The method of any one of claims 76-85, wherein the mutation occurs at X3 and is selected from the group consisting of A>U, G>U, and C>U.

87. The method of claim 86, wherein X1 is A and X2 is C.

88. The method of claim 86 or 87, wherein X1 is A and X2 is U.

89. The method of any one of claims 76-88, wherein the mutation occurs at X3 and is selected from the group consisting of U>C, A>C, and G>C.

90. The method of claim 89, wherein X1 is A and X2 is U.

91. The method of claim 74-90, wherein the anticodon sequence complementary to the ochre stop codon is 5′-UUA-3′.

92. The method of claim 74-91, wherein the anticodon sequence complementary to the opal stop codon is 5′-UCA-3′.

93. The method of claim 74-92, wherein the anticodon sequence complementary to the amber stop codon is 5′-CUA-3′.

94. The method of claim 74-93, wherein the disease is selected from the group consisting of cystic fibrosis, beta thalassaemia, Hurler syndrome, Dravet syndrome, Duchenne muscular dystrophy, Usher syndrome, and hemophilia.

95. A method of editing a DNA sequence encoding an endogenous tRNA into a DNA sequence encoding a suppressor tRNA using a virus-like particle (VLP), wherein the VLP comprises a group-specific antigen (gag) protease (pro) polyprotein and a fusion protein, wherein the gag-pro polyprotein and the fusion protein are encapsulated by a lipid membrane and a viral envelope glycoprotein, and wherein the fusion protein comprises:
(i) a gag nucleocapsid protein;
(ii) a nuclear export sequence (NES);
(iii) a cleavable linker;
(iv) a nucleic acid programmable DNA binding protein (napDNAbp); and
(v) at least one domain comprising enzymatic activity.

96. The method of claim 95, wherein the napDNAbp is a Cas9 protein.

97. The method of claim 96, wherein the Cas9 protein is a Cas9 nickase.

98. The method of any one of claims 95-97, wherein the at least one domain is a adenine deaminase domain.

99. The method of any one of claims 95-98, wherein the at least one domain is a cytidine deaminase domain.

100. The method of any one of claims 95-99, wherein the at least one domain is a adenine oxidase domain.

101. The method of any one of claims 95-100, wherein the at least one domain is a guanine oxidase domain.

102. The method of any one of claims 95-101, where the at least one domain is a guanine methyltransferases domain.

103. The method of any one of claims 95-102, wherein the at least one domain is a transglycosylase domain.

104. The method of any one of claims 95-103, wherein the at least one domain is an adenosine methyltransferase domain.

105. The method of any one of claims 95-104, wherein the at least one domain is a glycosylase domain.

106. The method of any one of claims 95-105, wherein the at least one domain is a thymine alkyltransferase domain.

107. The method of any one of claims 96-106, wherein the Cas9 protein is bound to a guide RNA (gRNA).

108. The method of any one of claims 95-107, wherein the fusion protein comprises a prime editor.

109. The method of claim 108, wherein the prime editor comprises PE2, PE3, PE4, PE5, PE2max, PE3max, PE4max, or PE5max.
